SEQUENCE LISTING (7 sequences)

SEQ ID NO: 1 - DNA, 41654 bp, Homo sapiens
  NOD2 (1)..(41654)
ggggtgtatgtgtatcgggaagcatgtttgtggtggggggaagcagggcgtgtgtgtgtgtgtatgtgagtgttgggaagcaaggtatgtgtgttgggggaagcgagggctgtgtgtgtattgggaagcatgtttgtgtgtggggggaagcgagggctgtgtgtgtgtattgggaaacatgtttgcgtgtgggggggaagcagggcgtgtgtgtgtgttgcgaagcggtgtgtgtgtggtgtgtgtgtgtgcgcgtgtgtgctgggaagcggtgtgtgtggtgtgtggtgtgtgtgtgtgtgtatgtgtatgtgttgggaagcgggttgttccgaggactccccgattccctcctggttgaaacgtggcccagaaagagaggcacactggctagggcacacagccagggccgtccagccccgcaggaagccccggtcctgccctgtcccaggtaagcctggccagccctggagccggcgctgcggggctgcggggctgcggggctgcggggccacgggacaggagcgggtgctgcggtcccggcagagcaggggcggggtccgggcgggggcggagccgagcgcccgcgcacccggagccgcagcccgagccggagccggagccgggagtcgtggcccggagtgggccttggagtcggcgcgcaggcggctcgcggtgagtgctgtgcccagcgcctgggggtgggctggggggagactgtgggcgccgagggtcccacccccgggacttgaacacttggcttctgagccgtcctggccgcagggcttgggcaagggttgcctcggtttctcctgggggagtcccagaatgtagatgggaagtgagacggaaagggggcatctcgaggttggggaggcccctgggccactcttttgatttccttcctctctgtgggactcagtttccccagcttcaccatgccagtgagtgggtgggtataagtgagagagtctgcctggaggatcctgctgctgctctctgtacctcttatgggaatctcctgcctcctccttctcccttgttgggtgtgagatctaagttcaaatccatcccttcccagacagcccacaccaggacctgcgaggccacatcccccagctgtttgctctcctatgcttctcaggttgatggagcagccatctggtcctgctttcattccaacaaactgaaaagccctccaccccaatccccctggggaggacctcctgaagctgcatgagggctgggggctggacccctgcttctgagtttgctctttctgagcctctcctgccatggccttggtttccccatgggctgtgacctagcctcctccagggtccttgttctacagaggggaaacacctgccctgtcaccctcccctgaccccttagtgaaaaagaccagagtgacatccccaccacctgtacttgcctgtcccgatcactcagccatggggctgctggggcccctgagtttgttggctgctcaaggcccctttgcctcgctgcaccctgggatctgaggctggaagcatcctcatccagcccacctccctcctacccagtgcagagcccctctgtgacacatgcccagtggtggggagcttattactgatgaggtggctttttagtgccttgcagctcttgtttcctttagctaaaatctgtggcttacagattctacaccttggttccatgtctgtctcccggagccatgcagagcaagtcagttctttcattctttccagcagtcatcattcatgcactggatgagacacgagacagagcagtaggaaaatcagacaaaaccccctgccctcatggagtgcacactctagaaaggaatggacagcaatgaaataagtaaatacatggcgtagcaggtggcgggaagtactacgagtaaaacaaagctgggaagcaggatggggtgctggggctgggtattgcattgtatatagcacaaccagggaaggcttccccaagaccacgatagctgggcaaagtcataaggaattgaggggtgagccacatgagtatctggggggctgggaggctttggttttcactctgagcatgatgggagcacgggtttggaacagaggagtcacctgctttgacttggggtctggcaggcccatctagctgtggggtggagaatggatcatggggggggtggggtaagggcagaaggagagcccgttgagcggatcattgctgtcattctcgggagagaccatggtgtcttgcccatggcccagaggtggttaaaaaaaagaaaaccagacattcagatttgggatatatttcaaaggccgtgtcgatagggtttggggtgcaagagaaaggtagagtcaaagatgattctgagtgtttggctcgagcttctggaggtttgaagctgccatgaactgaaatgggagttgcttagcggagagatcaagagtttggttttgtgcttgtggagtttgagctgcccattaggcatcaatgatgagtaagcagttgggaaaagttaagggcggaggttggagttgaaaataaaactttgggcatcatcaccatataactggtatttaaagccacaagagcaggtgggctcatctagggatggagtgatatggagaagagaaggggtctaaccattgagtgctggggcccccagtgttaggaaccagccaagaagacagaaagagtgaaaatcagagagttggggtgtcctggaggaaatgaagaaaatgccccaaagaggaaggagggaacaaatatgaccaatgcccctggcagagcaagcaggctgagggctgaggattgagcaatgggaggtcactggtgacagtttcactggagctggatggggaactagagggaatgggaggggatgggaggacttggggacagcagtacaggcaacagacaagggggcctgctgtaaagggagcagataaatgggattggagccaaatgaagaaggggagtgtcaagagagtgctttacttttacaatggagaattagagtgcattgtgcactggtggggggatttgatctcttagggagagaacagtgttagggagggagaatgcaggatagctgggggagggtggggggcttggccccagcagagactcaggacacttgggaagttgagcttccctgggcttcccctcctctcctgtctgcaaggggtcagtgggctgagatttcagcacttaagcaaagcatttgctcttggccccagagaaaccgggctggctgtggtctcaggaaggaaggaggtgtccaggctcaggcctgggcctgggtttcagggagggcccacgtgggtcgccccttgaccctctctttcagcaaggaagtgatcctttctctacatgggcctcaccttggggaggacaatggtgtctttgaagttgtagtaactgaagtagagatcaaaaggcaatgcagatagactgacagatttcgcctgaagaggggaagcccgaccaggtaataaaggagtaagaggaaggatgttaaggacaattttaggaaacagataatgagtgaatattttttctctctctttcccaatttaaactgaagcaggagaaactgaagctagacataatgattaacttcccaagctggtgagcttcctgagctggttagtgagaacagcactaaggccaggttctcctccccagatgtttaagatgagacaggacaatgcctgctcagagacagggcctggctgaattggccctcaggattctctctgctctgaggtttctggaagaaggccagggcagaggtgtggtgatgtagctgctgggaggacagagctccgagtcacgtggcttgggcgggcctccccttcctggtgtccacagaagcccaacgtcactagctggggtgtgtatggctcacacgtaggccaggctgccctaggcttggtgtgcaagggaggggcccctacttacttgtggcctgtcccctcgtgaatgtgtctcatgtccccagtggggtttttcagtgagggtcatggtctccaggatgcacaaggctttgtgccagaattgcttggaattgcctagttctggaaggctggttggccaactctggcctccggcttttcctttgggaatttcccttgaaggtggggttggtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggtaagaggagcaggcattgtcccgtcccagcttgatcctcagccttctttcatccttggccgcgacatgctcccaggcctggggtcagatggggagtgctgactctgtttctgggctgttttctggggagaatgggtcggcgggtttttttccccaggacctgggcagggtcaatggtgggggccgctgtcgcatccttggctggtgtttccacagctgagaaccactccagggccaagcccagagcttattctacccttttttgtcctctcttcccctgtcctcggccaccccaccctcttggctcctctgcttagatgtgggcacaaggaggagaactccttggcctgagagaactaccttagatcctggcttccagtggcctctgcaggggggtacaccctctctcccaagcagccagacacacaagtaacctcattgcctcagtttccccatctgaccagcacagggccccctgtgccccagcagcgttctgagagattggagctttctccttttgcttaccttggctaccgtatgaggacggatacagagtgttccccccacccccagcccaggggatatttgattcatgaacattccctcagtgtctttgtgggggacaatgctgtgccaggctcagggatgccaggacgagtaagacccaggctcccacgtggcccaggcagggagagagacacataaacaaccatcaggaaagaggtaaaatccccaggccacttggcatctgctcccttgagtgtctgggaatgtccctgatttataaaaagaagctgacggccctctttgttgtccatgcctacaccctttcactttcgtttcttcggggcactgcagcagcccttgtccacagaccccatgacaatcgcagaactgaccatgctgagagattttcttggctgctcagggaccctgccagggcttgaagctcctggagggtcacttgccctcaaattcccagaacgcacagcaggtcactgatgatagcagtggcagcagtctgtgcacggtggtttcgagggcgtgggagggaggtgagggccctagggcaagtgtgtgtgggaagtgttgatgggggacaaggcaccagaacgctcggaaacaacttagtttgcaccgtaatttttcacttcgcctaggacaggacctttagagcaatattctgagtctaccccttggagtagcagtgtgcaaaacacacagcacgggcttggggcccccgtggggaacccaaatgtaagagttagagacatgcattccggagtcatacatggctcgtgttgaaatcctgactctgcctgtctagctgtgacacatcgtacaaatcacttagcttcttggtgcctcagtgtcttcctctgtagaatgggtagatcataggcactacttcagagtggctgggagggttcagtgaattcctgcaggagagcacttagaatggcacttggtgtgtagtttatgcttaattaatattagccgttactgaaactgctgtagcctgaatccagccagcatgaaagagcccctctcaccctgcttcgaagagaatgaattccctgattgtttggaagatctctctctctctctctgtcttttttttttttttttgagaaacggtcttgctctcttgcccaggctggagcgcaatggtgccatcttggctcactgcaacctctgcctcccgggttcaagtgattctcctgtctcagcctcctgagtagctgggattacaggcgctcgccaccacgcctggctaatttttgtatttttagtagagacagcgtttcaccgtgttggccgggctggtctagcgctcctgatctcaagtgaccttgggagatctcttgctcctaatattacctcaagcctttttaaacgttttaagccggagaccaagcatggatatgggagttaggggtcttgatttaattcttggttgcttcaaactctgtggaaccttgaggtgtttcttgccttctctgggtctcaattttcacatctatatggtggggagcttggattgggtaatgtctgaggctagaaccatggccaactcgggttctgctggggctgacttgccctggccttccctgaccaccctgcatctggcttctggagaagtcccgcactgaccttgttctcctccccaggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggtgaggcactcctggtgtgcatcacagagttctcaggaaaggggtgcttagtcaccaagactgatttgtcctcatgaagtcagcctgtggggtaacttggtccatgggatttcccctaaaaaggtagccaggcaggtaaaatttgctcttgactcttggcaggaaacgtacaactctttctttcttcttttcttttctttttctcactctgttaccctggctagaatgcagtggcacaatcatagctcactgtagccttgaattcctgcgctcaagtgatcttctggccttagagtagctgggactacggctgctgtaccaccatgaacagctaattttttttttttcttttagagatggggtgttgctatgttgcccaggctggtctccagctcctggctttaagcaatcctcccgccttggcctcccaaactgttgggattgcaggcatgagccactttgcctggccaacagaacacttctgccgagaggaagtgtgtggtggccaggaactcagattctggagccagaatggtgcaggctcaaggtcaaccctgtgtgatctcaggcttccctatggagcctctccagcctcagtctcccttgtttcagtttcctcatctacaaaacaatgttaatagtcaaatggtgcctatcctataaggctcttgggaggattcagtgagttaatttgagtaatgcttaggatagtgtctattaccactggctgctatttattatttctgttatgagtgatactctgtacttgtacacttttatttctgtctgttttaaattaacagcacaacagaccataacactgcagtatattgaatttattttataattaacatagcatattataaactaatatagcttaaatgtttatgtaggatttctgacatgaaattgcattagatcatagatgttcagagttggtatataacagcccctgagaatgtagtaactcagcagagaccagaaggtcagagaaatgaccactgagtatttttgaaactcttttgttttcttccaaatagtgattcttagggctcctgagaggcagatggaacaatcattaacattccactttataaatcgggaagttgagaccaaggaaagtagtttgaataagctcacagtagttaatgagggggccagtgctggaccaattggccagcactggtcattgacttattcatccatcattcatttattcagccagaatctattaggtgcttcatacatatttgcttaaagtttgttgtgttcatagagctttgcacacggtaggtactccataaacatttgttgatgaaataagtgagttactgaatgaatgattgaattagaatgacactgcagtgttaaaatgggctgggttggggaacattttagtttttgtttttgtctgttttccaaaaatgtatgtgttgttcacatgagtctggataaccctagattgagattgatgacataaataaatttgtcttcaaggctgcactaaagctggctcacatggctaggtatttacagagcagaagtggtgcagtcctctctgattagttgcacgtacagaagacatattcgttattggactgaccttagtttctcttataatttgttaggggaattgaatcagcccatctgagaagttacaagattgtgtcttgtcatctttaaaagttcagcaatgtgatgtggtacagatggtctgaggggtttggagaaggtagcctagatccctagggcccagagaagacaggatgtgaacagaggaagtacatggattggtgaagaaaagaaatgggataactcatgggtcaaagaagaaatcatgatggaaatcagaaaatattcagaaccatacaataatgagaatattatttatcaaaatctattggatgcagctaaagcaggacatagggggaaatttacaaccttaggtgcctagattaggaaagaaggaaggcatttgtttatttatttgtttatttatttatttgagatgggggtctcactgtgtcacccaggctgctggagtgcagtagcacgatcataaatcactgaagtctcgaacttctgggctgaagtgatcctcccgcctcagccttccaagtaggtgggacacaggctagcaccaccataccaggctaattttttttttgtagacacagggtcttgctatgttgaggtctcaaactcctgggctcaagtaatcctcctccctcggcttcccaaagtgctgggattacaggcatgagccactgcgcccatctaaggctgaattttaatgagctaagaattcatcttaagaaagggctaaatagacagcaaaagcaaacattgaaggttgggactgagctgagtgggtagcagggatgggagacaacagatctgaggagagcaggagattttgaaaggattgcactgcctgaggtttaagcctttagaatccagctctctctgagctccctttgagctctgacattctgtgactctgatttggtggccttcccttagtggccttactgatttcatttggatggtgcttgtggtatatccaaccaacatgtcttcccaaatggccttttaatttcctataaagaagtagttgtcattgattgcaggttagggacagaaaatgctgtggaatgaaacaaaatgcaagttaaagaactaaattccaaaaatacccattgctactattgactgagtgaattcctactgtgtgccagacactgtacccagtccattccctgtattgttttatttaagcctcacaagggtatagtgtgactacactgtttcttaacaatgaagaaactgcccaaatcgcccatctgggaagcggcccagctagaatttgaatccaggcctgttttcctccagagcttgtgctattctctgtctgtcataaaatgtgggggctttgtgtggtaaacttgctcagttgggcatagcagttgttaggaaacctgaggctggtaacaccagctgtaataccagctgtccgtctgactcatgcaactgttaaagttgatagggctgaggtgtcagactgagctctgaattgcctgattcctataacaatattaacttaaacattttttaaattgggaaatgcaccatgcatacagaagagtgtgtatatttcatatgtatagtgtaaactgttcccatcacccaggttaaaaaacaggatgttgccagtacctggggccttctttaactgcaactgctagaggtaaacactggcttgacttttgtgtaaatcatctctttgcctttctttaatgttttagcatcttttaaaataaatccccaaataatgtattgttctattttgaaaaactgagtagcaagccaaaaatagctgtgtaaagaaaggtcacttaaattaggctgggtgcagtggctcaagcctttaatcccagtactttgggaggctgaggcaggtggatcacaaggtcaggagatcgagaccatcctggccaacatggagaaaccccgtctctactaaaaatacaaaaaattagccaagaatagtggcatgtgcctgtagtcccagctactcgggaggctgaggcaggagaatcgcttgaacccgggaggcagatgttgcagtgagccgagatcgcactgcttgaacccgggaggcagaggttgcagtgagccaagatcgcaccactgcactctagcctgggtcacagagcaagactctgtctcaaaaaaaaaaaaaaaaaaaagaaaggttactattgccttttcttagatgaaggttcccaaggcagggaaagctaagtggagtctcagggacttggtctggcttttccttccctgggaatttataaggacctcttctgggaagtcagtcggcaatgccatgaatgagtctggggaaatattgggctcattgcaactggagggtctggtaggactgatgtgaattaggtgctgtgtccggaggaaaatggccagaggaagtgggctgctttgtacagtcagtggtaaagttgccaaaggctattatagctcacaggaatgggccaaggctaaacactcctgtggagtgaaatgaatgtcctcagctgactgaggcagcgggagttgaaaagaaacgatattagttcatggtgaagacaagtcaaatatagataaaggttagggtcaggcttgcctggacatctaggagataactgccctcaacttgtttgaatcttgagtcactgctccattttgtttgaactggtggccatctacttatagtatacagccatcaacctgagatttccctacatggtcttcctgccttggtctcctgtatcctgaatcctatggcctcttcttccctggtttactacattttgctagaccgtatcctccagtcaattccttagaatgaatgtatgaaagttaaaatttctgaggtctcacatgtcttaaagttccctcatactggattgatagtttggctgggtataaaattctgggctggccatcattttccttcagaattttgattgcattattccattatcctctcttttcaatattgcttctaagaattccaaaacctttttttttttttctttttgagacagtgtctcactctgtcacccaggctggaatgcagtagtgtgatctcagctcactgcaacctccacctcctgggtttaagtgattcttcttcctcagcctcctgagcagctgggattacaggcacccaccaccacaccctttagtagagatggggttttgctatgttggccaggctggtcttgaacttctgactttaggtgatctgcctacttcggcctcccaaagtgctgggattaaaggcgtgagccaccacacccagcctccaaaaccattttaaaactctttctggaagcttttaaaattttcttttagtccccagaattttaaaatttcaattatgtgccttggtgttcttccattatgttagtcacccaagaggtactttcaatctggaaacttctctatgttttgggaaatgttcttgattagtttacaggtgatttcttcctctccattttatctcttctcttttcatgaaactactattaattcaatgttagaattccttgactgatcatttaattttcttctattttccatctctgtgtctttttgctctacttttctatgatagtcacagctctatctttaaactcttgagtttttcatttttgatgtcatgattttaatttgcaaggggtaggtttgactgattcttttttgtagtatcttactcttgttttatggatgcaacatcttctttgacttaaggatcataagataggtgggttctttgtttgtttgtttgactgtttttcaccctatgtaaactttttctacaagtttctttccccttcccccctttttggcttctatctcccacattagatgctttctctgggctcatgatactctttggttttctttctcaagattgacaggtaggactttaaaacttgttgagcatgcgggtgaaacttgtctaccatgaatttcactgtagatattttggagattgacagtgtttatatctttagatctcgcctcctgggttgatcaagttatctgagtacaccacagaccttttgcctggggataaaccagaaatctgtttcagaaaccactttgattcagtcttccttgttttagtcatttccttcagttccggaggtccctcatgctgatcattccagagccctttacagatcctagggtacacactgcatggttttcaactttcttgttttggggttaagatttggctttcaggagtctcctcagtccgttactattcattcaatcagcaagtccttgagcacctgatttgtgccagacattcttctaggtgttagggatacctcagtgaacaaaacagacaaaaatctttgtcttggaaatacacacactccagtcaggggagagggacaataagccaaaggaaggaaattacagcgtgtgctagaaggtgataagtgctgtagaaagtaagtaaagtgggtttgggagttgagagtttgggaaggggataaatgatggcaattgtaaatagagtagtcagagttctcacttagaaggtgaaattcaagtaaagacttgaaggaggacagggaattagccacatggatggctaggggaaggcttccaagctgagaggacagccagagccaaggcccagaggcaggagcatacctggtagttttaggaaacaggaggccaggatgctgagtggagtaagagggggcatgaaaggagaaacttgggtccacgtggttctagacaggtatttttgtctgttttgggccctgaaggttactattggacttggactcttactctgaggaaatagggacgctattgggacgtttgtacaggagcaatgtgacctgagttttgtttgtaaaggattagactctggctgtggcattaaggctaggctgtgggggcaggaacagaagcagggggaccagttttgcagcctgtgcagctttccagataagcagggattgtggcttggaggaggatggtatagaggaggtgacaagaaatgactctatgtctggtatgtagatattggccacagatggcatttgagcactagagacctggctggtccacatggagtttccataagcacataatacacatcagatttcaaagacttaatatgaaagaaagaatgaaacgtaccttgctatttctttcttttttttttttttttgagacccagtcttgctctgtcacccaggctggagtgcagtggtgtgatctcggctcactgcaacctccgcctcccaggttcaagtgattctcctgcctcagcctcctgagtacctgggactacaggcacctgccaccacgcctggctaattttttgtatttttagtagtgatggggtttcaccatgttgtccaggctggtctggaactccggaccttaggggatctacccgccttggcctcccaaattgctgggattacaggcatgagccaccatgctcagccatatcttgctattttctacatggattacatgttgaaatggtaatgttttggctattgtggattaaatagaatatatgattaaagttgatttcatctatttcttttaactttaaaaaatatgtctgttagaggatttgaaattccacatgcggcttgcatttgtgacctgcatttcatttctgtggaacagtgccctttttgggacatgctttgaaggtggagtcaacaggatttggcagattacagacgagaggcttcaagggtgactccaagacttcggggcagagcacctggaagaaaggggttaatattagccaagatgaggaaggctgtcggtttggcaggtgcatgggcaggttaggagtttagttttgaatatgttggaggtgtttatgaaacttttaagtggagatggaaaataggcagttggatgtgcaagtccagggttcagggagacagttcaggctggagatgaagatgtgggagtctgaggagagattgtattcaaatattcaatccatgagacttgatgaaatcacttctcttccaaatgatttacagcctgcagaatcattttccctatctttgtaggtttatgtcttcattttgtttcatttatttttcagttattcactgttttagtgagttttgagtaggagccagattggatgcatgcgttcaattcaccatccaacactgtattaactacttgaaactcatgtggttgttcggttgtttttttgaccttttattctggatggaagagagatgcttatgaagttgcagtaatcagtaagccttcccacattgctccatcagccttcctggaagaataatgtcttctgcctttcctgtaggcaagaaggctgcttgatcttgccacggtgaaagcgaatggattggctgccttccttctacaacatgttcaggaattaccagtcccattggccctgcctttggaaggtaggtgtatgttctcagttaatcagaaagggaagggcagtcagtgcagatccatggttaagagcagaacacacctcggttaacatcccatatgctggcagtatagcctccctatgactcaatttccttgttttaaggctagcaccaccccgtctcattgggattttgggagcattaaaaggacaaaagcgtgtaatgttagctattagctttcattatctcccacacagtatactgacaattgggctaccatatattgagggctaactaaaggtgttacttaccatccaaactctcattatctgtaccgaaaagatatggacacatgttttgagttagggctggtatctcttgatctctgaaatttagcagctcacaatgggaaactcaagaaccaagtggatctagagactctggtatccctcagtgcccagggtcaccacccaaactcaggaacaggaggggcttggaccgcaccacttgaacataccaggcatcctgccaggtgctttatggacaatgtctaccctttgcaacaaccctgagaagtaggtggtgtttttttccaccttatagatgtggaaactgggcagggaggttaagtgacgagggaggggaagatgggtctgattgtaaattgtccccacctacactttctcttttcttgggagaagaaatgtcagttgtaaagagagagtgcaagcctggcactctttagggcttgttcctacaccactgtagggaaagctcattggcactgaagccccctgagctgtgtgtggtgctggcagatgggtctatcaccctgggctgtgtcctctgggcagcaagcaagcctgtgggcggggtggctggaagtctgtgcctggcactcgcgagtgcaccgtctcattgaagaacaggatctaaacatcagtgcgccacagcagggtgcgcggcacggagtgcaggccctggtttggcccttggttgaggtttgctgttgacatcatcaagcacagctagtcactgtaagaccaggccagggtgcaagattccccacacttctaaaggtgacaattggtgtgtttatttctctataaaatgacattttttttttctggagaattttagtatcattggtgatgactggaaaacctgcatcagaaatcaggtcggaagaggaagatatatatctgatatgtactggagaggaagatatctatcttatggtctaagttcagggatcctggtatattcagagggcagaaagctcagcaataatcatcaactctgggaacagaggtgacataaacacagggcgtcccctttgtgtgactgcagatagtcatcagtgagctcagagctctatgaaaattacttgctagtttttgggttgaaaatagtgggccagtgtttggttgggggcagtgaggctgtgatggcgggggaccatgccaagctcctaccagcctgggacgctaaaccagcacttccccatttcctgaaaggggaactaaactctgacacaggaaatggtttgcttgcattactttcaggatgagaaaggaagagcactggccttccaaacacaccccgtgcatgaaaactctccctgcatggggtgcatggggaggatggggaagtggaggcaggatcacagactcttgttcgagtgctcagctggggcaccccggtgaccccgaggccttcccttgctaggtccacccagatcaatcaggatcatctccccatctcgaagtttaactttatcacatctcagagttccttttgccacgtaaggtaacatattcacaggttctgagaatccggacatggacatctttgagggtctattgttgtgcctactatatccatgaataataatgataataagcaccattttttgagagtttgccatgtcagatattcttttaaactgtattttatctcgctgcctcctgaaaaaatccttccaggtgtatattgtccccatttttacagatgagagaactgaggcccagaaaggctaaatggcttgcccaagtgtatggtggacccaggttttcaaactcaggtgtgtctggcttcagagactgggctcctgagcccttaagccctttgttcccctttagaaaaagtcacctgaggctgagtggtgaagggatttatccaaagccacccggccactatggcaggacagatatcagaatacaggtcttccgatcccagcccagagccccttcccgtcatctagaactcctcctggtgtcagtaatgataacggcagtcactgatgtcttttgagcacttactttgtgttgagcacttacactgtgctaagcacttgacataggtcatcttagttgatccgtgtaaaactctgtgaggtagtgaccaacatttctcccaccttacagaggtggaaactgagggttaggaagtttccttgactgtcctcaaagtgcacagcttgtgaatggaggagccaggatgggcgcccgctggctctcctatcccttcagttatgtcagcgtcccccgcagcagcccattgtctggttaggtcccgtcttcaccatggtgccaccttcatctgcctcttcttctgccttccagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtagtgagtgttactgggcattgctgttcaggtatgggggagcaccatcaaggctaagtgtgggagcaccgagctgggctctagaagtctgggcccagcttcgcctctgccaccctgctttgcaacactgcccagatcccttcccttctgggccttaatttcaatatgtgatgatgacagccacactttattgactggcctatgtgctgggtctggtgctatgctttccggaatgacctcatctaatctctacaaccaccctggcgggtaggcaggaatgttattatctccattatccttgacttgaggctcagagaagtgaagtaacttgtccaggaaatggcagagctggggttcacaaattgcatcattctgattacaggttttctgcctcccaccagtctatggatacacttcagaggctccctgaaaaccttgaggtcacttgcagaaagttttgtgtagtatgtgtccgtatcaggaacaacaccaaatcagaggtgacttgtgccccatcagagactttaacaccccaaccagatgggaatttcaggacccaagaaatagaaagtggctgcagggttacaactactgttggattcctgaggtagcacagtgtccaaacaggatttcagcactacccgtattgcttagagccccagccaaagatgtgaggttttgccctttggagaatctgtgcccctgaactcgggggcctctttccacatcttgggggcaggcaagggcagagggtgtgcctaggcctgcggatcagcatgcgacagattccccaacatccttccagcttgaaaggggattgccctgcttctatttagaacctataggaaagcagaagttctagattgaagttaaaattgattcccagcctccaggggctttgggctacacctggatgaccttaattgaccctaagcatgggacaaaccacttcctgagagtattaggatggtatacatcttctctgggggcaaagcaacaagatttatttttcatcatggaccaaacacatggatacccactagaaactgtgtagtgaattttgttaaccctgacatagggaccatggtctttaggttaaagcataataacaacataatacataacatatatagcgaatatatatatgtattatatgcaatgaatgtaaatatgattatacccatcatggtcttggaggaaacagatgacacacttaaaatgggtgttttgaggagagtttgaaaaacagattgtttacaagccatgggcaggagttaggaagagtgagagggttggtgcaggggcctggggttagtaacagctgggggagggtagacttgaagggggaaggggagggagactaattagctggggggaaggtatggagacggctgcctgagcttctgcaaagtggaagaataccgcttggccctaactcctcaccccaactcttgctcctggccagcgccttccaccagctggacccatcagggaggccgagtgggctgtctgctggagtagtccccaggcatcagcctcccaggagccagggacgggtagagaagggggagagtggatctggccaggcaaatggaaaacagccagcaccaaactctatttccctaggagggaggatcatgatactttgagtgggaatttggaaacctgtctgttggagcaatttccctgatagaaataagaatgtgcattttcctgggtagtagactcagtttttaccccaagaggccaggcatcactggcctgtgtgatcctcataggccagtccatctctggaattcttgaatggatcatccatccttgattagggatgtccccgtgattaccagggtgtgcagaagggctctgggaaacctgtgggtctgtctctgtgttcagagaaaggtgagggtggcctggttctagctcatggtgctcagactgtggtgtgtaaaggcactcgtggcaatgcagattcctgggcctgcctctagtgattcccattcagtaggtttggggtggggcccaggaaatctatatttttcacagacacccctggtgattctgatacaagtggtctcgccctgggagaactactggtctgcagcaaccagcttggttttccattagcaattactgtccttgagcgagttttactgctcttcaccttacacacactaaaactgccaaggccgtaggggaggggaagcaaccatgaggttgctgtgagtgcactgtgtgtgtgtgtgtgtgtgtgtgtgtgtgtgtgtgtgtgtatgagagagagagagagattgagaaagagaggaagggaggaagggggagggcacaggctcctctcccacagtgccaacctgcctctctcccacttgaagtgtttccatgccaactgaaatcctcagcctctaggaaaccctatatacacagtgcccctatataggtttctttagactctggctctctcagactctagagtgatggctttaaaagttttttgaatgttacccacaatgattgagaaagagagagagagagcacgcaccaccatgtaaacatggaacctaagtttcacaaaatgacttcgctttatgaactctgagacactctgctctcttctgttctgttctatttccattttagaaatgctgctcaggaccttcaaaatgatttgcatgacctgcaacctgcagtctgaaaaatcactgcactacagaagtggccataagaggccctgagggagaagctgcacaatgtcatggttaagagtggggtttggagccaagccgcctaggctcaaagcctttatgtgccgtacaaccttggcaaagtcacttcgcttgtctgtgcctcagtttctttctcacgaatgctcataataatggttcccatttcactggcttgttgtgaggatgaaatagtgttattattgagaagtggtaagggtagtgatcagtgctagcgatcatgattctaggtgacttttactgtgtaccgggtgctcacaaggctttatgtgcacagcctggtgaggctgataatactattgttccctctttttttttttttgagacggagtctcgttctgttgcccaggcagggggtacagtggcacaatctcggctcatgcaagctctgcctcccgggttcacgccattctcctgcctcagcctcccaagtagctgggactacaggcgcctgccaccacgcccggctaatttttttgtatttttggtagcgacagggtttcactgtgttaaccaggatggtctcgatctcctgacctcgtgatccgcccgcctcggcctcccaaagtgctgggattacaggcgtgagccaccgtgcccggcctgttccctcttttatagatgaagagaccagcaaataactagtaagtcgctgatcaggatcacaatatccagctgaggcactccagagcctgagctgttaaccattcagtcagggcctcccaagtttgcctaaagataaagaatcatgtgcacagttgttaaaatatacagattcctgggccccaccccgcagatacttgattgccagctccagggtatgggcctgagaatctgtcttttagggaagctttcagatgatgttgtgatcaggtgagttttgggaatggtgccccaagaggagtggcagacagggcttgctcggcagggactagcctgttggagtggtgccattggggttaaggactgggcagcagggcctcactaaccacagcctatatgcctgtttctgaagttttggccactctcatccagctggtctactgtctgctgacctagatgatggtaaattgtccccaggggtagcctgtctagttcaggctgcacctttcgcatatatcagctcctttccaccatcatcccctttgtgaggctgctgtgattatcatgttccttttgcagagatggaaacattgcctcaaattagctctgtcatttcctaaggattccagggttctttagtagggggtctggatcctacgtcctgggccatccccatcatagtgcaccacgtcacctccctggccagggaccgtggggtctccacttttttggggtgctccatctatgcagggtttcctggaagcacagatgctggcacttcagggatgaatgaaagtctttttgggggatttgtagatttttttcttgtcttactagctccattttcaaatgtatttattttgtctctttagtttgcgcgataacaatatctcagaccgaggcatctgcaagctcattgaatgtgctcttcactgcgagcaattgcagaagttagcgtaagtcagcctgggctgtggacaatgggctccaagtgccctggtctcaccccaggtcgtgcagcctgggaagctgtgagtgatgggctggggcaggggctgtttgcatgatggggggtgcaggtgattcctgcccagaggggaagggcaaccctgggatttggtgctcactgtccaatgtgctttgcttctgtgtctcctctcttctggaactgaacagtctattcaacaacaaattgactgacggctgtgcacactccatggctaagctccttgcatgcaggcagaacttcttggcattgaggtgagcccaggttttccttattccctggaaactattttttgccccattcctgagtcagtctgatctggtcttggcctggcactgcccacactggctcctgacctcctgattgaatgcagggacagtgtctcattttaagcaggggttctctaatgctgtgatctccccagtaaactctggactagctctgctgaggacttcctgtcttttgacctttagcccgtagggcaagaaagcttttctaggcccctttccttttctgtgtctaagagtgtcacagctttctgggtttactgagttccacgatgcatgttgagctcgtcctggtgggggaggcatacacagttacttgccaccccagctgtggcagcgagttgctgcaacactcccaggaggtcctttcaccactcagagcatgcaaggtttgcagtccatctggttctgcatttctgctactccagtgtctcccagtttcaacaggagtctctctctctcctacctgatgcctttaaattgcccctctagctggccgctgggttggcctggcttctctctccttctctctctctcagatattcttgcctcctgtgatttgtgaggcagtaaaaaaagacaaagtaaagaattgcttccatctattcttttacctcttgggctgggtttgtggatgggagccgccattttaaaatggcgggccacatagctcagtctcggcaagggctactgagatcagaaccacaggtgccaatttgtacaaaggactcagtcctgctaccactgcctgatccctcagactcacaagcctggaataggctgtggccagacctggctggcccatccctgagaagggtgctagtttcagaaatggaggctgagtttgtggccaacacagtagtcctccggtatgtgcaggagagatgttctaagaccccagtggatgcctgaaaccatggagagtatcaagccctacacataccatgcttttcccaatacctacacacctgcaataaagtgtagtttataaattaggcacagtaagagagtaatagcaactcataataaaatagaacaattataacaatcaatgtactataataacactatgtgaatgtggactctctccatctccctcaaaatatcttcttgtactgtactcacccttcttcttgggaagatgtgtggtggtaaaatgcctgtgtgatgggaggaagtgaggtggatgacgcatgcagcactgtgctctagcgctgggctgctgttgacctgaccacacttcaggaggagaatcatctgctcccagagatccctaatctttgagcaacaatgaggtcggcagctggatgtcaggagcagacgatcttgatgattaccaaatgggagcgtatagagcgtggatgcgctggacggggggctgattcacgtcctgggtgggatggagctggatggcacgtgatcagaatagcatgcaatttaaaatgtatgaattgtttatctctagaattttccatttaatatttttggactgcagttgatttcagataactgaaaccatagaaggcgaagctgcggataagcagggggcagggattaccgtatatcattgtaatagagagcacaggctctggagccagactgcccgaggtttgaaccctcattagctgcgtgacctcaggtcagcccaatgtctgtgtgcctccgtttccccttctgtagaatggaggtaataaccctggctacctcacaggctgtagtgatgagcaagcaagttaatccacatgaagggctgcaccgtctggcaggggctttatatagtaagcgagtggctgaaagatgatgggtaaatcacacaagcactcagcttgtttctccttatgtgagtccggtcctccaagcagggattcaatgtgccacccatttattggggaaaagtcctaaaaggggaagtggggaagggagctgggggaggctgggaggtgtgtccctgagtgaaggagagagggaaggaaggaaggttgagactgggcaccttggacttcagtgcagtcctaagacatcttggcaaggctgatgaggagttcttgaaccaaattcaccaggcaggggagcctgatgtctcaggcaggggctggcaagtgcagatgcgaggatgttagattttggagcacagcagctggggcccttggctacctccaaggagctgaggctggagacctgaaaggcgagttctcctagctgccacaccccttctccaaggatacaataatatctgccttataggattgttgtgagctgagtggcttgacgttccttgaaagaatgaaagcgtatagttatcccaggaagcctagggttgcaggtgagagctctggggcttctccgaagctctccgaggtgtctggattcagttgcagcaggagccttccttgctgggatcttcccccacccctagccttggccctccctctctccttcctttctggaaggctcagtgggccccacccctccctccagccacctggacctgcccagcgctcttgtgcaacaggtaaagcctacctgtagcaacaacagatctgggaaggctgcagagggcacgatggggtctggatcgagggcggctgagaccagagggaaaggtgtgaccctgagtcaccctcgctgtcccggggaaaccacctcccaggacagctgcctactgtggctcctgcctggaattgtcacactgctgtgcaaacagcgtcccgctgcccctttccctttgctgggggaaaatgaagttgtgggagccgctgagtaaactagacctagcagcgagggcacctgatgtggctgctgcctcccgggcaggtcttcaatgctttcttcctgtgtttccctggccagggcacagacggccctccttttctgcctgccgctgtgttctctcagcctcctctgtcttcccttccaggctggggaataactacatcactgccgcgggagcccaagtgctggccgaggggctccgaggcaacacctccttgcagttcctggggtaggttggattccaggaagagggacctgcatggaggggcttgggacttttgaggatttaggggcaggtgaaactcttcagccaggaggccccagaggcagcccagctccagtggggaggacaagccagggagagagtgggcggcccttgactgccaccttcatacttggtctatgcctgacaaacaggaagtttgggatgttggggctaggggaggacagtgcccacgagctggtgacaggaagccctctgatcctcagggggcgctagggctgtactttagctgcatattaaaaccacctggaagcttctaaacactattgccaggcctcccaccccagactgatgaaatgcaaatatctaggtgcaaggcccaggtatcaggagttttaaaaagcttcccaggggatgtacagccaggggtgaggacccctgacctaagaaagagaaggaaatggggaaggataggaaggcacccaggataagaggggctgtgctaggtccctcggagctcttgctccctgtaggaccatgctagggcctgccagggaggggagtaccccaacctgcagccccagggtgggcttcctctgtttgctaggcacccaggcttgcacctgtgctgtttccagcagcctctctcctatcctgtcatgccctagtgtgaactggagtccatttgacaagaactgggagttttagaacctgggactgtaggaagagagaataaccttagggcctaggtgttccagcccatttcacagggaggcaagttgcccccaagctcagttttttgttttgttttgttttgtttgagatgtagtctcactctgttgcccaggctagagtgcagtggcacgatcttggctcactgcaacctccgcctccttggttcaagcgattcacctgcctcagcttctcaagtagctgggattataggcacccaccaccacgcccagctaatttttgtatttttagtagagacagggtttcaccatgttggcccggctggtcttgaactcctgatctcagatgatccgcccgcctcggcctcccaaagtgctgggattacaggtgtgagccaccgcacccggcccccaagctcagtttgagccacaaatgggactatgttgctctagaaatcaacatcttttccacactgcattagtagcaacagagtctagaacaaaggaggccacagccccactgaactctcttctgcttgaggtcacatctgccacatcaggggtatttacctctttcaacacatatttattagggcacctgtctgggccaggcgttgtgctaaaacccccaaacgctgtcatatgatacaaagtgttctgtaacttgcttggtttttttttttgtttgtttgtttgttttgttttgtttttgttgttgtttttttttgcttcgccatatattataggaatttttttaggtcattatgacctctttatttacttaattatctatttatttattttactaatatttacagaaagggtctcactctgtcacccaggctggagtgcagtggttgcaatcatagctcattgtagccttgaactcctgagctcaagtgatcttcctacctcggcctcctgagtagctgggactacaggcacaagccaccatgcctggccgatatttttatgttttgtagagacggggtctcactatgttgcccaggctggtctcaaactcctgggctcaggtgatcctccctcctttgcctcccaaagtattgggattacacaagtgagccaccttgctcagcctgacctcatttttcaaagagctgcagagtgttacataatgtatttaactggtcactttttgatgactattaagttgttttcaggttttttgttattacagtgtcatatccctggggcacagagcagtgctggcacatagccagagctcaatcgatacatacctaatgaatgaaagtacagtggacatcctaattcagccattctttgctaacttgtgtacatacctgtccagggtaggtccctagaatacagtcaataagtcagaaggtgtgagttgggatctaccttttggaaagggatgttttcaaactacagtgagtcagaggaggatggcccagaagctgggggagttgaagctgatggcgtgaaggaattaggggtgttaggaagaagcaggagataaagagctagcttgcagaagaagtgttagacttgttatgggcaggtactggagggtagctaaggacttgtgggtggcagttaccaggaagcgtatctgaactaagtgtcagaaaaagtgtcacaactgtaaattactcttgtcagtgagttcctgtccttaagggttagggctgggtagccctctactattctctaagtctgtaatgtaaagccactgaaaactcttgggttaagtttggccatcccacccaaaagatggaggcaggtccactttgctgggaccaggagccccagtgaggccactctgggattgagtggtcctgcccctctggctgggactgcagagggaggaggactgttagttcatgtctagaacacatatcaggtactcactgacactgtctgttgactcttttggccttttcagattctggggcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgatacagtatctgtaaattgaagatattgggctggatcaggtacattttatcttaaggaccaattccaatccattggtagtgggtgcccagtgcaccacattaaaaagaattctaaggctgcacctgggcttaaagaagagcactataatcaattagtgatgtctaaaaaagctaaaaaaaaaaaaaaaagagcactgcattcaattagtgatgtctaaaaagggtagaaaaaaaaaaaaaaagaaaaaagaaagagcaccgcaatcaattagtgatgtctgaaatggagcagaccaggagagcaccacgaattttgccctccataggttagctcatctctgaggtctttccctgctctgacatacttttgttccatgattacctccagcctggtggggaacaacattggcagtgtgggtgcccaagccttggcactgatgctggcaaagaacgtcatgctagaagaactctggtgagtttgggggattctctgctctggggaagtggatcacaatctctgttgatcccctggcctcatccataggagcggttgtgtggacagacaaaggtggatgattgagtgattgactgattgattgattgtgtttgtctttatatgtactgagtggtatgaagcttatagagcctggtatgtacatgctaatttttttatttaataaaatatatgggtttgctggtttggtgactgcctccacatggcataagtgttaagagcacagactctgtaatcaagcaggccgtgatcttaggcaagttaaataacaatttcagaatctcaagtttcatgtctgtaaaatgagggtaagaatacttccaaccataaaggatttttgcaagaattagataaagtagtgcctgtgaagaccttaatatagtgcctggcatatttgtaagtgctccataaatgttaaattagaataatggcagggttactactactattactgctgctgctgctgctgctgctgctacaactactatagtactgtgactactactactaataaagttttgttattttaaagtgattttgagttcctaggagcactgggtattcaagtcttaggtcattttggaaggtgtaatggagttttgatagttgaaagaggaaccatgaatcatgcttatactgttgacctgaagcagattctaagtttctcatcctttagatgccactagtatagttttctgacatgttctgggcagcttcagattatgtcagggagataaaatactgaatgtttgattttcccgggaagcagaaaggcactgcaacatatgggcattgccataaacagattttatggatggaccttggctgttgcagggcttactagctctactcaagtatgattgattctatcctgactggattttgccacttggaatttcttagtagaggagaaccttgttatgagagcatcagttatgattactgttaaaagaaaaactttaggcaaattaaatttagcagaactggtttgaacatacagcaatttatgaattgggcagcattcagaactgggagtgctccacccagcaaggtaggcaagcagtatctatagacaggaaaaggaagtgatgtacaaaacagcttgattggttgcagctgggcatttgccttatatgggcatggtgtgatgaggcattttctttatatggatatagactgatcagctggtagactgtgactgactgaagcctggctgctgtgattggctaagacttagctgtttgttataaggatatgttgttaggttgcagtttgctacataggaactcaaagtacagaggcagtctcaggccaaatttagtttaactatatgttaagctgcaggtgacagaatacctccatctatagaggtttaaacaaggaaagggtttattttttcctgtataggcagctggatgtaggcagtgtagggtttgtacagtggctacaagaggccaggaggggtctcagctctgtctcattctcttcctgttccatcattcttagcctgtgacttcattcacatggttggttgtctcatgatcacaggatggctgctccaggtgcagcactacttctgtattcccggattcgatctatatacccaggaaagccatctgggttctctcctttaaaaagcattcctggaagccccacctgtcgacttccccttatgtatcaaccatgtgtatgtcacttgaccaacccacttgtatgttgtttgaccagccctggctgcaatggagagtgggaaatacagttttttcaccaagtgcatggctgtccaaatgaaatgagacttccattaataaggaagaaaggaaagatggagatcaggaagctgggggatcagggaacttattacattgagagcccttggagtgaattctcttgcaaatatgtccctggaattgagaatccccacaacgtactttatctgttctttctttatccatgagtttgggttttcagatgttggatttcctatatggggggcatgtgagttcatcatcttccataatcaatgttgtatcaactggattttctctcttcttctcaccagcctggaggagaaccatctccaggatgaaggtgtatgttctctcgcagaaggactgaagaaaaattcaagtttgaaaatcctgaagtaaggaacccataagcaggaaacaggacaataattgctggcctttggaaggggcatttctgaataagatctgggccgctctccgctgggctaactcatgtgaggtggcctggtagaacagcttgccttggtctaggtggacaaggattccagtgcaagttgtttatctgggaggtggtcccagtaaatgctgataggagagtggtgaagtgagatggggaagtgaaggtaaccaataaaggggagttatcaagccagttatcaatgagggaaattggagctcagtactctggggcactcctggagccagtgcagaacacacatggtcacctacccaaccaatgggcaagaaagccatggcatttatccaccaaccctctgtccttcctatgttgatgtgcgctcatggggcactgattctccagcacttccagctcaccctcacccagctgaacatgcttctggggtcaggagaatggcctcaggcagagagtggaaggtcttctctgcaagcagtggctggggaggtgatgtgatggggagtactgtggcctcctccagtggctgactcagtggcttgggacttgtgccacaaagagatggacagctcaggtgaacatgaacccacctagtgaccatcatgggtttgtcagggtgctctctgaggctgatgccaaaattcttatttcaagtagacctcaggaaccccatcagatggctccttttgctggaggaaagtggcatctgcctaggcaaatgtggtcctaggaaaacgcttgcctttagagacagacagacagacagctgcctctgtgagtgccagctttgctgccaggctgctacccactctggcgacactcatttgtgttgctttcacaagctaggaagtttccaaatatttggagaaaacacttccactaattatttgggtggaaatgggctgggaagttggggtgaagcccggatgtgtctgagccagatgccagctttgcactgagggtcggcctttgggaataccaagcccattatcaaccaggtgtggatatggcaggtttgtcttccctccttgtcacagccttactccacttgactcccatggatgccaggcaatgaggctggggttggtcccatgccaccctgtcatcagccttatttttcagcatcctaaactatatcatcccccacaaaaattgaacttctgatatatcttttataaaaaagagaaatgcctacatctttcttttccaggcttagtttctgccaagagttggttgagagcccaggcttgctgggtgcagtggctcacacctgtaatcccagcactttgggaggctgaggcgggtggatcacctgaagtggggagttccataccagcctgaccaacatggagaaaccccatctctactaaaaatacaaaattagccgggcgtggtggcatacacctgtaatcccatctactcaggaagctgaggcaggagaatcacttgaacctgggaggtggaggttgccatgagccaagatcacaccattgcaccctagactggacaagagagaaacttccatctcaaaaaaaaaaaaaaaaaggatgagaaaaataataatttaaaaaaaagagtccaggctctggaaccagacagcctgggtcttacccctgctccaccattaccagccagttcttcttggatgagtgcctcagttgcctcaagtgtaaatggagataatggctggaccttcattataggccatgagcattcactgagagaatgtagctaacaaaagtgagttgtaggttggagcaaaagtaattgtggtttcagaccatgaactttaaattattataactaggctaaaatacatctttattaatcaaaataggaaccattaaaatcaacacatttttgccaataagaaataagtttgtttattcctgtagcataaaaattcatgcttcgggattcaacaaactcttggaaagcattttctgcatcctcctggttgtggaagcatttttcctgcagaaagttgtcaagattcttgaagaaatggtagtcagttggctagaggtcaggtaaatatggcggatgaggcaaaacttcatagtccaattcattcaacttttgaagctttggttgtgtgacatgcagtcgggtgttgtcgtggagaattggaccctttctgttgacgaatgccggctgcaggtgttgcagttttcagtgcatctcattgacttgccgagcatacttctcatatgtaatggtttcgcagggattcagaaagctgtaggggatcagactagcagcagaccaccagtgaccatgaccttttttttttggtgcgaatttgcctttgggaagtgctttggagcttcttctcggtccaaccactgagctagtcattgccagttgtataaaatccacttttcatcgcacgtcacaatcagatcaagaaatggttcgctgttgttgtgtagaataagagaagatgacacttcaaaatgacgattttcttggttttcactcagctcatgaggcacacacttatcgaggtttttcacctttccaatttgcttcaaatgctgaatgaccatggaatggtcgatgttgagttctcaagtagttgtaagaaaatcagctttgatgattgctctcaattggtcattgtcagcttctgatggcctgccagtacactcctcatcttcaaggctcttatctccttcgcaaaacttcttgaaccaccactgcactatacgttagttagcagttcctgggccaaatgcattgctgatgttgtgagttgtctccgctgctttacaacccattttgaattcaaataagaaaattgcttgaatttgctttttgtctaacatcattttcatagtctaaaataaatataaaataaacagaaagtattaagtcattagcaaaaaatcataaagtgagaattgtgcattaaaatgatgtatagcataaccacatttatttaagaatgtattccaatatcaaatggcaaatttcaacaatgcaaaaactgcaattacttttgcaccaatctaatagaagttcaataaatactggcaattacaattggcattgccttagggtcaacttgtaagacattcctgaaattgtgggaaagggggaggacctggagtggacattattggaaggcaaagctgtaaccaaaagagcaacctgggaaacacatgactcctctgttgctgtccctggccctatcctgtctcccctccctgttgtcagctacctcatatgttctctaatctctgtctctgtgccctcaaagacccccctgaaaatagaaatattactgctcattggttattttctatcaattaagtactgtattagtccgttttcatgctgatgataaagatatacccaagactgggcactttatgaaagaaagagttttattgaacttacagttccacgtggctggggaggtctcacaatcatggctgaaggtgaaaggcacatctcacatggcagcagacaggagaagagggcttgttcagggaaactcccctttttaaaaccatcagatatcatgaaacttatttactgtaatgagaacaggatgggattcaattaccttccactgggtcgctcccacaacacgtgggaattcaagagatttgggtggggacacagccaaaccatatcaagtactgtgcaagtgttttaggcatgcagagagtggtgggtcttcccagcaagcagagtgtggggaggtaatgggggactggtggctgacttaatggcccaggacccatgccacaaggagatggatggtggatgtgaataggagcctgcttacacccatcacaatttagattcttatgctcgatggcacgggtactcttttaggcccattttaccaatgaggagattgggactaatttgctcgagatcaaaaaagaagtggtgtaggtgggatttaaacccaggatgtctagcactaaaatgcaggtacttaaccactatcctaagggagtggctacttaatttgataaactcatctagtgaatggaagagagacggttacatttcactgatggtactgagcctttgttgatgagctcattgggaatctcagacatgagcaggatgtgtctaagggacaggtgggcttcagtagactggctaactcctgcagtctctttaactggacagtttcaagaggaaaaccaagaatccttgaagctcaccattgtatcttcttttccaggttgtccaataactgcatcacctacctaggggcagaagccctcctgcaggcccttgaaaggaatgacaccatcctggaagtctggtaaggcccctgggcaggcctgttttagctctccgaacctcagtttttctatctgtaaaatggggtgacgggagagaggaatggcagaattttgaggatcccttctgattctgacattcagtgagaatgattctgcatgtgaaggatctgattctctgtctaagaaagaagtctttacctctttaagtagggagcaatgatttcatttttaaaccttgactatttattcagcaacttctctgctctatgagatagtgtaggaatggggatgtggttgaagaatgaaaagaaaagtcagctcccgccctcctagaaattgcatctgccttcacaggtcaaggatattggatcagaccttctgcggttctgaatggagattacacaggttaggagcaggttgcacagtgtttccaattctctataattaaagccatagactttcatgtattgaaaaaagcaagaattgcattcttgacagattctttcattgccttaaaaagaatgactagccttgggagtctgggcagctgggtccagtgttgtagactttctctctgctgagccacagcttcaaagatttgtccttcttgtttccagggatctatttctcagacaataagtaaaggctttccctggcctaatgtgctgtaagtgaatgctactatatatgttccaggcactgggctagagactaatatttaaaagccaggaaatttcctatagaaaatctatatctcagggttttctcaaaagagctgggaactctggatgcccattcatgattccagtagttaaccagagtacaagaagggctgagtcttctcagatgggcaaacccactctggctgactgcagatccaccaagcctattgtcttagaccaggaccctttggcaactcattcccataagcctgtgacccttgctttaaatatgcaggccttgtcttctctcaaaaagcacatcaaggctgcagcgaatgcagatatcaaatgatgaagttaaaaacaaaagctttgctgggcgtggcagctcacacctgtaatcctagcactttgggaggctgaggcaggaggatcactttaggccagaggttcaacaccagaccttgtctctcaaaaaataaaaaattcagctgggtgcggtgtagttcctagccacttgggaggctgggatggaaggatcccttgaacccaggagttcaaggctgcagtgggccatgattgcatcactgcacaggcgacagaattagatcccatctcttaaaaaaataaaaaatttaaaagtgacttcaaaaatctatgctgtgatggagagatttttccttctgtatgattgtgatagctctgtggcctatgacgtcatcaggttctgggcaaagtgtaggttttctgtttctttgtttttgaaaccattgcacagtcctaagaaacatcacattctgggtcctgggcaccagccaacatgaggttgatattttaaggggtctgatgttatttgctgcctgttgatggaacttgggagggcaccagggtttgctcattgcattcttgacagattctcttattgccttaaaaagaatcactggccttggggagtctgtggctggctgggtgcagtgttgtggactctctctgcagagtcatggagccttgttcagaatgcttcctgagctgccctggttggccaagggtaaaaacagccctgacttccctgcaagaaacactgcagctgggccagagagtcagcccatcccaggcatgggtttaaaaagtggaggcttttgtttgaaagccctgctctaattttgtcctcactcaaacctctgttcacttgatctgctttaggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttgggggtttgagaccaacctgaccaacatggagaaaccctgtctctactaaaaatagtggaggttgcagtgagccaagatcacaccgttgtcctccagcctgggcaacaagagtggaactccatctcaaataaataaagaaaataaaaaataaaaatattactaaaacttgtcaaattttaaagcacaattttctacttgaagcggaggaaaatgatgttttcaacccgataatgtatagcttaaggcaaaagcaattcttagaaaatagccaggcaatcaaaacacttttggcttcagtttagaaaagtttaaccccgtgaagttttcagctgtgcaggagcctccccgtacccagcctaaaaggctgcaacacatgtgaaaaggccccttggcccccctcgattccctcaaagtggctccaggagtctttaacagaaggatggagaaggtttacttatggctggctctccacagcgagcagcctctcatgcaggccttgtgttaaaatagtgcttagctgcgtttaataagcaagggatcaggtggtcacccagcaaggtaaaatcctacttccttgaaaggttttgcgtctggctgactcggtggtgtggcatctccctggaccctgcatgggagtgtgtctatgggttttctttgtgattggtgccctgttctgcccagaccattgtttctgatacagcagatcaggaggaaggtggtgttgagtctcctttcgctgaggagtacacacagcagcccatttgcatagcactgtattgggttgtcagtttgtcatctttaaattcattcagcaatgaggccaggcatggtggctcatgcctgtaatctcagcactttgggaggctgaggcagctggatcacttgaggtcagaagttcaagaccagcctggccaacatggcaaaaccctgtctctaaaaatagaagaagaaaaaaaaaagttagctgggtgtggcagcgcacgcctgtaatcccagctacttgggaagcagaggcaggagaatcgcttgaatcttggaggaggaggttgcagtgagctgagatcatgccactgcactccagcctgggcgacacagcgagactgtcataaataaataaataaataaattcagcaatgatttattaaccagttatattaggatgaccagtggattgacaaaacagacatggtcctcaaggtgcttaggtgagggtggagggagaggcaggcagaaaacaagtcagttgaaaaagacatgaaaaagtcttcatccaggcactgtggctcatgcctgtaatctcagcactttgggaggccaaggtgggcagatcacttgcggtcgggagttcgagaccagcctggccaacatggtgaaaccctgtctctactaaaaatacaaaaattagccgggcatggtagcaggtgcctgtaatcccagctacttgggaggttgaa

SEQ ID NO: 2 - protein, 1040 aa, Homo sapiens
  NOD2 (1)..(1040)
MGEEGGSASHDEEERASVLLGHSPGCEMCSQEAFQAQRSQLVELLVSGSLEGFESVLDWLLSWEVLSWEDYEGFHLLGQPLSHLARRLLDTVWNKGTWACQKLIAAAQEAQADSQSPKLHGCWDPHSLHPARDLQSHRPAIVRRLHSHVENMLDLAWERGFVSQYECDEIRLPIFTPSQRARRLLDLATVKANGLAAFLLQHVQELPVPLALPLEAATCKKYMAKLRTTVSAQSRFLSTYDGAETLCLEDIYTENVLEVWADVGMAGPPQKSPATLGLEELFSTPGHLNDDADTVLVVGEAGSGKSTLLQRLHLLWAAGQDFQEFLFVFPFSCRQLQCMAKPLSVRTLLFEHCCWPDVGQEDIFQLLLDHPDRVLLTFDGFDEFKFRFTDRERHCSPTDPTSVQTLLFNLLQGNLLKNARKVVTSRPAAVSAFLRKYIRTEFNLKGFSEQGIELYLRKRHHEPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFLVRAKGVVPGSTAPLEFLHITFQCFFAAFYLALSADVPPALLRHLFNCGRPGNSPMARLLPTMCIQASEGKDSSVAALLQKAEPHNLQITAAFLAGLLSREHWGLLAECQTSEKALLRRQACARWCLARSLRKHFHSIPPAAPGEAKSVHAMPGFIWLIRSLYEMQEERLARKAARGLNVGHLKLTFCSVGPTECAALAFVLQHLRRPVALQLDYNSVGDIGVEQLLPCLGVCKALYLRDNNISDRGICKLIECALHCEQLQKLALFNNKLTDGCAHSMAKLLACRQNFLALRLGNNYITAAGAQVLAEGLRGNTSLQFLGFWGNRVGDEGAQALAEALGDHQSLRWLSLVGNNIGSVGAQALALMLAKNVMLEELCLEENHLQDEGVCSLAEGLKKNSSLKILKLSNNCITYLGAEALLQALERNDTILEVWLRGNTFSLEEVDKLGCRDTRLLL

SEQ ID NO: 3 - DNA, 41654 bp, Homo sapiens
ggggtgtatgtgtatcgggaagcatgtttgtggtggggggaagcagggcgtgtgtgtgtgtgtatgtgagtgttgggaagcaaggtatgtgtgttgggggaagcgagggctgtgtgtgtattgggaagcatgtttgtgtgtggggggaagcgagggctgtgtgtgtgtattgggaaacatgtttgcgtgtgggggggaagcagggcgtgtgtgtgtgttgcgaagcggtgtgtgtgtggtgtgtgtgtgtgcgcgtgtgtgctgggaagcggtgtgtgtggtgtgtggtgtgtgtgtgtgtgtatgtgtatgtgttgggaagcgggttgttccgaggactccccgattccctcctggttgaaacgtggcccagaaagagaggcacactggctagggcacacagccagggccgtccagccccgcaggaagccccggtcctgccctgtcccaggtaagcctggccagccctggagccggcgctgcggggctgcggggctgcggggctgcggggccacgggacaggagcgggtgctgcggtcccggcagagcaggggcggggtccgggcgggggcggagccgagcgcccgcgcacccggagccgcagcccgagccggagccggagccgggagtcgtggcccggagtgggccttggagtcggcgcgcaggcggctcgcggtgagtgctgtgcccagcgcctgggggtgggctggggggagactgtgggcgccgagggtcccacccccgggacttgaacacttggcttctgagccgtcctggccgcagggcttgggcaagggttgcctcggtttctcctgggggagtcccagaatgtagatgggaagtgagacggaaagggggcatctcgaggttggggaggcccctgggccactcttttgatttccttcctctctgtgggactcagtttccccagcttcaccatgccagtgagtgggtgggtataagtgagagagtctgcctggaggatcctgctgctgctctctgtacctcttatgggaatctcctgcctcctccttctcccttgttgggtgtgagatctaagttcaaatccatcccttcccagacagcccacaccaggacctgcgaggccacatcccccagctgtttgctctcctatgcttctcaggttgatggagcagccatctggtcctgctttcattccaacaaactgaaaagccctccaccccaatccccctggggaggacctcctgaagctgcatgagggctgggggctggacccctgcttctgagtttgctctttctgagcctctcctgccatggccttggtttccccatgggctgtgacctagcctcctccagggtccttgttctacagaggggaaacacctgccctgtcaccctcccctgaccccttagtgaaaaagaccagagtgacatccccaccacctgtacttgcctgtcccgatcactcagccatggggctgctggggcccctgagtttgttggctgctcaaggcccctttgcctcgctgcaccctgggatctgaggctggaagcatcctcatccagcccacctccctcctacccagtgcagagcccctctgtgacacatgcccagtggtggggagcttattactgatgaggtggctttttagtgccttgcagctcttgtttcctttagctaaaatctgtggcttacagattctacaccttggttccatgtctgtctcccggagccatgcagagcaagtcagttctttcattctttccagcagtcatcattcatgcactggatgagacacgagacagagcagtaggaaaatcagacaaaaccccctgccctcatggagtgcacactctagaaaggaatggacagcaatgaaataagtaaatacatggcgtagcaggtggcgggaagtactacgagtaaaacaaagctgggaagcaggatggggtgctggggctgggtattgcattgtatatagcacaaccagggaaggcttccccaagaccacgatagctgggcaaagtcataaggaattgaggggtgagccacatgagtatctggggggctgggaggctttggttttcactctgagcatgatgggagcacgggtttggaacagaggagtcacctgctttgacttggggtctggcaggcccatctagctgtggggtggagaatggatcatggggggggtggggtaagggcagaaggagagcccgttgagcggatcattgctgtcattctcgggagagaccatggtgtcttgcccatggcccagaggtggttaaaaaaaagaaaaccagacattcagatttgggatatatttcaaaggccgtgtcgatagggtttggggtgcaagagaaaggtagagtcaaagatgattctgagtgtttggctcgagcttctggaggtttgaagctgccatgaactgaaatgggagttgcttagcggagagatcaagagtttggttttgtgcttgtggagtttgagctgcccattaggcatcaatgatgagtaagcagttgggaaaagttaagggcggaggttggagttgaaaataaaactttgggcatcatcaccatataactggtatttaaagccacaagagcaggtgggctcatctagggatggagtgatatggagaagagaaggggtctaaccattgagtgctggggcccccagtgttaggaaccagccaagaagacagaaagagtgaaaatcagagagttggggtgtcctggaggaaatgaagaaaatgccccaaagaggaaggagggaacaaatatgaccaatgcccctggcagagcaagcaggctgagggctgaggattgagcaatgggaggtcactggtgacagtttcactggagctggatggggaactagagggaatgggaggggatgggaggacttggggacagcagtacaggcaacagacaagggggcctgctgtaaagggagcagataaatgggattggagccaaatgaagaaggggagtgtcaagagagtgctttacttttacaatggagaattagagtgcattgtgcactggtggggggatttgatctcttagggagagaacagtgttagggagggagaatgcaggatagctgggggagggtggggggcttggccccagcagagactcaggacacttgggaagttgagcttccctgggcttcccctcctctcctgtctgcaaggggtcagtgggctgagatttcagcacttaagcaaagcatttgctcttggccccagagaaaccgggctggctgtggtctcaggaaggaaggaggtgtccaggctcaggcctgggcctgggtttcagggagggcccacgtgggtcgccccttgaccctctctttcagcaaggaagtgatcctttctctacatgggcctcaccttggggaggacaatggtgtctttgaagttgtagtaactgaagtagagatcaaaaggcaatgcagatagactgacagatttcgcctgaagaggggaagcccgaccaggtaataaaggagtaagaggaaggatgttaaggacaattttaggaaacagataatgagtgaatattttttctctctctttcccaatttaaactgaagcaggagaaactgaagctagacataatgattaacttcccaagctggtgagcttcctgagctggttagtgagaacagcactaaggccaggttctcctccccagatgtttaagatgagacaggacaatgcctgctcagagacagggcctggctgaattggccctcaggattctctctgctctgaggtttctggaagaaggccagggcagaggtgtggtgatgtagctgctgggaggacagagctccgagtcacgtggcttgggcgggcctccccttcctggtgtccacagaagcccaacgtcactagctggggtgtgtatggctcacacgtaggccaggctgccctaggcttggtgtgcaagggaggggcccctacttacttgtggcctgtcccctcgtgaatgtgtctcatgtccccagtggggtttttcagtgagggtcatggtctccaggatgcacaaggctttgtgccagaattgcttggaattgcctagttctggaaggctggttggccaactctggcctccggcttttcctttgggaatttcccttgaaggtggggttggtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggtaagaggagcaggcattgtcccgtcccagcttgatcctcagccttctttcatccttggccgcgacatgctcccaggcctggggtcagatggggagtgctgactctgtttctgggctgttttctggggagaatgggtcggcgggtttttttccccaggacctgggcagggtcaatggtgggggccgctgtcgcatccttggctggtgtttccacagctgagaaccactccagggccaagcccagagcttattctacccttttttgtcctctcttcccctgtcctcggccaccccaccctcttggctcctctgcttagatgtgggcacaaggaggagaactccttggcctgagagaactaccttagatcctggcttccagtggcctctgcaggggggtacaccctctctcccaagcagccagacacacaagtaacctcattgcctcagtttccccatctgaccagcacagggccccctgtgccccagcagcgttctgagagattggagctttctccttttgcttaccttggctaccgtatgaggacggatacagagtgttccccccacccccagcccaggggatatttgattcatgaacattccctcagtgtctttgtgggggacaatgctgtgccaggctcagggatgccaggacgagtaagacccaggctcccacgtggcccaggcagggagagagacacataaacaaccatcaggaaagaggtaaaatccccaggccacttggcatctgctcccttgagtgtctgggaatgtccctgatttataaaaagaagctgacggccctctttgttgtccatgcctacaccctttcactttcgtttcttcggggcactgcagcagcccttgtccacagaccccatgacaatcgcagaactgaccatgctgagagattttcttggctgctcagggaccctgccagggcttgaagctcctggagggtcacttgccctcaaattcccagaacgcacagcaggtcactgatgatagcagtggcagcagtctgtgcacggtggtttcgagggcgtgggagggaggtgagggccctagggcaagtgtgtgtgggaagtgttgatgggggacaaggcaccagaacgctcggaaacaacttagtttgcaccgtaatttttcacttcgcctaggacaggacctttagagcaatattctgagtctaccccttggagtagcagtgtgcaaaacacacagcacgggcttggggcccccgtggggaacccaaatgtaagagttagagacatgcattccggagtcatacatggctcgtgttgaaatcctgactctgcctgtctagctgtgacacatcgtacaaatcacttagcttcttggtgcctcagtgtcttcctctgtagaatgggtagatcataggcactacttcagagtggctgggagggttcagtgaattcctgcaggagagcacttagaatggcacttggtgtgtagtttatgcttaattaatattagccgttactgaaactgctgtagcctgaatccagccagcatgaaagagcccctctcaccctgcttcgaagagaatgaattccctgattgtttggaagatctctctctctctctctgtcttttttttttttttttgagaaacggtcttgctctcttgcccaggctggagcgcaatggtgccatcttggctcactgcaacctctgcctcccgggttcaagtgattctcctgtctcagcctcctgagtagctgggattacaggcgctcgccaccacgcctggctaatttttgtatttttagtagagacagcgtttcaccgtgttggccgggctggtctagcgctcctgatctcaagtgaccttgggagatctcttgctcctaatattacctcaagcctttttaaacgttttaagccggagaccaagcatggatatgggagttaggggtcttgatttaattcttggttgcttcaaactctgtggaaccttgaggtgtttcttgccttctctgggtctcaattttcacatctatatggtggggagcttggattgggtaatgtctgaggctagaaccatggccaactcgggttctgctggggctgacttgccctggccttccctgaccaccctgcatctggcttctggagaagtcccgcactgaccttgttctcctccccaggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggtgaggcactcctggtgtgcatcacagagttctcaggaaaggggtgcttagtcaccaagactgatttgtcctcatgaagtcagcctgtggggtaacttggtccatgggatttcccctaaaaaggtagccaggcaggtaaaatttgctcttgactcttggcaggaaacgtacaactctttctttcttcttttcttttctttttctcactctgttaccctggctagaatgcagtggcacaatcatagctcactgtagccttgaattcctgcgctcaagtgatcttctggccttagagtagctgggactacggctgctgtaccaccatgaacagctaattttttttttttcttttagagatggggtgttgctatgttgcccaggctggtctccagctcctggctttaagcaatcctcccgccttggcctcccaaactgttgggattgcaggcatgagccactttgcctggccaacagaacacttctgccgagaggaagtgtgtggtggccaggaactcagattctggagccagaatggtgcaggctcaaggtcaaccctgtgtgatctcaggcttccctatggagcctctccagcctcagtctcccttgtttcagtttcctcatctacaaaacaatgttaatagtcaaatggtgcctatcctataaggctcttgggaggattcagtgagttaatttgagtaatgcttaggatagtgtctattaccactggctgctatttattatttctgttatgagtgatactctgtacttgtacacttttatttctgtctgttttaaattaacagcacaacagaccataacactgcagtatattgaatttattttataattaacatagcatattataaactaatatagcttaaatgtttatgtaggatttctgacatgaaattgcattagatcatagatgttcagagttggtatataacagcccctgagaatgtagtaactcagcagagaccagaaggtcagagaaatgaccactgagtatttttgaaactcttttgttttcttccaaatagtgattcttagggctcctgagaggcagatggaacaatcattaacattccactttataaatcgggaagttgagaccaaggaaagtagtttgaataagctcacagtagttaatgagggggccagtgctggaccaattggccagcactggtcattgacttattcatccatcattcatttattcagccagaatctattaggtgcttcatacatatttgcttaaagtttgttgtgttcatagagctttgcacacggtaggtactccataaacatttgttgatgaaataagtgagttactgaatgaatgattgaattagaatgacactgcagtgttaaaatgggctgggttggggaacattttagtttttgtttttgtctgttttccaaaaatgtatgtgttgttcacatgagtctggataaccctagattgagattgatgacataaataaatttgtcttcaaggctgcactaaagctggctcacatggctaggtatttacagagcagaagtggtgcagtcctctctgattagttgcacgtacagaagacatattcgttattggactgaccttagtttctcttataatttgttaggggaattgaatcagcccatctgagaagttacaagattgtgtcttgtcatctttaaaagttcagcaatgtgatgtggtacagatggtctgaggggtttggagaaggtagcctagatccctagggcccagagaagacaggatgtgaacagaggaagtacatggattggtgaagaaaagaaatgggataactcatgggtcaaagaagaaatcatgatggaaatcagaaaatattcagaaccatacaataatgagaatattatttatcaaaatctattggatgcagctaaagcaggacatagggggaaatttacaaccttaggtgcctagattaggaaagaaggaaggcatttgtttatttatttgtttatttatttatttgagatgggggtctcactgtgtcacccaggctgctggagtgcagtagcacgatcataaatcactgaagtctcgaacttctgggctgaagtgatcctcccgcctcagccttccaagtaggtgggacacaggctagcaccaccataccaggctaattttttttttgtagacacagggtcttgctatgttgaggtctcaaactcctgggctcaagtaatcctcctccctcggcttcccaaagtgctgggattacaggcatgagccactgcgcccatctaaggctgaattttaatgagctaagaattcatcttaagaaagggctaaatagacagcaaaagcaaacattgaaggttgggactgagctgagtgggtagcagggatgggagacaacagatctgaggagagcaggagattttgaaaggattgcactgcctgaggtttaagcctttagaatccagctctctctgagctccctttgagctctgacattctgtgactctgatttggtggccttcccttagtggccttactgatttcatttggatggtgcttgtggtatatccaaccaacatgtcttcccaaatggccttttaatttcctataaagaagtagttgtcattgattgcaggttagggacagaaaatgctgtggaatgaaacaaaatgcaagttaaagaactaaattccaaaaatacccattgctactattgactgagtgaattcctactgtgtgccagacactgtacccagtccattccctgtattgttttatttaagcctcacaagggtatagtgtgactacactgtttcttaacaatgaagaaactgcccaaatcgcccatctgggaagcggcccagctagaatttgaatccaggcctgttttcctccagagcttgtgctattctctgtctgtcataaaatgtgggggctttgtgtggtaaacttgctcagttgggcatagcagttgttaggaaacctgaggctggtaacaccagctgtaataccagctgtccgtctgactcatgcaactgttaaagttgatagggctgaggtgtcagactgagctctgaattgcctgattcctataacaatattaacttaaacattttttaaattgggaaatgcaccatgcatacagaagagtgtgtatatttcatatgtatagtgtaaactgttcccatcacccaggttaaaaaacaggatgttgccagtacctggggccttctttaactgcaactgctagaggtaaacactggcttgacttttgtgtaaatcatctctttgcctttctttaatgttttagcatcttttaaaataaatccccaaataatgtattgttctattttgaaaaactgagtagcaagccaaaaatagctgtgtaaagaaaggtcacttaaattaggctgggtgcagtggctcaagcctttaatcccagtactttgggaggctgaggcaggtggatcacaaggtcaggagatcgagaccatcctggccaacatggagaaaccccgtctctactaaaaatacaaaaaattagccaagaatagtggcatgtgcctgtagtcccagctactcgggaggctgaggcaggagaatcgcttgaacccgggaggcagatgttgcagtgagccgagatcgcactgcttgaacccgggaggcagaggttgcagtgagccaagatcgcaccactgcactctagcctgggtcacagagcaagactctgtctcaaaaaaaaaaaaaaaaaaaagaaaggttactattgccttttcttagatgaaggttcccaaggcagggaaagctaagtggagtctcagggacttggtctggcttttccttccctgggaatttataaggacctcttctgggaagtcagtcggcaatgccatgaatgagtctggggaaatattgggctcattgcaactggagggtctggtaggactgatgtgaattaggtgctgtgtccggaggaaaatggccagaggaagtgggctgctttgtacagtcagtggtaaagttgccaaaggctattatagctcacaggaatgggccaaggctaaacactcctgtggagtgaaatgaatgtcctcagctgactgaggcagcgggagttgaaaagaaacgatattagttcatggtgaagacaagtcaaatatagataaaggttagggtcaggcttgcctggacatctaggagataactgccctcaacttgtttgaatcttgagtcactgctccattttgtttgaactggtggccatctacttatagtatacagccatcaacctgagatttccctacatggtcttcctgccttggtctcctgtatcctgaatcctatggcctcttcttccctggtttactacattttgctagaccgtatcctccagtcaattccttagaatgaatgtatgaaagttaaaatttctgaggtctcacatgtcttaaagttccctcatactggattgatagtttggctgggtataaaattctgggctggccatcattttccttcagaattttgattgcattattccattatcctctcttttcaatattgcttctaagaattccaaaacctttttttttttttctttttgagacagtgtctcactctgtcacccaggctggaatgcagtagtgtgatctcagctcactgcaacctccacctcctgggtttaagtgattcttcttcctcagcctcctgagcagctgggattacaggcacccaccaccacaccctttagtagagatggggttttgctatgttggccaggctggtcttgaacttctgactttaggtgatctgcctacttcggcctcccaaagtgctgggattaaaggcgtgagccaccacacccagcctccaaaaccattttaaaactctttctggaagcttttaaaattttcttttagtccccagaattttaaaatttcaattatgtgccttggtgttcttccattatgttagtcacccaagaggtactttcaatctggaaacttctctatgttttgggaaatgttcttgattagtttacaggtgatttcttcctctccattttatctcttctcttttcatgaaactactattaattcaatgttagaattccttgactgatcatttaattttcttctattttccatctctgtgtctttttgctctacttttctatgatagtcacagctctatctttaaactcttgagtttttcatttttgatgtcatgattttaatttgcaaggggtaggtttgactgattcttttttgtagtatcttactcttgttttatggatgcaacatcttctttgacttaaggatcataagataggtgggttctttgtttgtttgtttgactgtttttcaccctatgtaaactttttctacaagtttctttccccttcccccctttttggcttctatctcccacattagatgctttctctgggctcatgatactctttggttttctttctcaagattgacaggtaggactttaaaacttgttgagcatgcgggtgaaacttgtctaccatgaatttcactgtagatattttggagattgacagtgtttatatctttagatctcgcctcctgggttgatcaagttatctgagtacaccacagaccttttgcctggggataaaccagaaatctgtttcagaaaccactttgattcagtcttccttgttttagtcatttccttcagttccggaggtccctcatgctgatcattccagagccctttacagatcctagggtacacactgcatggttttcaactttcttgttttggggttaagatttggctttcaggagtctcctcagtccgttactattcattcaatcagcaagtccttgagcacctgatttgtgccagacattcttctaggtgttagggatacctcagtgaacaaaacagacaaaaatctttgtcttggaaatacacacactccagtcaggggagagggacaataagccaaaggaaggaaattacagcgtgtgctagaaggtgataagtgctgtagaaagtaagtaaagtgggtttgggagttgagagtttgggaaggggataaatgatggcaattgtaaatagagtagtcagagttctcacttagaaggtgaaattcaagtaaagacttgaaggaggacagggaattagccacatggatggctaggggaaggcttccaagctgagaggacagccagagccaaggcccagaggcaggagcatacctggtagttttaggaaacaggaggccaggatgctgagtggagtaagagggggcatgaaaggagaaacttgggtccacgtggttctagacaggtatttttgtctgttttgggccctgaaggttactattggacttggactcttactctgaggaaatagggacgctattgggacgtttgtacaggagcaatgtgacctgagttttgtttgtaaaggattagactctggctgtggcattaaggctaggctgtgggggcaggaacagaagcagggggaccagttttgcagcctgtgcagctttccagataagcagggattgtggcttggaggaggatggtatagaggaggtgacaagaaatgactctatgtctggtatgtagatattggccacagatggcatttgagcactagagacctggctggtccacatggagtttccataagcacataatacacatcagatttcaaagacttaatatgaaagaaagaatgaaacgtaccttgctatttctttcttttttttttttttttgagacccagtcttgctctgtcacccaggctggagtgcagtggtgtgatctcggctcactgcaacctccgcctcccaggttcaagtgattctcctgcctcagcctcctgagtacctgggactacaggcacctgccaccacgcctggctaattttttgtatttttagtagtgatggggtttcaccatgttgtccaggctggtctggaactccggaccttaggggatctacccgccttggcctcccaaattgctgggattacaggcatgagccaccatgctcagccatatcttgctattttctacatggattacatgttgaaatggtaatgttttggctattgtggattaaatagaatatatgattaaagttgatttcatctatttcttttaactttaaaaaatatgtctgttagaggatttgaaattccacatgcggcttgcatttgtgacctgcatttcatttctgtggaacagtgccctttttgggacatgctttgaaggtggagtcaacaggatttggcagattacagacgagaggcttcaagggtgactccaagacttcggggcagagcacctggaagaaaggggttaatattagccaagatgaggaaggctgtcggtttggcaggtgcatgggcaggttaggagtttagttttgaatatgttggaggtgtttatgaaacttttaagtggagatggaaaataggcagttggatgtgcaagtccagggttcagggagacagttcaggctggagatgaagatgtgggagtctgaggagagattgtattcaaatattcaatccatgagacttgatgaaatcacttctcttccaaatgatttacagcctgcagaatcattttccctatctttgtaggtttatgtcttcattttgtttcatttatttttcagttattcactgttttagtgagttttgagtaggagccagattggatgcatgcgttcaattcaccatccaacactgtattaactacttgaaactcatgtggttgttcggttgtttttttgaccttttattctggatggaagagagatgcttatgaagttgcagtaatcagtaagccttcccacattgctccatcagccttcctggaagaataatgtcttctgcctttcctgtaggcaagaaggctgcttgatcttgccacggtgaaagcgaatggattggctgccttccttctacaacatgttcaggaattaccagtcccattggccctgcctttggaaggtaggtgtatgttctcagttaatcagaaagggaagggcagtcagtgcagatccatggttaagagcagaacacacctcggttaacatcccatatgctggcagtatagcctccctatgactcaatttccttgttttaaggctagcaccaccccgtctcattgggattttgggagcattaaaaggacaaaagcgtgtaatgttagctattagctttcattatctcccacacagtatactgacaattgggctaccatatattgagggctaactaaaggtgttacttaccatccaaactctcattatctgtaccgaaaagatatggacacatgttttgagttagggctggtatctcttgatctctgaaatttagcagctcacaatgggaaactcaagaaccaagtggatctagagactctggtatccctcagtgcccagggtcaccacccaaactcaggaacaggaggggcttggaccgcaccacttgaacataccaggcatcctgccaggtgctttatggacaatgtctaccctttgcaacaaccctgagaagtaggtggtgtttttttccaccttatagatgtggaaactgggcagggaggttaagtgacgagggaggggaagatgggtctgattgtaaattgtccccacctacactttctcttttcttgggagaagaaatgtcagttgtaaagagagagtgcaagcctggcactctttagggcttgttcctacaccactgtagggaaagctcattggcactgaagccccctgagctgtgtgtggtgctggcagatgggtctatcaccctgggctgtgtcctctgggcagcaagcaagcctgtgggcggggtggctggaagtctgtgcctggcactcgcgagtgcaccgtctcattgaagaacaggatctaaacatcagtgcgccacagcagggtgcgcggcacggagtgcaggccctggtttggcccttggttgaggtttgctgttgacatcatcaagcacagctagtcactgtaagaccaggccagggtgcaagattccccacacttctaaaggtgacaattggtgtgtttatttctctataaaatgacattttttttttctggagaattttagtatcattggtgatgactggaaaacctgcatcagaaatcaggtcggaagaggaagatatatatctgatatgtactggagaggaagatatctatcttatggtctaagttcagggatcctggtatattcagagggcagaaagctcagcaataatcatcaactctgggaacagaggtgacataaacacagggcgtcccctttgtgtgactgcagatagtcatcagtgagctcagagctctatgaaaattacttgctagtttttgggttgaaaatagtgggccagtgtttggttgggggcagtgaggctgtgatggcgggggaccatgccaagctcctaccagcctgggacgctaaaccagcacttccccatttcctgaaaggggaactaaactctgacacaggaaatggtttgcttgcattactttcaggatgagaaaggaagagcactggccttccaaacacaccccgtgcatgaaaactctccctgcatggggtgcatggggaggatggggaagtggaggcaggatcacagactcttgttcgagtgctcagctggggcaccccggtgaccccgaggccttcccttgctaggtccacccagatcaatcaggatcatctccccatctcgaagtttaactttatcacatctcagagttccttttgccacgtaaggtaacatattcacaggttctgagaatccggacatggacatctttgagggtctattgttgtgcctactatatccatgaataataatgataataagcaccattttttgagagtttgccatgtcagatattcttttaaactgtattttatctcgctgcctcctgaaaaaatccttccaggtgtatattgtccccatttttacagatgagagaactgaggcccagaaaggctaaatggcttgcccaagtgtatggtggacccaggttttcaaactcaggtgtgtctggcttcagagactgggctcctgagcccttaagccctttgttcccctttagaaaaagtcacctgaggctgagtggtgaagggatttatccaaagccacccggccactatggcaggacagatatcagaatacaggtcttccgatcccagcccagagccccttcccgtcatctagaactcctcctggtgtcagtaatgataacggcagtcactgatgtcttttgagcacttactttgtgttgagcacttacactgtgctaagcacttgacataggtcatcttagttgatccgtgtaaaactctgtgaggtagtgaccaacatttctcccaccttacagaggtggaaactgagggttaggaagtttccttgactgtcctcaaagtgcacagcttgtgaatggaggagccaggatgggcgcccgctggctctcctatcccttcagttatgtcagcgtcccccgcagcagcccattgtctggttaggtcccgtcttcaccatggtgccaccttcatctgcctcttcttctgccttccagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctcyggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtagtgagtgttactgggcattgctgttcaggtatgggggagcaccatcaaggctaagtgtgggagcaccgagctgggctctagaagtctgggcccagcttcgcctctgccaccctgctttgcaacactgcccagatcccttcccttctgggccttaatttcaatatgtgatgatgacagccacactttattgactggcctatgtgctgggtctggtgctatgctttccggaatgacctcatctaatctctacaaccaccctggcgggtaggcaggaatgttattatctccattatccttgacttgaggctcagagaagtgaagtaacttgtccaggaaatggcagagctggggttcacaaattgcatcattctgattacaggttttctgcctcccaccagtctatggatacacttcagaggctccctgaaaaccttgaggtcacttgcagaaagttttgtgtagtatgtgtccgtatcaggaacaacaccaaatcagaggtgacttgtgccccatcagagactttaacaccccaaccagatgggaatttcaggacccaagaaatagaaagtggctgcagggttacaactactgttggattcctgaggtagcacagtgtccaaacaggatttcagcactacccgtattgcttagagccccagccaaagatgtgaggttttgccctttggagaatctgtgcccctgaactcgggggcctctttccacatcttgggggcaggcaagggcagagggtgtgcctaggcctgcggatcagcatgcgacagattccccaacatccttccagcttgaaaggggattgccctgcttctatttagaacctataggaaagcagaagttctagattgaagttaaaattgattcccagcctccaggggctttgggctacacctggatgaccttaattgaccctaagcatgggacaaaccacttcctgagagtattaggatggtatacatcttctctgggggcaaagcaacaagatttatttttcatcatggaccaaacacatggatacccactagaaactgtgtagtgaattttgttaaccctgacatagggaccatggtctttaggttaaagcataataacaacataatacataacatatatagcgaatatatatatgtattatatgcaatgaatgtaaatatgattatacccatcatggtcttggaggaaacagatgacacacttaaaatgggtgttttgaggagagtttgaaaaacagattgtttacaagccatgggcaggagttaggaagagtgagagggttggtgcaggggcctggggttagtaacagctgggggagggtagacttgaagggggaaggggagggagactaattagctggggggaaggtatggagacggctgcctgagcttctgcaaagtggaagaataccgcttggccctaactcctcaccccaactcttgctcctggccagcgccttccaccagctggacccatcagggaggccgagtgggctgtctgctggagtagtccccaggcatcagcctcccaggagccagggacgggtagagaagggggagagtggatctggccaggcaaatggaaaacagccagcaccaaactctatttccctaggagggaggatcatgatactttgagtgggaatttggaaacctgtctgttggagcaatttccctgatagaaataagaatgtgcattttcctgggtagtagactcagtttttaccccaagaggccaggcatcactggcctgtgtgatcctcataggccagtccatctctggaattcttgaatggatcatccatccttgattagggatgtccccgtgattaccagggtgtgcagaagggctctgggaaacctgtgggtctgtctctgtgttcagagaaaggtgagggtggcctggttctagctcatggtgctcagactgtggtgtgtaaaggcactcgtggcaatgcagattcctgggcctgcctctagtgattcccattcagtaggtttggggtggggcccaggaaatctatatttttcacagacacccctggtgattctgatacaagtggtctcgccctgggagaactactggtctgcagcaaccagcttggttttccattagcaattactgtccttgagcgagttttactgctcttcaccttacacacactaaaactgccaaggccgtaggggaggggaagcaaccatgaggttgctgtgagtgcactgtgtgtgtgtgtgtgtgtgtgtgtgtgtgtgtgtgtgtgtatgagagagagagagagattgagaaagagaggaagggaggaagggggagggcacaggctcctctcccacagtgccaacctgcctctctcccacttgaagtgtttccatgccaactgaaatcctcagcctctaggaaaccctatatacacagtgcccctatataggtttctttagactctggctctctcagactctagagtgatggctttaaaagttttttgaatgttacccacaatgattgagaaagagagagagagagcacgcaccaccatgtaaacatggaacctaagtttcacaaaatgacttcgctttatgaactctgagacactctgctctcttctgttctgttctatttccattttagaaatgctgctcaggaccttcaaaatgatttgcatgacctgcaacctgcagtctgaaaaatcactgcactacagaagtggccataagaggccctgagggagaagctgcacaatgtcatggttaagagtggggtttggagccaagccgcctaggctcaaagcctttatgtgccgtacaaccttggcaaagtcacttcgcttgtctgtgcctcagtttctttctcacgaatgctcataataatggttcccatttcactggcttgttgtgaggatgaaatagtgttattattgagaagtggtaagggtagtgatcagtgctagcgatcatgattctaggtgacttttactgtgtaccgggtgctcacaaggctttatgtgcacagcctggtgaggctgataatactattgttccctctttttttttttttgagacggagtctcgttctgttgcccaggcagggggtacagtggcacaatctcggctcatgcaagctctgcctcccgggttcacgccattctcctgcctcagcctcccaagtagctgggactacaggcgcctgccaccacgcccggctaatttttttgtatttttggtagcgacagggtttcactgtgttaaccaggatggtctcgatctcctgacctcgtgatccgcccgcctcggcctcccaaagtgctgggattacaggcgtgagccaccgtgcccggcctgttccctcttttatagatgaagagaccagcaaataactagtaagtcgctgatcaggatcacaatatccagctgaggcactccagagcctgagctgttaaccattcagtcagggcctcccaagtttgcctaaagataaagaatcatgtgcacagttgttaaaatatacagattcctgggccccaccccgcagatacttgattgccagctccagggtatgggcctgagaatctgtcttttagggaagctttcagatgatgttgtgatcaggtgagttttgggaatggtgccccaagaggagtggcagacagggcttgctcggcagggactagcctgttggagtggtgccattggggttaaggactgggcagcagggcctcactaaccacagcctatatgcctgtttctgaagttttggccactctcatccagctggtctactgtctgctgacctagatgatggtaaattgtccccaggggtagcctgtctagttcaggctgcacctttcgcatatatcagctcctttccaccatcatcccctttgtgaggctgctgtgattatcatgttccttttgcagagatggaaacattgcctcaaattagctctgtcatttcctaaggattccagggttctttagtagggggtctggatcctacgtcctgggccatccccatcatagtgcaccacgtcacctccctggccagggaccgtggggtctccacttttttggggtgctccatctatgcagggtttcctggaagcacagatgctggcacttcagggatgaatgaaagtctttttgggggatttgtagatttttttcttgtcttactagctccattttcaaatgtatttattttgtctctttagtttgcgcgataacaatatctcagaccgaggcatctgcaagctcattgaatgtgctcttcactgcgagcaattgcagaagttagcgtaagtcagcctgggctgtggacaatgggctccaagtgccctggtctcaccccaggtcgtgcagcctgggaagctgtgagtgatgggctggggcaggggctgtttgcatgatggggggtgcaggtgattcctgcccagaggggaagggcaaccctgggatttggtgctcactgtccaatgtgctttgcttctgtgtctcctctcttctggaactgaacagtctattcaacaacaaattgactgacggctgtgcacactccatggctaagctccttgcatgcaggcagaacttcttggcattgaggtgagcccaggttttccttattccctggaaactattttttgccccattcctgagtcagtctgatctggtcttggcctggcactgcccacactggctcctgacctcctgattgaatgcagggacagtgtctcattttaagcaggggttctctaatgctgtgatctccccagtaaactctggactagctctgctgaggacttcctgtcttttgacctttagcccgtagggcaagaaagcttttctaggcccctttccttttctgtgtctaagagtgtcacagctttctgggtttactgagttccacgatgcatgttgagctcgtcctggtgggggaggcatacacagttacttgccaccccagctgtggcagcgagttgctgcaacactcccaggaggtcctttcaccactcagagcatgcaaggtttgcagtccatctggttctgcatttctgctactccagtgtctcccagtttcaacaggagtctctctctctcctacctgatgcctttaaattgcccctctagctggccgctgggttggcctggcttctctctccttctctctctctcagatattcttgcctcctgtgatttgtgaggcagtaaaaaaagacaaagtaaagaattgcttccatctattcttttacctcttgggctgggtttgtggatgggagccgccattttaaaatggcgggccacatagctcagtctcggcaagggctactgagatcagaaccacaggtgccaatttgtacaaaggactcagtcctgctaccactgcctgatccctcagactcacaagcctggaataggctgtggccagacctggctggcccatccctgagaagggtgctagtttcagaaatggaggctgagtttgtggccaacacagtagtcctccggtatgtgcaggagagatgttctaagaccccagtggatgcctgaaaccatggagagtatcaagccctacacataccatgcttttcccaatacctacacacctgcaataaagtgtagtttataaattaggcacagtaagagagtaatagcaactcataataaaatagaacaattataacaatcaatgtactataataacactatgtgaatgtggactctctccatctccctcaaaatatcttcttgtactgtactcacccttcttcttgggaagatgtgtggtggtaaaatgcctgtgtgatgggaggaagtgaggtggatgacgcatgcagcactgtgctctagcgctgggctgctgttgacctgaccacacttcaggaggagaatcatctgctcccagagatccctaatctttgagcaacaatgaggtcggcagctggatgtcaggagcagacgatcttgatgattaccaaatgggagcgtatagagcgtggatgcgctggacggggggctgattcacgtcctgggtgggatggagctggatggcacgtgatcagaatagcatgcaatttaaaatgtatgaattgtttatctctagaattttccatttaatatttttggactgcagttgatttcagataactgaaaccatagaaggcgaagctgcggataagcagggggcagggattaccgtatatcattgtaatagagagcacaggctctggagccagactgcccgaggtttgaaccctcattagctgcgtgacctcaggtcagcccaatgtctgtgtgcctccgtttccccttctgtagaatggaggtaataaccctggctacctcacaggctgtagtgatgagcaagcaagttaatccacatgaagggctgcaccgtctggcaggggctttatatagtaagcgagtggctgaaagatgatgggtaaatcacacaagcactcagcttgtttctccttatgtgagtccggtcctccaagcagggattcaatgtgccacccatttattggggaaaagtcctaaaaggggaagtggggaagggagctgggggaggctgggaggtgtgtccctgagtgaaggagagagggaaggaaggaaggttgagactgggcaccttggacttcagtgcagtcctaagacatcttggcaaggctgatgaggagttcttgaaccaaattcaccaggcaggggagcctgatgtctcaggcaggggctggcaagtgcagatgcgaggatgttagattttggagcacagcagctggggcccttggctacctccaaggagctgaggctggagacctgaaaggcgagttctcctagctgccacaccccttctccaaggatacaataatatctgccttataggattgttgtgagctgagtggcttgacgttccttgaaagaatgaaagcgtatagttatcccaggaagcctagggttgcaggtgagagctctggggcttctccgaagctctccgaggtgtctggattcagttgcagcaggagccttccttgctgggatcttcccccacccctagccttggccctccctctctccttcctttctggaaggctcagtgggccccacccctccctccagccacctggacctgcccagcgctcttgtgcaacaggtaaagcctacctgtagcaacaacagatctgggaaggctgcagagggcacgatggggtctggatcgagggcggctgagaccagagggaaaggtgtgaccctgagtcaccctcgctgtcccggggaaaccacctcccaggacagctgcctactgtggctcctgcctggaattgtcacactgctgtgcaaacagcgtcccgctgcccctttccctttgctgggggaaaatgaagttgtgggagccgctgagtaaactagacctagcagcgagggcacctgatgtggctgctgcctcccgggcaggtcttcaatgctttcttcctgtgtttccctggccagggcacagacggccctccttttctgcctgccgctgtgttctctcagcctcctctgtcttcccttccaggctggggaataactacatcactgccgcgggagcccaagtgctggccgaggggctccgaggcaacacctccttgcagttcctggggtaggttggattccaggaagagggacctgcatggaggggcttgggacttttgaggatttaggggcaggtgaaactcttcagccaggaggccccagaggcagcccagctccagtggggaggacaagccagggagagagtgggcggcccttgactgccaccttcatacttggtctatgcctgacaaacaggaagtttgggatgttggggctaggggaggacagtgcccacgagctggtgacaggaagccctctgatcctcagggggcgctagggctgtactttagctgcatattaaaaccacctggaagcttctaaacactattgccaggcctcccaccccagactgatgaaatgcaaatatctaggtgcaaggcccaggtatcaggagttttaaaaagcttcccaggggatgtacagccaggggtgaggacccctgacctaagaaagagaaggaaatggggaaggataggaaggcacccaggataagaggggctgtgctaggtccctcggagctcttgctccctgtaggaccatgctagggcctgccagggaggggagtaccccaacctgcagccccagggtgggcttcctctgtttgctaggcacccaggcttgcacctgtgctgtttccagcagcctctctcctatcctgtcatgccctagtgtgaactggagtccatttgacaagaactgggagttttagaacctgggactgtaggaagagagaataaccttagggcctaggtgttccagcccatttcacagggaggcaagttgcccccaagctcagttttttgttttgttttgttttgtttgagatgtagtctcactctgttgcccaggctagagtgcagtggcacgatcttggctcactgcaacctccgcctccttggttcaagcgattcacctgcctcagcttctcaagtagctgggattataggcacccaccaccacgcccagctaatttttgtatttttagtagagacagggtttcaccatgttggcccggctggtcttgaactcctgatctcagatgatccgcccgcctcggcctcccaaagtgctgggattacaggtgtgagccaccgcacccggcccccaagctcagtttgagccacaaatgggactatgttgctctagaaatcaacatcttttccacactgcattagtagcaacagagtctagaacaaaggaggccacagccccactgaactctcttctgcttgaggtcacatctgccacatcaggggtatttacctctttcaacacatatttattagggcacctgtctgggccaggcgttgtgctaaaacccccaaacgctgtcatatgatacaaagtgttctgtaacttgcttggtttttttttttgtttgtttgtttgttttgttttgtttttgttgttgtttttttttgcttcgccatatattataggaatttttttaggtcattatgacctctttatttacttaattatctatttatttattttactaatatttacagaaagggtctcactctgtcacccaggctggagtgcagtggttgcaatcatagctcattgtagccttgaactcctgagctcaagtgatcttcctacctcggcctcctgagtagctgggactacaggcacaagccaccatgcctggccgatatttttatgttttgtagagacggggtctcactatgttgcccaggctggtctcaaactcctgggctcaggtgatcctccctcctttgcctcccaaagtattgggattacacaagtgagccaccttgctcagcctgacctcatttttcaaagagctgcagagtgttacataatgtatttaactggtcactttttgatgactattaagttgttttcaggttttttgttattacagtgtcatatccctggggcacagagcagtgctggcacatagccagagctcaatcgatacatacctaatgaatgaaagtacagtggacatcctaattcagccattctttgctaacttgtgtacatacctgtccagggtaggtccctagaatacagtcaataagtcagaaggtgtgagttgggatctaccttttggaaagggatgttttcaaactacagtgagtcagaggaggatggcccagaagctgggggagttgaagctgatggcgtgaaggaattaggggtgttaggaagaagcaggagataaagagctagcttgcagaagaagtgttagacttgttatgggcaggtactggagggtagctaaggacttgtgggtggcagttaccaggaagcgtatctgaactaagtgtcagaaaaagtgtcacaactgtaaattactcttgtcagtgagttcctgtccttaagggttagggctgggtagccctctactattctctaagtctgtaatgtaaagccactgaaaactcttgggttaagtttggccatcccacccaaaagatggaggcaggtccactttgctgggaccaggagccccagtgaggccactctgggattgagtggtcctgcccctctggctgggactgcagagggaggaggactgttagttcatgtctagaacacatatcaggtactcactgacactgtctgttgactcttttggccttttcagattctggggcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgatacagtatctgtaaattgaagatattgggctggatcaggtacattttatcttaaggaccaattccaatccattggtagtgggtgcccagtgcaccacattaaaaagaattctaaggctgcacctgggcttaaagaagagcactataatcaattagtgatgtctaaaaaagctaaaaaaaaaaaaaaaagagcactgcattcaattagtgatgtctaaaaagggtagaaaaaaaaaaaaaaagaaaaaagaaagagcaccgcaatcaattagtgatgtctgaaatggagcagaccaggagagcaccacgaattttgccctccataggttagctcatctctgaggtctttccctgctctgacatacttttgttccatgattacctccagcctggtggggaacaacattggcagtgtgggtgcccaagccttggcactgatgctggcaaagaacgtcatgctagaagaactctggtgagtttgggggattctctgctctggggaagtggatcacaatctctgttgatcccctggcctcatccataggagcggttgtgtggacagacaaaggtggatgattgagtgattgactgattgattgattgtgtttgtctttatatgtactgagtggtatgaagcttatagagcctggtatgtacatgctaatttttttatttaataaaatatatgggtttgctggtttggtgactgcctccacatggcataagtgttaagagcacagactctgtaatcaagcaggccgtgatcttaggcaagttaaataacaatttcagaatctcaagtttcatgtctgtaaaatgagggtaagaatacttccaaccataaaggatttttgcaagaattagataaagtagtgcctgtgaagaccttaatatagtgcctggcatatttgtaagtgctccataaatgttaaattagaataatggcagggttactactactattactgctgctgctgctgctgctgctgctacaactactatagtactgtgactactactactaataaagttttgttattttaaagtgattttgagttcctaggagcactgggtattcaagtcttaggtcattttggaaggtgtaatggagttttgatagttgaaagaggaaccatgaatcatgcttatactgttgacctgaagcagattctaagtttctcatcctttagatgccactagtatagttttctgacatgttctgggcagcttcagattatgtcagggagataaaatactgaatgtttgattttcccgggaagcagaaaggcactgcaacatatgggcattgccataaacagattttatggatggaccttggctgttgcagggcttactagctctactcaagtatgattgattctatcctgactggattttgccacttggaatttcttagtagaggagaaccttgttatgagagcatcagttatgattactgttaaaagaaaaactttaggcaaattaaatttagcagaactggtttgaacatacagcaatttatgaattgggcagcattcagaactgggagtgctccacccagcaaggtaggcaagcagtatctatagacaggaaaaggaagtgatgtacaaaacagcttgattggttgcagctgggcatttgccttatatgggcatggtgtgatgaggcattttctttatatggatatagactgatcagctggtagactgtgactgactgaagcctggctgctgtgattggctaagacttagctgtttgttataaggatatgttgttaggttgcagtttgctacataggaactcaaagtacagaggcagtctcaggccaaatttagtttaactatatgttaagctgcaggtgacagaatacctccatctatagaggtttaaacaaggaaagggtttattttttcctgtataggcagctggatgtaggcagtgtagggtttgtacagtggctacaagaggccaggaggggtctcagctctgtctcattctcttcctgttccatcattcttagcctgtgacttcattcacatggttggttgtctcatgatcacaggatggctgctccaggtgcagcactacttctgtattcccggattcgatctatatacccaggaaagccatctgggttctctcctttaaaaagcattcctggaagccccacctgtcgacttccccttatgtatcaaccatgtgtatgtcacttgaccaacccacttgtatgttgtttgaccagccctggctgcaatggagagtgggaaatacagttttttcaccaagtgcatggctgtccaaatgaaatgagacttccattaataaggaagaaaggaaagatggagatcaggaagctgggggatcagggaacttattacattgagagcccttggagtgaattctcttgcaaatatgtccctggaattgagaatccccacaacgtactttatctgttctttctttatccatgagtttgggttttcagatgttggatttcctatatggggggcatgtgagttcatcatcttccataatcaatgttgtatcaactggattttctctcttcttctcaccagcctggaggagaaccatctccaggatgaaggtgtatgttctctcgcagaaggactgaagaaaaattcaagtttgaaaatcctgaagtaaggaacccataagcaggaaacaggacaataattgctggcctttggaaggggcatttctgaataagatctgggccgctctccgctgggctaactcatgtgaggtggcctggtagaacagcttgccttggtctaggtggacaaggattccagtgcaagttgtttatctgggaggtggtcccagtaaatgctgataggagagtggtgaagtgagatggggaagtgaaggtaaccaataaaggggagttatcaagccagttatcaatgagggaaattggagctcagtactctggggcactcctggagccagtgcagaacacacatggtcacctacccaaccaatgggcaagaaagccatggcatttatccaccaaccctctgtccttcctatgttgatgtgcgctcatggggcactgattctccagcacttccagctcaccctcacccagctgaacatgcttctggggtcaggagaatggcctcaggcagagagtggaaggtcttctctgcaagcagtggctggggaggtgatgtgatggggagtactgtggcctcctccagtggctgactcagtggcttgggacttgtgccacaaagagatggacagctcaggtgaacatgaacccacctagtgaccatcatgggtttgtcagggtgctctctgaggctgatgccaaaattcttatttcaagtagacctcaggaaccccatcagatggctccttttgctggaggaaagtggcatctgcctaggcaaatgtggtcctaggaaaacgcttgcctttagagacagacagacagacagctgcctctgtgagtgccagctttgctgccaggctgctacccactctggcgacactcatttgtgttgctttcacaagctaggaagtttccaaatatttggagaaaacacttccactaattatttgggtggaaatgggctgggaagttggggtgaagcccggatgtgtctgagccagatgccagctttgcactgagggtcggcctttgggaataccaagcccattatcaaccaggtgtggatatggcaggtttgtcttccctccttgtcacagccttactccacttgactcccatggatgccaggcaatgaggctggggttggtcccatgccaccctgtcatcagccttatttttcagcatcctaaactatatcatcccccacaaaaattgaacttctgatatatcttttataaaaaagagaaatgcctacatctttcttttccaggcttagtttctgccaagagttggttgagagcccaggcttgctgggtgcagtggctcacacctgtaatcccagcactttgggaggctgaggcgggtggatcacctgaagtggggagttccataccagcctgaccaacatggagaaaccccatctctactaaaaatacaaaattagccgggcgtggtggcatacacctgtaatcccatctactcaggaagctgaggcaggagaatcacttgaacctgggaggtggaggttgccatgagccaagatcacaccattgcaccctagactggacaagagagaaacttccatctcaaaaaaaaaaaaaaaaaggatgagaaaaataataatttaaaaaaaagagtccaggctctggaaccagacagcctgggtcttacccctgctccaccattaccagccagttcttcttggatgagtgcctcagttgcctcaagtgtaaatggagataatggctggaccttcattataggccatgagcattcactgagagaatgtagctaacaaaagtgagttgtaggttggagcaaaagtaattgtggtttcagaccatgaactttaaattattataactaggctaaaatacatctttattaatcaaaataggaaccattaaaatcaacacatttttgccaataagaaataagtttgtttattcctgtagcataaaaattcatgcttcgggattcaacaaactcttggaaagcattttctgcatcctcctggttgtggaagcatttttcctgcagaaagttgtcaagattcttgaagaaatggtagtcagttggctagaggtcaggtaaatatggcggatgaggcaaaacttcatagtccaattcattcaacttttgaagctttggttgtgtgacatgcagtcgggtgttgtcgtggagaattggaccctttctgttgacgaatgccggctgcaggtgttgcagttttcagtgcatctcattgacttgccgagcatacttctcatatgtaatggtttcgcagggattcagaaagctgtaggggatcagactagcagcagaccaccagtgaccatgaccttttttttttggtgcgaatttgcctttgggaagtgctttggagcttcttctcggtccaaccactgagctagtcattgccagttgtataaaatccacttttcatcgcacgtcacaatcagatcaagaaatggttcgctgttgttgtgtagaataagagaagatgacacttcaaaatgacgattttcttggttttcactcagctcatgaggcacacacttatcgaggtttttcacctttccaatttgcttcaaatgctgaatgaccatggaatggtcgatgttgagttctcaagtagttgtaagaaaatcagctttgatgattgctctcaattggtcattgtcagcttctgatggcctgccagtacactcctcatcttcaaggctcttatctccttcgcaaaacttcttgaaccaccactgcactatacgttagttagcagttcctgggccaaatgcattgctgatgttgtgagttgtctccgctgctttacaacccattttgaattcaaataagaaaattgcttgaatttgctttttgtctaacatcattttcatagtctaaaataaatataaaataaacagaaagtattaagtcattagcaaaaaatcataaagtgagaattgtgcattaaaatgatgtatagcataaccacatttatttaagaatgtattccaatatcaaatggcaaatttcaacaatgcaaaaactgcaattacttttgcaccaatctaatagaagttcaataaatactggcaattacaattggcattgccttagggtcaacttgtaagacattcctgaaattgtgggaaagggggaggacctggagtggacattattggaaggcaaagctgtaaccaaaagagcaacctgggaaacacatgactcctctgttgctgtccctggccctatcctgtctcccctccctgttgtcagctacctcatatgttctctaatctctgtctctgtgccctcaaagacccccctgaaaatagaaatattactgctcattggttattttctatcaattaagtactgtattagtccgttttcatgctgatgataaagatatacccaagactgggcactttatgaaagaaagagttttattgaacttacagttccacgtggctggggaggtctcacaatcatggctgaaggtgaaaggcacatctcacatggcagcagacaggagaagagggcttgttcagggaaactcccctttttaaaaccatcagatatcatgaaacttatttactgtaatgagaacaggatgggattcaattaccttccactgggtcgctcccacaacacgtgggaattcaagagatttgggtggggacacagccaaaccatatcaagtactgtgcaagtgttttaggcatgcagagagtggtgggtcttcccagcaagcagagtgtggggaggtaatgggggactggtggctgacttaatggcccaggacccatgccacaaggagatggatggtggatgtgaataggagcctgcttacacccatcacaatttagattcttatgctcgatggcacgggtactcttttaggcccattttaccaatgaggagattgggactaatttgctcgagatcaaaaaagaagtggtgtaggtgggatttaaacccaggatgtctagcactaaaatgcaggtacttaaccactatcctaagggagtggctacttaatttgataaactcatctagtgaatggaagagagacggttacatttcactgatggtactgagcctttgttgatgagctcattgggaatctcagacatgagcaggatgtgtctaagggacaggtgggcttcagtagactggctaactcctgcagtctctttaactggacagtttcaagaggaaaaccaagaatccttgaagctcaccattgtatcttcttttccaggttgtccaataactgcatcacctacctaggggcagaagccctcctgcaggcccttgaaaggaatgacaccatcctggaagtctggtaaggcccctgggcaggcctgttttagctctccgaacctcagtttttctatctgtaaaatggggtgacgggagagaggaatggcagaattttgaggatcccttctgattctgacattcagtgagaatgattctgcatgtgaaggatctgattctctgtctaagaaagaagtctttacctctttaagtagggagcaatgatttcatttttaaaccttgactatttattcagcaacttctctgctctatgagatagtgtaggaatggggatgtggttgaagaatgaaaagaaaagtcagctcccgccctcctagaaattgcatctgccttcacaggtcaaggatattggatcagaccttctgcggttctgaatggagattacacaggttaggagcaggttgcacagtgtttccaattctctataattaaagccatagactttcatgtattgaaaaaagcaagaattgcattcttgacagattctttcattgccttaaaaagaatgactagccttgggagtctgggcagctgggtccagtgttgtagactttctctctgctgagccacagcttcaaagatttgtccttcttgtttccagggatctatttctcagacaataagtaaaggctttccctggcctaatgtgctgtaagtgaatgctactatatatgttccaggcactgggctagagactaatatttaaaagccaggaaatttcctatagaaaatctatatctcagggttttctcaaaagagctgggaactctggatgcccattcatgattccagtagttaaccagagtacaagaagggctgagtcttctcagatgggcaaacccactctggctgactgcagatccaccaagcctattgtcttagaccaggaccctttggcaactcattcccataagcctgtgacccttgctttaaatatgcaggccttgtcttctctcaaaaagcacatcaaggctgcagcgaatgcagatatcaaatgatgaagttaaaaacaaaagctttgctgggcgtggcagctcacacctgtaatcctagcactttgggaggctgaggcaggaggatcactttaggccagaggttcaacaccagaccttgtctctcaaaaaataaaaaattcagctgggtgcggtgtagttcctagccacttgggaggctgggatggaaggatcccttgaacccaggagttcaaggctgcagtgggccatgattgcatcactgcacaggcgacagaattagatcccatctcttaaaaaaataaaaaatttaaaagtgacttcaaaaatctatgctgtgatggagagatttttccttctgtatgattgtgatagctctgtggcctatgacgtcatcaggttctgggcaaagtgtaggttttctgtttctttgtttttgaaaccattgcacagtcctaagaaacatcacattctgggtcctgggcaccagccaacatgaggttgatattttaaggggtctgatgttatttgctgcctgttgatggaacttgggagggcaccagggtttgctcattgcattcttgacagattctcttattgccttaaaaagaatcactggccttggggagtctgtggctggctgggtgcagtgttgtggactctctctgcagagtcatggagccttgttcagaatgcttcctgagctgccctggttggccaagggtaaaaacagccctgacttccctgcaagaaacactgcagctgggccagagagtcagcccatcccaggcatgggtttaaaaagtggaggcttttgtttgaaagccctgctctaattttgtcctcactcaaacctctgttcacttgatctgctttaggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttgggggtttgagaccaacctgaccaacatggagaaaccctgtctctactaaaaatagtggaggttgcagtgagccaagatcacaccgttgtcctccagcctgggcaacaagagtggaactccatctcaaataaataaagaaaataaaaaataaaaatattactaaaacttgtcaaattttaaagcacaattttctacttgaagcggaggaaaatgatgttttcaacccgataatgtatagcttaaggcaaaagcaattcttagaaaatagccaggcaatcaaaacacttttggcttcagtttagaaaagtttaaccccgtgaagttttcagctgtgcaggagcctccccgtacccagcctaaaaggctgcaacacatgtgaaaaggccccttggcccccctcgattccctcaaagtggctccaggagtctttaacagaaggatggagaaggtttacttatggctggctctccacagcgagcagcctctcatgcaggccttgtgttaaaatagtgcttagctgcgtttaataagcaagggatcaggtggtcacccagcaaggtaaaatcctacttccttgaaaggttttgcgtctggctgactcggtggtgtggcatctccctggaccctgcatgggagtgtgtctatgggttttctttgtgattggtgccctgttctgcccagaccattgtttctgatacagcagatcaggaggaaggtggtgttgagtctcctttcgctgaggagtacacacagcagcccatttgcatagcactgtattgggttgtcagtttgtcatctttaaattcattcagcaatgaggccaggcatggtggctcatgcctgtaatctcagcactttgggaggctgaggcagctggatcacttgaggtcagaagttcaagaccagcctggccaacatggcaaaaccctgtctctaaaaatagaagaagaaaaaaaaaagttagctgggtgtggcagcgcacgcctgtaatcccagctacttgggaagcagaggcaggagaatcgcttgaatcttggaggaggaggttgcagtgagctgagatcatgccactgcactccagcctgggcgacacagcgagactgtcataaataaataaataaataaattcagcaatgatttattaaccagttatattaggatgaccagtggattgacaaaacagacatggtcctcaaggtgcttaggtgagggtggagggagaggcaggcagaaaacaagtcagttgaaaaagacatgaaaaagtcttcatccaggcactgtggctcatgcctgtaatctcagcactttgggaggccaaggtgggcagatcacttgcggtcgggagttcgagaccagcctggccaacatggtgaaaccctgtctctactaaaaatacaaaaattagccgggcatggtagcaggtgcctgtaatcccagctacttgggaggttgaa

SEQ ID NO: 4 - DNA, 41655 bp, Homo sapiens
ggggtgtatgtgtatcgggaagcatgtttgtggtggggggaagcagggcgtgtgtgtgtgtgtatgtgagtgttgggaagcaaggtatgtgtgttgggggaagcgagggctgtgtgtgtattgggaagcatgtttgtgtgtggggggaagcgagggctgtgtgtgtgtattgggaaacatgtttgcgtgtgggggggaagcagggcgtgtgtgtgtgttgcgaagcggtgtgtgtgtggtgtgtgtgtgtgcgcgtgtgtgctgggaagcggtgtgtgtggtgtgtggtgtgtgtgtgtgtgtatgtgtatgtgttgggaagcgggttgttccgaggactccccgattccctcctggttgaaacgtggcccagaaagagaggcacactggctagggcacacagccagggccgtccagccccgcaggaagccccggtcctgccctgtcccaggtaagcctggccagccctggagccggcgctgcggggctgcggggctgcggggctgcggggccacgggacaggagcgggtgctgcggtcccggcagagcaggggcggggtccgggcgggggcggagccgagcgcccgcgcacccggagccgcagcccgagccggagccggagccgggagtcgtggcccggagtgggccttggagtcggcgcgcaggcggctcgcggtgagtgctgtgcccagcgcctgggggtgggctggggggagactgtgggcgccgagggtcccacccccgggacttgaacacttggcttctgagccgtcctggccgcagggcttgggcaagggttgcctcggtttctcctgggggagtcccagaatgtagatgggaagtgagacggaaagggggcatctcgaggttggggaggcccctgggccactcttttgatttccttcctctctgtgggactcagtttccccagcttcaccatgccagtgagtgggtgggtataagtgagagagtctgcctggaggatcctgctgctgctctctgtacctcttatgggaatctcctgcctcctccttctcccttgttgggtgtgagatctaagttcaaatccatcccttcccagacagcccacaccaggacctgcgaggccacatcccccagctgtttgctctcctatgcttctcaggttgatggagcagccatctggtcctgctttcattccaacaaactgaaaagccctccaccccaatccccctggggaggacctcctgaagctgcatgagggctgggggctggacccctgcttctgagtttgctctttctgagcctctcctgccatggccttggtttccccatgggctgtgacctagcctcctccagggtccttgttctacagaggggaaacacctgccctgtcaccctcccctgaccccttagtgaaaaagaccagagtgacatccccaccacctgtacttgcctgtcccgatcactcagccatggggctgctggggcccctgagtttgttggctgctcaaggcccctttgcctcgctgcaccctgggatctgaggctggaagcatcctcatccagcccacctccctcctacccagtgcagagcccctctgtgacacatgcccagtggtggggagcttattactgatgaggtggctttttagtgccttgcagctcttgtttcctttagctaaaatctgtggcttacagattctacaccttggttccatgtctgtctcccggagccatgcagagcaagtcagttctttcattctttccagcagtcatcattcatgcactggatgagacacgagacagagcagtaggaaaatcagacaaaaccccctgccctcatggagtgcacactctagaaaggaatggacagcaatgaaataagtaaatacatggcgtagcaggtggcgggaagtactacgagtaaaacaaagctgggaagcaggatggggtgctggggctgggtattgcattgtatatagcacaaccagggaaggcttccccaagaccacgatagctgggcaaagtcataaggaattgaggggtgagccacatgagtatctggggggctgggaggctttggttttcactctgagcatgatgggagcacgggtttggaacagaggagtcacctgctttgacttggggtctggcaggcccatctagctgtggggtggagaatggatcatggggggggtggggtaagggcagaaggagagcccgttgagcggatcattgctgtcattctcgggagagaccatggtgtcttgcccatggcccagaggtggttaaaaaaaagaaaaccagacattcagatttgggatatatttcaaaggccgtgtcgatagggtttggggtgcaagagaaaggtagagtcaaagatgattctgagtgtttggctcgagcttctggaggtttgaagctgccatgaactgaaatgggagttgcttagcggagagatcaagagtttggttttgtgcttgtggagtttgagctgcccattaggcatcaatgatgagtaagcagttgggaaaagttaagggcggaggttggagttgaaaataaaactttgggcatcatcaccatataactggtatttaaagccacaagagcaggtgggctcatctagggatggagtgatatggagaagagaaggggtctaaccattgagtgctggggcccccagtgttaggaaccagccaagaagacagaaagagtgaaaatcagagagttggggtgtcctggaggaaatgaagaaaatgccccaaagaggaaggagggaacaaatatgaccaatgcccctggcagagcaagcaggctgagggctgaggattgagcaatgggaggtcactggtgacagtttcactggagctggatggggaactagagggaatgggaggggatgggaggacttggggacagcagtacaggcaacagacaagggggcctgctgtaaagggagcagataaatgggattggagccaaatgaagaaggggagtgtcaagagagtgctttacttttacaatggagaattagagtgcattgtgcactggtggggggatttgatctcttagggagagaacagtgttagggagggagaatgcaggatagctgggggagggtggggggcttggccccagcagagactcaggacacttgggaagttgagcttccctgggcttcccctcctctcctgtctgcaaggggtcagtgggctgagatttcagcacttaagcaaagcatttgctcttggccccagagaaaccgggctggctgtggtctcaggaaggaaggaggtgtccaggctcaggcctgggcctgggtttcagggagggcccacgtgggtcgccccttgaccctctctttcagcaaggaagtgatcctttctctacatgggcctcaccttggggaggacaatggtgtctttgaagttgtagtaactgaagtagagatcaaaaggcaatgcagatagactgacagatttcgcctgaagaggggaagcccgaccaggtaataaaggagtaagaggaaggatgttaaggacaattttaggaaacagataatgagtgaatattttttctctctctttcccaatttaaactgaagcaggagaaactgaagctagacataatgattaacttcccaagctggtgagcttcctgagctggttagtgagaacagcactaaggccaggttctcctccccagatgtttaagatgagacaggacaatgcctgctcagagacagggcctggctgaattggccctcaggattctctctgctctgaggtttctggaagaaggccagggcagaggtgtggtgatgtagctgctgggaggacagagctccgagtcacgtggcttgggcgggcctccccttcctggtgtccacagaagcccaacgtcactagctggggtgtgtatggctcacacgtaggccaggctgccctaggcttggtgtgcaagggaggggcccctacttacttgtggcctgtcccctcgtgaatgtgtctcatgtccccagtggggtttttcagtgagggtcatggtctccaggatgcacaaggctttgtgccagaattgcttggaattgcctagttctggaaggctggttggccaactctggcctccggcttttcctttgggaatttcccttgaaggtggggttggtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggtaagaggagcaggcattgtcccgtcccagcttgatcctcagccttctttcatccttggccgcgacatgctcccaggcctggggtcagatggggagtgctgactctgtttctgggctgttttctggggagaatgggtcggcgggtttttttccccaggacctgggcagggtcaatggtgggggccgctgtcgcatccttggctggtgtttccacagctgagaaccactccagggccaagcccagagcttattctacccttttttgtcctctcttcccctgtcctcggccaccccaccctcttggctcctctgcttagatgtgggcacaaggaggagaactccttggcctgagagaactaccttagatcctggcttccagtggcctctgcaggggggtacaccctctctcccaagcagccagacacacaagtaacctcattgcctcagtttccccatctgaccagcacagggccccctgtgccccagcagcgttctgagagattggagctttctccttttgcttaccttggctaccgtatgaggacggatacagagtgttccccccacccccagcccaggggatatttgattcatgaacattccctcagtgtctttgtgggggacaatgctgtgccaggctcagggatgccaggacgagtaagacccaggctcccacgtggcccaggcagggagagagacacataaacaaccatcaggaaagaggtaaaatccccaggccacttggcatctgctcccttgagtgtctgggaatgtccctgatttataaaaagaagctgacggccctctttgttgtccatgcctacaccctttcactttcgtttcttcggggcactgcagcagcccttgtccacagaccccatgacaatcgcagaactgaccatgctgagagattttcttggctgctcagggaccctgccagggcttgaagctcctggagggtcacttgccctcaaattcccagaacgcacagcaggtcactgatgatagcagtggcagcagtctgtgcacggtggtttcgagggcgtgggagggaggtgagggccctagggcaagtgtgtgtgggaagtgttgatgggggacaaggcaccagaacgctcggaaacaacttagtttgcaccgtaatttttcacttcgcctaggacaggacctttagagcaatattctgagtctaccccttggagtagcagtgtgcaaaacacacagcacgggcttggggcccccgtggggaacccaaatgtaagagttagagacatgcattccggagtcatacatggctcgtgttgaaatcctgactctgcctgtctagctgtgacacatcgtacaaatcacttagcttcttggtgcctcagtgtcttcctctgtagaatgggtagatcataggcactacttcagagtggctgggagggttcagtgaattcctgcaggagagcacttagaatggcacttggtgtgtagtttatgcttaattaatattagccgttactgaaactgctgtagcctgaatccagccagcatgaaagagcccctctcaccctgcttcgaagagaatgaattccctgattgtttggaagatctctctctctctctctgtcttttttttttttttttgagaaacggtcttgctctcttgcccaggctggagcgcaatggtgccatcttggctcactgcaacctctgcctcccgggttcaagtgattctcctgtctcagcctcctgagtagctgggattacaggcgctcgccaccacgcctggctaatttttgtatttttagtagagacagcgtttcaccgtgttggccgggctggtctagcgctcctgatctcaagtgaccttgggagatctcttgctcctaatattacctcaagcctttttaaacgttttaagccggagaccaagcatggatatgggagttaggggtcttgatttaattcttggttgcttcaaactctgtggaaccttgaggtgtttcttgccttctctgggtctcaattttcacatctatatggtggggagcttggattgggtaatgtctgaggctagaaccatggccaactcgggttctgctggggctgacttgccctggccttccctgaccaccctgcatctggcttctggagaagtcccgcactgaccttgttctcctccccaggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggtgaggcactcctggtgtgcatcacagagttctcaggaaaggggtgcttagtcaccaagactgatttgtcctcatgaagtcagcctgtggggtaacttggtccatgggatttcccctaaaaaggtagccaggcaggtaaaatttgctcttgactcttggcaggaaacgtacaactctttctttcttcttttcttttctttttctcactctgttaccctggctagaatgcagtggcacaatcatagctcactgtagccttgaattcctgcgctcaagtgatcttctggccttagagtagctgggactacggctgctgtaccaccatgaacagctaattttttttttttcttttagagatggggtgttgctatgttgcccaggctggtctccagctcctggctttaagcaatcctcccgccttggcctcccaaactgttgggattgcaggcatgagccactttgcctggccaacagaacacttctgccgagaggaagtgtgtggtggccaggaactcagattctggagccagaatggtgcaggctcaaggtcaaccctgtgtgatctcaggcttccctatggagcctctccagcctcagtctcccttgtttcagtttcctcatctacaaaacaatgttaatagtcaaatggtgcctatcctataaggctcttgggaggattcagtgagttaatttgagtaatgcttaggatagtgtctattaccactggctgctatttattatttctgttatgagtgatactctgtacttgtacacttttatttctgtctgttttaaattaacagcacaacagaccataacactgcagtatattgaatttattttataattaacatagcatattataaactaatatagcttaaatgtttatgtaggatttctgacatgaaattgcattagatcatagatgttcagagttggtatataacagcccctgagaatgtagtaactcagcagagaccagaaggtcagagaaatgaccactgagtatttttgaaactcttttgttttcttccaaatagtgattcttagggctcctgagaggcagatggaacaatcattaacattccactttataaatcgggaagttgagaccaaggaaagtagtttgaataagctcacagtagttaatgagggggccagtgctggaccaattggccagcactggtcattgacttattcatccatcattcatttattcagccagaatctattaggtgcttcatacatatttgcttaaagtttgttgtgttcatagagctttgcacacggtaggtactccataaacatttgttgatgaaataagtgagttactgaatgaatgattgaattagaatgacactgcagtgttaaaatgggctgggttggggaacattttagtttttgtttttgtctgttttccaaaaatgtatgtgttgttcacatgagtctggataaccctagattgagattgatgacataaataaatttgtcttcaaggctgcactaaagctggctcacatggctaggtatttacagagcagaagtggtgcagtcctctctgattagttgcacgtacagaagacatattcgttattggactgaccttagtttctcttataatttgttaggggaattgaatcagcccatctgagaagttacaagattgtgtcttgtcatctttaaaagttcagcaatgtgatgtggtacagatggtctgaggggtttggagaaggtagcctagatccctagggcccagagaagacaggatgtgaacagaggaagtacatggattggtgaagaaaagaaatgggataactcatgggtcaaagaagaaatcatgatggaaatcagaaaatattcagaaccatacaataatgagaatattatttatcaaaatctattggatgcagctaaagcaggacatagggggaaatttacaaccttaggtgcctagattaggaaagaaggaaggcatttgtttatttatttgtttatttatttatttgagatgggggtctcactgtgtcacccaggctgctggagtgcagtagcacgatcataaatcactgaagtctcgaacttctgggctgaagtgatcctcccgcctcagccttccaagtaggtgggacacaggctagcaccaccataccaggctaattttttttttgtagacacagggtcttgctatgttgaggtctcaaactcctgggctcaagtaatcctcctccctcggcttcccaaagtgctgggattacaggcatgagccactgcgcccatctaaggctgaattttaatgagctaagaattcatcttaagaaagggctaaatagacagcaaaagcaaacattgaaggttgggactgagctgagtgggtagcagggatgggagacaacagatctgaggagagcaggagattttgaaaggattgcactgcctgaggtttaagcctttagaatccagctctctctgagctccctttgagctctgacattctgtgactctgatttggtggccttcccttagtggccttactgatttcatttggatggtgcttgtggtatatccaaccaacatgtcttcccaaatggccttttaatttcctataaagaagtagttgtcattgattgcaggttagggacagaaaatgctgtggaatgaaacaaaatgcaagttaaagaactaaattccaaaaatacccattgctactattgactgagtgaattcctactgtgtgccagacactgtacccagtccattccctgtattgttttatttaagcctcacaagggtatagtgtgactacactgtttcttaacaatgaagaaactgcccaaatcgcccatctgggaagcggcccagctagaatttgaatccaggcctgttttcctccagagcttgtgctattctctgtctgtcataaaatgtgggggctttgtgtggtaaacttgctcagttgggcatagcagttgttaggaaacctgaggctggtaacaccagctgtaataccagctgtccgtctgactcatgcaactgttaaagttgatagggctgaggtgtcagactgagctctgaattgcctgattcctataacaatattaacttaaacattttttaaattgggaaatgcaccatgcatacagaagagtgtgtatatttcatatgtatagtgtaaactgttcccatcacccaggttaaaaaacaggatgttgccagtacctggggccttctttaactgcaactgctagaggtaaacactggcttgacttttgtgtaaatcatctctttgcctttctttaatgttttagcatcttttaaaataaatccccaaataatgtattgttctattttgaaaaactgagtagcaagccaaaaatagctgtgtaaagaaaggtcacttaaattaggctgggtgcagtggctcaagcctttaatcccagtactttgggaggctgaggcaggtggatcacaaggtcaggagatcgagaccatcctggccaacatggagaaaccccgtctctactaaaaatacaaaaaattagccaagaatagtggcatgtgcctgtagtcccagctactcgggaggctgaggcaggagaatcgcttgaacccgggaggcagatgttgcagtgagccgagatcgcactgcttgaacccgggaggcagaggttgcagtgagccaagatcgcaccactgcactctagcctgggtcacagagcaagactctgtctcaaaaaaaaaaaaaaaaaaaagaaaggttactattgccttttcttagatgaaggttcccaaggcagggaaagctaagtggagtctcagggacttggtctggcttttccttccctgggaatttataaggacctcttctgggaagtcagtcggcaatgccatgaatgagtctggggaaatattgggctcattgcaactggagggtctggtaggactgatgtgaattaggtgctgtgtccggaggaaaatggccagaggaagtgggctgctttgtacagtcagtggtaaagttgccaaaggctattatagctcacaggaatgggccaaggctaaacactcctgtggagtgaaatgaatgtcctcagctgactgaggcagcgggagttgaaaagaaacgatattagttcatggtgaagacaagtcaaatatagataaaggttagggtcaggcttgcctggacatctaggagataactgccctcaacttgtttgaatcttgagtcactgctccattttgtttgaactggtggccatctacttatagtatacagccatcaacctgagatttccctacatggtcttcctgccttggtctcctgtatcctgaatcctatggcctcttcttccctggtttactacattttgctagaccgtatcctccagtcaattccttagaatgaatgtatgaaagttaaaatttctgaggtctcacatgtcttaaagttccctcatactggattgatagtttggctgggtataaaattctgggctggccatcattttccttcagaattttgattgcattattccattatcctctcttttcaatattgcttctaagaattccaaaacctttttttttttttctttttgagacagtgtctcactctgtcacccaggctggaatgcagtagtgtgatctcagctcactgcaacctccacctcctgggtttaagtgattcttcttcctcagcctcctgagcagctgggattacaggcacccaccaccacaccctttagtagagatggggttttgctatgttggccaggctggtcttgaacttctgactttaggtgatctgcctacttcggcctcccaaagtgctgggattaaaggcgtgagccaccacacccagcctccaaaaccattttaaaactctttctggaagcttttaaaattttcttttagtccccagaattttaaaatttcaattatgtgccttggtgttcttccattatgttagtcacccaagaggtactttcaatctggaaacttctctatgttttgggaaatgttcttgattagtttacaggtgatttcttcctctccattttatctcttctcttttcatgaaactactattaattcaatgttagaattccttgactgatcatttaattttcttctattttccatctctgtgtctttttgctctacttttctatgatagtcacagctctatctttaaactcttgagtttttcatttttgatgtcatgattttaatttgcaaggggtaggtttgactgattcttttttgtagtatcttactcttgttttatggatgcaacatcttctttgacttaaggatcataagataggtgggttctttgtttgtttgtttgactgtttttcaccctatgtaaactttttctacaagtttctttccccttcccccctttttggcttctatctcccacattagatgctttctctgggctcatgatactctttggttttctttctcaagattgacaggtaggactttaaaacttgttgagcatgcgggtgaaacttgtctaccatgaatttcactgtagatattttggagattgacagtgtttatatctttagatctcgcctcctgggttgatcaagttatctgagtacaccacagaccttttgcctggggataaaccagaaatctgtttcagaaaccactttgattcagtcttccttgttttagtcatttccttcagttccggaggtccctcatgctgatcattccagagccctttacagatcctagggtacacactgcatggttttcaactttcttgttttggggttaagatttggctttcaggagtctcctcagtccgttactattcattcaatcagcaagtccttgagcacctgatttgtgccagacattcttctaggtgttagggatacctcagtgaacaaaacagacaaaaatctttgtcttggaaatacacacactccagtcaggggagagggacaataagccaaaggaaggaaattacagcgtgtgctagaaggtgataagtgctgtagaaagtaagtaaagtgggtttgggagttgagagtttgggaaggggataaatgatggcaattgtaaatagagtagtcagagttctcacttagaaggtgaaattcaagtaaagacttgaaggaggacagggaattagccacatggatggctaggggaaggcttccaagctgagaggacagccagagccaaggcccagaggcaggagcatacctggtagttttaggaaacaggaggccaggatgctgagtggagtaagagggggcatgaaaggagaaacttgggtccacgtggttctagacaggtatttttgtctgttttgggccctgaaggttactattggacttggactcttactctgaggaaatagggacgctattgggacgtttgtacaggagcaatgtgacctgagttttgtttgtaaaggattagactctggctgtggcattaaggctaggctgtgggggcaggaacagaagcagggggaccagttttgcagcctgtgcagctttccagataagcagggattgtggcttggaggaggatggtatagaggaggtgacaagaaatgactctatgtctggtatgtagatattggccacagatggcatttgagcactagagacctggctggtccacatggagtttccataagcacataatacacatcagatttcaaagacttaatatgaaagaaagaatgaaacgtaccttgctatttctttcttttttttttttttttgagacccagtcttgctctgtcacccaggctggagtgcagtggtgtgatctcggctcactgcaacctccgcctcccaggttcaagtgattctcctgcctcagcctcctgagtacctgggactacaggcacctgccaccacgcctggctaattttttgtatttttagtagtgatggggtttcaccatgttgtccaggctggtctggaactccggaccttaggggatctacccgccttggcctcccaaattgctgggattacaggcatgagccaccatgctcagccatatcttgctattttctacatggattacatgttgaaatggtaatgttttggctattgtggattaaatagaatatatgattaaagttgatttcatctatttcttttaactttaaaaaatatgtctgttagaggatttgaaattccacatgcggcttgcatttgtgacctgcatttcatttctgtggaacagtgccctttttgggacatgctttgaaggtggagtcaacaggatttggcagattacagacgagaggcttcaagggtgactccaagacttcggggcagagcacctggaagaaaggggttaatattagccaagatgaggaaggctgtcggtttggcaggtgcatgggcaggttaggagtttagttttgaatatgttggaggtgtttatgaaacttttaagtggagatggaaaataggcagttggatgtgcaagtccagggttcagggagacagttcaggctggagatgaagatgtgggagtctgaggagagattgtattcaaatattcaatccatgagacttgatgaaatcacttctcttccaaatgatttacagcctgcagaatcattttccctatctttgtaggtttatgtcttcattttgtttcatttatttttcagttattcactgttttagtgagttttgagtaggagccagattggatgcatgcgttcaattcaccatccaacactgtattaactacttgaaactcatgtggttgttcggttgtttttttgaccttttattctggatggaagagagatgcttatgaagttgcagtaatcagtaagccttcccacattgctccatcagccttcctggaagaataatgtcttctgcctttcctgtaggcaagaaggctgcttgatcttgccacggtgaaagcgaatggattggctgccttccttctacaacatgttcaggaattaccagtcccattggccctgcctttggaaggtaggtgtatgttctcagttaatcagaaagggaagggcagtcagtgcagatccatggttaagagcagaacacacctcggttaacatcccatatgctggcagtatagcctccctatgactcaatttccttgttttaaggctagcaccaccccgtctcattgggattttgggagcattaaaaggacaaaagcgtgtaatgttagctattagctttcattatctcccacacagtatactgacaattgggctaccatatattgagggctaactaaaggtgttacttaccatccaaactctcattatctgtaccgaaaagatatggacacatgttttgagttagggctggtatctcttgatctctgaaatttagcagctcacaatgggaaactcaagaaccaagtggatctagagactctggtatccctcagtgcccagggtcaccacccaaactcaggaacaggaggggcttggaccgcaccacttgaacataccaggcatcctgccaggtgctttatggacaatgtctaccctttgcaacaaccctgagaagtaggtggtgtttttttccaccttatagatgtggaaactgggcagggaggttaagtgacgagggaggggaagatgggtctgattgtaaattgtccccacctacactttctcttttcttgggagaagaaatgtcagttgtaaagagagagtgcaagcctggcactctttagggcttgttcctacaccactgtagggaaagctcattggcactgaagccccctgagctgtgtgtggtgctggcagatgggtctatcaccctgggctgtgtcctctgggcagcaagcaagcctgtgggcggggtggctggaagtctgtgcctggcactcgcgagtgcaccgtctcattgaagaacaggatctaaacatcagtgcgccacagcagggtgcgcggcacggagtgcaggccctggtttggcccttggttgaggtttgctgttgacatcatcaagcacagctagtcactgtaagaccaggccagggtgcaagattccccacacttctaaaggtgacaattggtgtgtttatttctctataaaatgacattttttttttctggagaattttagtatcattggtgatgactggaaaacctgcatcagaaatcaggtcggaagaggaagatatatatctgatatgtactggagaggaagatatctatcttatggtctaagttcagggatcctggtatattcagagggcagaaagctcagcaataatcatcaactctgggaacagaggtgacataaacacagggcgtcccctttgtgtgactgcagatagtcatcagtgagctcagagctctatgaaaattacttgctagtttttgggttgaaaatagtgggccagtgtttggttgggggcagtgaggctgtgatggcgggggaccatgccaagctcctaccagcctgggacgctaaaccagcacttccccatttcctgaaaggggaactaaactctgacacaggaaatggtttgcttgcattactttcaggatgagaaaggaagagcactggccttccaaacacaccccgtgcatgaaaactctccctgcatggggtgcatggggaggatggggaagtggaggcaggatcacagactcttgttcgagtgctcagctggggcaccccggtgaccccgaggccttcccttgctaggtccacccagatcaatcaggatcatctccccatctcgaagtttaactttatcacatctcagagttccttttgccacgtaaggtaacatattcacaggttctgagaatccggacatggacatctttgagggtctattgttgtgcctactatatccatgaataataatgataataagcaccattttttgagagtttgccatgtcagatattcttttaaactgtattttatctcgctgcctcctgaaaaaatccttccaggtgtatattgtccccatttttacagatgagagaactgaggcccagaaaggctaaatggcttgcccaagtgtatggtggacccaggttttcaaactcaggtgtgtctggcttcagagactgggctcctgagcccttaagccctttgttcccctttagaaaaagtcacctgaggctgagtggtgaagggatttatccaaagccacccggccactatggcaggacagatatcagaatacaggtcttccgatcccagcccagagccccttcccgtcatctagaactcctcctggtgtcagtaatgataacggcagtcactgatgtcttttgagcacttactttgtgttgagcacttacactgtgctaagcacttgacataggtcatcttagttgatccgtgtaaaactctgtgaggtagtgaccaacatttctcccaccttacagaggtggaaactgagggttaggaagtttccttgactgtcctcaaagtgcacagcttgtgaatggaggagccaggatgggcgcccgctggctctcctatcccttcagttatgtcagcgtcccccgcagcagcccattgtctggttaggtcccgtcttcaccatggtgccaccttcatctgcctcttcttctgccttccagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtagtgagtgttactgggcattgctgttcaggtatgggggagcaccatcaaggctaagtgtgggagcaccgagctgggctctagaagtctgggcccagcttcgcctctgccaccctgctttgcaacactgcccagatcccttcccttctgggccttaatttcaatatgtgatgatgacagccacactttattgactggcctatgtgctgggtctggtgctatgctttccggaatgacctcatctaatctctacaaccaccctggcgggtaggcaggaatgttattatctccattatccttgacttgaggctcagagaagtgaagtaacttgtccaggaaatggcagagctggggttcacaaattgcatcattctgattacaggttttctgcctcccaccagtctatggatacacttcagaggctccctgaaaaccttgaggtcacttgcagaaagttttgtgtagtatgtgtccgtatcaggaacaacaccaaatcagaggtgacttgtgccccatcagagactttaacaccccaaccagatgggaatttcaggacccaagaaatagaaagtggctgcagggttacaactactgttggattcctgaggtagcacagtgtccaaacaggatttcagcactacccgtattgcttagagccccagccaaagatgtgaggttttgccctttggagaatctgtgcccctgaactcgggggcctctttccacatcttgggggcaggcaagggcagagggtgtgcctaggcctgcggatcagcatgcgacagattccccaacatccttccagcttgaaaggggattgccctgcttctatttagaacctataggaaagcagaagttctagattgaagttaaaattgattcccagcctccaggggctttgggctacacctggatgaccttaattgaccctaagcatgggacaaaccacttcctgagagtattaggatggtatacatcttctctgggggcaaagcaacaagatttatttttcatcatggaccaaacacatggatacccactagaaactgtgtagtgaattttgttaaccctgacatagggaccatggtctttaggttaaagcataataacaacataatacataacatatatagcgaatatatatatgtattatatgcaatgaatgtaaatatgattatacccatcatggtcttggaggaaacagatgacacacttaaaatgggtgttttgaggagagtttgaaaaacagattgtttacaagccatgggcaggagttaggaagagtgagagggttggtgcaggggcctggggttagtaacagctgggggagggtagacttgaagggggaaggggagggagactaattagctggggggaaggtatggagacggctgcctgagcttctgcaaagtggaagaataccgcttggccctaactcctcaccccaactcttgctcctggccagcgccttccaccagctggacccatcagggaggccgagtgggctgtctgctggagtagtccccaggcatcagcctcccaggagccagggacgggtagagaagggggagagtggatctggccaggcaaatggaaaacagccagcaccaaactctatttccctaggagggaggatcatgatactttgagtgggaatttggaaacctgtctgttggagcaatttccctgatagaaataagaatgtgcattttcctgggtagtagactcagtttttaccccaagaggccaggcatcactggcctgtgtgatcctcataggccagtccatctctggaattcttgaatggatcatccatccttgattagggatgtccccgtgattaccagggtgtgcagaagggctctgggaaacctgtgggtctgtctctgtgttcagagaaaggtgagggtggcctggttctagctcatggtgctcagactgtggtgtgtaaaggcactcgtggcaatgcagattcctgggcctgcctctagtgattcccattcagtaggtttggggtggggcccaggaaatctatatttttcacagacacccctggtgattctgatacaagtggtctcgccctgggagaactactggtctgcagcaaccagcttggttttccattagcaattactgtccttgagcgagttttactgctcttcaccttacacacactaaaactgccaaggccgtaggggaggggaagcaaccatgaggttgctgtgagtgcactgtgtgtgtgtgtgtgtgtgtgtgtgtgtgtgtgtgtgtgtatgagagagagagagagattgagaaagagaggaagggaggaagggggagggcacaggctcctctcccacagtgccaacctgcctctctcccacttgaagtgtttccatgccaactgaaatcctcagcctctaggaaaccctatatacacagtgcccctatataggtttctttagactctggctctctcagactctagagtgatggctttaaaagttttttgaatgttacccacaatgattgagaaagagagagagagagcacgcaccaccatgtaaacatggaacctaagtttcacaaaatgacttcgctttatgaactctgagacactctgctctcttctgttctgttctatttccattttagaaatgctgctcaggaccttcaaaatgatttgcatgacctgcaacctgcagtctgaaaaatcactgcactacagaagtggccataagaggccctgagggagaagctgcacaatgtcatggttaagagtggggtttggagccaagccgcctaggctcaaagcctttatgtgccgtacaaccttggcaaagtcacttcgcttgtctgtgcctcagtttctttctcacgaatgctcataataatggttcccatttcactggcttgttgtgaggatgaaatagtgttattattgagaagtggtaagggtagtgatcagtgctagcgatcatgattctaggtgacttttactgtgtaccgggtgctcacaaggctttatgtgcacagcctggtgaggctgataatactattgttccctctttttttttttttgagacggagtctcgttctgttgcccaggcagggggtacagtggcacaatctcggctcatgcaagctctgcctcccgggttcacgccattctcctgcctcagcctcccaagtagctgggactacaggcgcctgccaccacgcccggctaatttttttgtatttttggtagcgacagggtttcactgtgttaaccaggatggtctcgatctcctgacctcgtgatccgcccgcctcggcctcccaaagtgctgggattacaggcgtgagccaccgtgcccggcctgttccctcttttatagatgaagagaccagcaaataactagtaagtcgctgatcaggatcacaatatccagctgaggcactccagagcctgagctgttaaccattcagtcagggcctcccaagtttgcctaaagataaagaatcatgtgcacagttgttaaaatatacagattcctgggccccaccccgcagatacttgattgccagctccagggtatgggcctgagaatctgtcttttagggaagctttcagatgatgttgtgatcaggtgagttttgggaatggtgccccaagaggagtggcagacagggcttgctcggcagggactagcctgttggagtggtgccattggggttaaggactgggcagcagggcctcactaaccacagcctatatgcctgtttctgaagttttggccactctcatccagctggtctactgtctgctgacctagatgatggtaaattgtccccaggggtagcctgtctagttcaggctgcacctttcgcatatatcagctcctttccaccatcatcccctttgtgaggctgctgtgattatcatgttccttttgcagagatggaaacattgcctcaaattagctctgtcatttcctaaggattccagggttctttagtagggggtctggatcctacgtcctgggccatccccatcatagtgcaccacgtcacctccctggccagggaccgtggggtctccacttttttggggtgctccatctatgcagggtttcctggaagcacagatgctggcacttcagggatgaatgaaagtctttttgggggatttgtagatttttttcttgtcttactagctccattttcaaatgtatttattttgtctctttagtttgcgcgataacaatatctcagaccgaggcatctgcaagctcattgaatgtgctcttcactgcgagcaattgcagaagttagcgtaagtcagcctgggctgtggacaatgggctccaagtgccctggtctcaccccaggtcgtgcagcctgggaagctgtgagtgatgggctggggcaggggctgtttgcatgatggggggtgcaggtgattcctgcccagaggggaagggcaaccctgggatttggtgctcactgtccaatgtgctttgcttctgtgtctcctctcttctggaactgaacagtctattcaacaacaaattgactgacggctgtgcacactccatggctaagctccttgcatgcaggcagaacttcttggcattgaggtgagcccaggttttccttattccctggaaactattttttgccccattcctgagtcagtctgatctggtcttggcctggcactgcccacactggctcctgacctcctgattgaatgcagggacagtgtctcattttaagcaggggttctctaatgctgtgatctccccagtaaactctggactagctctgctgaggacttcctgtcttttgacctttagcccgtagggcaagaaagcttttctaggcccctttccttttctgtgtctaagagtgtcacagctttctgggtttactgagttccacgatgcatgttgagctcgtcctggtgggggaggcatacacagttacttgccaccccagctgtggcagcgagttgctgcaacactcccaggaggtcctttcaccactcagagcatgcaaggtttgcagtccatctggttctgcatttctgctactccagtgtctcccagtttcaacaggagtctctctctctcctacctgatgcctttaaattgcccctctagctggccgctgggttggcctggcttctctctccttctctctctctcagatattcttgcctcctgtgatttgtgaggcagtaaaaaaagacaaagtaaagaattgcttccatctattcttttacctcttgggctgggtttgtggatgggagccgccattttaaaatggcgggccacatagctcagtctcggcaagggctactgagatcagaaccacaggtgccaatttgtacaaaggactcagtcctgctaccactgcctgatccctcagactcacaagcctggaataggctgtggccagacctggctggcccatccctgagaagggtgctagtttcagaaatggaggctgagtttgtggccaacacagtagtcctccggtatgtgcaggagagatgttctaagaccccagtggatgcctgaaaccatggagagtatcaagccctacacataccatgcttttcccaatacctacacacctgcaataaagtgtagtttataaattaggcacagtaagagagtaatagcaactcataataaaatagaacaattataacaatcaatgtactataataacactatgtgaatgtggactctctccatctccctcaaaatatcttcttgtactgtactcacccttcttcttgggaagatgtgtggtggtaaaatgcctgtgtgatgggaggaagtgaggtggatgacgcatgcagcactgtgctctagcgctgggctgctgttgacctgaccacacttcaggaggagaatcatctgctcccagagatccctaatctttgagcaacaatgaggtcggcagctggatgtcaggagcagacgatcttgatgattaccaaatgggagcgtatagagcgtggatgcgctggacggggggctgattcacgtcctgggtgggatggagctggatggcacgtgatcagaatagcatgcaatttaaaatgtatgaattgtttatctctagaattttccatttaatatttttggactgcagttgatttcagataactgaaaccatagaaggcgaagctgcggataagcagggggcagggattaccgtatatcattgtaatagagagcacaggctctggagccagactgcccgaggtttgaaccctcattagctgcgtgacctcaggtcagcccaatgtctgtgtgcctccgtttccccttctgtagaatggaggtaataaccctggctacctcacaggctgtagtgatgagcaagcaagttaatccacatgaagggctgcaccgtctggcaggggctttatatagtaagcgagtggctgaaagatgatgggtaaatcacacaagcactcagcttgtttctccttatgtgagtccggtcctccaagcagggattcaatgtgccacccatttattggggaaaagtcctaaaaggggaagtggggaagggagctgggggaggctgggaggtgtgtccctgagtgaaggagagagggaaggaaggaaggttgagactgggcaccttggacttcagtgcagtcctaagacatcttggcaaggctgatgaggagttcttgaaccaaattcaccaggcaggggagcctgatgtctcaggcaggggctggcaagtgcagatgcgaggatgttagattttggagcacagcagctggggcccttggctacctccaaggagctgaggctggagacctgaaaggcgagttctcctagctgccacaccccttctccaaggatacaataatatctgccttataggattgttgtgagctgagtggcttgacgttccttgaaagaatgaaagcgtatagttatcccaggaagcctagggttgcaggtgagagctctggggcttctccgaagctctccgaggtgtctggattcagttgcagcaggagccttccttgctgggatcttcccccacccctagccttggccctccctctctccttcctttctggaaggctcagtgggccccacccctccctccagccacctggacctgcccagcgctcttgtgcaacaggtaaagcctacctgtagcaacaacagatctgggaaggctgcagagggcacgatggggtctggatcgagggcggctgagaccagagggaaaggtgtgaccctgagtcaccctcgctgtcccggggaaaccacctcccaggacagctgcctactgtggctcctgcctggaattgtcacactgctgtgcaaacagcgtcccgctgcccctttccctttgctgggggaaaatgaagttgtgggagccgctgagtaaactagacctagcagcgagggcacctgatgtggctgctgcctcccgggcaggtcttcaatgctttcttcctgtgtttccctggccagggcacagacggccctccttttctgcctgccgctgtgttctctcagcctcctctgtcttcccttccaggctggggaataactacatcactgccgcgggagcccaagtgctggccgaggggctccgaggcaacacctccttgcagttcctggggtaggttggattccaggaagagggacctgcatggaggggcttgggacttttgaggatttaggggcaggtgaaactcttcagccaggaggccccagaggcagcccagctccagtggggaggacaagccagggagagagtgggcggcccttgactgccaccttcatacttggtctatgcctgacaaacaggaagtttgggatgttggggctaggggaggacagtgcccacgagctggtgacaggaagccctctgatcctcagggggcgctagggctgtactttagctgcatattaaaaccacctggaagcttctaaacactattgccaggcctcccaccccagactgatgaaatgcaaatatctaggtgcaaggcccaggtatcaggagttttaaaaagcttcccaggggatgtacagccaggggtgaggacccctgacctaagaaagagaaggaaatggggaaggataggaaggcacccaggataagaggggctgtgctaggtccctcggagctcttgctccctgtaggaccatgctagggcctgccagggaggggagtaccccaacctgcagccccagggtgggcttcctctgtttgctaggcacccaggcttgcacctgtgctgtttccagcagcctctctcctatcctgtcatgccctagtgtgaactggagtccatttgacaagaactgggagttttagaacctgggactgtaggaagagagaataaccttagggcctaggtgttccagcccatttcacagggaggcaagttgcccccaagctcagttttttgttttgttttgttttgtttgagatgtagtctcactctgttgcccaggctagagtgcagtggcacgatcttggctcactgcaacctccgcctccttggttcaagcgattcacctgcctcagcttctcaagtagctgggattataggcacccaccaccacgcccagctaatttttgtatttttagtagagacagggtttcaccatgttggcccggctggtcttgaactcctgatctcagatgatccgcccgcctcggcctcccaaagtgctgggattacaggtgtgagccaccgcacccggcccccaagctcagtttgagccacaaatgggactatgttgctctagaaatcaacatcttttccacactgcattagtagcaacagagtctagaacaaaggaggccacagccccactgaactctcttctgcttgaggtcacatctgccacatcaggggtatttacctctttcaacacatatttattagggcacctgtctgggccaggcgttgtgctaaaacccccaaacgctgtcatatgatacaaagtgttctgtaacttgcttggtttttttttttgtttgtttgtttgttttgttttgtttttgttgttgtttttttttgcttcgccatatattataggaatttttttaggtcattatgacctctttatttacttaattatctatttatttattttactaatatttacagaaagggtctcactctgtcacccaggctggagtgcagtggttgcaatcatagctcattgtagccttgaactcctgagctcaagtgatcttcctacctcggcctcctgagtagctgggactacaggcacaagccaccatgcctggccgatatttttatgttttgtagagacggggtctcactatgttgcccaggctggtctcaaactcctgggctcaggtgatcctccctcctttgcctcccaaagtattgggattacacaagtgagccaccttgctcagcctgacctcatttttcaaagagctgcagagtgttacataatgtatttaactggtcactttttgatgactattaagttgttttcaggttttttgttattacagtgtcatatccctggggcacagagcagtgctggcacatagccagagctcaatcgatacatacctaatgaatgaaagtacagtggacatcctaattcagccattctttgctaacttgtgtacatacctgtccagggtaggtccctagaatacagtcaataagtcagaaggtgtgagttgggatctaccttttggaaagggatgttttcaaactacagtgagtcagaggaggatggcccagaagctgggggagttgaagctgatggcgtgaaggaattaggggtgttaggaagaagcaggagataaagagctagcttgcagaagaagtgttagacttgttatgggcaggtactggagggtagctaaggacttgtgggtggcagttaccaggaagcgtatctgaactaagtgtcagaaaaagtgtcacaactgtaaattactcttgtcagtgagttcctgtccttaagggttagggctgggtagccctctactattctctaagtctgtaatgtaaagccactgaaaactcttgggttaagtttggccatcccacccaaaagatggaggcaggtccactttgctgggaccaggagccccagtgaggccactctgggattgagtggtcctgcccctctggctgggactgcagagggaggaggactgttagttcatgtctagaacacatatcaggtactcactgacactgtctgttgactcttttggccttttcagattctggggcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgatacagtatctgtaaattgaagatattgggctggatcaggtacattttatcttaaggaccaattccaatccattggtagtgggtgcccagtgcaccacattaaaaagaattctaaggctgcacctgggcttaaagaagagcactataatcaattagtgatgtctaaaaaagctaaaaaaaaaaaaaaaagagcactgcattcaattagtgatgtctaaaaagggtagaaaaaaaaaaaaaaagaaaaaagaaagagcaccgcaatcaattagtgatgtctgaaatggagcagaccaggagagcaccacgaattttgccctccataggttagctcatctctgaggtctttccctgctctgacatacttttgttccatgattacctccagcctggtggggaacaacattggcagtgtgggtgcccaagccttggcactgatgctggcaaagaacgtcatgctagaagaactctggtgagtttgggggattctctgctctggggaagtggatcacaatctctgttgatcccctggcctcatccataggagcggttgtgtggacagacaaaggtggatgattgagtgattgactgattgattgattgtgtttgtctttatatgtactgagtggtatgaagcttatagagcctggtatgtacatgctaatttttttatttaataaaatatatgggtttgctggtttggtgactgcctccacatggcataagtgttaagagcacagactctgtaatcaagcaggccgtgatcttaggcaagttaaataacaatttcagaatctcaagtttcatgtctgtaaaatgagggtaagaatacttccaaccataaaggatttttgcaagaattagataaagtagtgcctgtgaagaccttaatatagtgcctggcatatttgtaagtgctccataaatgttaaattagaataatggcagggttactactactattactgctgctgctgctgctgctgctgctacaactactatagtactgtgactactactactaataaagttttgttattttaaagtgattttgagttcctaggagcactgggtattcaagtcttaggtcattttggaaggtgtaatggagttttgatagttgaaagaggaaccatgaatcatgcttatactgttgacctgaagcagattctaagtttctcatcctttagatgccactagtatagttttctgacatgttctgggcagcttcagattatgtcagggagataaaatactgaatgtttgattttcccgggaagcagaaaggcactgcaacatatgggcattgccataaacagattttatggatggaccttggctgttgcagggcttactagctctactcaagtatgattgattctatcctgactggattttgccacttggaatttcttagtagaggagaaccttgttatgagagcatcagttatgattactgttaaaagaaaaactttaggcaaattaaatttagcagaactggtttgaacatacagcaatttatgaattgggcagcattcagaactgggagtgctccacccagcaaggtaggcaagcagtatctatagacaggaaaaggaagtgatgtacaaaacagcttgattggttgcagctgggcatttgccttatatgggcatggtgtgatgaggcattttctttatatggatatagactgatcagctggtagactgtgactgactgaagcctggctgctgtgattggctaagacttagctgtttgttataaggatatgttgttaggttgcagtttgctacataggaactcaaagtacagaggcagtctcaggccaaatttagtttaactatatgttaagctgcaggtgacagaatacctccatctatagaggtttaaacaaggaaagggtttattttttcctgtataggcagctggatgtaggcagtgtagggtttgtacagtggctacaagaggccaggaggggtctcagctctgtctcattctcttcctgttccatcattcttagcctgtgacttcattcacatggttggttgtctcatgatcacaggatggctgctccaggtgcagcactacttctgtattcccggattcgatctatatacccaggaaagccatctgggttctctcctttaaaaagcattcctggaagccccacctgtcgacttccccttatgtatcaaccatgtgtatgtcacttgaccaacccacttgtatgttgtttgaccagccctggctgcaatggagagtgggaaatacagttttttcaccaagtgcatggctgtccaaatgaaatgagacttccattaataaggaagaaaggaaagatggagatcaggaagctgggggatcagggaacttattacattgagagcccttggagtgaattctcttgcaaatatgtccctggaattgagaatccccacaacgtactttatctgttctttctttatccatgagtttgggttttcagatgttggatttcctatatggggggcatgtgagttcatcatcttccataatcaatgttgtatcaactggattttctctcttcttctcaccagcctggaggagaaccatctccaggatgaaggtgtatgttctctcgcagaaggactgaagaaaaattcaagtttgaaaatcctgaagtaaggaacccataagcaggaaacaggacaataattgctggcctttggaaggggcatttctgaataagatctgggccgctctccgctgggctaactcatgtgaggtggcctggtagaacagcttgccttggtctaggtggacaaggattccagtgcaagttgtttatctgggaggtggtcccagtaaatgctgataggagagtggtgaagtgagatggggaagtgaaggtaaccaataaaggggagttatcaagccagttatcaatgagggaaattggagctcagtactctggggcactcctggagccagtgcagaacacacatggtcacctacccaaccaatgggcaagaaagccatggcatttatccaccaaccctctgtccttcctatgttgatgtgcgctcatggggcactgattctccagcacttccagctcaccctcacccagctgaacatgcttctggggtcaggagaatggcctcaggcagagagtggaaggtcttctctgcaagcagtggctggggaggtgatgtgatggggagtactgtggcctcctccagtggctgactcagtggcttgggacttgtgccacaaagagatggacagctcaggtgaacatgaacccacctagtgaccatcatgggtttgtcagggtgctctctgaggctgatgccaaaattcttatttcaagtagacctcaggaaccccatcagatggctccttttgctggaggaaagtggcatctgcctaggcaaatgtggtcctaggaaaacgcttgcctttagagacagacagacagacagctgcctctgtgagtgccagctttgctgccaggctgctacccactctggcgacactcatttgtgttgctttcacaagctaggaagtttccaaatatttggagaaaacacttccactaattatttgggtggaaatgggctgggaagttggggtgaagcccggatgtgtctgagccagatgccagctttgcactgagggtcggcctttgggaataccaagcccattatcaaccaggtgtggatatggcaggtttgtcttccctccttgtcacagccttactccacttgactcccatggatgccaggcaatgaggctggggttggtcccatgccaccctgtcatcagccttatttttcagcatcctaaactatatcatcccccacaaaaattgaacttctgatatatcttttataaaaaagagaaatgcctacatctttcttttccaggcttagtttctgccaagagttggttgagagcccaggcttgctgggtgcagtggctcacacctgtaatcccagcactttgggaggctgaggcgggtggatcacctgaagtggggagttccataccagcctgaccaacatggagaaaccccatctctactaaaaatacaaaattagccgggcgtggtggcatacacctgtaatcccatctactcaggaagctgaggcaggagaatcacttgaacctgggaggtggaggttgccatgagccaagatcacaccattgcaccctagactggacaagagagaaacttccatctcaaaaaaaaaaaaaaaaaggatgagaaaaataataatttaaaaaaaagagtccaggctctggaaccagacagcctgggtcttacccctgctccaccattaccagccagttcttcttggatgagtgcctcagttgcctcaagtgtaaatggagataatggctggaccttcattataggccatgagcattcactgagagaatgtagctaacaaaagtgagttgtaggttggagcaaaagtaattgtggtttcagaccatgaactttaaattattataactaggctaaaatacatctttattaatcaaaataggaaccattaaaatcaacacatttttgccaataagaaataagtttgtttattcctgtagcataaaaattcatgcttcgggattcaacaaactcttggaaagcattttctgcatcctcctggttgtggaagcatttttcctgcagaaagttgtcaagattcttgaagaaatggtagtcagttggctagaggtcaggtaaatatggcggatgaggcaaaacttcatagtccaattcattcaacttttgaagctttggttgtgtgacatgcagtcgggtgttgtcgtggagaattggaccctttctgttgacgaatgccggctgcaggtgttgcagttttcagtgcatctcattgacttgccgagcatacttctcatatgtaatggtttcgcagggattcagaaagctgtaggggatcagactagcagcagaccaccagtgaccatgaccttttttttttggtgcgaatttgcctttgggaagtgctttggagcttcttctcggtccaaccactgagctagtcattgccagttgtataaaatccacttttcatcgcacgtcacaatcagatcaagaaatggttcgctgttgttgtgtagaataagagaagatgacacttcaaaatgacgattttcttggttttcactcagctcatgaggcacacacttatcgaggtttttcacctttccaatttgcttcaaatgctgaatgaccatggaatggtcgatgttgagttctcaagtagttgtaagaaaatcagctttgatgattgctctcaattggtcattgtcagcttctgatggcctgccagtacactcctcatcttcaaggctcttatctccttcgcaaaacttcttgaaccaccactgcactatacgttagttagcagttcctgggccaaatgcattgctgatgttgtgagttgtctccgctgctttacaacccattttgaattcaaataagaaaattgcttgaatttgctttttgtctaacatcattttcatagtctaaaataaatataaaataaacagaaagtattaagtcattagcaaaaaatcataaagtgagaattgtgcattaaaatgatgtatagcataaccacatttatttaagaatgtattccaatatcaaatggcaaatttcaacaatgcaaaaactgcaattacttttgcaccaatctaatagaagttcaataaatactggcaattacaattggcattgccttagggtcaacttgtaagacattcctgaaattgtgggaaagggggaggacctggagtggacattattggaaggcaaagctgtaaccaaaagagcaacctgggaaacacatgactcctctgttgctgtccctggccctatcctgtctcccctccctgttgtcagctacctcatatgttctctaatctctgtctctgtgccctcaaagacccccctgaaaatagaaatattactgctcattggttattttctatcaattaagtactgtattagtccgttttcatgctgatgataaagatatacccaagactgggcactttatgaaagaaagagttttattgaacttacagttccacgtggctggggaggtctcacaatcatggctgaaggtgaaaggcacatctcacatggcagcagacaggagaagagggcttgttcagggaaactcccctttttaaaaccatcagatatcatgaaacttatttactgtaatgagaacaggatgggattcaattaccttccactgggtcgctcccacaacacgtgggaattcaagagatttgggtggggacacagccaaaccatatcaagtactgtgcaagtgttttaggcatgcagagagtggtgggtcttcccagcaagcagagtgtggggaggtaatgggggactggtggctgacttaatggcccaggacccatgccacaaggagatggatggtggatgtgaataggagcctgcttacacccatcacaatttagattcttatgctcgatggcacgggtactcttttaggcccattttaccaatgaggagattgggactaatttgctcgagatcaaaaaagaagtggtgtaggtgggatttaaacccaggatgtctagcactaaaatgcaggtacttaaccactatcctaagggagtggctacttaatttgataaactcatctagtgaatggaagagagacggttacatttcactgatggtactgagcctttgttgatgagctcattgggaatctcagacatgagcaggatgtgtctaagggacaggtgggcttcagtagactggctaactcctgcagtctctttaactggacagtttcaagaggaaaaccaagaatccttgaagctcaccattgtatcttcttttccaggttgtccaataactgcatcacctacctaggggcagaagccctcctgcaggccccttgaaaggaatgacaccatcctggaagtctggtaaggcccctgggcaggcctgttttagctctccgaacctcagtttttctatctgtaaaatggggtgacgggagagaggaatggcagaattttgaggatcccttctgattctgacattcagtgagaatgattctgcatgtgaaggatctgattctctgtctaagaaagaagtctttacctctttaagtagggagcaatgatttcatttttaaaccttgactatttattcagcaacttctctgctctatgagatagtgtaggaatggggatgtggttgaagaatgaaaagaaaagtcagctcccgccctcctagaaattgcatctgccttcacaggtcaaggatattggatcagaccttctgcggttctgaatggagattacacaggttaggagcaggttgcacagtgtttccaattctctataattaaagccatagactttcatgtattgaaaaaagcaagaattgcattcttgacagattctttcattgccttaaaaagaatgactagccttgggagtctgggcagctgggtccagtgttgtagactttctctctgctgagccacagcttcaaagatttgtccttcttgtttccagggatctatttctcagacaataagtaaaggctttccctggcctaatgtgctgtaagtgaatgctactatatatgttccaggcactgggctagagactaatatttaaaagccaggaaatttcctatagaaaatctatatctcagggttttctcaaaagagctgggaactctggatgcccattcatgattccagtagttaaccagagtacaagaagggctgagtcttctcagatgggcaaacccactctggctgactgcagatccaccaagcctattgtcttagaccaggaccctttggcaactcattcccataagcctgtgacccttgctttaaatatgcaggccttgtcttctctcaaaaagcacatcaaggctgcagcgaatgcagatatcaaatgatgaagttaaaaacaaaagctttgctgggcgtggcagctcacacctgtaatcctagcactttgggaggctgaggcaggaggatcactttaggccagaggttcaacaccagaccttgtctctcaaaaaataaaaaattcagctgggtgcggtgtagttcctagccacttgggaggctgggatggaaggatcccttgaacccaggagttcaaggctgcagtgggccatgattgcatcactgcacaggcgacagaattagatcccatctcttaaaaaaataaaaaatttaaaagtgacttcaaaaatctatgctgtgatggagagatttttccttctgtatgattgtgatagctctgtggcctatgacgtcatcaggttctgggcaaagtgtaggttttctgtttctttgtttttgaaaccattgcacagtcctaagaaacatcacattctgggtcctgggcaccagccaacatgaggttgatattttaaggggtctgatgttatttgctgcctgttgatggaacttgggagggcaccagggtttgctcattgcattcttgacagattctcttattgccttaaaaagaatcactggccttggggagtctgtggctggctgggtgcagtgttgtggactctctctgcagagtcatggagccttgttcagaatgcttcctgagctgccctggttggccaagggtaaaaacagccctgacttccctgcaagaaacactgcagctgggccagagagtcagcccatcccaggcatgggtttaaaaagtggaggcttttgtttgaaagccctgctctaattttgtcctcactcaaacctctgttcacttgatctgctttaggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttgggggtttgagaccaacctgaccaacatggagaaaccctgtctctactaaaaatagtggaggttgcagtgagccaagatcacaccgttgtcctccagcctgggcaacaagagtggaactccatctcaaataaataaagaaaataaaaaataaaaatattactaaaacttgtcaaattttaaagcacaattttctacttgaagcggaggaaaatgatgttttcaacccgataatgtatagcttaaggcaaaagcaattcttagaaaatagccaggcaatcaaaacacttttggcttcagtttagaaaagtttaaccccgtgaagttttcagctgtgcaggagcctccccgtacccagcctaaaaggctgcaacacatgtgaaaaggccccttggcccccctcgattccctcaaagtggctccaggagtctttaacagaaggatggagaaggtttacttatggctggctctccacagcgagcagcctctcatgcaggccttgtgttaaaatagtgcttagctgcgtttaataagcaagggatcaggtggtcacccagcaaggtaaaatcctacttccttgaaaggttttgcgtctggctgactcggtggtgtggcatctccctggaccctgcatgggagtgtgtctatgggttttctttgtgattggtgccctgttctgcccagaccattgtttctgatacagcagatcaggaggaaggtggtgttgagtctcctttcgctgaggagtacacacagcagcccatttgcatagcactgtattgggttgtcagtttgtcatctttaaattcattcagcaatgaggccaggcatggtggctcatgcctgtaatctcagcactttgggaggctgaggcagctggatcacttgaggtcagaagttcaagaccagcctggccaacatggcaaaaccctgtctctaaaaatagaagaagaaaaaaaaaagttagctgggtgtggcagcgcacgcctgtaatcccagctacttgggaagcagaggcaggagaatcgcttgaatcttggaggaggaggttgcagtgagctgagatcatgccactgcactccagcctgggcgacacagcgagactgtcataaataaataaataaataaattcagcaatgatttattaaccagttatattaggatgaccagtggattgacaaaacagacatggtcctcaaggtgcttaggtgagggtggagggagaggcaggcagaaaacaagtcagttgaaaaagacatgaaaaagtcttcatccaggcactgtggctcatgcctgtaatctcagcactttgggaggccaaggtgggcagatcacttgcggtcgggagttcgagaccagcctggccaacatggtgaaaccctgtctctactaaaaatacaaaaattagccgggcatggtagcaggtgcctgtaatcccagctacttgggaggttgaa

SEQ ID NO: 5 - DNA, 41653 bp, Homo sapiens
ggggtgtatgtgtatcgggaagcatgtttgtggtggggggaagcagggcgtgtgtgtgtgtgtatgtgagtgttgggaagcaaggtatgtgtgttgggggaagcgagggctgtgtgtgtattgggaagcatgtttgtgtgtggggggaagcgagggctgtgtgtgtgtattgggaaacatgtttgcgtgtgggggggaagcagggcgtgtgtgtgtgttgcgaagcggtgtgtgtgtggtgtgtgtgtgtgcgcgtgtgtgctgggaagcggtgtgtgtggtgtgtggtgtgtgtgtgtgtgtatgtgtatgtgttgggaagcgggttgttccgaggactccccgattccctcctggttgaaacgtggcccagaaagagaggcacactggctagggcacacagccagggccgtccagccccgcaggaagccccggtcctgccctgtcccaggtaagcctggccagccctggagccggcgctgcggggctgcggggctgcggggctgcggggccacgggacaggagcgggtgctgcggtcccggcagagcaggggcggggtccgggcgggggcggagccgagcgcccgcgcacccggagccgcagcccgagccggagccggagccgggagtcgtggcccggagtgggccttggagtcggcgcgcaggcggctcgcggtgagtgctgtgcccagcgcctgggggtgggctggggggagactgtgggcgccgagggtcccacccccgggacttgaacacttggcttctgagccgtcctggccgcagggcttgggcaagggttgcctcggtttctcctgggggagtcccagaatgtagatgggaagtgagacggaaagggggcatctcgaggttggggaggcccctgggccactcttttgatttccttcctctctgtgggactcagtttccccagcttcaccatgccagtgagtgggtgggtataagtgagagagtctgcctggaggatcctgctgctgctctctgtacctcttatgggaatctcctgcctcctccttctcccttgttgggtgtgagatctaagttcaaatccatcccttcccagacagcccacaccaggacctgcgaggccacatcccccagctgtttgctctcctatgcttctcaggttgatggagcagccatctggtcctgctttcattccaacaaactgaaaagccctccaccccaatccccctggggaggacctcctgaagctgcatgagggctgggggctggacccctgcttctgagtttgctctttctgagcctctcctgccatggccttggtttccccatgggctgtgacctagcctcctccagggtccttgttctacagaggggaaacacctgccctgtcaccctcccctgaccccttagtgaaaaagaccagagtgacatccccaccacctgtacttgcctgtcccgatcactcagccatggggctgctggggcccctgagtttgttggctgctcaaggcccctttgcctcgctgcaccctgggatctgaggctggaagcatcctcatccagcccacctccctcctacccagtgcagagcccctctgtgacacatgcccagtggtggggagcttattactgatgaggtggctttttagtgccttgcagctcttgtttcctttagctaaaatctgtggcttacagattctacaccttggttccatgtctgtctcccggagccatgcagagcaagtcagttctttcattctttccagcagtcatcattcatgcactggatgagacacgagacagagcagtaggaaaatcagacaaaaccccctgccctcatggagtgcacactctagaaaggaatggacagcaatgaaataagtaaatacatggcgtagcaggtggcgggaagtactacgagtaaaacaaagctgggaagcaggatggggtgctggggctgggtattgcattgtatatagcacaaccagggaaggcttccccaagaccacgatagctgggcaaagtcataaggaattgaggggtgagccacatgagtatctggggggctgggaggctttggttttcactctgagcatgatgggagcacgggtttggaacagaggagtcacctgctttgacttggggtctggcaggcccatctagctgtggggtggagaatggatcatggggggggtggggtaagggcagaaggagagcccgttgagcggatcattgctgtcattctcgggagagaccatggtgtcttgcccatggcccagaggtggttaaaaaaaagaaaaccagacattcagatttgggatatatttcaaaggccgtgtcgatagggtttggggtgcaagagaaaggtagagtcaaagatgattctgagtgtttggctcgagcttctggaggtttgaagctgccatgaactgaaatgggagttgcttagcggagagatcaagagtttggttttgtgcttgtggagtttgagctgcccattaggcatcaatgatgagtaagcagttgggaaaagttaagggcggaggttggagttgaaaataaaactttgggcatcatcaccatataactggtatttaaagccacaagagcaggtgggctcatctagggatggagtgatatggagaagagaaggggtctaaccattgagtgctggggcccccagtgttaggaaccagccaagaagacagaaagagtgaaaatcagagagttggggtgtcctggaggaaatgaagaaaatgccccaaagaggaaggagggaacaaatatgaccaatgcccctggcagagcaagcaggctgagggctgaggattgagcaatgggaggtcactggtgacagtttcactggagctggatggggaactagagggaatgggaggggatgggaggacttggggacagcagtacaggcaacagacaagggggcctgctgtaaagggagcagataaatgggattggagccaaatgaagaaggggagtgtcaagagagtgctttacttttacaatggagaattagagtgcattgtgcactggtggggggatttgatctcttagggagagaacagtgttagggagggagaatgcaggatagctgggggagggtggggggcttggccccagcagagactcaggacacttgggaagttgagcttccctgggcttcccctcctctcctgtctgcaaggggtcagtgggctgagatttcagcacttaagcaaagcatttgctcttggccccagagaaaccgggctggctgtggtctcaggaaggaaggaggtgtccaggctcaggcctgggcctgggtttcagggagggcccacgtgggtcgccccttgaccctctctttcagcaaggaagtgatcctttctctacatgggcctcaccttggggaggacaatggtgtctttgaagttgtagtaactgaagtagagatcaaaaggcaatgcagatagactgacagatttcgcctgaagaggggaagcccgaccaggtaataaaggagtaagaggaaggatgttaaggacaattttaggaaacagataatgagtgaatattttttctctctctttcccaatttaaactgaagcaggagaaactgaagctagacataatgattaacttcccaagctggtgagcttcctgagctggttagtgagaacagcactaaggccaggttctcctccccagatgtttaagatgagacaggacaatgcctgctcagagacagggcctggctgaattggccctcaggattctctctgctctgaggtttctggaagaaggccagggcagaggtgtggtgatgtagctgctgggaggacagagctccgagtcacgtggcttgggcgggcctccccttcctggtgtccacagaagcccaacgtcactagctggggtgtgtatggctcacacgtaggccaggctgccctaggcttggtgtgcaagggaggggcccctacttacttgtggcctgtcccctcgtgaatgtgtctcatgtccccagtggggtttttcagtgagggtcatggtctccaggatgcacaaggctttgtgccagaattgcttggaattgcctagttctggaaggctggttggccaactctggcctccggcttttcctttgggaatttcccttgaaggtggggttggtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggtaagaggagcaggcattgtcccgtcccagcttgatcctcagccttctttcatccttggccgcgacatgctcccaggcctggggtcagatggggagtgctgactctgtttctgggctgttttctggggagaatgggtcggcgggtttttttccccaggacctgggcagggtcaatggtgggggccgctgtcgcatccttggctggtgtttccacagctgagaaccactccagggccaagcccagagcttattctacccttttttgtcctctcttcccctgtcctcggccaccccaccctcttggctcctctgcttagatgtgggcacaaggaggagaactccttggcctgagagaactaccttagatcctggcttccagtggcctctgcaggggggtacaccctctctcccaagcagccagacacacaagtaacctcattgcctcagtttccccatctgaccagcacagggccccctgtgccccagcagcgttctgagagattggagctttctccttttgcttaccttggctaccgtatgaggacggatacagagtgttccccccacccccagcccaggggatatttgattcatgaacattccctcagtgtctttgtgggggacaatgctgtgccaggctcagggatgccaggacgagtaagacccaggctcccacgtggcccaggcagggagagagacacataaacaaccatcaggaaagaggtaaaatccccaggccacttggcatctgctcccttgagtgtctgggaatgtccctgatttataaaaagaagctgacggccctctttgttgtccatgcctacaccctttcactttcgtttcttcggggcactgcagcagcccttgtccacagaccccatgacaatcgcagaactgaccatgctgagagattttcttggctgctcagggaccctgccagggcttgaagctcctggagggtcacttgccctcaaattcccagaacgcacagcaggtcactgatgatagcagtggcagcagtctgtgcacggtggtttcgagggcgtgggagggaggtgagggccctagggcaagtgtgtgtgggaagtgttgatgggggacaaggcaccagaacgctcggaaacaacttagtttgcaccgtaatttttcacttcgcctaggacaggacctttagagcaatattctgagtctaccccttggagtagcagtgtgcaaaacacacagcacgggcttggggcccccgtggggaacccaaatgtaagagttagagacatgcattccggagtcatacatggctcgtgttgaaatcctgactctgcctgtctagctgtgacacatcgtacaaatcacttagcttcttggtgcctcagtgtcttcctctgtagaatgggtagatcataggcactacttcagagtggctgggagggttcagtgaattcctgcaggagagcacttagaatggcacttggtgtgtagtttatgcttaattaatattagccgttactgaaactgctgtagcctgaatccagccagcatgaaagagcccctctcaccctgcttcgaagagaatgaattccctgattgtttggaagatctctctctctctctctgtcttttttttttttttttgagaaacggtcttgctctcttgcccaggctggagcgcaatggtgccatcttggctcactgcaacctctgcctcccgggttcaagtgattctcctgtctcagcctcctgagtagctgggattacaggcgctcgccaccacgcctggctaatttttgtatttttagtagagacagcgtttcaccgtgttggccgggctggtctagcgctcctgatctcaagtgaccttgggagatctcttgctcctaatattacctcaagcctttttaaacgttttaagccggagaccaagcatggatatgggagttaggggtcttgatttaattcttggttgcttcaaactctgtggaaccttgaggtgtttcttgccttctctgggtctcaattttcacatctatatggtggggagcttggattgggtaatgtctgaggctagaaccatggccaactcgggttctgctggggctgacttgccctggccttccctgaccaccctgcatctggcttctggagaagtcccgcactgaccttgttctcctccccaggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggtgaggcactcctggtgtgcatcacagagttctcaggaaaggggtgcttagtcaccaagactgatttgtcctcatgaagtcagcctgtggggtaacttggtccatgggatttcccctaaaaaggtagccaggcaggtaaaatttgctcttgactcttggcaggaaacgtacaactctttctttcttcttttcttttctttttctcactctgttaccctggctagaatgcagtggcacaatcatagctcactgtagccttgaattcctgcgctcaagtgatcttctggccttagagtagctgggactacggctgctgtaccaccatgaacagctaattttttttttttcttttagagatggggtgttgctatgttgcccaggctggtctccagctcctggctttaagcaatcctcccgccttggcctcccaaactgttgggattgcaggcatgagccactttgcctggccaacagaacacttctgccgagaggaagtgtgtggtggccaggaactcagattctggagccagaatggtgcaggctcaaggtcaaccctgtgtgatctcaggcttccctatggagcctctccagcctcagtctcccttgtttcagtttcctcatctacaaaacaatgttaatagtcaaatggtgcctatcctataaggctcttgggaggattcagtgagttaatttgagtaatgcttaggatagtgtctattaccactggctgctatttattatttctgttatgagtgatactctgtacttgtacacttttatttctgtctgttttaaattaacagcacaacagaccataacactgcagtatattgaatttattttataattaacatagcatattataaactaatatagcttaaatgtttatgtaggatttctgacatgaaattgcattagatcatagatgttcagagttggtatataacagcccctgagaatgtagtaactcagcagagaccagaaggtcagagaaatgaccactgagtatttttgaaactcttttgttttcttccaaatagtgattcttagggctcctgagaggcagatggaacaatcattaacattccactttataaatcgggaagttgagaccaaggaaagtagtttgaataagctcacagtagttaatgagggggccagtgctggaccaattggccagcactggtcattgacttattcatccatcattcatttattcagccagaatctattaggtgcttcatacatatttgcttaaagtttgttgtgttcatagagctttgcacacggtaggtactccataaacatttgttgatgaaataagtgagttactgaatgaatgattgaattagaatgacactgcagtgttaaaatgggctgggttggggaacattttagtttttgtttttgtctgttttccaaaaatgtatgtgttgttcacatgagtctggataaccctagattgagattgatgacataaataaatttgtcttcaaggctgcactaaagctggctcacatggctaggtatttacagagcagaagtggtgcagtcctctctgattagttgcacgtacagaagacatattcgttattggactgaccttagtttctcttataatttgttaggggaattgaatcagcccatctgagaagttacaagattgtgtcttgtcatctttaaaagttcagcaatgtgatgtggtacagatggtctgaggggtttggagaaggtagcctagatccctagggcccagagaagacaggatgtgaacagaggaagtacatggattggtgaagaaaagaaatgggataactcatgggtcaaagaagaaatcatgatggaaatcagaaaatattcagaaccatacaataatgagaatattatttatcaaaatctattggatgcagctaaagcaggacatagggggaaatttacaaccttaggtgcctagattaggaaagaaggaaggcatttgtttatttatttgtttatttatttatttgagatgggggtctcactgtgtcacccaggctgctggagtgcagtagcacgatcataaatcactgaagtctcgaacttctgggctgaagtgatcctcccgcctcagccttccaagtaggtgggacacaggctagcaccaccataccaggctaattttttttttgtagacacagggtcttgctatgttgaggtctcaaactcctgggctcaagtaatcctcctccctcggcttcccaaagtgctgggattacaggcatgagccactgcgcccatctaaggctgaattttaatgagctaagaattcatcttaagaaagggctaaatagacagcaaaagcaaacattgaaggttgggactgagctgagtgggtagcagggatgggagacaacagatctgaggagagcaggagattttgaaaggattgcactgcctgaggtttaagcctttagaatccagctctctctgagctccctttgagctctgacattctgtgactctgatttggtggccttcccttagtggccttactgatttcatttggatggtgcttgtggtatatccaaccaacatgtcttcccaaatggccttttaatttcctataaagaagtagttgtcattgattgcaggttagggacagaaaatgctgtggaatgaaacaaaatgcaagttaaagaactaaattccaaaaatacccattgctactattgactgagtgaattcctactgtgtgccagacactgtacccagtccattccctgtattgttttatttaagcctcacaagggtatagtgtgactacactgtttcttaacaatgaagaaactgcccaaatcgcccatctgggaagcggcccagctagaatttgaatccaggcctgttttcctccagagcttgtgctattctctgtctgtcataaaatgtgggggctttgtgtggtaaacttgctcagttgggcatagcagttgttaggaaacctgaggctggtaacaccagctgtaataccagctgtccgtctgactcatgcaactgttaaagttgatagggctgaggtgtcagactgagctctgaattgcctgattcctataacaatattaacttaaacattttttaaattgggaaatgcaccatgcatacagaagagtgtgtatatttcatatgtatagtgtaaactgttcccatcacccaggttaaaaaacaggatgttgccagtacctggggccttctttaactgcaactgctagaggtaaacactggcttgacttttgtgtaaatcatctctttgcctttctttaatgttttagcatcttttaaaataaatccccaaataatgtattgttctattttgaaaaactgagtagcaagccaaaaatagctgtgtaaagaaaggtcacttaaattaggctgggtgcagtggctcaagcctttaatcccagtactttgggaggctgaggcaggtggatcacaaggtcaggagatcgagaccatcctggccaacatggagaaaccccgtctctactaaaaatacaaaaaattagccaagaatagtggcatgtgcctgtagtcccagctactcgggaggctgaggcaggagaatcgcttgaacccgggaggcagatgttgcagtgagccgagatcgcactgcttgaacccgggaggcagaggttgcagtgagccaagatcgcaccactgcactctagcctgggtcacagagcaagactctgtctcaaaaaaaaaaaaaaaaaaaagaaaggttactattgccttttcttagatgaaggttcccaaggcagggaaagctaagtggagtctcagggacttggtctggcttttccttccctgggaatttataaggacctcttctgggaagtcagtcggcaatgccatgaatgagtctggggaaatattgggctcattgcaactggagggtctggtaggactgatgtgaattaggtgctgtgtccggaggaaaatggccagaggaagtgggctgctttgtacagtcagtggtaaagttgccaaaggctattatagctcacaggaatgggccaaggctaaacactcctgtggagtgaaatgaatgtcctcagctgactgaggcagcgggagttgaaaagaaacgatattagttcatggtgaagacaagtcaaatatagataaaggttagggtcaggcttgcctggacatctaggagataactgccctcaacttgtttgaatcttgagtcactgctccattttgtttgaactggtggccatctacttatagtatacagccatcaacctgagatttccctacatggtcttcctgccttggtctcctgtatcctgaatcctatggcctcttcttccctggtttactacattttgctagaccgtatcctccagtcaattccttagaatgaatgtatgaaagttaaaatttctgaggtctcacatgtcttaaagttccctcatactggattgatagtttggctgggtataaaattctgggctggccatcattttccttcagaattttgattgcattattccattatcctctcttttcaatattgcttctaagaattccaaaacctttttttttttttctttttgagacagtgtctcactctgtcacccaggctggaatgcagtagtgtgatctcagctcactgcaacctccacctcctgggtttaagtgattcttcttcctcagcctcctgagcagctgggattacaggcacccaccaccacaccctttagtagagatggggttttgctatgttggccaggctggtcttgaacttctgactttaggtgatctgcctacttcggcctcccaaagtgctgggattaaaggcgtgagccaccacacccagcctccaaaaccattttaaaactctttctggaagcttttaaaattttcttttagtccccagaattttaaaatttcaattatgtgccttggtgttcttccattatgttagtcacccaagaggtactttcaatctggaaacttctctatgttttgggaaatgttcttgattagtttacaggtgatttcttcctctccattttatctcttctcttttcatgaaactactattaattcaatgttagaattccttgactgatcatttaattttcttctattttccatctctgtgtctttttgctctacttttctatgatagtcacagctctatctttaaactcttgagtttttcatttttgatgtcatgattttaatttgcaaggggtaggtttgactgattcttttttgtagtatcttactcttgttttatggatgcaacatcttctttgacttaaggatcataagataggtgggttctttgtttgtttgtttgactgtttttcaccctatgtaaactttttctacaagtttctttccccttcccccctttttggcttctatctcccacattagatgctttctctgggctcatgatactctttggttttctttctcaagattgacaggtaggactttaaaacttgttgagcatgcgggtgaaacttgtctaccatgaatttcactgtagatattttggagattgacagtgtttatatctttagatctcgcctcctgggttgatcaagttatctgagtacaccacagaccttttgcctggggataaaccagaaatctgtttcagaaaccactttgattcagtcttccttgttttagtcatttccttcagttccggaggtccctcatgctgatcattccagagccctttacagatcctagggtacacactgcatggttttcaactttcttgttttggggttaagatttggctttcaggagtctcctcagtccgttactattcattcaatcagcaagtccttgagcacctgatttgtgccagacattcttctaggtgttagggatacctcagtgaacaaaacagacaaaaatctttgtcttggaaatacacacactccagtcaggggagagggacaataagccaaaggaaggaaattacagcgtgtgctagaaggtgataagtgctgtagaaagtaagtaaagtgggtttgggagttgagagtttgggaaggggataaatgatggcaattgtaaatagagtagtcagagttctcacttagaaggtgaaattcaagtaaagacttgaaggaggacagggaattagccacatggatggctaggggaaggcttccaagctgagaggacagccagagccaaggcccagaggcaggagcatacctggtagttttaggaaacaggaggccaggatgctgagtggagtaagagggggcatgaaaggagaaacttgggtccacgtggttctagacaggtatttttgtctgttttgggccctgaaggttactattggacttggactcttactctgaggaaatagggacgctattgggacgtttgtacaggagcaatgtgacctgagttttgtttgtaaaggattagactctggctgtggcattaaggctaggctgtgggggcaggaacagaagcagggggaccagttttgcagcctgtgcagctttccagataagcagggattgtggcttggaggaggatggtatagaggaggtgacaagaaatgactctatgtctggtatgtagatattggccacagatggcatttgagcactagagacctggctggtccacatggagtttccataagcacataatacacatcagatttcaaagacttaatatgaaagaaagaatgaaacgtaccttgctatttctttcttttttttttttttttgagacccagtcttgctctgtcacccaggctggagtgcagtggtgtgatctcggctcactgcaacctccgcctcccaggttcaagtgattctcctgcctcagcctcctgagtacctgggactacaggcacctgccaccacgcctggctaattttttgtatttttagtagtgatggggtttcaccatgttgtccaggctggtctggaactccggaccttaggggatctacccgccttggcctcccaaattgctgggattacaggcatgagccaccatgctcagccatatcttgctattttctacatggattacatgttgaaatggtaatgttttggctattgtggattaaatagaatatatgattaaagttgatttcatctatttcttttaactttaaaaaatatgtctgttagaggatttgaaattccacatgcggcttgcatttgtgacctgcatttcatttctgtggaacagtgccctttttgggacatgctttgaaggtggagtcaacaggatttggcagattacagacgagaggcttcaagggtgactccaagacttcggggcagagcacctggaagaaaggggttaatattagccaagatgaggaaggctgtcggtttggcaggtgcatgggcaggttaggagtttagttttgaatatgttggaggtgtttatgaaacttttaagtggagatggaaaataggcagttggatgtgcaagtccagggttcagggagacagttcaggctggagatgaagatgtgggagtctgaggagagattgtattcaaatattcaatccatgagacttgatgaaatcacttctcttccaaatgatttacagcctgcagaatcattttccctatctttgtaggtttatgtcttcattttgtttcatttatttttcagttattcactgttttagtgagttttgagtaggagccagattggatgcatgcgttcaattcaccatccaacactgtattaactacttgaaactcatgtggttgttcggttgtttttttgaccttttattctggatggaagagagatgcttatgaagttgcagtaatcagtaagccttcccacattgctccatcagccttcctggaagaataatgtcttctgcctttcctgtaggcaagaaggctgcttgatcttgccacggtgaaagcgaatggattggctgccttccttctacaacatgttcaggaattaccagtcccattggccctgcctttggaaggtaggtgtatgttctcagttaatcagaaagggaagggcagtcagtgcagatccatggttaagagcagaacacacctcggttaacatcccatatgctggcagtatagcctccctatgactcaatttccttgttttaaggctagcaccaccccgtctcattgggattttgggagcattaaaaggacaaaagcgtgtaatgttagctattagctttcattatctcccacacagtatactgacaattgggctaccatatattgagggctaactaaaggtgttacttaccatccaaactctcattatctgtaccgaaaagatatggacacatgttttgagttagggctggtatctcttgatctctgaaatttagcagctcacaatgggaaactcaagaaccaagtggatctagagactctggtatccctcagtgcccagggtcaccacccaaactcaggaacaggaggggcttggaccgcaccacttgaacataccaggcatcctgccaggtgctttatggacaatgtctaccctttgcaacaaccctgagaagtaggtggtgtttttttccaccttatagatgtggaaactgggcagggaggttaagtgacgagggaggggaagatgggtctgattgtaaattgtccccacctacactttctcttttcttgggagaagaaatgtcagttgtaaagagagagtgcaagcctggcactctttagggcttgttcctacaccactgtagggaaagctcattggcactgaagccccctgagctgtgtgtggtgctggcagatgggtctatcaccctgggctgtgtcctctgggcagcaagcaagcctgtgggcggggtggctggaagtctgtgcctggcactcgcgagtgcaccgtctcattgaagaacaggatctaaacatcagtgcgccacagcagggtgcgcggcacggagtgcaggccctggtttggcccttggttgaggtttgctgttgacatcatcaagcacagctagtcactgtaagaccaggccagggtgcaagattccccacacttctaaaggtgacaattggtgtgtttatttctctataaaatgacattttttttttctggagaattttagtatcattggtgatgactggaaaacctgcatcagaaatcaggtcggaagaggaagatatatatctgatatgtactggagaggaagatatctatcttatggtctaagttcagggatcctggtatattcagagggcagaaagctcagcaataatcatcaactctgggaacagaggtgacataaacacagggcgtcccctttgtgtgactgcagatagtcatcagtgagctcagagctctatgaaaattacttgctagtttttgggttgaaaatagtgggccagtgtttggttgggggcagtgaggctgtgatggcgggggaccatgccaagctcctaccagcctgggacgctaaaccagcacttccccatttcctgaaaggggaactaaactctgacacaggaaatggtttgcttgcattactttcaggatgagaaaggaagagcactggccttccaaacacaccccgtgcatgaaaactctccctgcatggggtgcatggggaggatggggaagtggaggcaggatcacagactcttgttcgagtgctcagctggggcaccccggtgaccccgaggccttcccttgctaggtccacccagatcaatcaggatcatctccccatctcgaagtttaactttatcacatctcagagttccttttgccacgtaaggtaacatattcacaggttctgagaatccggacatggacatctttgagggtctattgttgtgcctactatatccatgaataataatgataataagcaccattttttgagagtttgccatgtcagatattcttttaaactgtattttatctcgctgcctcctgaaaaaatccttccaggtgtatattgtccccatttttacagatgagagaactgaggcccagaaaggctaaatggcttgcccaagtgtatggtggacccaggttttcaaactcaggtgtgtctggcttcagagactgggctcctgagcccttaagccctttgttcccctttagaaaaagtcacctgaggctgagtggtgaagggatttatccaaagccacccggccactatggcaggacagatatcagaatacaggtcttccgatcccagcccagagccccttcccgtcatctagaactcctcctggtgtcagtaatgataacggcagtcactgatgtcttttgagcacttactttgtgttgagcacttacactgtgctaagcacttgacataggtcatcttagttgatccgtgtaaaactctgtgaggtagtgaccaacatttctcccaccttacagaggtggaaactgagggttaggaagtttccttgactgtcctcaaagtgcacagcttgtgaatggaggagccaggatgggcgcccgctggctctcctatcccttcagttatgtcagcgtcccccgcagcagcccattgtctggttaggtcccgtcttcaccatggtgccaccttcatctgcctcttcttctgccttccagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtagtgagtgttactgggcattgctgttcaggtatgggggagcaccatcaaggctaagtgtgggagcaccgagctgggctctagaagtctgggcccagcttcgcctctgccaccctgctttgcaacactgcccagatcccttcccttctgggccttaatttcaatatgtgatgatgacagccacactttattgactggcctatgtgctgggtctggtgctatgctttccggaatgacctcatctaatctctacaaccaccctggcgggtaggcaggaatgttattatctccattatccttgacttgaggctcagagaagtgaagtaacttgtccaggaaatggcagagctggggttcacaaattgcatcattctgattacaggttttctgcctcccaccagtctatggatacacttcagaggctccctgaaaaccttgaggtcacttgcagaaagttttgtgtagtatgtgtccgtatcaggaacaacaccaaatcagaggtgacttgtgccccatcagagactttaacaccccaaccagatgggaatttcaggacccaagaaatagaaagtggctgcagggttacaactactgttggattcctgaggtagcacagtgtccaaacaggatttcagcactacccgtattgcttagagccccagccaaagatgtgaggttttgccctttggagaatctgtgcccctgaactcgggggcctctttccacatcttgggggcaggcaagggcagagggtgtgcctaggcctgcggatcagcatgcgacagattccccaacatccttccagcttgaaaggggattgccctgcttctatttagaacctataggaaagcagaagttctagattgaagttaaaattgattcccagcctccaggggctttgggctacacctggatgaccttaattgaccctaagcatgggacaaaccacttcctgagagtattaggatggtatacatcttctctgggggcaaagcaacaagatttatttttcatcatggaccaaacacatggatacccactagaaactgtgtagtgaattttgttaaccctgacatagggaccatggtctttaggttaaagcataataacaacataatacataacatatatagcgaatatatatatgtattatatgcaatgaatgtaaatatgattatacccatcatggtcttggaggaaacagatgacacacttaaaatgggtgttttgaggagagtttgaaaaacagattgtttacaagccatgggcaggagttaggaagagtgagagggttggtgcaggggcctggggttagtaacagctgggggagggtagacttgaagggggaaggggagggagactaattagctggggggaaggtatggagacggctgcctgagcttctgcaaagtggaagaataccgcttggccctaactcctcaccccaactcttgctcctggccagcgccttccaccagctggacccatcagggaggccgagtgggctgtctgctggagtagtccccaggcatcagcctcccaggagccagggacgggtagagaagggggagagtggatctggccaggcaaatggaaaacagccagcaccaaactctatttccctaggagggaggatcatgatactttgagtgggaatttggaaacctgtctgttggagcaatttccctgatagaaataagaatgtgcattttcctgggtagtagactcagtttttaccccaagaggccaggcatcactggcctgtgtgatcctcataggccagtccatctctggaattcttgaatggatcatccatccttgattagggatgtccccgtgattaccagggtgtgcagaagggctctgggaaacctgtgggtctgtctctgtgttcagagaaaggtgagggtggcctggttctagctcatggtgctcagactgtggtgtgtaaaggcactcgtggcaatgcagattcctgggcctgcctctagtgattcccattcagtaggtttggggtggggcccaggaaatctatatttttcacagacacccctggtgattctgatacaagtggtctcgccctgggagaactactggtctgcagcaaccagcttggttttccattagcaattactgtccttgagcgagttttactgctcttcaccttacacacactaaaactgccaaggccgtaggggaggggaagcaaccatgaggttgctgtgagtgcactgtgtgtgtgtgtgtgtgtgtgtgtgtgtgtgtgtgtgtgtatgagagagagagagagattgagaaagagaggaagggaggaagggggagggcacaggctcctctcccacagtgccaacctgcctctctcccacttgaagtgtttccatgccaactgaaatcctcagcctctaggaaaccctatatacacagtgcccctatataggtttctttagactctggctctctcagactctagagtgatggctttaaaagttttttgaatgttacccacaatgattgagaaagagagagagagagcacgcaccaccatgtaaacatggaacctaagtttcacaaaatgacttcgctttatgaactctgagacactctgctctcttctgttctgttctatttccattttagaaatgctgctcaggaccttcaaaatgatttgcatgacctgcaacctgcagtctgaaaaatcactgcactacagaagtggccataagaggccctgagggagaagctgcacaatgtcatggttaagagtggggtttggagccaagccgcctaggctcaaagcctttatgtgccgtacaaccttggcaaagtcacttcgcttgtctgtgcctcagtttctttctcacgaatgctcataataatggttcccatttcactggcttgttgtgaggatgaaatagtgttattattgagaagtggtaagggtagtgatcagtgctagcgatcatgattctaggtgacttttactgtgtaccgggtgctcacaaggctttatgtgcacagcctggtgaggctgataatactattgttccctctttttttttttttgagacggagtctcgttctgttgcccaggcagggggtacagtggcacaatctcggctcatgcaagctctgcctcccgggttcacgccattctcctgcctcagcctcccaagtagctgggactacaggcgcctgccaccacgcccggctaatttttttgtatttttggtagcgacagggtttcactgtgttaaccaggatggtctcgatctcctgacctcgtgatccgcccgcctcggcctcccaaagtgctgggattacaggcgtgagccaccgtgcccggcctgttccctcttttatagatgaagagaccagcaaataactagtaagtcgctgatcaggatcacaatatccagctgaggcactccagagcctgagctgttaaccattcagtcagggcctcccaagtttgcctaaagataaagaatcatgtgcacagttgttaaaatatacagattcctgggccccaccccgcagatacttgattgccagctccagggtatgggcctgagaatctgtcttttagggaagctttcagatgatgttgtgatcaggtgagttttgggaatggtgccccaagaggagtggcagacagggcttgctcggcagggactagcctgttggagtggtgccattggggttaaggactgggcagcagggcctcactaaccacagcctatatgcctgtttctgaagttttggccactctcatccagctggtctactgtctgctgacctagatgatggtaaattgtccccaggggtagcctgtctagttcaggctgcacctttcgcatatatcagctcctttccaccatcatcccctttgtgaggctgctgtgattatcatgttccttttgcagagatggaaacattgcctcaaattagctctgtcatttcctaaggattccagggttctttagtagggggtctggatcctacgtcctgggccatccccatcatagtgcaccacgtcacctccctggccagggaccgtggggtctccacttttttggggtgctccatctatgcagggtttcctggaagcacagatgctggcacttcagggatgaatgaaagtctttttgggggatttgtagatttttttcttgtcttactagctccattttcaaatgtatttattttgtctctttagtttgcgcgataacaatatctcagaccgaggcatctgcaagctcattgaatgtgctcttcactgcgagcaattgcagaagttagcgtaagtcagcctgggctgtggacaatgggctccaagtgccctggtctcaccccaggtcgtgcagcctgggaagctgtgagtgatgggctggggcaggggctgtttgcatgatggggggtgcaggtgattcctgcccagaggggaagggcaaccctgggatttggtgctcactgtccaatgtgctttgcttctgtgtctcctctcttctggaactgaacagtctattcaacaacaaattgactgacggctgtgcacactccatggctaagctccttgcatgcaggcagaacttcttggcattgaggtgagcccaggttttccttattccctggaaactattttttgccccattcctgagtcagtctgatctggtcttggcctggcactgcccacactggctcctgacctcctgattgaatgcagggacagtgtctcattttaagcaggggttctctaatgctgtgatctccccagtaaactctggactagctctgctgaggacttcctgtcttttgacctttagcccgtagggcaagaaagcttttctaggcccctttccttttctgtgtctaagagtgtcacagctttctgggtttactgagttccacgatgcatgttgagctcgtcctggtgggggaggcatacacagttacttgccaccccagctgtggcagcgagttgctgcaacactcccaggaggtcctttcaccactcagagcatgcaaggtttgcagtccatctggttctgcatttctgctactccagtgtctcccagtttcaacaggagtctctctctctcctacctgatgcctttaaattgcccctctagctggccgctgggttggcctggcttctctctccttctctctctctcagatattcttgcctcctgtgatttgtgaggcagtaaaaaaagacaaagtaaagaattgcttccatctattcttttacctcttgggctgggtttgtggatgggagccgccattttaaaatggcgggccacatagctcagtctcggcaagggctactgagatcagaaccacaggtgccaatttgtacaaaggactcagtcctgctaccactgcctgatccctcagactcacaagcctggaataggctgtggccagacctggctggcccatccctgagaagggtgctagtttcagaaatggaggctgagtttgtggccaacacagtagtcctccggtatgtgcaggagagatgttctaagaccccagtggatgcctgaaaccatggagagtatcaagccctacacataccatgcttttcccaatacctacacacctgcaataaagtgtagtttataaattaggcacagtaagagagtaatagcaactcataataaaatagaacaattataacaatcaatgtactataataacactatgtgaatgtggactctctccatctccctcaaaatatcttcttgtactgtactcacccttcttcttgggaagatgtgtggtggtaaaatgcctgtgtgatgggaggaagtgaggtggatgacgcatgcagcactgtgctctagcgctgggctgctgttgacctgaccacacttcaggaggagaatcatctgctcccagagatccctaatctttgagcaacaatgaggtcggcagctggatgtcaggagcagacgatcttgatgattaccaaatgggagcgtatagagcgtggatgcgctggacggggggctgattcacgtcctgggtgggatggagctggatggcacgtgatcagaatagcatgcaatttaaaatgtatgaattgtttatctctagaattttccatttaatatttttggactgcagttgatttcagataactgaaaccatagaaggcgaagctgcggataagcagggggcagggattaccgtatatcattgtaatagagagcacaggctctggagccagactgcccgaggtttgaaccctcattagctgcgtgacctcaggtcagcccaatgtctgtgtgcctccgtttccccttctgtagaatggaggtaataaccctggctacctcacaggctgtagtgatgagcaagcaagttaatccacatgaagggctgcaccgtctggcaggggctttatatagtaagcgagtggctgaaagatgatgggtaaatcacacaagcactcagcttgtttctccttatgtgagtccggtcctccaagcagggattcaatgtgccacccatttattggggaaaagtcctaaaaggggaagtggggaagggagctgggggaggctgggaggtgtgtccctgagtgaaggagagagggaaggaaggaaggttgagactgggcaccttggacttcagtgcagtcctaagacatcttggcaaggctgatgaggagttcttgaaccaaattcaccaggcaggggagcctgatgtctcaggcaggggctggcaagtgcagatgcgaggatgttagattttggagcacagcagctggggcccttggctacctccaaggagctgaggctggagacctgaaaggcgagttctcctagctgccacaccccttctccaaggatacaataatatctgccttataggattgttgtgagctgagtggcttgacgttccttgaaagaatgaaagcgtatagttatcccaggaagcctagggttgcaggtgagagctctggggcttctccgaagctctccgaggtgtctggattcagttgcagcaggagccttccttgctgggatcttcccccacccctagccttggccctccctctctccttcctttctggaaggctcagtgggccccacccctccctccagccacctggacctgcccagcgctcttgtgcaacaggtaaagcctacctgtagcaacaacagatctgggaaggctgcagagggcacgatggggtctggatcgagggcggctgagaccagagggaaaggtgtgaccctgagtcaccctcgctgtcccggggaaaccacctcccaggacagctgcctactgtggctcctgcctggaattgtcacactgctgtgcaaacagcgtcccgctgcccctttccctttgctgggggaaaatgaagttgtgggagccgctgagtaaactagacctagcagcgagggcacctgatgtggctgctgcctcccgggcaggtcttcaatgctttcttcctgtgtttccctggccagggcacagacggccctccttttctgcctgccgctgtgttctctcagcctcctctgtcttcccttccaggctggggaataactacatcactgccgcgggagcccaagtgctggccgaggggctccgaggcaacacctccttgcagttcctggggtaggttggattccaggaagagggacctgcatggaggggcttgggacttttgaggatttaggggcaggtgaaactcttcagccaggaggccccagaggcagcccagctccagtggggaggacaagccagggagagagtgggcggcccttgactgccaccttcatacttggtctatgcctgacaaacaggaagtttgggatgttggggctaggggaggacagtgcccacgagctggtgacaggaagccctctgatcctcagggggcgctagggctgtactttagctgcatattaaaaccacctggaagcttctaaacactattgccaggcctcccaccccagactgatgaaatgcaaatatctaggtgcaaggcccaggtatcaggagttttaaaaagcttcccaggggatgtacagccaggggtgaggacccctgacctaagaaagagaaggaaatggggaaggataggaaggcacccaggataagaggggctgtgctaggtccctcggagctcttgctccctgtaggaccatgctagggcctgccagggaggggagtaccccaacctgcagccccagggtgggcttcctctgtttgctaggcacccaggcttgcacctgtgctgtttccagcagcctctctcctatcctgtcatgccctagtgtgaactggagtccatttgacaagaactgggagttttagaacctgggactgtaggaagagagaataaccttagggcctaggtgttccagcccatttcacagggaggcaagttgcccccaagctcagttttttgttttgttttgttttgtttgagatgtagtctcactctgttgcccaggctagagtgcagtggcacgatcttggctcactgcaacctccgcctccttggttcaagcgattcacctgcctcagcttctcaagtagctgggattataggcacccaccaccacgcccagctaatttttgtatttttagtagagacagggtttcaccatgttggcccggctggtcttgaactcctgatctcagatgatccgcccgcctcggcctcccaaagtgctgggattacaggtgtgagccaccgcacccggcccccaagctcagtttgagccacaaatgggactatgttgctctagaaatcaacatcttttccacactgcattagtagcaacagagtctagaacaaaggaggccacagccccactgaactctcttctgcttgaggtcacatctgccacatcaggggtatttacctctttcaacacatatttattagggcacctgtctgggccaggcgttgtgctaaaacccccaaacgctgtcatatgatacaaagtgttctgtaacttgcttggtttttttttttgtttgtttgtttgttttgttttgtttttgttgttgtttttttttgcttcgccatatattataggaatttttttaggtcattatgacctctttatttacttaattatctatttatttattttactaatatttacagaaagggtctcactctgtcacccaggctggagtgcagtggttgcaatcatagctcattgtagccttgaactcctgagctcaagtgatcttcctacctcggcctcctgagtagctgggactacaggcacaagccaccatgcctggccgatatttttatgttttgtagagacggggtctcactatgttgcccaggctggtctcaaactcctgggctcaggtgatcctccctcctttgcctcccaaagtattgggattacacaagtgagccaccttgctcagcctgacctcatttttcaaagagctgcagagtgttacataatgtatttaactggtcactttttgatgactattaagttgttttcaggttttttgttattacagtgtcatatccctggggcacagagcagtgctggcacatagccagagctcaatcgatacatacctaatgaatgaaagtacagtggacatcctaattcagccattctttgctaacttgtgtacatacctgtccagggtaggtccctagaatacagtcaataagtcagaaggtgtgagttgggatctaccttttggaaagggatgttttcaaactacagtgagtcagaggaggatggcccagaagctgggggagttgaagctgatggcgtgaaggaattaggggtgttaggaagaagcaggagataaagagctagcttgcagaagaagtgttagacttgttatgggcaggtactggagggtagctaaggacttgtgggtggcagttaccaggaagcgtatctgaactaagtgtcagaaaaagtgtcacaactgtaaattactcttgtcagtgagttcctgtccttaagggttagggctgggtagccctctactattctctaagtctgtaatgtaaagccactgaaaactcttgggttaagtttggccatcccacccaaaagatggaggcaggtccactttgctgggaccaggagccccagtgaggccactctgggattgagtggtcctgcccctctggctgggactgcagagggaggaggactgttagttcatgtctagaacacatatcaggtactcactgacactgtctgttgactcttttggccttttcagattctggggcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgatacagtatctgtaaattgaagatattgggctggatcaggtacattttatcttaaggaccaattccaatccattggtagtgggtgcccagtgcaccacattaaaaagaattctaaggctgcacctgggcttaaagaagagcactataatcaattagtgatgtctaaaaaagctaaaaaaaaaaaaaaaagagcactgcattcaattagtgatgtctaaaaagggtagaaaaaaaaaaaaaaagaaaaaagaaagagcaccgcaatcaattagtgatgtctgaaatggagcagaccaggagagcaccacgaattttgccctccataggttagctcatctctgaggtctttccctgctctgacatacttttgttccatgattacctccagcctggtggggaacaacattggcagtgtgggtgcccaagccttggcactgatgctggcaaagaacgtcatgctagaagaactctggtgagtttgggggattctctgctctggggaagtggatcacaatctctgttgatcccctggcctcatccataggagcggttgtgtggacagacaaaggtggatgattgagtgattgactgattgattgattgtgtttgtctttatatgtactgagtggtatgaagcttatagagcctggtatgtacatgctaatttttttatttaataaaatatatgggtttgctggtttggtgactgcctccacatggcataagtgttaagagcacagactctgtaatcaagcaggccgtgatcttaggcaagttaaataacaatttcagaatctcaagtttcatgtctgtaaaatgagggtaagaatacttccaaccataaaggatttttgcaagaattagataaagtagtgcctgtgaagaccttaatatagtgcctggcatatttgtaagtgctccataaatgttaaattagaataatggcagggttactactactattactgctgctgctgctgctgctgctgctacaactactatagtactgtgactactactactaataaagttttgttattttaaagtgattttgagttcctaggagcactgggtattcaagtcttaggtcattttggaaggtgtaatggagttttgatagttgaaagaggaaccatgaatcatgcttatactgttgacctgaagcagattctaagtttctcatcctttagatgccactagtatagttttctgacatgttctgggcagcttcagattatgtcagggagataaaatactgaatgtttgattttcccgggaagcagaaaggcactgcaacatatgggcattgccataaacagattttatggatggaccttggctgttgcagggcttactagctctactcaagtatgattgattctatcctgactggattttgccacttggaatttcttagtagaggagaaccttgttatgagagcatcagttatgattactgttaaaagaaaaactttaggcaaattaaatttagcagaactggtttgaacatacagcaatttatgaattgggcagcattcagaactgggagtgctccacccagcaaggtaggcaagcagtatctatagacaggaaaaggaagtgatgtacaaaacagcttgattggttgcagctgggcatttgccttatatgggcatggtgtgatgaggcattttctttatatggatatagactgatcagctggtagactgtgactgactgaagcctggctgctgtgattggctaagacttagctgtttgttataaggatatgttgttaggttgcagtttgctacataggaactcaaagtacagaggcagtctcaggccaaatttagtttaactatatgttaagctgcaggtgacagaatacctccatctatagaggtttaaacaaggaaagggtttattttttcctgtataggcagctggatgtaggcagtgtagggtttgtacagtggctacaagaggccaggaggggtctcagctctgtctcattctcttcctgttccatcattcttagcctgtgacttcattcacatggttggttgtctcatgatcacaggatggctgctccaggtgcagcactacttctgtattcccggattcgatctatatacccaggaaagccatctgggttctctcctttaaaaagcattcctggaagccccacctgtcgacttccccttatgtatcaaccatgtgtatgtcacttgaccaacccacttgtatgttgtttgaccagccctggctgcaatggagagtgggaaatacagttttttcaccaagtgcatggctgtccaaatgaaatgagacttccattaataaggaagaaaggaaagatggagatcaggaagctgggggatcagggaacttattacattgagagcccttggagtgaattctcttgcaaatatgtccctggaattgagaatccccacaacgtactttatctgttctttctttatccatgagtttgggttttcagatgttggatttcctatatggggggcatgtgagttcatcatcttccataatcaatgttgtatcaactggattttctctcttcttctcaccagcctggaggagaaccatctccaggatgaaggtgtatgttctctcgcagaaggactgaagaaaaattcaagtttgaaaatcctgaagtaaggaacccataagcaggaaacaggacaataattgctggcctttggaaggggcatttctgaataagatctgggccgctctccgctgggctaactcatgtgaggtggcctggtagaacagcttgccttggtctaggtggacaaggattccagtgcaagttgtttatctgggaggtggtcccagtaaatgctgataggagagtggtgaagtgagatggggaagtgaaggtaaccaataaaggggagttatcaagccagttatcaatgagggaaattggagctcagtactctggggcactcctggagccagtgcagaacacacatggtcacctacccaaccaatgggcaagaaagccatggcatttatccaccaaccctctgtccttcctatgttgatgtgcgctcatggggcactgattctccagcacttccagctcaccctcacccagctgaacatgcttctggggtcaggagaatggcctcaggcagagagtggaaggtcttctctgcaagcagtggctggggaggtgatgtgatggggagtactgtggcctcctccagtggctgactcagtggcttgggacttgtgccacaaagagatggacagctcaggtgaacatgaacccacctagtgaccatcatgggtttgtcagggtgctctctgaggctgatgccaaaattcttatttcaagtagacctcaggaaccccatcagatggctccttttgctggaggaaagtggcatctgcctaggcaaatgtggtcctaggaaaacgcttgcctttagagacagacagacagacagctgcctctgtgagtgccagctttgctgccaggctgctacccactctggcgacactcatttgtgttgctttcacaagctaggaagtttccaaatatttggagaaaacacttccactaattatttgggtggaaatgggctgggaagttggggtgaagcccggatgtgtctgagccagatgccagctttgcactgagggtcggcctttgggaataccaagcccattatcaaccaggtgtggatatggcaggtttgtcttccctccttgtcacagccttactccacttgactcccatggatgccaggcaatgaggctggggttggtcccatgccaccctgtcatcagccttatttttcagcatcctaaactatatcatcccccacaaaaattgaacttctgatatatcttttataaaaaagagaaatgcctacatctttcttttccaggcttagtttctgccaagagttggttgagagcccaggcttgctgggtgcagtggctcacacctgtaatcccagcactttgggaggctgaggcgggtggatcacctgaagtggggagttccataccagcctgaccaacatggagaaaccccatctctactaaaaatacaaaattagccgggcgtggtggcatacacctgtaatcccatctactcaggaagctgaggcaggagaatcacttgaacctgggaggtggaggttgccatgagccaagatcacaccattgcaccctagactggacaagagagaaacttccatctcaaaaaaaaaaaaaaaaaggatgagaaaaataataatttaaaaaaaagagtccaggctctggaaccagacagcctgggtcttacccctgctccaccattaccagccagttcttcttggatgagtgcctcagttgcctcaagtgtaaatggagataatggctggaccttcattataggccatgagcattcactgagagaatgtagctaacaaaagtgagttgtaggttggagcaaaagtaattgtggtttcagaccatgaactttaaattattataactaggctaaaatacatctttattaatcaaaataggaaccattaaaatcaacacatttttgccaataagaaataagtttgtttattcctgtagcataaaaattcatgcttcgggattcaacaaactcttggaaagcattttctgcatcctcctggttgtggaagcatttttcctgcagaaagttgtcaagattcttgaagaaatggtagtcagttggctagaggtcaggtaaatatggcggatgaggcaaaacttcatagtccaattcattcaacttttgaagctttggttgtgtgacatgcagtcgggtgttgtcgtggagaattggaccctttctgttgacgaatgccggctgcaggtgttgcagttttcagtgcatctcattgacttgccgagcatacttctcatatgtaatggtttcgcagggattcagaaagctgtaggggatcagactagcagcagaccaccagtgaccatgaccttttttttttggtgcgaatttgcctttgggaagtgctttggagcttcttctcggtccaaccactgagctagtcattgccagttgtataaaatccacttttcatcgcacgtcacaatcagatcaagaaatggttcgctgttgttgtgtagaataagagaagatgacacttcaaaatgacgattttcttggttttcactcagctcatgaggcacacacttatcgaggtttttcacctttccaatttgcttcaaatgctgaatgaccatggaatggtcgatgttgagttctcaagtagttgtaagaaaatcagctttgatgattgctctcaattggtcattgtcagcttctgatggcctgccagtacactcctcatcttcaaggctcttatctccttcgcaaaacttcttgaaccaccactgcactatacgttagttagcagttcctgggccaaatgcattgctgatgttgtgagttgtctccgctgctttacaacccattttgaattcaaataagaaaattgcttgaatttgctttttgtctaacatcattttcatagtctaaaataaatataaaataaacagaaagtattaagtcattagcaaaaaatcataaagtgagaattgtgcattaaaatgatgtatagcataaccacatttatttaagaatgtattccaatatcaaatggcaaatttcaacaatgcaaaaactgcaattacttttgcaccaatctaatagaagttcaataaatactggcaattacaattggcattgccttagggtcaacttgtaagacattcctgaaattgtgggaaagggggaggacctggagtggacattattggaaggcaaagctgtaaccaaaagagcaacctgggaaacacatgactcctctgttgctgtccctggccctatcctgtctcccctccctgttgtcagctacctcatatgttctctaatctctgtctctgtgccctcaaagacccccctgaaaatagaaatattactgctcattggttattttctatcaattaagtactgtattagtccgttttcatgctgatgataaagatatacccaagactgggcactttatgaaagaaagagttttattgaacttacagttccacgtggctggggaggtctcacaatcatggctgaaggtgaaaggcacatctcacatggcagcagacaggagaagagggcttgttcagggaaactcccctttttaaaaccatcagatatcatgaaacttatttactgtaatgagaacaggatgggattcaattaccttccactgggtcgctcccacaacacgtgggaattcaagagatttgggtggggacacagccaaaccatatcaagtactgtgcaagtgttttaggcatgcagagagtggtgggtcttcccagcaagcagagtgtggggaggtaatgggggactggtggctgacttaatggcccaggacccatgccacaaggagatggatggtggatgtgaataggagcctgcttacacccatcacaatttagattcttatgctcgatggcacgggtactcttttaggcccattttaccaatgaggagattgggactaatttgctcgagatcaaaaaagaagtggtgtaggtgggatttaaacccaggatgtctagcactaaaatgcaggtacttaaccactatcctaagggagtggctacttaatttgataaactcatctagtgaatggaagagagacggttacatttcactgatggtactgagcctttgttgatgagctcattgggaatctcagacatgagcaggatgtgtctaagggacaggtgggcttcagtagactggctaactcctgcagtctctttaactggacagtttcaagaggaaaaccaagaatccttgaagctcaccattgtatcttcttttccaggttgtccaataactgcatcacctacctaggggcagaagccctcctgcaggccttgaaaggaatgacaccatcctggaagtctggtaaggcccctgggcaggcctgttttagctctccgaacctcagtttttctatctgtaaaatggggtgacgggagagaggaatggcagaattttgaggatcccttctgattctgacattcagtgagaatgattctgcatgtgaaggatctgattctctgtctaagaaagaagtctttacctctttaagtagggagcaatgatttcatttttaaaccttgactatttattcagcaacttctctgctctatgagatagtgtaggaatggggatgtggttgaagaatgaaaagaaaagtcagctcccgccctcctagaaattgcatctgccttcacaggtcaaggatattggatcagaccttctgcggttctgaatggagattacacaggttaggagcaggttgcacagtgtttccaattctctataattaaagccatagactttcatgtattgaaaaaagcaagaattgcattcttgacagattctttcattgccttaaaaagaatgactagccttgggagtctgggcagctgggtccagtgttgtagactttctctctgctgagccacagcttcaaagatttgtccttcttgtttccagggatctatttctcagacaataagtaaaggctttccctggcctaatgtgctgtaagtgaatgctactatatatgttccaggcactgggctagagactaatatttaaaagccaggaaatttcctatagaaaatctatatctcagggttttctcaaaagagctgggaactctggatgcccattcatgattccagtagttaaccagagtacaagaagggctgagtcttctcagatgggcaaacccactctggctgactgcagatccaccaagcctattgtcttagaccaggaccctttggcaactcattcccataagcctgtgacccttgctttaaatatgcaggccttgtcttctctcaaaaagcacatcaaggctgcagcgaatgcagatatcaaatgatgaagttaaaaacaaaagctttgctgggcgtggcagctcacacctgtaatcctagcactttgggaggctgaggcaggaggatcactttaggccagaggttcaacaccagaccttgtctctcaaaaaataaaaaattcagctgggtgcggtgtagttcctagccacttgggaggctgggatggaaggatcccttgaacccaggagttcaaggctgcagtgggccatgattgcatcactgcacaggcgacagaattagatcccatctcttaaaaaaataaaaaatttaaaagtgacttcaaaaatctatgctgtgatggagagatttttccttctgtatgattgtgatagctctgtggcctatgacgtcatcaggttctgggcaaagtgtaggttttctgtttctttgtttttgaaaccattgcacagtcctaagaaacatcacattctgggtcctgggcaccagccaacatgaggttgatattttaaggggtctgatgttatttgctgcctgttgatggaacttgggagggcaccagggtttgctcattgcattcttgacagattctcttattgccttaaaaagaatcactggccttggggagtctgtggctggctgggtgcagtgttgtggactctctctgcagagtcatggagccttgttcagaatgcttcctgagctgccctggttggccaagggtaaaaacagccctgacttccctgcaagaaacactgcagctgggccagagagtcagcccatcccaggcatgggtttaaaaagtggaggcttttgtttgaaagccctgctctaattttgtcctcactcaaacctctgttcacttgatctgctttaggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttgggggtttgagaccaacctgaccaacatggagaaaccctgtctctactaaaaatagtggaggttgcagtgagccaagatcacaccgttgtcctccagcctgggcaacaagagtggaactccatctcaaataaataaagaaaataaaaaataaaaatattactaaaacttgtcaaattttaaagcacaattttctacttgaagcggaggaaaatgatgttttcaacccgataatgtatagcttaaggcaaaagcaattcttagaaaatagccaggcaatcaaaacacttttggcttcagtttagaaaagtttaaccccgtgaagttttcagctgtgcaggagcctccccgtacccagcctaaaaggctgcaacacatgtgaaaaggccccttggcccccctcgattccctcaaagtggctccaggagtctttaacagaaggatggagaaggtttacttatggctggctctccacagcgagcagcctctcatgcaggccttgtgttaaaatagtgcttagctgcgtttaataagcaagggatcaggtggtcacccagcaaggtaaaatcctacttccttgaaaggttttgcgtctggctgactcggtggtgtggcatctccctggaccctgcatgggagtgtgtctatgggttttctttgtgattggtgccctgttctgcccagaccattgtttctgatacagcagatcaggaggaaggtggtgttgagtctcctttcgctgaggagtacacacagcagcccatttgcatagcactgtattgggttgtcagtttgtcatctttaaattcattcagcaatgaggccaggcatggtggctcatgcctgtaatctcagcactttgggaggctgaggcagctggatcacttgaggtcagaagttcaagaccagcctggccaacatggcaaaaccctgtctctaaaaatagaagaagaaaaaaaaaagttagctgggtgtggcagcgcacgcctgtaatcccagctacttgggaagcagaggcaggagaatcgcttgaatcttggaggaggaggttgcagtgagctgagatcatgccactgcactccagcctgggcgacacagcgagactgtcataaataaataaataaataaattcagcaatgatttattaaccagttatattaggatgaccagtggattgacaaaacagacatggtcctcaaggtgcttaggtgagggtggagggagaggcaggcagaaaacaagtcagttgaaaaagacatgaaaaagtcttcatccaggcactgtggctcatgcctgtaatctcagcactttgggaggccaaggtgggcagatcacttgcggtcgggagttcgagaccagcctggccaacatggtgaaaccctgtctctactaaaaatacaaaaattagccgggcatggtagcaggtgcctgtaatcccagctacttgggaggttgaa

SEQ ID NO: 6 - DNA, 41654 bp, Homo sapiens
ggggtgtatgtgtatcgggaagcatgtttgtggtggggggaagcagggcgtgtgtgtgtgtgtatgtgagtgttgggaagcaaggtatgtgtgttgggggaagcgagggctgtgtgtgtattgggaagcatgtttgtgtgtggggggaagcgagggctgtgtgtgtgtattgggaaacatgtttgcgtgtgggggggaagcagggcgtgtgtgtgtgttgcgaagcggtgtgtgtgtggtgtgtgtgtgtgcgcgtgtgtgctgggaagcggtgtgtgtggtgtgtggtgtgtgtgtgtgtgtatgtgtatgtgttgggaagcgggttgttccgaggactccccgattccctcctggttgaaacgtggcccagaaagagaggcacactggctagggcacacagccagggccgtccagccccgcaggaagccccggtcctgccctgtcccaggtaagcctggccagccctggagccggcgctgcggggctgcggggctgcggggctgcggggccacgggacaggagcgggtgctgcggtcccggcagagcaggggcggggtccgggcgggggcggagccgagcgcccgcgcacccggagccgcagcccgagccggagccggagccgggagtcgtggcccggagtgggccttggagtcggcgcgcaggcggctcgcggtgagtgctgtgcccagcgcctgggggtgggctggggggagactgtgggcgccgagggtcccacccccgggacttgaacacttggcttctgagccgtcctggccgcagggcttgggcaagggttgcctcggtttctcctgggggagtcccagaatgtagatgggaagtgagacggaaagggggcatctcgaggttggggaggcccctgggccactcttttgatttccttcctctctgtgggactcagtttccccagcttcaccatgccagtgagtgggtgggtataagtgagagagtctgcctggaggatcctgctgctgctctctgtacctcttatgggaatctcctgcctcctccttctcccttgttgggtgtgagatctaagttcaaatccatcccttcccagacagcccacaccaggacctgcgaggccacatcccccagctgtttgctctcctatgcttctcaggttgatggagcagccatctggtcctgctttcattccaacaaactgaaaagccctccaccccaatccccctggggaggacctcctgaagctgcatgagggctgggggctggacccctgcttctgagtttgctctttctgagcctctcctgccatggccttggtttccccatgggctgtgacctagcctcctccagggtccttgttctacagaggggaaacacctgccctgtcaccctcccctgaccccttagtgaaaaagaccagagtgacatccccaccacctgtacttgcctgtcccgatcactcagccatggggctgctggggcccctgagtttgttggctgctcaaggcccctttgcctcgctgcaccctgggatctgaggctggaagcatcctcatccagcccacctccctcctacccagtgcagagcccctctgtgacacatgcccagtggtggggagcttattactgatgaggtggctttttagtgccttgcagctcttgtttcctttagctaaaatctgtggcttacagattctacaccttggttccatgtctgtctcccggagccatgcagagcaagtcagttctttcattctttccagcagtcatcattcatgcactggatgagacacgagacagagcagtaggaaaatcagacaaaaccccctgccctcatggagtgcacactctagaaaggaatggacagcaatgaaataagtaaatacatggcgtagcaggtggcgggaagtactacgagtaaaacaaagctgggaagcaggatggggtgctggggctgggtattgcattgtatatagcacaaccagggaaggcttccccaagaccacgatagctgggcaaagtcataaggaattgaggggtgagccacatgagtatctggggggctgggaggctttggttttcactctgagcatgatgggagcacgggtttggaacagaggagtcacctgctttgacttggggtctggcaggcccatctagctgtggggtggagaatggatcatggggggggtggggtaagggcagaaggagagcccgttgagcggatcattgctgtcattctcgggagagaccatggtgtcttgcccatggcccagaggtggttaaaaaaaagaaaaccagacattcagatttgggatatatttcaaaggccgtgtcgatagggtttggggtgcaagagaaaggtagagtcaaagatgattctgagtgtttggctcgagcttctggaggtttgaagctgccatgaactgaaatgggagttgcttagcggagagatcaagagtttggttttgtgcttgtggagtttgagctgcccattaggcatcaatgatgagtaagcagttgggaaaagttaagggcggaggttggagttgaaaataaaactttgggcatcatcaccatataactggtatttaaagccacaagagcaggtgggctcatctagggatggagtgatatggagaagagaaggggtctaaccattgagtgctggggcccccagtgttaggaaccagccaagaagacagaaagagtgaaaatcagagagttggggtgtcctggaggaaatgaagaaaatgccccaaagaggaaggagggaacaaatatgaccaatgcccctggcagagcaagcaggctgagggctgaggattgagcaatgggaggtcactggtgacagtttcactggagctggatggggaactagagggaatgggaggggatgggaggacttggggacagcagtacaggcaacagacaagggggcctgctgtaaagggagcagataaatgggattggagccaaatgaagaaggggagtgtcaagagagtgctttacttttacaatggagaattagagtgcattgtgcactggtggggggatttgatctcttagggagagaacagtgttagggagggagaatgcaggatagctgggggagggtggggggcttggccccagcagagactcaggacacttgggaagttgagcttccctgggcttcccctcctctcctgtctgcaaggggtcagtgggctgagatttcagcacttaagcaaagcatttgctcttggccccagagaaaccgggctggctgtggtctcaggaaggaaggaggtgtccaggctcaggcctgggcctgggtttcagggagggcccacgtgggtcgccccttgaccctctctttcagcaaggaagtgatcctttctctacatgggcctcaccttggggaggacaatggtgtctttgaagttgtagtaactgaagtagagatcaaaaggcaatgcagatagactgacagatttcgcctgaagaggggaagcccgaccaggtaataaaggagtaagaggaaggatgttaaggacaattttaggaaacagataatgagtgaatattttttctctctctttcccaatttaaactgaagcaggagaaactgaagctagacataatgattaacttcccaagctggtgagcttcctgagctggttagtgagaacagcactaaggccaggttctcctccccagatgtttaagatgagacaggacaatgcctgctcagagacagggcctggctgaattggccctcaggattctctctgctctgaggtttctggaagaaggccagggcagaggtgtggtgatgtagctgctgggaggacagagctccgagtcacgtggcttgggcgggcctccccttcctggtgtccacagaagcccaacgtcactagctggggtgtgtatggctcacacgtaggccaggctgccctaggcttggtgtgcaagggaggggcccctacttacttgtggcctgtcccctcgtgaatgtgtctcatgtccccagtggggtttttcagtgagggtcatggtctccaggatgcacaaggctttgtgccagaattgcttggaattgcctagttctggaaggctggttggccaactctggcctccggcttttcctttgggaatttcccttgaaggtggggttggtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggtaagaggagcaggcattgtcccgtcccagcttgatcctcagccttctttcatccttggccgcgacatgctcccaggcctggggtcagatggggagtgctgactctgtttctgggctgttttctggggagaatgggtcggcgggtttttttccccaggacctgggcagggtcaatggtgggggccgctgtcgcatccttggctggtgtttccacagctgagaaccactccagggccaagcccagagcttattctacccttttttgtcctctcttcccctgtcctcggccaccccaccctcttggctcctctgcttagatgtgggcacaaggaggagaactccttggcctgagagaactaccttagatcctggcttccagtggcctctgcaggggggtacaccctctctcccaagcagccagacacacaagtaacctcattgcctcagtttccccatctgaccagcacagggccccctgtgccccagcagcgttctgagagattggagctttctccttttgcttaccttggctaccgtatgaggacggatacagagtgttccccccacccccagcccaggggatatttgattcatgaacattccctcagtgtctttgtgggggacaatgctgtgccaggctcagggatgccaggacgagtaagacccaggctcccacgtggcccaggcagggagagagacacataaacaaccatcaggaaagaggtaaaatccccaggccacttggcatctgctcccttgagtgtctgggaatgtccctgatttataaaaagaagctgacggccctctttgttgtccatgcctacaccctttcactttcgtttcttcggggcactgcagcagcccttgtccacagaccccatgacaatcgcagaactgaccatgctgagagattttcttggctgctcagggaccctgccagggcttgaagctcctggagggtcacttgccctcaaattcccagaacgcacagcaggtcactgatgatagcagtggcagcagtctgtgcacggtggtttcgagggcgtgggagggaggtgagggccctagggcaagtgtgtgtgggaagtgttgatgggggacaaggcaccagaacgctcggaaacaacttagtttgcaccgtaatttttcacttcgcctaggacaggacctttagagcaatattctgagtctaccccttggagtagcagtgtgcaaaacacacagcacgggcttggggcccccgtggggaacccaaatgtaagagttagagacatgcattccggagtcatacatggctcgtgttgaaatcctgactctgcctgtctagctgtgacacatcgtacaaatcacttagcttcttggtgcctcagtgtcttcctctgtagaatgggtagatcataggcactacttcagagtggctgggagggttcagtgaattcctgcaggagagcacttagaatggcacttggtgtgtagtttatgcttaattaatattagccgttactgaaactgctgtagcctgaatccagccagcatgaaagagcccctctcaccctgcttcgaagagaatgaattccctgattgtttggaagatctctctctctctctctgtcttttttttttttttttgagaaacggtcttgctctcttgcccaggctggagcgcaatggtgccatcttggctcactgcaacctctgcctcccgggttcaagtgattctcctgtctcagcctcctgagtagctgggattacaggcgctcgccaccacgcctggctaatttttgtatttttagtagagacagcgtttcaccgtgttggccgggctggtctagcgctcctgatctcaagtgaccttgggagatctcttgctcctaatattacctcaagcctttttaaacgttttaagccggagaccaagcatggatatgggagttaggggtcttgatttaattcttggttgcttcaaactctgtggaaccttgaggtgtttcttgccttctctgggtctcaattttcacatctatatggtggggagcttggattgggtaatgtctgaggctagaaccatggccaactcgggttctgctggggctgacttgccctggccttccctgaccaccctgcatctggcttctggagaagtcccgcactgaccttgttctcctccccaggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggtgaggcactcctggtgtgcatcacagagttctcaggaaaggggtgcttagtcaccaagactgatttgtcctcatgaagtcagcctgtggggtaacttggtccatgggatttcccctaaaaaggtagccaggcaggtaaaatttgctcttgactcttggcaggaaacgtacaactctttctttcttcttttcttttctttttctcactctgttaccctggctagaatgcagtggcacaatcatagctcactgtagccttgaattcctgcgctcaagtgatcttctggccttagagtagctgggactacggctgctgtaccaccatgaacagctaattttttttttttcttttagagatggggtgttgctatgttgcccaggctggtctccagctcctggctttaagcaatcctcccgccttggcctcccaaactgttgggattgcaggcatgagccactttgcctggccaacagaacacttctgccgagaggaagtgtgtggtggccaggaactcagattctggagccagaatggtgcaggctcaaggtcaaccctgtgtgatctcaggcttccctatggagcctctccagcctcagtctcccttgtttcagtttcctcatctacaaaacaatgttaatagtcaaatggtgcctatcctataaggctcttgggaggattcagtgagttaatttgagtaatgcttaggatagtgtctattaccactggctgctatttattatttctgttatgagtgatactctgtacttgtacacttttatttctgtctgttttaaattaacagcacaacagaccataacactgcagtatattgaatttattttataattaacatagcatattataaactaatatagcttaaatgtttatgtaggatttctgacatgaaattgcattagatcatagatgttcagagttggtatataacagcccctgagaatgtagtaactcagcagagaccagaaggtcagagaaatgaccactgagtatttttgaaactcttttgttttcttccaaatagtgattcttagggctcctgagaggcagatggaacaatcattaacattccactttataaatcgggaagttgagaccaaggaaagtagtttgaataagctcacagtagttaatgagggggccagtgctggaccaattggccagcactggtcattgacttattcatccatcattcatttattcagccagaatctattaggtgcttcatacatatttgcttaaagtttgttgtgttcatagagctttgcacacggtaggtactccataaacatttgttgatgaaataagtgagttactgaatgaatgattgaattagaatgacactgcagtgttaaaatgggctgggttggggaacattttagtttttgtttttgtctgttttccaaaaatgtatgtgttgttcacatgagtctggataaccctagattgagattgatgacataaataaatttgtcttcaaggctgcactaaagctggctcacatggctaggtatttacagagcagaagtggtgcagtcctctctgattagttgcacgtacagaagacatattcgttattggactgaccttagtttctcttataatttgttaggggaattgaatcagcccatctgagaagttacaagattgtgtcttgtcatctttaaaagttcagcaatgtgatgtggtacagatggtctgaggggtttggagaaggtagcctagatccctagggcccagagaagacaggatgtgaacagaggaagtacatggattggtgaagaaaagaaatgggataactcatgggtcaaagaagaaatcatgatggaaatcagaaaatattcagaaccatacaataatgagaatattatttatcaaaatctattggatgcagctaaagcaggacatagggggaaatttacaaccttaggtgcctagattaggaaagaaggaaggcatttgtttatttatttgtttatttatttatttgagatgggggtctcactgtgtcacccaggctgctggagtgcagtagcacgatcataaatcactgaagtctcgaacttctgggctgaagtgatcctcccgcctcagccttccaagtaggtgggacacaggctagcaccaccataccaggctaattttttttttgtagacacagggtcttgctatgttgaggtctcaaactcctgggctcaagtaatcctcctccctcggcttcccaaagtgctgggattacaggcatgagccactgcgcccatctaaggctgaattttaatgagctaagaattcatcttaagaaagggctaaatagacagcaaaagcaaacattgaaggttgggactgagctgagtgggtagcagggatgggagacaacagatctgaggagagcaggagattttgaaaggattgcactgcctgaggtttaagcctttagaatccagctctctctgagctccctttgagctctgacattctgtgactctgatttggtggccttcccttagtggccttactgatttcatttggatggtgcttgtggtatatccaaccaacatgtcttcccaaatggccttttaatttcctataaagaagtagttgtcattgattgcaggttagggacagaaaatgctgtggaatgaaacaaaatgcaagttaaagaactaaattccaaaaatacccattgctactattgactgagtgaattcctactgtgtgccagacactgtacccagtccattccctgtattgttttatttaagcctcacaagggtatagtgtgactacactgtttcttaacaatgaagaaactgcccaaatcgcccatctgggaagcggcccagctagaatttgaatccaggcctgttttcctccagagcttgtgctattctctgtctgtcataaaatgtgggggctttgtgtggtaaacttgctcagttgggcatagcagttgttaggaaacctgaggctggtaacaccagctgtaataccagctgtccgtctgactcatgcaactgttaaagttgatagggctgaggtgtcagactgagctctgaattgcctgattcctataacaatattaacttaaacattttttaaattgggaaatgcaccatgcatacagaagagtgtgtatatttcatatgtatagtgtaaactgttcccatcacccaggttaaaaaacaggatgttgccagtacctggggccttctttaactgcaactgctagaggtaaacactggcttgacttttgtgtaaatcatctctttgcctttctttaatgttttagcatcttttaaaataaatccccaaataatgtattgttctattttgaaaaactgagtagcaagccaaaaatagctgtgtaaagaaaggtcacttaaattaggctgggtgcagtggctcaagcctttaatcccagtactttgggaggctgaggcaggtggatcacaaggtcaggagatcgagaccatcctggccaacatggagaaaccccgtctctactaaaaatacaaaaaattagccaagaatagtggcatgtgcctgtagtcccagctactcgggaggctgaggcaggagaatcgcttgaacccgggaggcagatgttgcagtgagccgagatcgcactgcttgaacccgggaggcagaggttgcagtgagccaagatcgcaccactgcactctagcctgggtcacagagcaagactctgtctcaaaaaaaaaaaaaaaaaaaagaaaggttactattgccttttcttagatgaaggttcccaaggcagggaaagctaagtggagtctcagggacttggtctggcttttccttccctgggaatttataaggacctcttctgggaagtcagtcggcaatgccatgaatgagtctggggaaatattgggctcattgcaactggagggtctggtaggactgatgtgaattaggtgctgtgtccggaggaaaatggccagaggaagtgggctgctttgtacagtcagtggtaaagttgccaaaggctattatagctcacaggaatgggccaaggctaaacactcctgtggagtgaaatgaatgtcctcagctgactgaggcagcgggagttgaaaagaaacgatattagttcatggtgaagacaagtcaaatatagataaaggttagggtcaggcttgcctggacatctaggagataactgccctcaacttgtttgaatcttgagtcactgctccattttgtttgaactggtggccatctacttatagtatacagccatcaacctgagatttccctacatggtcttcctgccttggtctcctgtatcctgaatcctatggcctcttcttccctggtttactacattttgctagaccgtatcctccagtcaattccttagaatgaatgtatgaaagttaaaatttctgaggtctcacatgtcttaaagttccctcatactggattgatagtttggctgggtataaaattctgggctggccatcattttccttcagaattttgattgcattattccattatcctctcttttcaatattgcttctaagaattccaaaacctttttttttttttctttttgagacagtgtctcactctgtcacccaggctggaatgcagtagtgtgatctcagctcactgcaacctccacctcctgggtttaagtgattcttcttcctcagcctcctgagcagctgggattacaggcacccaccaccacaccctttagtagagatggggttttgctatgttggccaggctggtcttgaacttctgactttaggtgatctgcctacttcggcctcccaaagtgctgggattaaaggcgtgagccaccacacccagcctccaaaaccattttaaaactctttctggaagcttttaaaattttcttttagtccccagaattttaaaatttcaattatgtgccttggtgttcttccattatgttagtcacccaagaggtactttcaatctggaaacttctctatgttttgggaaatgttcttgattagtttacaggtgatttcttcctctccattttatctcttctcttttcatgaaactactattaattcaatgttagaattccttgactgatcatttaattttcttctattttccatctctgtgtctttttgctctacttttctatgatagtcacagctctatctttaaactcttgagtttttcatttttgatgtcatgattttaatttgcaaggggtaggtttgactgattcttttttgtagtatcttactcttgttttatggatgcaacatcttctttgacttaaggatcataagataggtgggttctttgtttgtttgtttgactgtttttcaccctatgtaaactttttctacaagtttctttccccttcccccctttttggcttctatctcccacattagatgctttctctgggctcatgatactctttggttttctttctcaagattgacaggtaggactttaaaacttgttgagcatgcgggtgaaacttgtctaccatgaatttcactgtagatattttggagattgacagtgtttatatctttagatctcgcctcctgggttgatcaagttatctgagtacaccacagaccttttgcctggggataaaccagaaatctgtttcagaaaccactttgattcagtcttccttgttttagtcatttccttcagttccggaggtccctcatgctgatcattccagagccctttacagatcctagggtacacactgcatggttttcaactttcttgttttggggttaagatttggctttcaggagtctcctcagtccgttactattcattcaatcagcaagtccttgagcacctgatttgtgccagacattcttctaggtgttagggatacctcagtgaacaaaacagacaaaaatctttgtcttggaaatacacacactccagtcaggggagagggacaataagccaaaggaaggaaattacagcgtgtgctagaaggtgataagtgctgtagaaagtaagtaaagtgggtttgggagttgagagtttgggaaggggataaatgatggcaattgtaaatagagtagtcagagttctcacttagaaggtgaaattcaagtaaagacttgaaggaggacagggaattagccacatggatggctaggggaaggcttccaagctgagaggacagccagagccaaggcccagaggcaggagcatacctggtagttttaggaaacaggaggccaggatgctgagtggagtaagagggggcatgaaaggagaaacttgggtccacgtggttctagacaggtatttttgtctgttttgggccctgaaggttactattggacttggactcttactctgaggaaatagggacgctattgggacgtttgtacaggagcaatgtgacctgagttttgtttgtaaaggattagactctggctgtggcattaaggctaggctgtgggggcaggaacagaagcagggggaccagttttgcagcctgtgcagctttccagataagcagggattgtggcttggaggaggatggtatagaggaggtgacaagaaatgactctatgtctggtatgtagatattggccacagatggcatttgagcactagagacctggctggtccacatggagtttccataagcacataatacacatcagatttcaaagacttaatatgaaagaaagaatgaaacgtaccttgctatttctttcttttttttttttttttgagacccagtcttgctctgtcacccaggctggagtgcagtggtgtgatctcggctcactgcaacctccgcctcccaggttcaagtgattctcctgcctcagcctcctgagtacctgggactacaggcacctgccaccacgcctggctaattttttgtatttttagtagtgatggggtttcaccatgttgtccaggctggtctggaactccggaccttaggggatctacccgccttggcctcccaaattgctgggattacaggcatgagccaccatgctcagccatatcttgctattttctacatggattacatgttgaaatggtaatgttttggctattgtggattaaatagaatatatgattaaagttgatttcatctatttcttttaactttaaaaaatatgtctgttagaggatttgaaattccacatgcggcttgcatttgtgacctgcatttcatttctgtggaacagtgccctttttgggacatgctttgaaggtggagtcaacaggatttggcagattacagacgagaggcttcaagggtgactccaagacttcggggcagagcacctggaagaaaggggttaatattagccaagatgaggaaggctgtcggtttggcaggtgcatgggcaggttaggagtttagttttgaatatgttggaggtgtttatgaaacttttaagtggagatggaaaataggcagttggatgtgcaagtccagggttcagggagacagttcaggctggagatgaagatgtgggagtctgaggagagattgtattcaaatattcaatccatgagacttgatgaaatcacttctcttccaaatgatttacagcctgcagaatcattttccctatctttgtaggtttatgtcttcattttgtttcatttatttttcagttattcactgttttagtgagttttgagtaggagccagattggatgcatgcgttcaattcaccatccaacactgtattaactacttgaaactcatgtggttgttcggttgtttttttgaccttttattctggatggaagagagatgcttatgaagttgcagtaatcagtaagccttcccacattgctccatcagccttcctggaagaataatgtcttctgcctttcctgtaggcaagaaggctgcttgatcttgccacggtgaaagcgaatggattggctgccttccttctacaacatgttcaggaattaccagtcccattggccctgcctttggaaggtaggtgtatgttctcagttaatcagaaagggaagggcagtcagtgcagatccatggttaagagcagaacacacctcggttaacatcccatatgctggcagtatagcctccctatgactcaatttccttgttttaaggctagcaccaccccgtctcattgggattttgggagcattaaaaggacaaaagcgtgtaatgttagctattagctttcattatctcccacacagtatactgacaattgggctaccatatattgagggctaactaaaggtgttacttaccatccaaactctcattatctgtaccgaaaagatatggacacatgttttgagttagggctggtatctcttgatctctgaaatttagcagctcacaatgggaaactcaagaaccaagtggatctagagactctggtatccctcagtgcccagggtcaccacccaaactcaggaacaggaggggcttggaccgcaccacttgaacataccaggcatcctgccaggtgctttatggacaatgtctaccctttgcaacaaccctgagaagtaggtggtgtttttttccaccttatagatgtggaaactgggcagggaggttaagtgacgagggaggggaagatgggtctgattgtaaattgtccccacctacactttctcttttcttgggagaagaaatgtcagttgtaaagagagagtgcaagcctggcactctttagggcttgttcctacaccactgtagggaaagctcattggcactgaagccccctgagctgtgtgtggtgctggcagatgggtctatcaccctgggctgtgtcctctgggcagcaagcaagcctgtgggcggggtggctggaagtctgtgcctggcactcgcgagtgcaccgtctcattgaagaacaggatctaaacatcagtgcgccacagcagggtgcgcggcacggagtgcaggccctggtttggcccttggttgaggtttgctgttgacatcatcaagcacagctagtcactgtaagaccaggccagggtgcaagattccccacacttctaaaggtgacaattggtgtgtttatttctctataaaatgacattttttttttctggagaattttagtatcattggtgatgactggaaaacctgcatcagaaatcaggtcggaagaggaagatatatatctgatatgtactggagaggaagatatctatcttatggtctaagttcagggatcctggtatattcagagggcagaaagctcagcaataatcatcaactctgggaacagaggtgacataaacacagggcgtcccctttgtgtgactgcagatagtcatcagtgagctcagagctctatgaaaattacttgctagtttttgggttgaaaatagtgggccagtgtttggttgggggcagtgaggctgtgatggcgggggaccatgccaagctcctaccagcctgggacgctaaaccagcacttccccatttcctgaaaggggaactaaactctgacacaggaaatggtttgcttgcattactttcaggatgagaaaggaagagcactggccttccaaacacaccccgtgcatgaaaactctccctgcatggggtgcatggggaggatggggaagtggaggcaggatcacagactcttgttcgagtgctcagctggggcaccccggtgaccccgaggccttcccttgctaggtccacccagatcaatcaggatcatctccccatctcgaagtttaactttatcacatctcagagttccttttgccacgtaaggtaacatattcacaggttctgagaatccggacatggacatctttgagggtctattgttgtgcctactatatccatgaataataatgataataagcaccattttttgagagtttgccatgtcagatattcttttaaactgtattttatctcgctgcctcctgaaaaaatccttccaggtgtatattgtccccatttttacagatgagagaactgaggcccagaaaggctaaatggcttgcccaagtgtatggtggacccaggttttcaaactcaggtgtgtctggcttcagagactgggctcctgagcccttaagccctttgttcccctttagaaaaagtcacctgaggctgagtggtgaagggatttatccaaagccacccggccactatggcaggacagatatcagaatacaggtcttccgatcccagcccagagccccttcccgtcatctagaactcctcctggtgtcagtaatgataacggcagtcactgatgtcttttgagcacttactttgtgttgagcacttacactgtgctaagcacttgacataggtcatcttagttgatccgtgtaaaactctgtgaggtagtgaccaacatttctcccaccttacagaggtggaaactgagggttaggaagtttccttgactgtcctcaaagtgcacagcttgtgaatggaggagccaggatgggcgcccgctggctctcctatcccttcagttatgtcagcgtcccccgcagcagcccattgtctggttaggtcccgtcttcaccatggtgccaccttcatctgcctcttcttctgccttccagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtagtgagtgttactgggcattgctgttcaggtatgggggagcaccatcaaggctaagtgtgggagcaccgagctgggctctagaagtctgggcccagcttcgcctctgccaccctgctttgcaacactgcccagatcccttcccttctgggccttaatttcaatatgtgatgatgacagccacactttattgactggcctatgtgctgggtctggtgctatgctttccggaatgacctcatctaatctctacaaccaccctggcgggtaggcaggaatgttattatctccattatccttgacttgaggctcagagaagtgaagtaacttgtccaggaaatggcagagctggggttcacaaattgcatcattctgattacaggttttctgcctcccaccagtctatggatacacttcagaggctccctgaaaaccttgaggtcacttgcagaaagttttgtgtagtatgtgtccgtatcaggaacaacaccaaatcagaggtgacttgtgccccatcagagactttaacaccccaaccagatgggaatttcaggacccaagaaatagaaagtggctgcagggttacaactactgttggattcctgaggtagcacagtgtccaaacaggatttcagcactacccgtattgcttagagccccagccaaagatgtgaggttttgccctttggagaatctgtgcccctgaactcgggggcctctttccacatcttgggggcaggcaagggcagagggtgtgcctaggcctgcggatcagcatgcgacagattccccaacatccttccagcttgaaaggggattgccctgcttctatttagaacctataggaaagcagaagttctagattgaagttaaaattgattcccagcctccaggggctttgggctacacctggatgaccttaattgaccctaagcatgggacaaaccacttcctgagagtattaggatggtatacatcttctctgggggcaaagcaacaagatttatttttcatcatggaccaaacacatggatacccactagaaactgtgtagtgaattttgttaaccctgacatagggaccatggtctttaggttaaagcataataacaacataatacataacatatatagcgaatatatatatgtattatatgcaatgaatgtaaatatgattatacccatcatggtcttggaggaaacagatgacacacttaaaatgggtgttttgaggagagtttgaaaaacagattgtttacaagccatgggcaggagttaggaagagtgagagggttggtgcaggggcctggggttagtaacagctgggggagggtagacttgaagggggaaggggagggagactaattagctggggggaaggtatggagacggctgcctgagcttctgcaaagtggaagaataccgcttggccctaactcctcaccccaactcttgctcctggccagcgccttccaccagctggacccatcagggaggccgagtgggctgtctgctggagtagtccccaggcatcagcctcccaggagccagggacgggtagagaagggggagagtggatctggccaggcaaatggaaaacagccagcaccaaactctatttccctaggagggaggatcatgatactttgagtgggaatttggaaacctgtctgttggagcaatttccctgatagaaataagaatgtgcattttcctgggtagtagactcagtttttaccccaagaggccaggcatcactggcctgtgtgatcctcataggccagtccatctctggaattcttgaatggatcatccatccttgattagggatgtccccgtgattaccagggtgtgcagaagggctctgggaaacctgtgggtctgtctctgtgttcagagaaaggtgagggtggcctggttctagctcatggtgctcagactgtggtgtgtaaaggcactcgtggcaatgcagattcctgggcctgcctctagtgattcccattcagtaggtttggggtggggcccaggaaatctatatttttcacagacacccctggtgattctgatacaagtggtctcgccctgggagaactactggtctgcagcaaccagcttggttttccattagcaattactgtccttgagcgagttttactgctcttcaccttacacacactaaaactgccaaggccgtaggggaggggaagcaaccatgaggttgctgtgagtgcactgtgtgtgtgtgtgtgtgtgtgtgtgtgtgtgtgtgtgtgtatgagagagagagagagattgagaaagagaggaagggaggaagggggagggcacaggctcctctcccacagtgccaacctgcctctctcccacttgaagtgtttccatgccaactgaaatcctcagcctctaggaaaccctatatacacagtgcccctatataggtttctttagactctggctctctcagactctagagtgatggctttaaaagttttttgaatgttacccacaatgattgagaaagagagagagagagcacgcaccaccatgtaaacatggaacctaagtttcacaaaatgacttcgctttatgaactctgagacactctgctctcttctgttctgttctatttccattttagaaatgctgctcaggaccttcaaaatgatttgcatgacctgcaacctgcagtctgaaaaatcactgcactacagaagtggccataagaggccctgagggagaagctgcacaatgtcatggttaagagtggggtttggagccaagccgcctaggctcaaagcctttatgtgccgtacaaccttggcaaagtcacttcgcttgtctgtgcctcagtttctttctcacgaatgctcataataatggttcccatttcactggcttgttgtgaggatgaaatagtgttattattgagaagtggtaagggtagtgatcagtgctagcgatcatgattctaggtgacttttactgtgtaccgggtgctcacaaggctttatgtgcacagcctggtgaggctgataatactattgttccctctttttttttttttgagacggagtctcgttctgttgcccaggcagggggtacagtggcacaatctcggctcatgcaagctctgcctcccgggttcacgccattctcctgcctcagcctcccaagtagctgggactacaggcgcctgccaccacgcccggctaatttttttgtatttttggtagcgacagggtttcactgtgttaaccaggatggtctcgatctcctgacctcgtgatccgcccgcctcggcctcccaaagtgctgggattacaggcgtgagccaccgtgcccggcctgttccctcttttatagatgaagagaccagcaaataactagtaagtcgctgatcaggatcacaatatccagctgaggcactccagagcctgagctgttaaccattcagtcagggcctcccaagtttgcctaaagataaagaatcatgtgcacagttgttaaaatatacagattcctgggccccaccccgcagatacttgattgccagctccagggtatgggcctgagaatctgtcttttagggaagctttcagatgatgttgtgatcaggtgagttttgggaatggtgccccaagaggagtggcagacagggcttgctcggcagggactagcctgttggagtggtgccattggggttaaggactgggcagcagggcctcactaaccacagcctatatgcctgtttctgaagttttggccactctcatccagctggtctactgtctgctgacctagatgatggtaaattgtccccaggggtagcctgtctagttcaggctgcacctttcgcatatatcagctcctttccaccatcatcccctttgtgaggctgctgtgattatcatgttccttttgcagagatggaaacattgcctcaaattagctctgtcatttcctaaggattccagggttctttagtagggggtctggatcctacgtcctgggccatccccatcatagtgcaccacgtcacctccctggccagggaccgtggggtctccacttttttggggtgctccatctatgcagggtttcctggaagcacagatgctggcacttcagggatgaatgaaagtctttttgggggatttgtagatttttttcttgtcttactagctccattttcaaatgtatttattttgtctctttagtttgcgcgataacaatatctcagaccgaggcatctgcaagctcattgaatgtgctcttcactgcgagcaattgcagaagttagcgtaagtcagcctgggctgtggacaatgggctccaagtgccctggtctcaccccaggtcgtgcagcctgggaagctgtgagtgatgggctggggcaggggctgtttgcatgatggggggtgcaggtgattcctgcccagaggggaagggcaaccctgggatttggtgctcactgtccaatgtgctttgcttctgtgtctcctctcttctggaactgaacagtctattcaacaacaaattgactgacggctgtgcacactccatggctaagctccttgcatgcaggcagaacttcttggcattgaggtgagcccaggttttccttattccctggaaactattttttgccccattcctgagtcagtctgatctggtcttggcctggcactgcccacactggctcctgacctcctgattgaatgcagggacagtgtctcattttaagcaggggttctctaatgctgtgatctccccagtaaactctggactagctctgctgaggacttcctgtcttttgacctttagcccgtagggcaagaaagcttttctaggcccctttccttttctgtgtctaagagtgtcacagctttctgggtttactgagttccacgatgcatgttgagctcgtcctggtgggggaggcatacacagttacttgccaccccagctgtggcagcgagttgctgcaacactcccaggaggtcctttcaccactcagagcatgcaaggtttgcagtccatctggttctgcatttctgctactccagtgtctcccagtttcaacaggagtctctctctctcctacctgatgcctttaaattgcccctctagctggccgctgggttggcctggcttctctctccttctctctctctcagatattcttgcctcctgtgatttgtgaggcagtaaaaaaagacaaagtaaagaattgcttccatctattcttttacctcttgggctgggtttgtggatgggagccgccattttaaaatggcgggccacatagctcagtctcggcaagggctactgagatcagaaccacaggtgccaatttgtacaaaggactcagtcctgctaccactgcctgatccctcagactcacaagcctggaataggctgtggccagacctggctggcccatccctgagaagggtgctagtttcagaaatggaggctgagtttgtggccaacacagtagtcctccggtatgtgcaggagagatgttctaagaccccagtggatgcctgaaaccatggagagtatcaagccctacacataccatgcttttcccaatacctacacacctgcaataaagtgtagtttataaattaggcacagtaagagagtaatagcaactcataataaaatagaacaattataacaatcaatgtactataataacactatgtgaatgtggactctctccatctccctcaaaatatcttcttgtactgtactcacccttcttcttgggaagatgtgtggtggtaaaatgcctgtgtgatgggaggaagtgaggtggatgacgcatgcagcactgtgctctagcgctgggctgctgttgacctgaccacacttcaggaggagaatcatctgctcccagagatccctaatctttgagcaacaatgaggtcggcagctggatgtcaggagcagacgatcttgatgattaccaaatgggagcgtatagagcgtggatgcgctggacggggggctgattcacgtcctgggtgggatggagctggatggcacgtgatcagaatagcatgcaatttaaaatgtatgaattgtttatctctagaattttccatttaatatttttggactgcagttgatttcagataactgaaaccatagaaggcgaagctgcggataagcagggggcagggattaccgtatatcattgtaatagagagcacaggctctggagccagactgcccgaggtttgaaccctcattagctgcgtgacctcaggtcagcccaatgtctgtgtgcctccgtttccccttctgtagaatggaggtaataaccctggctacctcacaggctgtagtgatgagcaagcaagttaatccacatgaagggctgcaccgtctggcaggggctttatatagtaagcgagtggctgaaagatgatgggtaaatcacacaagcactcagcttgtttctccttatgtgagtccggtcctccaagcagggattcaatgtgccacccatttattggggaaaagtcctaaaaggggaagtggggaagggagctgggggaggctgggaggtgtgtccctgagtgaaggagagagggaaggaaggaaggttgagactgggcaccttggacttcagtgcagtcctaagacatcttggcaaggctgatgaggagttcttgaaccaaattcaccaggcaggggagcctgatgtctcaggcaggggctggcaagtgcagatgcgaggatgttagattttggagcacagcagctggggcccttggctacctccaaggagctgaggctggagacctgaaaggcgagttctcctagctgccacaccccttctccaaggatacaataatatctgccttataggattgttgtgagctgagtggcttgacgttccttgaaagaatgaaagcgtatagttatcccaggaagcctagggttgcaggtgagagctctggggcttctccgaagctctccgaggtgtctggattcagttgcagcaggagccttccttgctgggatcttcccccacccctagccttggccctccctctctccttcctttctggaaggctcagtgggccccacccctccctccagccacctggacctgcccagcgctcttgtgcaacaggtaaagcctacctgtagcaacaacagatctgggaaggctgcagagggcacgatggggtctggatcgagggcggctgagaccagagggaaaggtgtgaccctgagtcaccctcgctgtcccggggaaaccacctcccaggacagctgcctactgtggctcctgcctggaattgtcacactgctgtgcaaacagcgtcccgctgcccctttccctttgctgggggaaaatgaagttgtgggagccgctgagtaaactagacctagcagcgagggcacctgatgtggctgctgcctcccgggcaggtcttcaatgctttcttcctgtgtttccctggccagggcacagacggccctccttttctgcctgccgctgtgttctctcagcctcctctgtcttcccttccaggctggggaataactacatcactgccgcgggagcccaagtgctggccgaggggctccgaggcaacacctccttgcagttcctggggtaggttggattccaggaagagggacctgcatggaggggcttgggacttttgaggatttaggggcaggtgaaactcttcagccaggaggccccagaggcagcccagctccagtggggaggacaagccagggagagagtgggcggcccttgactgccaccttcatacttggtctatgcctgacaaacaggaagtttgggatgttggggctaggggaggacagtgcccacgagctggtgacaggaagccctctgatcctcagggggcgctagggctgtactttagctgcatattaaaaccacctggaagcttctaaacactattgccaggcctcccaccccagactgatgaaatgcaaatatctaggtgcaaggcccaggtatcaggagttttaaaaagcttcccaggggatgtacagccaggggtgaggacccctgacctaagaaagagaaggaaatggggaaggataggaaggcacccaggataagaggggctgtgctaggtccctcggagctcttgctccctgtaggaccatgctagggcctgccagggaggggagtaccccaacctgcagccccagggtgggcttcctctgtttgctaggcacccaggcttgcacctgtgctgtttccagcagcctctctcctatcctgtcatgccctagtgtgaactggagtccatttgacaagaactgggagttttagaacctgggactgtaggaagagagaataaccttagggcctaggtgttccagcccatttcacagggaggcaagttgcccccaagctcagttttttgttttgttttgttttgtttgagatgtagtctcactctgttgcccaggctagagtgcagtggcacgatcttggctcactgcaacctccgcctccttggttcaagcgattcacctgcctcagcttctcaagtagctgggattataggcacccaccaccacgcccagctaatttttgtatttttagtagagacagggtttcaccatgttggcccggctggtcttgaactcctgatctcagatgatccgcccgcctcggcctcccaaagtgctgggattacaggtgtgagccaccgcacccggcccccaagctcagtttgagccacaaatgggactatgttgctctagaaatcaacatcttttccacactgcattagtagcaacagagtctagaacaaaggaggccacagccccactgaactctcttctgcttgaggtcacatctgccacatcaggggtatttacctctttcaacacatatttattagggcacctgtctgggccaggcgttgtgctaaaacccccaaacgctgtcatatgatacaaagtgttctgtaacttgcttggtttttttttttgtttgtttgtttgttttgttttgtttttgttgttgtttttttttgcttcgccatatattataggaatttttttaggtcattatgacctctttatttacttaattatctatttatttattttactaatatttacagaaagggtctcactctgtcacccaggctggagtgcagtggttgcaatcatagctcattgtagccttgaactcctgagctcaagtgatcttcctacctcggcctcctgagtagctgggactacaggcacaagccaccatgcctggccgatatttttatgttttgtagagacggggtctcactatgttgcccaggctggtctcaaactcctgggctcaggtgatcctccctcctttgcctcccaaagtattgggattacacaagtgagccaccttgctcagcctgacctcatttttcaaagagctgcagagtgttacataatgtatttaactggtcactttttgatgactattaagttgttttcaggttttttgttattacagtgtcatatccctggggcacagagcagtgctggcacatagccagagctcaatcgatacatacctaatgaatgaaagtacagtggacatcctaattcagccattctttgctaacttgtgtacatacctgtccagggtaggtccctagaatacagtcaataagtcagaaggtgtgagttgggatctaccttttggaaagggatgttttcaaactacagtgagtcagaggaggatggcccagaagctgggggagttgaagctgatggcgtgaaggaattaggggtgttaggaagaagcaggagataaagagctagcttgcagaagaagtgttagacttgttatgggcaggtactggagggtagctaaggacttgtgggtggcagttaccaggaagcgtatctgaactaagtgtcagaaaaagtgtcacaactgtaaattactcttgtcagtgagttcctgtccttaagggttagggctgggtagccctctactattctctaagtctgtaatgtaaagccactgaaaactcttgggttaagtttggccatcccacccaaaagatggaggcaggtccactttgctgggaccaggagccccagtgaggccactctgggattgagtggtcctgcccctctggctgggactgcagagggaggaggactgttagttcatgtctagaacacatatcaggtactcactgacactgtctgttgactcttttggccttttcagattctggbgcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgatacagtatctgtaaattgaagatattgggctggatcaggtacattttatcttaaggaccaattccaatccattggtagtgggtgcccagtgcaccacattaaaaagaattctaaggctgcacctgggcttaaagaagagcactataatcaattagtgatgtctaaaaaagctaaaaaaaaaaaaaaaagagcactgcattcaattagtgatgtctaaaaagggtagaaaaaaaaaaaaaaagaaaaaagaaagagcaccgcaatcaattagtgatgtctgaaatggagcagaccaggagagcaccacgaattttgccctccataggttagctcatctctgaggtctttccctgctctgacatacttttgttccatgattacctccagcctggtggggaacaacattggcagtgtgggtgcccaagccttggcactgatgctggcaaagaacgtcatgctagaagaactctggtgagtttgggggattctctgctctggggaagtggatcacaatctctgttgatcccctggcctcatccataggagcggttgtgtggacagacaaaggtggatgattgagtgattgactgattgattgattgtgtttgtctttatatgtactgagtggtatgaagcttatagagcctggtatgtacatgctaatttttttatttaataaaatatatgggtttgctggtttggtgactgcctccacatggcataagtgttaagagcacagactctgtaatcaagcaggccgtgatcttaggcaagttaaataacaatttcagaatctcaagtttcatgtctgtaaaatgagggtaagaatacttccaaccataaaggatttttgcaagaattagataaagtagtgcctgtgaagaccttaatatagtgcctggcatatttgtaagtgctccataaatgttaaattagaataatggcagggttactactactattactgctgctgctgctgctgctgctgctacaactactatagtactgtgactactactactaataaagttttgttattttaaagtgattttgagttcctaggagcactgggtattcaagtcttaggtcattttggaaggtgtaatggagttttgatagttgaaagaggaaccatgaatcatgcttatactgttgacctgaagcagattctaagtttctcatcctttagatgccactagtatagttttctgacatgttctgggcagcttcagattatgtcagggagataaaatactgaatgtttgattttcccgggaagcagaaaggcactgcaacatatgggcattgccataaacagattttatggatggaccttggctgttgcagggcttactagctctactcaagtatgattgattctatcctgactggattttgccacttggaatttcttagtagaggagaaccttgttatgagagcatcagttatgattactgttaaaagaaaaactttaggcaaattaaatttagcagaactggtttgaacatacagcaatttatgaattgggcagcattcagaactgggagtgctccacccagcaaggtaggcaagcagtatctatagacaggaaaaggaagtgatgtacaaaacagcttgattggttgcagctgggcatttgccttatatgggcatggtgtgatgaggcattttctttatatggatatagactgatcagctggtagactgtgactgactgaagcctggctgctgtgattggctaagacttagctgtttgttataaggatatgttgttaggttgcagtttgctacataggaactcaaagtacagaggcagtctcaggccaaatttagtttaactatatgttaagctgcaggtgacagaatacctccatctatagaggtttaaacaaggaaagggtttattttttcctgtataggcagctggatgtaggcagtgtagggtttgtacagtggctacaagaggccaggaggggtctcagctctgtctcattctcttcctgttccatcattcttagcctgtgacttcattcacatggttggttgtctcatgatcacaggatggctgctccaggtgcagcactacttctgtattcccggattcgatctatatacccaggaaagccatctgggttctctcctttaaaaagcattcctggaagccccacctgtcgacttccccttatgtatcaaccatgtgtatgtcacttgaccaacccacttgtatgttgtttgaccagccctggctgcaatggagagtgggaaatacagttttttcaccaagtgcatggctgtccaaatgaaatgagacttccattaataaggaagaaaggaaagatggagatcaggaagctgggggatcagggaacttattacattgagagcccttggagtgaattctcttgcaaatatgtccctggaattgagaatccccacaacgtactttatctgttctttctttatccatgagtttgggttttcagatgttggatttcctatatggggggcatgtgagttcatcatcttccataatcaatgttgtatcaactggattttctctcttcttctcaccagcctggaggagaaccatctccaggatgaaggtgtatgttctctcgcagaaggactgaagaaaaattcaagtttgaaaatcctgaagtaaggaacccataagcaggaaacaggacaataattgctggcctttggaaggggcatttctgaataagatctgggccgctctccgctgggctaactcatgtgaggtggcctggtagaacagcttgccttggtctaggtggacaaggattccagtgcaagttgtttatctgggaggtggtcccagtaaatgctgataggagagtggtgaagtgagatggggaagtgaaggtaaccaataaaggggagttatcaagccagttatcaatgagggaaattggagctcagtactctggggcactcctggagccagtgcagaacacacatggtcacctacccaaccaatgggcaagaaagccatggcatttatccaccaaccctctgtccttcctatgttgatgtgcgctcatggggcactgattctccagcacttccagctcaccctcacccagctgaacatgcttctggggtcaggagaatggcctcaggcagagagtggaaggtcttctctgcaagcagtggctggggaggtgatgtgatggggagtactgtggcctcctccagtggctgactcagtggcttgggacttgtgccacaaagagatggacagctcaggtgaacatgaacccacctagtgaccatcatgggtttgtcagggtgctctctgaggctgatgccaaaattcttatttcaagtagacctcaggaaccccatcagatggctccttttgctggaggaaagtggcatctgcctaggcaaatgtggtcctaggaaaacgcttgcctttagagacagacagacagacagctgcctctgtgagtgccagctttgctgccaggctgctacccactctggcgacactcatttgtgttgctttcacaagctaggaagtttccaaatatttggagaaaacacttccactaattatttgggtggaaatgggctgggaagttggggtgaagcccggatgtgtctgagccagatgccagctttgcactgagggtcggcctttgggaataccaagcccattatcaaccaggtgtggatatggcaggtttgtcttccctccttgtcacagccttactccacttgactcccatggatgccaggcaatgaggctggggttggtcccatgccaccctgtcatcagccttatttttcagcatcctaaactatatcatcccccacaaaaattgaacttctgatatatcttttataaaaaagagaaatgcctacatctttcttttccaggcttagtttctgccaagagttggttgagagcccaggcttgctgggtgcagtggctcacacctgtaatcccagcactttgggaggctgaggcgggtggatcacctgaagtggggagttccataccagcctgaccaacatggagaaaccccatctctactaaaaatacaaaattagccgggcgtggtggcatacacctgtaatcccatctactcaggaagctgaggcaggagaatcacttgaacctgggaggtggaggttgccatgagccaagatcacaccattgcaccctagactggacaagagagaaacttccatctcaaaaaaaaaaaaaaaaaggatgagaaaaataataatttaaaaaaaagagtccaggctctggaaccagacagcctgggtcttacccctgctccaccattaccagccagttcttcttggatgagtgcctcagttgcctcaagtgtaaatggagataatggctggaccttcattataggccatgagcattcactgagagaatgtagctaacaaaagtgagttgtaggttggagcaaaagtaattgtggtttcagaccatgaactttaaattattataactaggctaaaatacatctttattaatcaaaataggaaccattaaaatcaacacatttttgccaataagaaataagtttgtttattcctgtagcataaaaattcatgcttcgggattcaacaaactcttggaaagcattttctgcatcctcctggttgtggaagcatttttcctgcagaaagttgtcaagattcttgaagaaatggtagtcagttggctagaggtcaggtaaatatggcggatgaggcaaaacttcatagtccaattcattcaacttttgaagctttggttgtgtgacatgcagtcgggtgttgtcgtggagaattggaccctttctgttgacgaatgccggctgcaggtgttgcagttttcagtgcatctcattgacttgccgagcatacttctcatatgtaatggtttcgcagggattcagaaagctgtaggggatcagactagcagcagaccaccagtgaccatgaccttttttttttggtgcgaatttgcctttgggaagtgctttggagcttcttctcggtccaaccactgagctagtcattgccagttgtataaaatccacttttcatcgcacgtcacaatcagatcaagaaatggttcgctgttgttgtgtagaataagagaagatgacacttcaaaatgacgattttcttggttttcactcagctcatgaggcacacacttatcgaggtttttcacctttccaatttgcttcaaatgctgaatgaccatggaatggtcgatgttgagttctcaagtagttgtaagaaaatcagctttgatgattgctctcaattggtcattgtcagcttctgatggcctgccagtacactcctcatcttcaaggctcttatctccttcgcaaaacttcttgaaccaccactgcactatacgttagttagcagttcctgggccaaatgcattgctgatgttgtgagttgtctccgctgctttacaacccattttgaattcaaataagaaaattgcttgaatttgctttttgtctaacatcattttcatagtctaaaataaatataaaataaacagaaagtattaagtcattagcaaaaaatcataaagtgagaattgtgcattaaaatgatgtatagcataaccacatttatttaagaatgtattccaatatcaaatggcaaatttcaacaatgcaaaaactgcaattacttttgcaccaatctaatagaagttcaataaatactggcaattacaattggcattgccttagggtcaacttgtaagacattcctgaaattgtgggaaagggggaggacctggagtggacattattggaaggcaaagctgtaaccaaaagagcaacctgggaaacacatgactcctctgttgctgtccctggccctatcctgtctcccctccctgttgtcagctacctcatatgttctctaatctctgtctctgtgccctcaaagacccccctgaaaatagaaatattactgctcattggttattttctatcaattaagtactgtattagtccgttttcatgctgatgataaagatatacccaagactgggcactttatgaaagaaagagttttattgaacttacagttccacgtggctggggaggtctcacaatcatggctgaaggtgaaaggcacatctcacatggcagcagacaggagaagagggcttgttcagggaaactcccctttttaaaaccatcagatatcatgaaacttatttactgtaatgagaacaggatgggattcaattaccttccactgggtcgctcccacaacacgtgggaattcaagagatttgggtggggacacagccaaaccatatcaagtactgtgcaagtgttttaggcatgcagagagtggtgggtcttcccagcaagcagagtgtggggaggtaatgggggactggtggctgacttaatggcccaggacccatgccacaaggagatggatggtggatgtgaataggagcctgcttacacccatcacaatttagattcttatgctcgatggcacgggtactcttttaggcccattttaccaatgaggagattgggactaatttgctcgagatcaaaaaagaagtggtgtaggtgggatttaaacccaggatgtctagcactaaaatgcaggtacttaaccactatcctaagggagtggctacttaatttgataaactcatctagtgaatggaagagagacggttacatttcactgatggtactgagcctttgttgatgagctcattgggaatctcagacatgagcaggatgtgtctaagggacaggtgggcttcagtagactggctaactcctgcagtctctttaactggacagtttcaagaggaaaaccaagaatccttgaagctcaccattgtatcttcttttccaggttgtccaataactgcatcacctacctaggggcagaagccctcctgcaggcccttgaaaggaatgacaccatcctggaagtctggtaaggcccctgggcaggcctgttttagctctccgaacctcagtttttctatctgtaaaatggggtgacgggagagaggaatggcagaattttgaggatcccttctgattctgacattcagtgagaatgattctgcatgtgaaggatctgattctctgtctaagaaagaagtctttacctctttaagtagggagcaatgatttcatttttaaaccttgactatttattcagcaacttctctgctctatgagatagtgtaggaatggggatgtggttgaagaatgaaaagaaaagtcagctcccgccctcctagaaattgcatctgccttcacaggtcaaggatattggatcagaccttctgcggttctgaatggagattacacaggttaggagcaggttgcacagtgtttccaattctctataattaaagccatagactttcatgtattgaaaaaagcaagaattgcattcttgacagattctttcattgccttaaaaagaatgactagccttgggagtctgggcagctgggtccagtgttgtagactttctctctgctgagccacagcttcaaagatttgtccttcttgtttccagggatctatttctcagacaataagtaaaggctttccctggcctaatgtgctgtaagtgaatgctactatatatgttccaggcactgggctagagactaatatttaaaagccaggaaatttcctatagaaaatctatatctcagggttttctcaaaagagctgggaactctggatgcccattcatgattccagtagttaaccagagtacaagaagggctgagtcttctcagatgggcaaacccactctggctgactgcagatccaccaagcctattgtcttagaccaggaccctttggcaactcattcccataagcctgtgacccttgctttaaatatgcaggccttgtcttctctcaaaaagcacatcaaggctgcagcgaatgcagatatcaaatgatgaagttaaaaacaaaagctttgctgggcgtggcagctcacacctgtaatcctagcactttgggaggctgaggcaggaggatcactttaggccagaggttcaacaccagaccttgtctctcaaaaaataaaaaattcagctgggtgcggtgtagttcctagccacttgggaggctgggatggaaggatcccttgaacccaggagttcaaggctgcagtgggccatgattgcatcactgcacaggcgacagaattagatcccatctcttaaaaaaataaaaaatttaaaagtgacttcaaaaatctatgctgtgatggagagatttttccttctgtatgattgtgatagctctgtggcctatgacgtcatcaggttctgggcaaagtgtaggttttctgtttctttgtttttgaaaccattgcacagtcctaagaaacatcacattctgggtcctgggcaccagccaacatgaggttgatattttaaggggtctgatgttatttgctgcctgttgatggaacttgggagggcaccagggtttgctcattgcattcttgacagattctcttattgccttaaaaagaatcactggccttggggagtctgtggctggctgggtgcagtgttgtggactctctctgcagagtcatggagccttgttcagaatgcttcctgagctgccctggttggccaagggtaaaaacagccctgacttccctgcaagaaacactgcagctgggccagagagtcagcccatcccaggcatgggtttaaaaagtggaggcttttgtttgaaagccctgctctaattttgtcctcactcaaacctctgttcacttgatctgctttaggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttgggggtttgagaccaacctgaccaacatggagaaaccctgtctctactaaaaatagtggaggttgcagtgagccaagatcacaccgttgtcctccagcctgggcaacaagagtggaactccatctcaaataaataaagaaaataaaaaataaaaatattactaaaacttgtcaaattttaaagcacaattttctacttgaagcggaggaaaatgatgttttcaacccgataatgtatagcttaaggcaaaagcaattcttagaaaatagccaggcaatcaaaacacttttggcttcagtttagaaaagtttaaccccgtgaagttttcagctgtgcaggagcctccccgtacccagcctaaaaggctgcaacacatgtgaaaaggccccttggcccccctcgattccctcaaagtggctccaggagtctttaacagaaggatggagaaggtttacttatggctggctctccacagcgagcagcctctcatgcaggccttgtgttaaaatagtgcttagctgcgtttaataagcaagggatcaggtggtcacccagcaaggtaaaatcctacttccttgaaaggttttgcgtctggctgactcggtggtgtggcatctccctggaccctgcatgggagtgtgtctatgggttttctttgtgattggtgccctgttctgcccagaccattgtttctgatacagcagatcaggaggaaggtggtgttgagtctcctttcgctgaggagtacacacagcagcccatttgcatagcactgtattgggttgtcagtttgtcatctttaaattcattcagcaatgaggccaggcatggtggctcatgcctgtaatctcagcactttgggaggctgaggcagctggatcacttgaggtcagaagttcaagaccagcctggccaacatggcaaaaccctgtctctaaaaatagaagaagaaaaaaaaaagttagctgggtgtggcagcgcacgcctgtaatcccagctacttgggaagcagaggcaggagaatcgcttgaatcttggaggaggaggttgcagtgagctgagatcatgccactgcactccagcctgggcgacacagcgagactgtcataaataaataaataaataaattcagcaatgatttattaaccagttatattaggatgaccagtggattgacaaaacagacatggtcctcaaggtgcttaggtgagggtggagggagaggcaggcagaaaacaagtcagttgaaaaagacatgaaaaagtcttcatccaggcactgtggctcatgcctgtaatctcagcactttgggaggccaaggtgggcagatcacttgcggtcgggagttcgagaccagcctggccaacatggtgaaaccctgtctctactaaaaatacaaaaattagccgggcatggtagcaggtgcctgtaatcccagctacttgggaggttgaa

SEQ ID NO: 7 - DNA, 41654 bp, Homo sapiens
ggggtgtatgtgtatcgggaagcatgtttgtggtggggggaagcagggcgtgtgtgtgtgtgtatgtgagtgttgggaagcaaggtatgtgtgttgggggaagcgagggctgtgtgtgtattgggaagcatgtttgtgtgtggggggaagcgagggctgtgtgtgtgtattgggaaacatgtttgcgtgtgggggggaagcagggcgtgtgtgtgtgttgcgaagcggtgtgtgtgtggtgtgtgtgtgtgcgcgtgtgtgctgggaagcggtgtgtgtggtgtgtggtgtgtgtgtgtgtgtatgtgtatgtgttgggaagcgggttgttccgaggactccccgattccctcctggttgaaacgtggcccagaaagagaggcacactggctagggcacacagccagggccgtccagccccgcaggaagccccggtcctgccctgtcccaggtaagcctggccagccctggagccggcgctgcggggctgcggggctgcggggctgcggggccacgggacaggagcgggtgctgcggtcccggcagagcaggggcggggtccgggcgggggcggagccgagcgcccgcgcacccggagccgcagcccgagccggagccggagccgggagtcgtggcccggagtgggccttggagtcggcgcgcaggcggctcgcggtgagtgctgtgcccagcgcctgggggtgggctggggggagactgtgggcgccgagggtcccacccccgggacttgaacacttggcttctgagccgtcctggccgcagggcttgggcaagggttgcctcggtttctcctgggggagtcccagaatgtagatgggaagtgagacggaaagggggcatctcgaggttggggaggcccctgggccactcttttgatttccttcctctctgtgggactcagtttccccagcttcaccatgccagtgagtgggtgggtataagtgagagagtctgcctggaggatcctgctgctgctctctgtacctcttatgggaatctcctgcctcctccttctcccttgttgggtgtgagatctaagttcaaatccatcccttcccagacagcccacaccaggacctgcgaggccacatcccccagctgtttgctctcctatgcttctcaggttgatggagcagccatctggtcctgctttcattccaacaaactgaaaagccctccaccccaatccccctggggaggacctcctgaagctgcatgagggctgggggctggacccctgcttctgagtttgctctttctgagcctctcctgccatggccttggtttccccatgggctgtgacctagcctcctccagggtccttgttctacagaggggaaacacctgccctgtcaccctcccctgaccccttagtgaaaaagaccagagtgacatccccaccacctgtacttgcctgtcccgatcactcagccatggggctgctggggcccctgagtttgttggctgctcaaggcccctttgcctcgctgcaccctgggatctgaggctggaagcatcctcatccagcccacctccctcctacccagtgcagagcccctctgtgacacatgcccagtggtggggagcttattactgatgaggtggctttttagtgccttgcagctcttgtttcctttagctaaaatctgtggcttacagattctacaccttggttccatgtctgtctcccggagccatgcagagcaagtcagttctttcattctttccagcagtcatcattcatgcactggatgagacacgagacagagcagtaggaaaatcagacaaaaccccctgccctcatggagtgcacactctagaaaggaatggacagcaatgaaataagtaaatacatggcgtagcaggtggcgggaagtactacgagtaaaacaaagctgggaagcaggatggggtgctggggctgggtattgcattgtatatagcacaaccagggaaggcttccccaagaccacgatagctgggcaaagtcataaggaattgaggggtgagccacatgagtatctggggggctgggaggctttggttttcactctgagcatgatgggagcacgggtttggaacagaggagtcacctgctttgacttggggtctggcaggcccatctagctgtggggtggagaatggatcatggggggggtggggtaagggcagaaggagagcccgttgagcggatcattgctgtcattctcgggagagaccatggtgtcttgcccatggcccagaggtggttaaaaaaaagaaaaccagacattcagatttgggatatatttcaaaggccgtgtcgatagggtttggggtgcaagagaaaggtagagtcaaagatgattctgagtgtttggctcgagcttctggaggtttgaagctgccatgaactgaaatgggagttgcttagcggagagatcaagagtttggttttgtgcttgtggagtttgagctgcccattaggcatcaatgatgagtaagcagttgggaaaagttaagggcggaggttggagttgaaaataaaactttgggcatcatcaccatataactggtatttaaagccacaagagcaggtgggctcatctagggatggagtgatatggagaagagaaggggtctaaccattgagtgctggggcccccagtgttaggaaccagccaagaagacagaaagagtgaaaatcagagagttggggtgtcctggaggaaatgaagaaaatgccccaaagaggaaggagggaacaaatatgaccaatgcccctggcagagcaagcaggctgagggctgaggattgagcaatgggaggtcactggtgacagtttcactggagctggatggggaactagagggaatgggaggggatgggaggacttggggacagcagtacaggcaacagacaagggggcctgctgtaaagggagcagataaatgggattggagccaaatgaagaaggggagtgtcaagagagtgctttacttttacaatggagaattagagtgcattgtgcactggtggggggatttgatctcttagggagagaacagtgttagggagggagaatgcaggatagctgggggagggtggggggcttggccccagcagagactcaggacacttgggaagttgagcttccctgggcttcccctcctctcctgtctgcaaggggtcagtgggctgagatttcagcacttaagcaaagcatttgctcttggccccagagaaaccgggctggctgtggtctcaggaaggaaggaggtgtccaggctcaggcctgggcctgggtttcagggagggcccacgtgggtcgccccttgaccctctctttcagcaaggaagtgatcctttctctacatgggcctcaccttggggaggacaatggtgtctttgaagttgtagtaactgaagtagagatcaaaaggcaatgcagatagactgacagatttcgcctgaagaggggaagcccgaccaggtaataaaggagtaagaggaaggatgttaaggacaattttaggaaacagataatgagtgaatattttttctctctctttcccaatttaaactgaagcaggagaaactgaagctagacataatgattaacttcccaagctggtgagcttcctgagctggttagtgagaacagcactaaggccaggttctcctccccagatgtttaagatgagacaggacaatgcctgctcagagacagggcctggctgaattggccctcaggattctctctgctctgaggtttctggaagaaggccagggcagaggtgtggtgatgtagctgctgggaggacagagctccgagtcacgtggcttgggcgggcctccccttcctggtgtccacagaagcccaacgtcactagctggggtgtgtatggctcacacgtaggccaggctgccctaggcttggtgtgcaagggaggggcccctacttacttgtggcctgtcccctcgtgaatgtgtctcatgtccccagtggggtttttcagtgagggtcatggtctccaggatgcacaaggctttgtgccagaattgcttggaattgcctagttctggaaggctggttggccaactctggcctccggcttttcctttgggaatttcccttgaaggtggggttggtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggtaagaggagcaggcattgtcccgtcccagcttgatcctcagccttctttcatccttggccgcgacatgctcccaggcctggggtcagatggggagtgctgactctgtttctgggctgttttctggggagaatgggtcggcgggtttttttccccaggacctgggcagggtcaatggtgggggccgctgtcgcatccttggctggtgtttccacagctgagaaccactccagggccaagcccagagcttattctacccttttttgtcctctcttcccctgtcctcggccaccccaccctcttggctcctctgcttagatgtgggcacaaggaggagaactccttggcctgagagaactaccttagatcctggcttccagtggcctctgcaggggggtacaccctctctcccaagcagccagacacacaagtaacctcattgcctcagtttccccatctgaccagcacagggccccctgtgccccagcagcgttctgagagattggagctttctccttttgcttaccttggctaccgtatgaggacggatacagagtgttccccccacccccagcccaggggatatttgattcatgaacattccctcagtgtctttgtgggggacaatgctgtgccaggctcagggatgccaggacgagtaagacccaggctcccacgtggcccaggcagggagagagacacataaacaaccatcaggaaagaggtaaaatccccaggccacttggcatctgctcccttgagtgtctgggaatgtccctgatttataaaaagaagctgacggccctctttgttgtccatgcctacaccctttcactttcgtttcttcggggcactgcagcagcccttgtccacagaccccatgacaatcgcagaactgaccatgctgagagattttcttggctgctcagggaccctgccagggcttgaagctcctggagggtcacttgccctcaaattcccagaacgcacagcaggtcactgatgatagcagtggcagcagtctgtgcacggtggtttcgagggcgtgggagggaggtgagggccctagggcaagtgtgtgtgggaagtgttgatgggggacaaggcaccagaacgctcggaaacaacttagtttgcaccgtaatttttcacttcgcctaggacaggacctttagagcaatattctgagtctaccccttggagtagcagtgtgcaaaacacacagcacgggcttggggcccccgtggggaacccaaatgtaagagttagagacatgcattccggagtcatacatggctcgtgttgaaatcctgactctgcctgtctagctgtgacacatcgtacaaatcacttagcttcttggtgcctcagtgtcttcctctgtagaatgggtagatcataggcactacttcagagtggctgggagggttcagtgaattcctgcaggagagcacttagaatggcacttggtgtgtagtttatgcttaattaatattagccgttactgaaactgctgtagcctgaatccagccagcatgaaagagcccctctcaccctgcttcgaagagaatgaattccctgattgtttggaagatctctctctctctctctgtcttttttttttttttttgagaaacggtcttgctctcttgcccaggctggagcgcaatggtgccatcttggctcactgcaacctctgcctcccgggttcaagtgattctcctgtctcagcctcctgagtagctgggattacaggcgctcgccaccacgcctggctaatttttgtatttttagtagagacagcgtttcaccgtgttggccgggctggtctagcgctcctgatctcaagtgaccttgggagatctcttgctcctaatattacctcaagcctttttaaacgttttaagccggagaccaagcatggatatgggagttaggggtcttgatttaattcttggttgcttcaaactctgtggaaccttgaggtgtttcttgccttctctgggtctcaattttcacatctatatggtggggagcttggattgggtaatgtctgaggctagaaccatggccaactcgggttctgctggggctgacttgccctggccttccctgaccaccctgcatctggcttctggagaagtcccgcactgaccttgttctcctccccaggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggtgaggcactcctggtgtgcatcacagagttctcaggaaaggggtgcttagtcaccaagactgatttgtcctcatgaagtcagcctgtggggtaacttggtccatgggatttcccctaaaaaggtagccaggcaggtaaaatttgctcttgactcttggcaggaaacgtacaactctttctttcttcttttcttttctttttctcactctgttaccctggctagaatgcagtggcacaatcatagctcactgtagccttgaattcctgcgctcaagtgatcttctggccttagagtagctgggactacggctgctgtaccaccatgaacagctaattttttttttttcttttagagatggggtgttgctatgttgcccaggctggtctccagctcctggctttaagcaatcctcccgccttggcctcccaaactgttgggattgcaggcatgagccactttgcctggccaacagaacacttctgccgagaggaagtgtgtggtggccaggaactcagattctggagccagaatggtgcaggctcaaggtcaaccctgtgtgatctcaggcttccctatggagcctctccagcctcagtctcccttgtttcagtttcctcatctacaaaacaatgttaatagtcaaatggtgcctatcctataaggctcttgggaggattcagtgagttaatttgagtaatgcttaggatagtgtctattaccactggctgctatttattatttctgttatgagtgatactctgtacttgtacacttttatttctgtctgttttaaattaacagcacaacagaccataacactgcagtatattgaatttattttataattaacatagcatattataaactaatatagcttaaatgtttatgtaggatttctgacatgaaattgcattagatcatagatgttcagagttggtatataacagcccctgagaatgtagtaactcagcagagaccagaaggtcagagaaatgaccactgagtatttttgaaactcttttgttttcttccaaatagtgattcttagggctcctgagaggcagatggaacaatcattaacattccactttataaatcgggaagttgagaccaaggaaagtagtttgaataagctcacagtagttaatgagggggccagtgctggaccaattggccagcactggtcattgacttattcatccatcattcatttattcagccagaatctattaggtgcttcatacatatttgcttaaagtttgttgtgttcatagagctttgcacacggtaggtactccataaacatttgttgatgaaataagtgagttactgaatgaatgattgaattagaatgacactgcagtgttaaaatgggctgggttggggaacattttagtttttgtttttgtctgttttccaaaaatgtatgtgttgttcacatgagtctggataaccctagattgagattgatgacataaataaatttgtcttcaaggctgcactaaagctggctcacatggctaggtatttacagagcagaagtggtgcagtcctctctgattagttgcacgtacagaagacatattcgttattggactgaccttagtttctcttataatttgttaggggaattgaatcagcccatctgagaagttacaagattgtgtcttgtcatctttaaaagttcagcaatgtgatgtggtacagatggtctgaggggtttggagaaggtagcctagatccctagggcccagagaagacaggatgtgaacagaggaagtacatggattggtgaagaaaagaaatgggataactcatgggtcaaagaagaaatcatgatggaaatcagaaaatattcagaaccatacaataatgagaatattatttatcaaaatctattggatgcagctaaagcaggacatagggggaaatttacaaccttaggtgcctagattaggaaagaaggaaggcatttgtttatttatttgtttatttatttatttgagatgggggtctcactgtgtcacccaggctgctggagtgcagtagcacgatcataaatcactgaagtctcgaacttctgggctgaagtgatcctcccgcctcagccttccaagtaggtgggacacaggctagcaccaccataccaggctaattttttttttgtagacacagggtcttgctatgttgaggtctcaaactcctgggctcaagtaatcctcctccctcggcttcccaaagtgctgggattacaggcatgagccactgcgcccatctaaggctgaattttaatgagctaagaattcatcttaagaaagggctaaatagacagcaaaagcaaacattgaaggttgggactgagctgagtgggtagcagggatgggagacaacagatctgaggagagcaggagattttgaaaggattgcactgcctgaggtttaagcctttagaatccagctctctctgagctccctttgagctctgacattctgtgactctgatttggtggccttcccttagtggccttactgatttcatttggatggtgcttgtggtatatccaaccaacatgtcttcccaaatggccttttaatttcctataaagaagtagttgtcattgattgcaggttagggacagaaaatgctgtggaatgaaacaaaatgcaagttaaagaactaaattccaaaaatacccattgctactattgactgagtgaattcctactgtgtgccagacactgtacccagtccattccctgtattgttttatttaagcctcacaagggtatagtgtgactacactgtttcttaacaatgaagaaactgcccaaatcgcccatctgggaagcggcccagctagaatttgaatccaggcctgttttcctccagagcttgtgctattctctgtctgtcataaaatgtgggggctttgtgtggtaaacttgctcagttgggcatagcagttgttaggaaacctgaggctggtaacaccagctgtaataccagctgtccgtctgactcatgcaactgttaaagttgatagggctgaggtgtcagactgagctctgaattgcctgattcctataacaatattaacttaaacattttttaaattgggaaatgcaccatgcatacagaagagtgtgtatatttcatatgtatagtgtaaactgttcccatcacccaggttaaaaaacaggatgttgccagtacctggggccttctttaactgcaactgctagaggtaaacactggcttgacttttgtgtaaatcatctctttgcctttctttaatgttttagcatcttttaaaataaatccccaaataatgtattgttctattttgaaaaactgagtagcaagccaaaaatagctgtgtaaagaaaggtcacttaaattaggctgggtgcagtggctcaagcctttaatcccagtactttgggaggctgaggcaggtggatcacaaggtcaggagatcgagaccatcctggccaacatggagaaaccccgtctctactaaaaatacaaaaaattagccaagaatagtggcatgtgcctgtagtcccagctactcgggaggctgaggcaggagaatcgcttgaacccgggaggcagatgttgcagtgagccgagatcgcactgcttgaacccgggaggcagaggttgcagtgagccaagatcgcaccactgcactctagcctgggtcacagagcaagactctgtctcaaaaaaaaaaaaaaaaaaaagaaaggttactattgccttttcttagatgaaggttcccaaggcagggaaagctaagtggagtctcagggacttggtctggcttttccttccctgggaatttataaggacctcttctgggaagtcagtcggcaatgccatgaatgagtctggggaaatattgggctcattgcaactggagggtctggtaggactgatgtgaattaggtgctgtgtccggaggaaaatggccagaggaagtgggctgctttgtacagtcagtggtaaagttgccaaaggctattatagctcacaggaatgggccaaggctaaacactcctgtggagtgaaatgaatgtcctcagctgactgaggcagcgggagttgaaaagaaacgatattagttcatggtgaagacaagtcaaatatagataaaggttagggtcaggcttgcctggacatctaggagataactgccctcaacttgtttgaatcttgagtcactgctccattttgtttgaactggtggccatctacttatagtatacagccatcaacctgagatttccctacatggtcttcctgccttggtctcctgtatcctgaatcctatggcctcttcttccctggtttactacattttgctagaccgtatcctccagtcaattccttagaatgaatgtatgaaagttaaaatttctgaggtctcacatgtcttaaagttccctcatactggattgatagtttggctgggtataaaattctgggctggccatcattttccttcagaattttgattgcattattccattatcctctcttttcaatattgcttctaagaattccaaaacctttttttttttttctttttgagacagtgtctcactctgtcacccaggctggaatgcagtagtgtgatctcagctcactgcaacctccacctcctgggtttaagtgattcttcttcctcagcctcctgagcagctgggattacaggcacccaccaccacaccctttagtagagatggggttttgctatgttggccaggctggtcttgaacttctgactttaggtgatctgcctacttcggcctcccaaagtgctgggattaaaggcgtgagccaccacacccagcctccaaaaccattttaaaactctttctggaagcttttaaaattttcttttagtccccagaattttaaaatttcaattatgtgccttggtgttcttccattatgttagtcacccaagaggtactttcaatctggaaacttctctatgttttgggaaatgttcttgattagtttacaggtgatttcttcctctccattttatctcttctcttttcatgaaactactattaattcaatgttagaattccttgactgatcatttaattttcttctattttccatctctgtgtctttttgctctacttttctatgatagtcacagctctatctttaaactcttgagtttttcatttttgatgtcatgattttaatttgcaaggggtaggtttgactgattcttttttgtagtatcttactcttgttttatggatgcaacatcttctttgacttaaggatcataagataggtgggttctttgtttgtttgtttgactgtttttcaccctatgtaaactttttctacaagtttctttccccttcccccctttttggcttctatctcccacattagatgctttctctgggctcatgatactctttggttttctttctcaagattgacaggtaggactttaaaacttgttgagcatgcgggtgaaacttgtctaccatgaatttcactgtagatattttggagattgacagtgtttatatctttagatctcgcctcctgggttgatcaagttatctgagtacaccacagaccttttgcctggggataaaccagaaatctgtttcagaaaccactttgattcagtcttccttgttttagtcatttccttcagttccggaggtccctcatgctgatcattccagagccctttacagatcctagggtacacactgcatggttttcaactttcttgttttggggttaagatttggctttcaggagtctcctcagtccgttactattcattcaatcagcaagtccttgagcacctgatttgtgccagacattcttctaggtgttagggatacctcagtgaacaaaacagacaaaaatctttgtcttggaaatacacacactccagtcaggggagagggacaataagccaaaggaaggaaattacagcgtgtgctagaaggtgataagtgctgtagaaagtaagtaaagtgggtttgggagttgagagtttgggaaggggataaatgatggcaattgtaaatagagtagtcagagttctcacttagaaggtgaaattcaagtaaagacttgaaggaggacagggaattagccacatggatggctaggggaaggcttccaagctgagaggacagccagagccaaggcccagaggcaggagcatacctggtagttttaggaaacaggaggccaggatgctgagtggagtaagagggggcatgaaaggagaaacttgggtccacgtggttctagacaggtatttttgtctgttttgggccctgaaggttactattggacttggactcttactctgaggaaatagggacgctattgggacgtttgtacaggagcaatgtgacctgagttttgtttgtaaaggattagactctggctgtggcattaaggctaggctgtgggggcaggaacagaagcagggggaccagttttgcagcctgtgcagctttccagataagcagggattgtggcttggaggaggatggtatagaggaggtgacaagaaatgactctatgtctggtatgtagatattggccacagatggcatttgagcactagagacctggctggtccacatggagtttccataagcacataatacacatcagatttcaaagacttaatatgaaagaaagaatgaaacgtaccttgctatttctttcttttttttttttttttgagacccagtcttgctctgtcacccaggctggagtgcagtggtgtgatctcggctcactgcaacctccgcctcccaggttcaagtgattctcctgcctcagcctcctgagtacctgggactacaggcacctgccaccacgcctggctaattttttgtatttttagtagtgatggggtttcaccatgttgtccaggctggtctggaactccggaccttaggggatctacccgccttggcctcccaaattgctgggattacaggcatgagccaccatgctcagccatatcttgctattttctacatggattacatgttgaaatggtaatgttttggctattgtggattaaatagaatatatgattaaagttgatttcatctatttcttttaactttaaaaaatatgtctgttagaggatttgaaattccacatgcggcttgcatttgtgacctgcatttcatttctgtggaacagtgccctttttgggacatgctttgaaggtggagtcaacaggatttggcagattacagacgagaggcttcaagggtgactccaagacttcggggcagagcacctggaagaaaggggttaatattagccaagatgaggaaggctgtcggtttggcaggtgcatgggcaggttaggagtttagttttgaatatgttggaggtgtttatgaaacttttaagtggagatggaaaataggcagttggatgtgcaagtccagggttcagggagacagttcaggctggagatgaagatgtgggagtctgaggagagattgtattcaaatattcaatccatgagacttgatgaaatcacttctcttccaaatgatttacagcctgcagaatcattttccctatctttgtaggtttatgtcttcattttgtttcatttatttttcagttattcactgttttagtgagttttgagtaggagccagattggatgcatgcgttcaattcaccatccaacactgtattaactacttgaaactcatgtggttgttcggttgtttttttgaccttttattctggatggaagagagatgcttatgaagttgcagtaatcagtaagccttcccacattgctccatcagccttcctggaagaataatgtcttctgcctttcctgtaggcaagaaggctgcttgatcttgccacggtgaaagcgaatggattggctgccttccttctacaacatgttcaggaattaccagtcccattggccctgcctttggaaggtaggtgtatgttctcagttaatcagaaagggaagggcagtcagtgcagatccatggttaagagcagaacacacctcggttaacatcccatatgctggcagtatagcctccctatgactcaatttccttgttttaaggctagcaccaccccgtctcattgggattttgggagcattaaaaggacaaaagcgtgtaatgttagctattagctttcattatctcccacacagtatactgacaattgggctaccatatattgagggctaactaaaggtgttacttaccatccaaactctcattatctgtaccgaaaagatatggacacatgttttgagttagggctggtatctcttgatctctgaaatttagcagctcacaatgggaaactcaagaaccaagtggatctagagactctggtatccctcagtgcccagggtcaccacccaaactcaggaacaggaggggcttggaccgcaccacttgaacataccaggcatcctgccaggtgctttatggacaatgtctaccctttgcaacaaccctgagaagtaggtggtgtttttttccaccttatagatgtggaaactgggcagggaggttaagtgacgagggaggggaagatgggtctgattgtaaattgtccccacctacactttctcttttcttgggagaagaaatgtcagttgtaaagagagagtgcaagcctggcactctttagggcttgttcctacaccactgtagggaaagctcattggcactgaagccccctgagctgtgtgtggtgctggcagatgggtctatcaccctgggctgtgtcctctgggcagcaagcaagcctgtgggcggggtggctggaagtctgtgcctggcactcgcgagtgcaccgtctcattgaagaacaggatctaaacatcagtgcgccacagcagggtgcgcggcacggagtgcaggccctggtttggcccttggttgaggtttgctgttgacatcatcaagcacagctagtcactgtaagaccaggccagggtgcaagattccccacacttctaaaggtgacaattggtgtgtttatttctctataaaatgacattttttttttctggagaattttagtatcattggtgatgactggaaaacctgcatcagaaatcaggtcggaagaggaagatatatatctgatatgtactggagaggaagatatctatcttatggtctaagttcagggatcctggtatattcagagggcagaaagctcagcaataatcatcaactctgggaacagaggtgacataaacacagggcgtcccctttgtgtgactgcagatagtcatcagtgagctcagagctctatgaaaattacttgctagtttttgggttgaaaatagtgggccagtgtttggttgggggcagtgaggctgtgatggcgggggaccatgccaagctcctaccagcctgggacgctaaaccagcacttccccatttcctgaaaggggaactaaactctgacacaggaaatggtttgcttgcattactttcaggatgagaaaggaagagcactggccttccaaacacaccccgtgcatgaaaactctccctgcatggggtgcatggggaggatggggaagtggaggcaggatcacagactcttgttcgagtgctcagctggggcaccccggtgaccccgaggccttcccttgctaggtccacccagatcaatcaggatcatctccccatctcgaagtttaactttatcacatctcagagttccttttgccacgtaaggtaacatattcacaggttctgagaatccggacatggacatctttgagggtctattgttgtgcctactatatccatgaataataatgataataagcaccattttttgagagtttgccatgtcagatattcttttaaactgtattttatctcgctgcctcctgaaaaaatccttccaggtgtatattgtccccatttttacagatgagagaactgaggcccagaaaggctaaatggcttgcccaagtgtatggtggacccaggttttcaaactcaggtgtgtctggcttcagagactgggctcctgagcccttaagccctttgttcccctttagaaaaagtcacctgaggctgagtggtgaagggatttatccaaagccacccggccactatggcaggacagatatcagaatacaggtcttccgatcccagcccagagccccttcccgtcatctagaactcctcctggtgtcagtaatgataacggcagtcactgatgtcttttgagcacttactttgtgttgagcacttacactgtgctaagcacttgacataggtcatcttagttgatccgtgtaaaactctgtgaggtagtgaccaacatttctcccaccttacagaggtggaaactgagggttaggaagtttccttgactgtcctcaaagtgcacagcttgtgaatggaggagccaggatgggcgcccgctggctctcctatcccttcagttatgtcagcgtcccccgcagcagcccattgtctggttaggtcccgtcttcaccatggtgccaccttcatctgcctcttcttctgccttccagctgccacatgcaagaagtatatggccaagctgaggaccacggtgtctgctcagtctcgcttcctcagtacctatgatggagcagagacgctctgcckggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtagtgagtgttactgggcattgctgttcaggtatgggggagcaccatcaaggctaagtgtgggagcaccgagctgggctctagaagtctgggcccagcttcgcctctgccaccctgctttgcaacactgcccagatcccttcccttctgggccttaatttcaatatgtgatgatgacagccacactttattgactggcctatgtgctgggtctggtgctatgctttccggaatgacctcatctaatctctacaaccaccctggcgggtaggcaggaatgttattatctccattatccttgacttgaggctcagagaagtgaagtaacttgtccaggaaatggcagagctggggttcacaaattgcatcattctgattacaggttttctgcctcccaccagtctatggatacacttcagaggctccctgaaaaccttgaggtcacttgcagaaagttttgtgtagtatgtgtccgtatcaggaacaacaccaaatcagaggtgacttgtgccccatcagagactttaacaccccaaccagatgggaatttcaggacccaagaaatagaaagtggctgcagggttacaactactgttggattcctgaggtagcacagtgtccaaacaggatttcagcactacccgtattgcttagagccccagccaaagatgtgaggttttgccctttggagaatctgtgcccctgaactcgggggcctctttccacatcttgggggcaggcaagggcagagggtgtgcctaggcctgcggatcagcatgcgacagattccccaacatccttccagcttgaaaggggattgccctgcttctatttagaacctataggaaagcagaagttctagattgaagttaaaattgattcccagcctccaggggctttgggctacacctggatgaccttaattgaccctaagcatgggacaaaccacttcctgagagtattaggatggtatacatcttctctgggggcaaagcaacaagatttatttttcatcatggaccaaacacatggatacccactagaaactgtgtagtgaattttgttaaccctgacatagggaccatggtctttaggttaaagcataataacaacataatacataacatatatagcgaatatatatatgtattatatgcaatgaatgtaaatatgattatacccatcatggtcttggaggaaacagatgacacacttaaaatgggtgttttgaggagagtttgaaaaacagattgtttacaagccatgggcaggagttaggaagagtgagagggttggtgcaggggcctggggttagtaacagctgggggagggtagacttgaagggggaaggggagggagactaattagctggggggaaggtatggagacggctgcctgagcttctgcaaagtggaagaataccgcttggccctaactcctcaccccaactcttgctcctggccagcgccttccaccagctggacccatcagggaggccgagtgggctgtctgctggagtagtccccaggcatcagcctcccaggagccagggacgggtagagaagggggagagtggatctggccaggcaaatggaaaacagccagcaccaaactctatttccctaggagggaggatcatgatactttgagtgggaatttggaaacctgtctgttggagcaatttccctgatagaaataagaatgtgcattttcctgggtagtagactcagtttttaccccaagaggccaggcatcactggcctgtgtgatcctcataggccagtccatctctggaattcttgaatggatcatccatccttgattagggatgtccccgtgattaccagggtgtgcagaagggctctgggaaacctgtgggtctgtctctgtgttcagagaaaggtgagggtggcctggttctagctcatggtgctcagactgtggtgtgtaaaggcactcgtggcaatgcagattcctgggcctgcctctagtgattcccattcagtaggtttggggtggggcccaggaaatctatatttttcacagacacccctggtgattctgatacaagtggtctcgccctgggagaactactggtctgcagcaaccagcttggttttccattagcaattactgtccttgagcgagttttactgctcttcaccttacacacactaaaactgccaaggccgtaggggaggggaagcaaccatgaggttgctgtgagtgcactgtgtgtgtgtgtgtgtgtgtgtgtgtgtgtgtgtgtgtgtatgagagagagagagagattgagaaagagaggaagggaggaagggggagggcacaggctcctctcccacagtgccaacctgcctctctcccacttgaagtgtttccatgccaactgaaatcctcagcctctaggaaaccctatatacacagtgcccctatataggtttctttagactctggctctctcagactctagagtgatggctttaaaagttttttgaatgttacccacaatgattgagaaagagagagagagagcacgcaccaccatgtaaacatggaacctaagtttcacaaaatgacttcgctttatgaactctgagacactctgctctcttctgttctgttctatttccattttagaaatgctgctcaggaccttcaaaatgatttgcatgacctgcaacctgcagtctgaaaaatcactgcactacagaagtggccataagaggccctgagggagaagctgcacaatgtcatggttaagagtggggtttggagccaagccgcctaggctcaaagcctttatgtgccgtacaaccttggcaaagtcacttcgcttgtctgtgcctcagtttctttctcacgaatgctcataataatggttcccatttcactggcttgttgtgaggatgaaatagtgttattattgagaagtggtaagggtagtgatcagtgctagcgatcatgattctaggtgacttttactgtgtaccgggtgctcacaaggctttatgtgcacagcctggtgaggctgataatactattgttccctctttttttttttttgagacggagtctcgttctgttgcccaggcagggggtacagtggcacaatctcggctcatgcaagctctgcctcccgggttcacgccattctcctgcctcagcctcccaagtagctgggactacaggcgcctgccaccacgcccggctaatttttttgtatttttggtagcgacagggtttcactgtgttaaccaggatggtctcgatctcctgacctcgtgatccgcccgcctcggcctcccaaagtgctgggattacaggcgtgagccaccgtgcccggcctgttccctcttttatagatgaagagaccagcaaataactagtaagtcgctgatcaggatcacaatatccagctgaggcactccagagcctgagctgttaaccattcagtcagggcctcccaagtttgcctaaagataaagaatcatgtgcacagttgttaaaatatacagattcctgggccccaccccgcagatacttgattgccagctccagggtatgggcctgagaatctgtcttttagggaagctttcagatgatgttgtgatcaggtgagttttgggaatggtgccccaagaggagtggcagacagggcttgctcggcagggactagcctgttggagtggtgccattggggttaaggactgggcagcagggcctcactaaccacagcctatatgcctgtttctgaagttttggccactctcatccagctggtctactgtctgctgacctagatgatggtaaattgtccccaggggtagcctgtctagttcaggctgcacctttcgcatatatcagctcctttccaccatcatcccctttgtgaggctgctgtgattatcatgttccttttgcagagatggaaacattgcctcaaattagctctgtcatttcctaaggattccagggttctttagtagggggtctggatcctacgtcctgggccatccccatcatagtgcaccacgtcacctccctggccagggaccgtggggtctccacttttttggggtgctccatctatgcagggtttcctggaagcacagatgctggcacttcagggatgaatgaaagtctttttgggggatttgtagatttttttcttgtcttactagctccattttcaaatgtatttattttgtctctttagtttgcgcgataacaatatctcagaccgaggcatctgcaagctcattgaatgtgctcttcactgcgagcaattgcagaagttagcgtaagtcagcctgggctgtggacaatgggctccaagtgccctggtctcaccccaggtcgtgcagcctgggaagctgtgagtgatgggctggggcaggggctgtttgcatgatggggggtgcaggtgattcctgcccagaggggaagggcaaccctgggatttggtgctcactgtccaatgtgctttgcttctgtgtctcctctcttctggaactgaacagtctattcaacaacaaattgactgacggctgtgcacactccatggctaagctccttgcatgcaggcagaacttcttggcattgaggtgagcccaggttttccttattccctggaaactattttttgccccattcctgagtcagtctgatctggtcttggcctggcactgcccacactggctcctgacctcctgattgaatgcagggacagtgtctcattttaagcaggggttctctaatgctgtgatctccccagtaaactctggactagctctgctgaggacttcctgtcttttgacctttagcccgtagggcaagaaagcttttctaggcccctttccttttctgtgtctaagagtgtcacagctttctgggtttactgagttccacgatgcatgttgagctcgtcctggtgggggaggcatacacagttacttgccaccccagctgtggcagcgagttgctgcaacactcccaggaggtcctttcaccactcagagcatgcaaggtttgcagtccatctggttctgcatttctgctactccagtgtctcccagtttcaacaggagtctctctctctcctacctgatgcctttaaattgcccctctagctggccgctgggttggcctggcttctctctccttctctctctctcagatattcttgcctcctgtgatttgtgaggcagtaaaaaaagacaaagtaaagaattgcttccatctattcttttacctcttgggctgggtttgtggatgggagccgccattttaaaatggcgggccacatagctcagtctcggcaagggctactgagatcagaaccacaggtgccaatttgtacaaaggactcagtcctgctaccactgcctgatccctcagactcacaagcctggaataggctgtggccagacctggctggcccatccctgagaagggtgctagtttcagaaatggaggctgagtttgtggccaacacagtagtcctccggtatgtgcaggagagatgttctaagaccccagtggatgcctgaaaccatggagagtatcaagccctacacataccatgcttttcccaatacctacacacctgcaataaagtgtagtttataaattaggcacagtaagagagtaatagcaactcataataaaatagaacaattataacaatcaatgtactataataacactatgtgaatgtggactctctccatctccctcaaaatatcttcttgtactgtactcacccttcttcttgggaagatgtgtggtggtaaaatgcctgtgtgatgggaggaagtgaggtggatgacgcatgcagcactgtgctctagcgctgggctgctgttgacctgaccacacttcaggaggagaatcatctgctcccagagatccctaatctttgagcaacaatgaggtcggcagctggatgtcaggagcagacgatcttgatgattaccaaatgggagcgtatagagcgtggatgcgctggacggggggctgattcacgtcctgggtgggatggagctggatggcacgtgatcagaatagcatgcaatttaaaatgtatgaattgtttatctctagaattttccatttaatatttttggactgcagttgatttcagataactgaaaccatagaaggcgaagctgcggataagcagggggcagggattaccgtatatcattgtaatagagagcacaggctctggagccagactgcccgaggtttgaaccctcattagctgcgtgacctcaggtcagcccaatgtctgtgtgcctccgtttccccttctgtagaatggaggtaataaccctggctacctcacaggctgtagtgatgagcaagcaagttaatccacatgaagggctgcaccgtctggcaggggctttatatagtaagcgagtggctgaaagatgatgggtaaatcacacaagcactcagcttgtttctccttatgtgagtccggtcctccaagcagggattcaatgtgccacccatttattggggaaaagtcctaaaaggggaagtggggaagggagctgggggaggctgggaggtgtgtccctgagtgaaggagagagggaaggaaggaaggttgagactgggcaccttggacttcagtgcagtcctaagacatcttggcaaggctgatgaggagttcttgaaccaaattcaccaggcaggggagcctgatgtctcaggcaggggctggcaagtgcagatgcgaggatgttagattttggagcacagcagctggggcccttggctacctccaaggagctgaggctggagacctgaaaggcgagttctcctagctgccacaccccttctccaaggatacaataatatctgccttataggattgttgtgagctgagtggcttgacgttccttgaaagaatgaaagcgtatagttatcccaggaagcctagggttgcaggtgagagctctggggcttctccgaagctctccgaggtgtctggattcagttgcagcaggagccttccttgctgggatcttcccccacccctagccttggccctccctctctccttcctttctggaaggctcagtgggccccacccctccctccagccacctggacctgcccagcgctcttgtgcaacaggtaaagcctacctgtagcaacaacagatctgggaaggctgcagagggcacgatggggtctggatcgagggcggctgagaccagagggaaaggtgtgaccctgagtcaccctcgctgtcccggggaaaccacctcccaggacagctgcctactgtggctcctgcctggaattgtcacactgctgtgcaaacagcgtcccgctgcccctttccctttgctgggggaaaatgaagttgtgggagccgctgagtaaactagacctagcagcgagggcacctgatgtggctgctgcctcccgggcaggtcttcaatgctttcttcctgtgtttccctggccagggcacagacggccctccttttctgcctgccgctgtgttctctcagcctcctctgtcttcccttccaggctggggaataactacatcactgccgcgggagcccaagtgctggccgaggggctccgaggcaacacctccttgcagttcctggggtaggttggattccaggaagagggacctgcatggaggggcttgggacttttgaggatttaggggcaggtgaaactcttcagccaggaggccccagaggcagcccagctccagtggggaggacaagccagggagagagtgggcggcccttgactgccaccttcatacttggtctatgcctgacaaacaggaagtttgggatgttggggctaggggaggacagtgcccacgagctggtgacaggaagccctctgatcctcagggggcgctagggctgtactttagctgcatattaaaaccacctggaagcttctaaacactattgccaggcctcccaccccagactgatgaaatgcaaatatctaggtgcaaggcccaggtatcaggagttttaaaaagcttcccaggggatgtacagccaggggtgaggacccctgacctaagaaagagaaggaaatggggaaggataggaaggcacccaggataagaggggctgtgctaggtccctcggagctcttgctccctgtaggaccatgctagggcctgccagggaggggagtaccccaacctgcagccccagggtgggcttcctctgtttgctaggcacccaggcttgcacctgtgctgtttccagcagcctctctcctatcctgtcatgccctagtgtgaactggagtccatttgacaagaactgggagttttagaacctgggactgtaggaagagagaataaccttagggcctaggtgttccagcccatttcacagggaggcaagttgcccccaagctcagttttttgttttgttttgttttgtttgagatgtagtctcactctgttgcccaggctagagtgcagtggcacgatcttggctcactgcaacctccgcctccttggttcaagcgattcacctgcctcagcttctcaagtagctgggattataggcacccaccaccacgcccagctaatttttgtatttttagtagagacagggtttcaccatgttggcccggctggtcttgaactcctgatctcagatgatccgcccgcctcggcctcccaaagtgctgggattacaggtgtgagccaccgcacccggcccccaagctcagtttgagccacaaatgggactatgttgctctagaaatcaacatcttttccacactgcattagtagcaacagagtctagaacaaaggaggccacagccccactgaactctcttctgcttgaggtcacatctgccacatcaggggtatttacctctttcaacacatatttattagggcacctgtctgggccaggcgttgtgctaaaacccccaaacgctgtcatatgatacaaagtgttctgtaacttgcttggtttttttttttgtttgtttgtttgttttgttttgtttttgttgttgtttttttttgcttcgccatatattataggaatttttttaggtcattatgacctctttatttacttaattatctatttatttattttactaatatttacagaaagggtctcactctgtcacccaggctggagtgcagtggttgcaatcatagctcattgtagccttgaactcctgagctcaagtgatcttcctacctcggcctcctgagtagctgggactacaggcacaagccaccatgcctggccgatatttttatgttttgtagagacggggtctcactatgttgcccaggctggtctcaaactcctgggctcaggtgatcctccctcctttgcctcccaaagtattgggattacacaagtgagccaccttgctcagcctgacctcatttttcaaagagctgcagagtgttacataatgtatttaactggtcactttttgatgactattaagttgttttcaggttttttgttattacagtgtcatatccctggggcacagagcagtgctggcacatagccagagctcaatcgatacatacctaatgaatgaaagtacagtggacatcctaattcagccattctttgctaacttgtgtacatacctgtccagggtaggtccctagaatacagtcaataagtcagaaggtgtgagttgggatctaccttttggaaagggatgttttcaaactacagtgagtcagaggaggatggcccagaagctgggggagttgaagctgatggcgtgaaggaattaggggtgttaggaagaagcaggagataaagagctagcttgcagaagaagtgttagacttgttatgggcaggtactggagggtagctaaggacttgtgggtggcagttaccaggaagcgtatctgaactaagtgtcagaaaaagtgtcacaactgtaaattactcttgtcagtgagttcctgtccttaagggttagggctgggtagccctctactattctctaagtctgtaatgtaaagccactgaaaactcttgggttaagtttggccatcccacccaaaagatggaggcaggtccactttgctgggaccaggagccccagtgaggccactctgggattgagtggtcctgcccctctggctgggactgcagagggaggaggactgttagttcatgtctagaacacatatcaggtactcactgacactgtctgttgactcttttggccttttcagattctggggcaacagagtgggtgacgagggggcccaggccctggctgaagccttgggtgatcaccagagcttgaggtggctcaggtaagcttcagagtctatcctgcagttttcttggggagatcaggtgaagagggaggagctggggccagttctgaaggtctttgaactttatttctaccccacaatgttaggcaatggagtaaggaaaaaagaccattggatttcaagagaggacactcgagtctttctgggtgacttggaaatgtcccttgtcctctcagggttttgatacagtatctgtaaattgaagatattgggctggatcaggtacattttatcttaaggaccaattccaatccattggtagtgggtgcccagtgcaccacattaaaaagaattctaaggctgcacctgggcttaaagaagagcactataatcaattagtgatgtctaaaaaagctaaaaaaaaaaaaaaaagagcactgcattcaattagtgatgtctaaaaagggtagaaaaaaaaaaaaaaagaaaaaagaaagagcaccgcaatcaattagtgatgtctgaaatggagcagaccaggagagcaccacgaattttgccctccataggttagctcatctctgaggtctttccctgctctgacatacttttgttccatgattacctccagcctggtggggaacaacattggcagtgtgggtgcccaagccttggcactgatgctggcaaagaacgtcatgctagaagaactctggtgagtttgggggattctctgctctggggaagtggatcacaatctctgttgatcccctggcctcatccataggagcggttgtgtggacagacaaaggtggatgattgagtgattgactgattgattgattgtgtttgtctttatatgtactgagtggtatgaagcttatagagcctggtatgtacatgctaatttttttatttaataaaatatatgggtttgctggtttggtgactgcctccacatggcataagtgttaagagcacagactctgtaatcaagcaggccgtgatcttaggcaagttaaataacaatttcagaatctcaagtttcatgtctgtaaaatgagggtaagaatacttccaaccataaaggatttttgcaagaattagataaagtagtgcctgtgaagaccttaatatagtgcctggcatatttgtaagtgctccataaatgttaaattagaataatggcagggttactactactattactgctgctgctgctgctgctgctgctacaactactatagtactgtgactactactactaataaagttttgttattttaaagtgattttgagttcctaggagcactgggtattcaagtcttaggtcattttggaaggtgtaatggagttttgatagttgaaagaggaaccatgaatcatgcttatactgttgacctgaagcagattctaagtttctcatcctttagatgccactagtatagttttctgacatgttctgggcagcttcagattatgtcagggagataaaatactgaatgtttgattttcccgggaagcagaaaggcactgcaacatatgggcattgccataaacagattttatggatggaccttggctgttgcagggcttactagctctactcaagtatgattgattctatcctgactggattttgccacttggaatttcttagtagaggagaaccttgttatgagagcatcagttatgattactgttaaaagaaaaactttaggcaaattaaatttagcagaactggtttgaacatacagcaatttatgaattgggcagcattcagaactgggagtgctccacccagcaaggtaggcaagcagtatctatagacaggaaaaggaagtgatgtacaaaacagcttgattggttgcagctgggcatttgccttatatgggcatggtgtgatgaggcattttctttatatggatatagactgatcagctggtagactgtgactgactgaagcctggctgctgtgattggctaagacttagctgtttgttataaggatatgttgttaggttgcagtttgctacataggaactcaaagtacagaggcagtctcaggccaaatttagtttaactatatgttaagctgcaggtgacagaatacctccatctatagaggtttaaacaaggaaagggtttattttttcctgtataggcagctggatgtaggcagtgtagggtttgtacagtggctacaagaggccaggaggggtctcagctctgtctcattctcttcctgttccatcattcttagcctgtgacttcattcacatggttggttgtctcatgatcacaggatggctgctccaggtgcagcactacttctgtattcccggattcgatctatatacccaggaaagccatctgggttctctcctttaaaaagcattcctggaagccccacctgtcgacttccccttatgtatcaaccatgtgtatgtcacttgaccaacccacttgtatgttgtttgaccagccctggctgcaatggagagtgggaaatacagttttttcaccaagtgcatggctgtccaaatgaaatgagacttccattaataaggaagaaaggaaagatggagatcaggaagctgggggatcagggaacttattacattgagagcccttggagtgaattctcttgcaaatatgtccctggaattgagaatccccacaacgtactttatctgttctttctttatccatgagtttgggttttcagatgttggatttcctatatggggggcatgtgagttcatcatcttccataatcaatgttgtatcaactggattttctctcttcttctcaccagcctggaggagaaccatctccaggatgaaggtgtatgttctctcgcagaaggactgaagaaaaattcaagtttgaaaatcctgaagtaaggaacccataagcaggaaacaggacaataattgctggcctttggaaggggcatttctgaataagatctgggccgctctccgctgggctaactcatgtgaggtggcctggtagaacagcttgccttggtctaggtggacaaggattccagtgcaagttgtttatctgggaggtggtcccagtaaatgctgataggagagtggtgaagtgagatggggaagtgaaggtaaccaataaaggggagttatcaagccagttatcaatgagggaaattggagctcagtactctggggcactcctggagccagtgcagaacacacatggtcacctacccaaccaatgggcaagaaagccatggcatttatccaccaaccctctgtccttcctatgttgatgtgcgctcatggggcactgattctccagcacttccagctcaccctcacccagctgaacatgcttctggggtcaggagaatggcctcaggcagagagtggaaggtcttctctgcaagcagtggctggggaggtgatgtgatggggagtactgtggcctcctccagtggctgactcagtggcttgggacttgtgccacaaagagatggacagctcaggtgaacatgaacccacctagtgaccatcatgggtttgtcagggtgctctctgaggctgatgccaaaattcttatttcaagtagacctcaggaaccccatcagatggctccttttgctggaggaaagtggcatctgcctaggcaaatgtggtcctaggaaaacgcttgcctttagagacagacagacagacagctgcctctgtgagtgccagctttgctgccaggctgctacccactctggcgacactcatttgtgttgctttcacaagctaggaagtttccaaatatttggagaaaacacttccactaattatttgggtggaaatgggctgggaagttggggtgaagcccggatgtgtctgagccagatgccagctttgcactgagggtcggcctttgggaataccaagcccattatcaaccaggtgtggatatggcaggtttgtcttccctccttgtcacagccttactccacttgactcccatggatgccaggcaatgaggctggggttggtcccatgccaccctgtcatcagccttatttttcagcatcctaaactatatcatcccccacaaaaattgaacttctgatatatcttttataaaaaagagaaatgcctacatctttcttttccaggcttagtttctgccaagagttggttgagagcccaggcttgctgggtgcagtggctcacacctgtaatcccagcactttgggaggctgaggcgggtggatcacctgaagtggggagttccataccagcctgaccaacatggagaaaccccatctctactaaaaatacaaaattagccgggcgtggtggcatacacctgtaatcccatctactcaggaagctgaggcaggagaatcacttgaacctgggaggtggaggttgccatgagccaagatcacaccattgcaccctagactggacaagagagaaacttccatctcaaaaaaaaaaaaaaaaaggatgagaaaaataataatttaaaaaaaagagtccaggctctggaaccagacagcctgggtcttacccctgctccaccattaccagccagttcttcttggatgagtgcctcagttgcctcaagtgtaaatggagataatggctggaccttcattataggccatgagcattcactgagagaatgtagctaacaaaagtgagttgtaggttggagcaaaagtaattgtggtttcagaccatgaactttaaattattataactaggctaaaatacatctttattaatcaaaataggaaccattaaaatcaacacatttttgccaataagaaataagtttgtttattcctgtagcataaaaattcatgcttcgggattcaacaaactcttggaaagcattttctgcatcctcctggttgtggaagcatttttcctgcagaaagttgtcaagattcttgaagaaatggtagtcagttggctagaggtcaggtaaatatggcggatgaggcaaaacttcatagtccaattcattcaacttttgaagctttggttgtgtgacatgcagtcgggtgttgtcgtggagaattggaccctttctgttgacgaatgccggctgcaggtgttgcagttttcagtgcatctcattgacttgccgagcatacttctcatatgtaatggtttcgcagggattcagaaagctgtaggggatcagactagcagcagaccaccagtgaccatgaccttttttttttggtgcgaatttgcctttgggaagtgctttggagcttcttctcggtccaaccactgagctagtcattgccagttgtataaaatccacttttcatcgcacgtcacaatcagatcaagaaatggttcgctgttgttgtgtagaataagagaagatgacacttcaaaatgacgattttcttggttttcactcagctcatgaggcacacacttatcgaggtttttcacctttccaatttgcttcaaatgctgaatgaccatggaatggtcgatgttgagttctcaagtagttgtaagaaaatcagctttgatgattgctctcaattggtcattgtcagcttctgatggcctgccagtacactcctcatcttcaaggctcttatctccttcgcaaaacttcttgaaccaccactgcactatacgttagttagcagttcctgggccaaatgcattgctgatgttgtgagttgtctccgctgctttacaacccattttgaattcaaataagaaaattgcttgaatttgctttttgtctaacatcattttcatagtctaaaataaatataaaataaacagaaagtattaagtcattagcaaaaaatcataaagtgagaattgtgcattaaaatgatgtatagcataaccacatttatttaagaatgtattccaatatcaaatggcaaatttcaacaatgcaaaaactgcaattacttttgcaccaatctaatagaagttcaataaatactggcaattacaattggcattgccttagggtcaacttgtaagacattcctgaaattgtgggaaagggggaggacctggagtggacattattggaaggcaaagctgtaaccaaaagagcaacctgggaaacacatgactcctctgttgctgtccctggccctatcctgtctcccctccctgttgtcagctacctcatatgttctctaatctctgtctctgtgccctcaaagacccccctgaaaatagaaatattactgctcattggttattttctatcaattaagtactgtattagtccgttttcatgctgatgataaagatatacccaagactgggcactttatgaaagaaagagttttattgaacttacagttccacgtggctggggaggtctcacaatcatggctgaaggtgaaaggcacatctcacatggcagcagacaggagaagagggcttgttcagggaaactcccctttttaaaaccatcagatatcatgaaacttatttactgtaatgagaacaggatgggattcaattaccttccactgggtcgctcccacaacacgtgggaattcaagagatttgggtggggacacagccaaaccatatcaagtactgtgcaagtgttttaggcatgcagagagtggtgggtcttcccagcaagcagagtgtggggaggtaatgggggactggtggctgacttaatggcccaggacccatgccacaaggagatggatggtggatgtgaataggagcctgcttacacccatcacaatttagattcttatgctcgatggcacgggtactcttttaggcccattttaccaatgaggagattgggactaatttgctcgagatcaaaaaagaagtggtgtaggtgggatttaaacccaggatgtctagcactaaaatgcaggtacttaaccactatcctaagggagtggctacttaatttgataaactcatctagtgaatggaagagagacggttacatttcactgatggtactgagcctttgttgatgagctcattgggaatctcagacatgagcaggatgtgtctaagggacaggtgggcttcagtagactggctaactcctgcagtctctttaactggacagtttcaagaggaaaaccaagaatccttgaagctcaccattgtatcttcttttccaggttgtccaataactgcatcacctacctaggggcagaagccctcctgcaggcccttgaaaggaatgacaccatcctggaagtctggtaaggcccctgggcaggcctgttttagctctccgaacctcagtttttctatctgtaaaatggggtgacgggagagaggaatggcagaattttgaggatcccttctgattctgacattcagtgagaatgattctgcatgtgaaggatctgattctctgtctaagaaagaagtctttacctctttaagtagggagcaatgatttcatttttaaaccttgactatttattcagcaacttctctgctctatgagatagtgtaggaatggggatgtggttgaagaatgaaaagaaaagtcagctcccgccctcctagaaattgcatctgccttcacaggtcaaggatattggatcagaccttctgcggttctgaatggagattacacaggttaggagcaggttgcacagtgtttccaattctctataattaaagccatagactttcatgtattgaaaaaagcaagaattgcattcttgacagattctttcattgccttaaaaagaatgactagccttgggagtctgggcagctgggtccagtgttgtagactttctctctgctgagccacagcttcaaagatttgtccttcttgtttccagggatctatttctcagacaataagtaaaggctttccctggcctaatgtgctgtaagtgaatgctactatatatgttccaggcactgggctagagactaatatttaaaagccaggaaatttcctatagaaaatctatatctcagggttttctcaaaagagctgggaactctggatgcccattcatgattccagtagttaaccagagtacaagaagggctgagtcttctcagatgggcaaacccactctggctgactgcagatccaccaagcctattgtcttagaccaggaccctttggcaactcattcccataagcctgtgacccttgctttaaatatgcaggccttgtcttctctcaaaaagcacatcaaggctgcagcgaatgcagatatcaaatgatgaagttaaaaacaaaagctttgctgggcgtggcagctcacacctgtaatcctagcactttgggaggctgaggcaggaggatcactttaggccagaggttcaacaccagaccttgtctctcaaaaaataaaaaattcagctgggtgcggtgtagttcctagccacttgggaggctgggatggaaggatcccttgaacccaggagttcaaggctgcagtgggccatgattgcatcactgcacaggcgacagaattagatcccatctcttaaaaaaataaaaaatttaaaagtgacttcaaaaatctatgctgtgatggagagatttttccttctgtatgattgtgatagctctgtggcctatgacgtcatcaggttctgggcaaagtgtaggttttctgtttctttgtttttgaaaccattgcacagtcctaagaaacatcacattctgggtcctgggcaccagccaacatgaggttgatattttaaggggtctgatgttatttgctgcctgttgatggaacttgggagggcaccagggtttgctcattgcattcttgacagattctcttattgccttaaaaagaatcactggccttggggagtctgtggctggctgggtgcagtgttgtggactctctctgcagagtcatggagccttgttcagaatgcttcctgagctgccctggttggccaagggtaaaaacagccctgacttccctgcaagaaacactgcagctgggccagagagtcagcccatcccaggcatgggtttaaaaagtggaggcttttgtttgaaagccctgctctaattttgtcctcactcaaacctctgttcacttgatctgctttaggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaacagccatcttccttgtgaccaaacatttaaaaatattctggctgggcacagtggctcacgcctgtaatcccagcactttgggaggccgaggtgggcagatcacctgaggttgggggtttgagaccaacctgaccaacatggagaaaccctgtctctactaaaaatagtggaggttgcagtgagccaagatcacaccgttgtcctccagcctgggcaacaagagtggaactccatctcaaataaataaagaaaataaaaaataaaaatattactaaaacttgtcaaattttaaagcacaattttctacttgaagcggaggaaaatgatgttttcaacccgataatgtatagcttaaggcaaaagcaattcttagaaaatagccaggcaatcaaaacacttttggcttcagtttagaaaagtttaaccccgtgaagttttcagctgtgcaggagcctccccgtacccagcctaaaaggctgcaacacatgtgaaaaggccccttggcccccctcgattccctcaaagtggctccaggagtctttaacagaaggatggagaaggtttacttatggctggctctccacagcgagcagcctctcatgcaggccttgtgttaaaatagtgcttagctgcgtttaataagcaagggatcaggtggtcacccagcaaggtaaaatcctacttccttgaaaggttttgcgtctggctgactcggtggtgtggcatctccctggaccctgcatgggagtgtgtctatgggttttctttgtgattggtgccctgttctgcccagaccattgtttctgatacagcagatcaggaggaaggtggtgttgagtctcctttcgctgaggagtacacacagcagcccatttgcatagcactgtattgggttgtcagtttgtcatctttaaattcattcagcaatgaggccaggcatggtggctcatgcctgtaatctcagcactttgggaggctgaggcagctggatcacttgaggtcagaagttcaagaccagcctggccaacatggcaaaaccctgtctctaaaaatagaagaagaaaaaaaaaagttagctgggtgtggcagcgcacgcctgtaatcccagctacttgggaagcagaggcaggagaatcgcttgaatcttggaggaggaggttgcagtgagctgagatcatgccactgcactccagcctgggcgacacagcgagactgtcataaataaataaataaataaattcagcaatgatttattaaccagttatattaggatgaccagtggattgacaaaacagacatggtcctcaaggtgcttaggtgagggtggagggagaggcaggcagaaaacaagtcagttgaaaaagacatgaaaaagtcttcatccaggcactgtggctcatgcctgtaatctcagcactttgggaggccaaggtgggcagatcacttgcggtcgggagttcgagaccagcctggccaacatggtgaaaccctgtctctactaaaaatacaaaaattagccgggcatggtagcaggtgcctgtaatcccagctacttgggaggttgaa

(19) United States
(12) Patent Application Publication
(10) Pub. No.: US 2022/0228222 A1
(43) Pub. Date: Jul. 21, 2022

(54) Cancer stratification and treatment based on Inhibition of NOD-2

(71) Applicants: Christopher Goodnow, Darlinghurst, New South Wales (AU); Megan Barnet, Darlinghurst, New South Wales (AU); Katherine Jackson, Darlinghurst, New South Wales (AU); Jonathan Cebon, Darlinghurst, New South Wales (AU); Andreas Behren, Darlinghurst, New South Wales (AU); Garvan Institute of Medical Research, Darlinghurst, New South Wales (AU); Olivia Newton-John Cancer Research Institute, Heidelberg, Victoria (AU)

(72) Inventors: Christopher Goodnow, Darlinghurst, New South Wales (AU); Megan Barnet, Darlinghurst, New South Wales (AU); Katherine Jackson, Darlinghurst, New South Wales (AU); Jonathan Cebon, Darlinghurst, New South Wales (AU); Andreas Behren, Darlinghurst, New South Wales (AU)

(21) Appl. No.: 17/609,517

(22) PCT Filed: May 8, 2020
(86) PCT No.: PCT/AU2020/050460

§ 371 Date: Nov. 8, 2021

(30) Foreign Application Priority Data
May 8, 2019 (AU) 2019901562
May 8, 2019 (AU) 2019901563

(51) Int. Cl. C12Q 1/6886; A61P 35/04; C07K 16/28; C07K 16/18; A61P 35/00

(52) U.S. Cl. CPC C12Q 1/6886; A61P 35/04; C07K 16/2818; C07K 16/2827; C07K 2317/76; A61P 35/00; C12Q 2600/106; C12Q 2600/156; C07K 16/18

(57) ABSTRACT

The present disclosure relates to predicting a patient's response to cancer therapy based on the presence or absence of loss of function NOD-2 variants, as well as methods of treating cancer comprising inhibiting NOD-2 function.

### CROSS-REFERENCE TO RELATED APPLICATIONS

The present application claims priority from Australian Provisional Patent Application No 2019901562 filed on 8 May 2019 and Australian Provisional Patent Application No 2019901563 filed on 8 May 2019, the content of each of which is incorporated herein by reference in its entirety.

### TECHNICAL FIELD

The present disclosure relates to the field of predicting a patient's response to cancer therapy. The present disclosure also relates to methods of treating cancer.

### BACKGROUND

Chemotherapy and radiotherapy have been extensively used to eradicate cancer based on their direct cytocidal effects on rapidly proliferating tumour cells. Several types of immunotherapy are also used to treat cancer. These treatments can either help the immune system attack the cancer directly or stimulate the immune system by enhancing the body's immune response to fight the cancer.

Despite the excitement surrounding cancer immunotherapy, most patients do not respond to immune checkpoint inhibitors. Responses to immune checkpoint inhibitors (CPI) may vary between individuals because of somatic mutation differences in the tumour and/or germ-line differences in immunological tolerance. Furthermore, there are patients for whom chemotherapy and radiotherapy simply do not work.

Hence, there is need to predict and improve a patient's treatment outcome in response to anti-cancer therapy.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

NOD2 is a receptor for muramyl dipeptide (MDP), a peptidoglycan component of bacterial cell walls, serving as a cytoplasmic sensor of infection with invasive bacteria and triggering a signalling pathway that activates inflammatory responses. However, little was known about the role of NOD2 in the immune response against cancer cells.

The inventors have identified that mutations that inhibit or reduce NOD2 function (herein referred to as “loss-of-function mutations”) are present in people who have an exceptional response to anti-cancer therapy. On the basis of these findings, the inventors have developed new methods for predicting a patient's response to anti-cancer therapy and/or selecting a patient suitable for anti-cancer therapy on the basis of their NOD2 status. The inventors have also developed new methods for treating cancer comprising inhibiting NOD2 in a subject.

Accordingly, in one aspect, the present disclosure provides a method of selecting a subject for anti-cancer therapy, the method comprising:
(i) determining the sequence of a NOD2 encoding nucleotide sequence in the subject;
(ii) comparing the nucleotide sequence determined at (i) to a reference NOD2 encoding nucleotide sequence;

wherein the presence of a loss-of function mutation in the sequence determined at (i) relative to the reference NOD2 encoding nucleotide sequence indicates that the subject is likely to be responsive to anti-cancer therapy.

In another aspect, the present disclosure provides a method of predicting the response of a subject to anti-cancer therapy, the method comprising:
(i) determining the sequence of a NOD2 encoding nucleotide sequence in the subject;
(ii) comparing the nucleotide sequence nucleotide sequence determined at (i) to a reference NOD2 encoding nucleotide sequence;
   wherein the presence of a loss-of-function mutation in the sequence determined at (i) relative to the reference NOD2 encoding nucleotide sequence indicates that the subject's response to anti-cancer therapy is likely to be improved, relative to if the subject had the reference NOD2 encoding nucleotide sequence.

In another aspect, the present disclosure provides a method of stratifying a subject according to their likely response to anti-cancer therapy, the method comprising:
(i) determining the sequence of a NOD2 encoding nucleotide sequence in the subject; and
(ii) comparing the nucleotide sequence determined at (i) to a reference NOD2 encoding nucleotide sequence; and
(iii) stratifying the subject according to their predicted response to anti-cancer therapy, wherein the presence of a loss-of-function mutation in the sequence determined at (i) relative to the reference NOD2 encoding nucleotide sequence indicates that the subject's response to anti-cancer therapy is likely to be improved, relative to if the subject had the reference NOD2 encoding nucleotide sequence, and wherein the absence of a loss-of-function mutation in the sequence determined at (i) relative to the reference NOD2 encoding nucleotide sequence indicates that the subject's response to anti-cancer therapy is likely to be substantially the same as if the subject had the reference NOD2 encoding nucleotide sequence or a poorer than a subject who has a loss-of-function mutation.

In another aspect, the present disclosure provides method of selecting a subject for anti-cancer therapy, the method comprising:
(i) determining the protein sequence and/or activity of NOD2 in the subject;
(ii) comparing the protein sequence and/or activity of NOD2 determined at (i) to a reference NOD2 protein sequence and/or activity, wherein the presence of a loss-of function mutation in the subject's NOD2 protein sequence and/or reduced NOD2 protein activity indicates that the subject is likely to be responsive to anti-cancer therapy.

In another aspect, the present disclosure provides a method of selecting a subject for anti-cancer therapy, the method comprising:
(i) determining the level of expression and/or activity of NOD2 in the subject;
(ii) comparing the level of expression and/or activity of NOD2 determined at (i) to a reference level of expression and/or activity NOD2;
   wherein a reduced level of expression and/or activity of NOD2 determined in (i) relative to the reference level indicates that the subject is likely to be responsive to anti-cancer therapy.

In another aspect, the present disclosure provides a kit for determining the sequence of a NOD2 encoding nucleotide sequence in a subject, and/or for determining the level of expression and/or activity of NOD2 in a subject. In one example, the kit comprises one or more reagents configured to determine the sequence of a NOD2 encoding nucleotide sequence in a subject. Alternatively, or in addition, the kit comprises one or more reagents configured to detect the presence or absence of a loss-of-function NOD2 protein variant. Alternatively, or in addition, the kit comprises one or more reagents configured to determine a level of expression and/or activity of NOD2 in a subject.

The one or more reagents configured to determine the sequence of a NOD2 encoding nucleotide may be a primer or an oligonucleotide probe. The one or more reagents configured to detect the presence or absence of a loss-of-function NOD2 protein variant may be primers flanking a NOD2 sequence comprising one or more mutations associated with the response to anti-cancer therapy. The one or more reagents configured to determine a level of expression and/or activity of NOD2 in a subject may be primers or probes for qPCR to determine the level of NOD2 protein expression or an antibody which binds to a NOD2 loss-of-function variant.

In another aspect, the present disclosure provides an antibody which binds to a NOD2 loss-of-function protein variant.

In one embodiment, the method further comprises preparing a reference NOD2 encoding nucleotide sequence from a population of individuals.

In one embodiment, the subject is receiving, has been prescribed or has received anti-cancer therapy. For example, the subject may be receiving anti-cancer therapy. For example, the subject may have been prescribed anti-cancer therapy. For example, the subject may have received anti-cancer therapy.

In one embodiment, the methods described herein further comprise treating a subject determined as being likely to be responsive to anti-cancer therapy, wherein treating the subject comprises administering the anti-cancer therapy to the subject.

In another embodiment, the methods described herein further comprise administering a NOD2 inhibitor to a subject determined as being less likely to be responsive to anti-cancer therapy based on their NOD2 status. In one example, the subject to which the NOD2 inhibitor is administered is receiving, has been prescribed or has received anti-cancer therapy. The method may also comprise administering the anti-cancer therapy to the subject. The anti-cancer therapy and the NOD2 therapy may be administered together or separately. In accordance with an example in which the anti-cancer therapy and the NOD2 therapy are administered separately, they may be administered simultaneously or sequentially.

In another aspect, the present disclosure provides a method of treating cancer, comprising inhibiting NOD2 function in a subject in need thereof, wherein the subject is receiving, has been prescribed, or has received anti-cancer therapy.

In another aspect, the present disclosure provides the use of an inhibitor of NOD2 function in the manufacture of a medicament for treating cancer in a subject, wherein the subject is receiving, has been prescribed, or has received anti-cancer therapy.

In another aspect, the present disclosure provides an inhibitor of NOD2 function for use in treating cancer in a subject, wherein the subject is receiving, has been prescribed, or has received anti-cancer therapy.

In another aspect, the present disclosure provides a method of treating cancer comprising administering an inhibitor of NOD2 function to a subject suffering from cancer, wherein the subject expresses a functional NOD2 protein.

In one example, inhibiting NOD2 comprises administering to the subject a NOD2 inhibitor, an RIPK2 inhibitor, an LRRK2 inhibitor, inhibiting a ubiquitin ligase required for NOD2 signalling activity, or any combination thereof. In one example, inhibiting NOD2 comprises administering to the subject a NOD2 inhibitor. The NOD2 inhibitor may be a direct inhibitor of the NOD2 protein. For example, the NOD2 inhibitor may be GSK717 (N-(2-(1-(2-(2,3-Dihydro-1H-inden-5-ylamino)-2-oxoethyl)-1H-benzo[d]imidazol-2-yl)ethyl)-N-methylbenzamide). In one example, inhibiting NOD2 comprises administering to the subject a RIPK2 inhibitor. For example, the RIPK2 inhibitor may be gefitinib; small molecule inhibitors of p38a (SB203580) and Src kinases (PP2); OD36; WEHI-345 (N-(2-(4-amino-3-(p-tolyl)-1H-pyrazolo[3,4-d]pyrimidin-1-yl)-2 methylpropyl)isonicotinamide); erlotinib; Type II small molecule inhibitors of protein kinases, such as ponatinib, regorafenib or sorafenib. In one example, inhibiting NOD2 comprises administering to the subject an LRRK2 inhibitor. For example, the LRKK2 inhibitor may be CZC 52146, CZC 54252 hydrochloride, GNE-9605, GNE-0877, GNE-7915, GSK2578215A, JH-II-127, LRRK2-IN-1, MLi-2, PF06447475 or URMC-099. In one example, inhibiting NOD2 comprises inhibiting a ubiquitin ligase required for NOD2 signalling activity.

In one example, the ubiquitin ligase is selected from the group consisting of: XIAP, cIAP2, cIAP1 and Pellino 3. For example, the ubiquitin ligase is XIAP. For example, the ubiquitin ligase is cIAP2. For example, the ubiquitin ligase is cIAP1. For example, the ubiquitin ligase is Pellino 3.

In one example, inhibiting NOD2 function comprises genetic inhibition of NOD2.

In one example, the anti-cancer therapy is chemotherapy, immunotherapy, radiotherapy, or any combination thereof. For example, the anti-cancer therapy may comprise chemotherapy. For example, the anti-cancer therapy may comprise immunotherapy. For example, the anti-cancer therapy may comprise radiotherapy.

In one example, the immunotherapy comprises administering a checkpoint inhibitor selected from the group consisting of PD-1/PD-L1 targeting agents and CTLA-4 targeting agents. The PD-1/PD-L1 targeting agents may be selected from the group consisting of pembrolizumab, atezolizumab, avelumab, durvalumab and nivolumab. The CTLA-4 targeting agents may be selected from the group consisting of ipilimumab and tremelimumab.

In one example, the subject is suffering from a cancer selected from the group consisting of: thoracic cancer, head and neck cancer, melanoma, skin cancer, neurological cancer, germ cell cancer, sarcoma, hepatobiliary cancer, upper gastrointestinal cancer, lower gastrointestinal cancer, breast Cancer, CNS cancer, gynaecological cancer, genitourinary cancer; neuroendocrine and adrenal cancers, cancer of unknown primary, lymphoma, leukaemia, colon cancer and plasma cell neoplasms. In one example, the cancer is colon cancer, lung cancer or melanoma. For example, the cancer may be lung cancer. For example, the cancer may be melanoma. For example, the cancer may be colon cancer

In one example, the method further comprises determining the NOD2 status of the subject prior to treating the cancer. In accordance with this example, the subject to whom the NOD2 inhibitor is administered has been determined as having functional NOD2.

For example, determining the status of NOD2 in the subject may comprise:

(i) determining the sequence of a NOD2 encoding nucleotide sequence in the subject;

(ii) comparing the nucleotide sequence determined at (i) to a reference NOD2 encoding nucleotide sequence which does not comprise a loss-of-function mutation;

wherein the absence of a loss-of-function mutation in the subject's NOD2 encoding nucleotide sequence is indicative that the subject should be administered the NOD2 inhibitor to improve their response to anti-cancer therapy.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1 is a box plot showing the burden of common susceptibility DNA variants in each of the exceptional responders (coloured circles) and in each of the 1144 well-elderly people in the Medical Genome Reference Bank (MGRB).

FIG. 2 is a box plot showing the burden of common susceptibility DNA variants in each of the exceptional responders and in the lung adenocarcinoma patients (grey box, above) and the 1144 well-elderly people in the MGRB (black box, below). The percentiles are 10, 25, mean, 75, 90.

FIG. 3 shows the ranking and the mean burden of each DNA variant in the exceptional responders and in the MGRB. Seven variants out of the top fifteen identified affect either NOD2 or its functional partners, LRRK2 and IRGM, which encode proteins that interact with NOD2 in subcellular complexes and promote NOD2 signalling.

FIG. 4 shows the germinal centre B cell response to immune stimulation. A. Gating strategy for analysing germinal centre B cell response to intra-ocular sheep red blood cell (SRBC) immunisation (day 7). B. Column scatterplot depicting germinal centre B cells as a proportion of B cells in spleen, day 7 post SRBC immunisation in homozygous wt, A162M and frameshift animals. Animals harbouring a NOD2 functional variant have reduced germinal centre B cell response to immune stimulation.

FIG. 5 shows a growth curve of MC38 mean tumour size in C57BL/6 wt mice treated with 300 μg IP every 3 days×4 doses: active drug (BioXCell InVivo anti-mouse PD-1) or isotype control/vehicle (BioXCell InVivo MAb rat IgG2a).

FIG. 6 shows the response of C57BL/6 wild-type (wt) versus NOD2 loss-of-function (NOD2) mice to treatment with anti-PD1 A. Growth curve of MC38 mean tumour size in C57BL/6 wild-type (wt) versus NOD2 loss-of-function (NOD2) mice treated with anti-PD1. Cull T1 (timepoint 1) after two doses, cull T2 (timepoint 2) after four doses. B. Dot plot analysis of tumour immune-cell infiltrate of NOD2 vs wt mice treated with anti-PD1. Parameters are CD44 (y-axis) and CD62L (x-axis). Q1: Effector memory (EM), Q2: Central memory (CM), Q3 Naïve CD8+ T cells. C. Column scatterplot depicting the combined values (n=6 at T1, T2) of CD8 effector memory cells in NOD2 vs WT mice, as a proportion of total CD8 lymphocytes. T1=timepoint 1 (after two doses of anti-PD1), 2=timepoint 2 (after four doses of anti-PD1). EM: Effector memory, CM: Central memory.

### KEY TO THE SEQUENCE LISTING

SEQ ID NO: 1 Nucleotide sequence for a reference human NOD2 encoding gene sequence.
SEQ ID NO: 2 Amino acid sequence for a reference human NOD2 protein.
SEQ ID NO: 3 Nucleotide sequence for an exemplary human NOD2 variant encoding gene sequence (rs2066844; p.Arg702Trp).
SEQ ID NO: 4 Nucleotide sequence for an exemplary human NOD2 variant encoding gene sequence (rs2066847; p.Leu1007ProfsTer2).
SEQ ID NO: 5 Nucleotide sequence for an exemplary human NOD2 variant encoding gene sequence (rs2066847; p.Leu1007ProfsTer2).
SEQ ID NO: 6 Nucleotide sequence for an exemplary human NOD2 variant encoding gene sequence (rs2066845; p.Gly908Arg).
SEQ ID NO: 7 Nucleotide sequence for an exemplary human NOD2 variant encoding gene sequence (rs104895423; p.Leu248Arg).

### DETAILED DESCRIPTION

### General Techniques and Definitions

Unless specifically defined otherwise, all technical and scientific terms used herein shall be taken to have the same meaning as commonly understood by one of ordinary skill in the art (e.g. in genomics, immunology, molecular biology, immunohistochemistry, biochemistry, oncology, and pharmacology).

The present disclosure is performed without undue experimentation using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only.

Functionally equivalent products, compositions and methods are clearly within the scope of the disclosure, as described herein.

Each feature of any particular aspect or embodiment or embodiment of the present disclosure may be applied mutatis mutandis to any other aspect or embodiment or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise. For example, a reference to “a bacterium” includes a plurality of such bacteria, and a reference to “an allergen” is a reference to one or more allergens.

The term “and/or”, e.g., “X and/or Y” shall be understood to mean either “X and Y” or “X or Y” and shall be taken to provide explicit support for both meanings or for either meaning.

Throughout this specification, the word “comprise’ or variations such as “comprises” or “comprising” will be understood to imply the inclusion of a stated element, integer or step, or group of elements, integers or steps, but not the exclusion of any other element, integer or step, or group of elements, integers or steps.

Response to Anti-Cancer Therapy

The inventors have surprisingly shown for the first time that the presence of a loss-of-function mutation in a subject's NOD2 encoding nucleotide sequence is indicative of a subject's likely response to anti-cancer therapy. Based on this finding, the inventors have developed and provide herein (i) methods for determining or predicting whether a subject is likely to respond to anti-cancer therapy based on NOD2 status, (ii) methods for stratifying a subject according to their likely response to anti-cancer therapy based on NOD2 status and (iii) methods for selecting a subject suitable for anti-cancer therapy based on NOD2 status.

As used herein, the term “subject” refers to any animal, for example, a mammalian animal, including, but not limited to humans, non-human primates, livestock (e.g. sheep, horses, cattle, pigs, donkeys), companion animals (e.g. pets such as dogs and cats), laboratory test animals (e.g. mice, rabbits, rats, guinea pigs), performance animals (e.g. racehorses, camels, greyhounds) or captive wild animals. In one embodiment, the “subject” is a human. Typically, the terms “subject” and “patient” are used interchangeably, particularly in reference to a human subject. The subject may be receiving, or may have been prescribed or may have received anti-cancer therapy. In one example, the subject is receiving anti-cancer therapy. In another example, the subject has been prescribed anti-cancer therapy. In another example, the subject has received anti-cancer therapy.

As used herein, the term “stratify”, “stratifying” or similar in the context of a method of the disclosure shall be understood to mean classification or division of subjects tested into groups or categories indicative of the likely response of the subject to anti-cancer therapy. For example, a subject on which the method of the disclosure is performed may be classified or identified as an individual who is likely to respond to anti-cancer therapy relative to the response of an individual having a reference NOD2 encoding nucleotide sequence. In another example, a subject on which the method of the disclosure is performed may be classified or identified as an individual who is likely to respond poorly to anti-cancer therapy relative to the response of an individual having loss-of-function mutation in their NOD2 encoding nucleotide sequence.

NOD2

NOD2 is an intracellular pattern recognition receptor that assembles with receptor-interacting protein (RIP)-2 kinase in response to the presence of bacterial muramyl dipeptide (MDP) in the host cell cytoplasm, thereby inducing signals leading to the production of pro-inflammatory cytokines.

The NOD2 gene (previously known as CARD15) provides instructions for making a protein that plays an important role in immune system function. The NOD2 protein is active in some types of immune system cells (including monocytes, macrophages, and dendritic cells), which help protect the body against foreign invaders such as bacteria and viruses. The protein is also active in several types of epithelial cells, including Paneth cells, which are found in the lining of the intestine. These cells help defend the intestinal wall against bacterial infection.

The NOD2 protein has several critical functions in defending the body against foreign invaders. The protein is involved in recognizing certain bacteria and stimulating the immune system to respond appropriately. When triggered by specific substances produced by bacteria, the NOD2 protein turns on (activates) a protein complex called nuclear factor-kappa-B. This protein complex regulates the activity of multiple genes, including genes that control immune responses and inflammatory reactions. An inflammatory reaction occurs when the immune system sends signalling molecules and white blood cells to a site of injury or disease to fight microbial invaders and facilitate tissue repair. The dysregulation of NOD2 signalling has been associated with various inflammatory disorders suggesting that small-molecule inhibitors of this signalling complex may have therapeutic utility.

Surprisingly, the inventors have shown that the presence of a loss-of-function mutation in a subject's NOD2 encoding nucleotide sequence or a mutation which inhibits NOD2 signalling through other genes involved in the NOD2 signalling pathway can improve the subject's response to anti-cancer therapy relative to if the subject had a reference NOD2 encoding nucleotide sequence. Based on this finding, the inventors contemplate that a subject's NOD2 status i.e., whether or not the subject has a loss-of-function mutation in their NOD2 encoding nucleotide sequence, may be predictive of how that subject will respond to anti-cancer therapy.

As used herein, the term “reference NOD2 encoding nucleotide sequence” will be understood to mean a NOD2 encoding nucleotide sequence which does not possess a loss-of-function mutation. Thus, a subject with a reference NOD2 encoding nucleotide sequence will be understood to have a reference level of expression and/or activity of NOD2. For example, the reference NOD2 encoding nucleotide sequence may be a wildtype NOD2 encoding nucleotide sequence e.g., a wildtype human NOD2 encoding nucleotide sequence. In another example, the reference NOD2 encoding nucleotide sequence may be a consensus sequence constructed from a population of individuals which do not possess loss-of-function mutations in their NOD2 encoding nucleotide sequence. An exemplary reference NOD2 encoding nucleotide sequence is set forth in SEQ ID NO: 1.

Determining a subject's NOD2 status is well within the means of a person skilled in the art. In accordance with the methods of the present disclosure, the NOD2 encoding nucleotide sequence in the subject may be determined by any suitable sequencing methods known in the art. For example, the NOD2 encoding nucleotide sequence may be determined by Sanger sequencing or high-throughput sequencing methods such as next-gen sequencing. The NOD2 encoding nucleotide sequence may be determined by PCR, such as quantitative, real time PCR (qRT-PCR). Suitable primers for performing PCR may be design by conventional means, based on the sequence of the NOD2 gene. The subject's NOD2 encoding nucleotide sequence may then be compared to a reference functional NOD2 encoding nucleotide sequence by any suitable method known in the art. For example, the subject's NOD2 encoding nucleotide sequence may be aligned (using any suitable sequence alignment or bioinformatics software) with the reference NOD2 encoding nucleotide sequence to identify differences between the subject's NOD2 encoding nucleotide sequence and the reference NOD2 encoding nucleotide sequence or the subject's NOD2 protein sequence may be aligned (using any suitable sequence alignment or bioinformatics software) with the reference NOD2 protein sequence to identify differences between the subject's NOD2 protein sequence and the reference NOD2 sequence. It may also be readily determined whether any differences in NOD2 encoding nucleotide sequence translate into differences at the amino acid level (i.e., loss-of-function mutations). The amino acid sequence of the subject's NOD2 protein may also be determined by any suitable method known in the art. For example, the amino acid sequence of the subject's NOD2 protein may be determined through Edman degradation, mass spectrometry or through determination of the subject's NOD2 encoding nucleotide sequence and subsequent translation of this nucleotide sequence.

In one embodiment, there is provided a kit for determining the status of a NOD2 encoding nucleotide sequence in a subject. In one embodiment, there is a provided a kit for determining the NOD2 protein sequence in a subject. In one embodiment, there is a provided a kit for determining the presence or absence of loss-of-function mutations in NOD2. In one embodiment, there is a provided a kit for determining the level of expression of NOD2 in a subject. In one embodiment, there is provided a kit for determining the activity of NOD2 in a subject. In one example, the kit comprises one or more reagents configured to determine the sequence of a NOD2 encoding nucleotide sequence in a subject. Alternatively, or in addition, the kit comprises one or more reagents configured to detect the presence or absence of a loss-of-function NOD2 protein variant. Alternatively, or in addition, the kit comprises one or more reagents configured to determine a level of expression and/or activity of NOD2 in a subject. The one or more reagents configured to determine the sequence of a NOD2 encoding nucleotide may be a primer or an oligonucleotide probe. The one or more reagents configured to detect the presence or absence of a loss-of-function NOD2 protein variant may be primers flanking a NOD2 sequence comprising one or more mutations associated with the response to anti-cancer therapy. The one or more reagents configured to determine a level of expression and/or activity of NOD2 in a subject may be primers or probes for qPCR to determine the level of NOD2 protein expression or an antibody which binds to a NOD2 loss-of-function variant.

In one embodiment, the presence of a loss-of-function mutation in the subject's NOD2 encoding sequence is indicative that the subject is likely to be responsive to anti-cancer therapy. In one embodiment, the presence of a loss-of-function mutation in the subject's NOD2 encoding sequence indicates that the subject's response to anti-cancer therapy is likely to be improved, relative to if the subject possessed a NOD2 encoding nucleotide sequence that did not comprises the loss-of-function mutation e.g., such as if the subject had the reference NOD2 encoding nucleotide sequence. In one embodiment, the absence of a loss-of-function mutation in the subject's NOD2 encoding nucleotide sequence is indicative that the subject's response to anti-cancer therapy is likely to be substantially similar relative to if the subject had the reference NOD2 sequence. In one embodiment, the absence of a loss-of-function mutation in the subject's NOD2 encoding sequence is indicative that the subject's response to anti-cancer therapy is likely to be poorer than if the subject possessed of a loss-of-function mutation in their NOD2 encoding nucleotide sequence.

The presence of a loss-of-function mutation in a subject's NOD2 encoding nucleotide sequence may result in a change in the level of expression and/or activity of NOD2. Accordingly, in one example, the method may comprise determining a level of expression and/or activity of NOD2 in a subject. The level of expression of NOD2 may be determined by any suitable method known in the art. For example, the level of expression of NOD2 may be quantified by measuring the level of mRNA transcripts encoding for NOD2 by qRT-PCR or by RNA seq. The presence of a loss-of-function mutation in a subject's NOD2 encoding nucleotide sequence may result in a change in the level of functional NOD2 protein or NOD2 protein activity. The level of functional NOD2 protein may be determined through Western blot or mass spectrometric techniques. The activity of NOD2 may also be determined by any suitable method known in the art. For example, an antibody may be used to detect a NOD2 loss-of-function protein variant or an assay may be used to determine NOD2 activity. An example of a suitable assay to measure NOD2 activity is through the determination of cytokine activity by in vitro stimulation using muramyl dipeptide (MDP) (Hsu et al., 2008).

In one embodiment, there is provided an antibody, or a functional fragment thereof, or other binding agent which binds to a NOD2 loss-of-function protein variant. An antibody, functional fragment thereof, or other binding agent which binds to a NOD2 loss-of-function protein variant may conveniently be provided in a kit of the disclosure.

It will be understood by a person skilled in the art that an antibody refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes as well as the myriad immunoglobulin variable region genes. Light chains are either classified as either kappa or lambda. Heavy chains are classified as gamma, mu, alpha, delta or epsilon, which in turn define the immunoglobulin classes IgG, IgM, IgA, IgD and IgE, respectively. The basic immunoglobulin structural unit is generally at tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (V_{L}) and “variable heavy chain” (V_{H}) refer, respectively, to these light and heavy chains.

Antibodies may exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}l by a disulfide bond. The F(ab)′₂ may be reduced under mild conditions to break the disulfide linkage in the hinge region thereby converting the F(ab)′₂ dimer into an Fab′ monomer. The Fab′ monomer is essentially an Fab with part of the hinge region (see, Fundamental Immunology, W. E. Paul, ed., Raven Press, N.Y., 1993). While various antibody fragments are defined in terms of the digestion of an intact antibody, it will be appreciated that Fab′ fragments may be synthesized de nova either chemically or by utilizing recombinant DNA methodology. Thus, the term antibody as used herein also includes antibody fragments either produced by the modification of whole antibodies or synthesized de novo using recombinant DNA methodologies.

As used herein, a “loss-of-function mutation” in NOD2 refers to mutation in the NOD2 encoding nucleotide sequence which results in reduced function of NOD2 compared to if the subject had the reference NOD2 encoding nucleotide sequence. In some embodiments, a loss-of-function mutation in NOD2 results in a complete loss of function of NOD2. In some embodiments, a loss-of-function mutation in NOD2 is a partial loss-of-function e.g., a mutation which results in a reduction in NOD2 activity or function. Non-limiting examples of NOD2 loss of function mutations are disclosed herein. Any of the NOD2 loss of function mutations disclosed herein may be present in the subject as a homozygous or heterozygous mutation. The NOD2 loss of function mutation may be any mutation identified in Tables 1, 2, 3, 4 and 5 herein. Thus, the NOD2 loss of function mutation may be any one or more of: G908R, R38M, R138Q, R702W, L248R, W355, D379A, L550V, N825K, L1007fs, L1007P, A105T, D113N, V162I, T189M, D357A, I363F, P463A, P727C, A755V, E778K, R790W, A849V, W907R, G908R and R1019. For example, the NOD2 loss of function mutation in the subject may be any one or more of: L1007fs, G908R, R702W or T189M. In one example, the NOD2 loss of function mutation may be L1007fs. In another example, the NOD2 loss of function mutation may be G908R. In another example, the NOD2 loss of function mutation may be R702W. In another example, the NOD2 loss of function mutation may be T189M. The subject may be homozygous or heterozygous for this mutation, as with any of the other mutations disclosed herein. Thus, in one example, the NOD2 loss of function mutation may be L1007fs/wt. In another example, the NOD2 loss of function mutation may be L1007fs/T189M. In another example, the NOD2 loss of function mutation may be G908R/wt. In another example, the NOD2 loss of function mutation may be R702W/wt. In another example, the NOD2 loss of function mutation may be G908R/R702W. In another example, the NOD2 loss of function mutation may be L248R/wt.

The methods disclosed herein may additionally or alternatively comprise determining a loss of function mutation in a NOD2 associated protein. NOD2 associated protein may be, for example, a NOD2 binding partner or an upstream regulator and/or downstream target of NOD2. Such NOD2 associated proteins are known in the art, including those disclosed herein. A loss of function mutation in the NOD2-associating protein may be any of those disclosed herein. For example, the loss of function mutation in the NOD2-associating protein may be any one or more of rs11175593, rs7714584, rs11747270, rs11564258, rs1000113, rs12422544 and rs11741861.

Any of the NOD2 loss of function mutations disclosed herein may be present in the subject in any combination or permutation. The mutations may be detected by determining either or both of a subject's nucleotide sequence or protein sequence.

Where mutations are disclosed herein as amino acid variations (e.g., substitutions), the nucleotide variations which result in these amino acid variations will be apparent to the person skilled in the art. Examples of nucleic acid variants representing NOD2 loss of function mutations are provided in SEQ ID NOs: 3-7.

As used herein, “functional NOD2” refers to the protein encoded by wildtype NOD2 or proteins encoded by variants of NOD2 that exhibit normal NOD2 activity.

The methods of the present disclosure may be performed prior to receiving anti-cancer therapy, while the subject is receiving anti-cancer therapy, or after the subject has received anti-cancer therapy. In one example, the subject is receiving anti-cancer therapy. In one example, the subject has been prescribed anti-cancer therapy e.g., but is yet to receive the anti-cancer therapy. In one example, the subject has received anti-cancer therapy e.g., is undergoing anti-cancer therapy.

As used herein, the term “improved” shall be understood to mean decreased mortality, increased magnitude of response, decreased timing of treatment, decreased disease progression, decrease of pathological symptoms for the subject, relative to if the subject had a reference NOD2 encoding nucleotide sequence (which did not include a loss-of-function mutation). Desirable effects of treatment include decreasing the rate of disease progression, ameliorating or palliating the disease state, and remission or improved prognosis. Accordingly, an improved response to anti-cancer therapy includes an improvement in one or more effects described herein.

In one example, the methods of the present disclosure may further comprise preparing a reference NOD2 encoding nucleotide sequence from a population of individuals. For example, the reference NOD2 encoding nucleotide sequence may be a consensus sequence constructed from a population of individuals which do not possess loss-of-function mutations in their NOD2 encoding nucleotide sequence. The preparation of a reference NOD2 encoding nucleotide sequence may be prepared by any method known in the art. For example, the NOD2 encoding nucleotide sequences of a population of individuals may be collected to prepare a reference genome.

Method of Treating Cancer

The present disclosure also provides methods of treating cancer in a subject who is receiving, has been prescribed, or has received anti-cancer therapy. In this regard, the present inventors have surprisingly shown for the first time that inhibiting NOD2 function can improve a subject's response to anti-cancer therapy. On this basis, the inventors have developed new therapies for treating cancer comprising inhibition of NOD2 function.

As used herein, the terms “treating”, “treat” or “treatment” and variations thereof, refer to clinical intervention designed to alter the natural course of the individual or cell being treated during the course of clinical pathology. Desirable effects of treatment include decreasing the rate of disease progression, reducing size of the cancer, inhibiting tumour growth, inhibiting cancer progression or metastasis, ameliorating or palliating the disease state, and remission or improved prognosis. As used herein, the term “improved” shall be understood to mean decreased mortality, increased magnitude of response, decreased timing of treatment, decreased disease progression, decrease of pathological symptoms for the subject. Accordingly, an improved response to anti-cancer therapy includes an improvement in one or more effects described herein.

In one example, the present disclosure provides a method of treating cancer, comprising inhibiting NOD2 function in a subject in need thereof, wherein the subject is receiving anti-cancer therapy.

In one example, the present disclosure provides a method of treating cancer, comprising inhibiting NOD2 function in a subject in need thereof, wherein the subject has been prescribed anti-cancer therapy.

In one example, the present disclosure provides a method of treating cancer, comprising inhibiting NOD2 function in a subject in need thereof, wherein the subject has received anti-cancer therapy.

In one example, the present disclosure provides use of an inhibitor of NOD2 function in the manufacture of a medicament for treating cancer in a subject, wherein the subject is receiving anti-cancer therapy.

In one example, the present disclosure provides use of an inhibitor of NOD2 function in the manufacture of a medicament for treating cancer in a subject, wherein the subject has been prescribed anti-cancer therapy.

In one example, the present disclosure provides use of an inhibitor of NOD2 function in the manufacture of a medicament for treating cancer in a subject, wherein the subject has received anti-cancer therapy.

In one example, the present disclosure provides an inhibitor of NOD2 for use in treating cancer in a subject, wherein the subject is receiving anti-cancer therapy.

In one example, the present disclosure provides an inhibitor of NOD2 for use in treating cancer in a subject, wherein the subject has been prescribed anti-cancer therapy.

In one example, the present disclosure provides an inhibitor of NOD2 for use in treating cancer in a subject, wherein the subject has received anti-cancer therapy.

In one example, the present disclosure provides a method of treating cancer comprising administering an inhibitor of NOD2 function to a subject, wherein the subject has a functional NOD2 protein.

The methods of the present disclosure may further comprise the step determining the NOD2 status of the subject prior to treating the cancer.

In one example, determining the status of NOD2 in the subject comprises:

(i) determining the sequence of a NOD2 encoding nucleotide sequence in the subject;

(ii) comparing the nucleotide sequence determined at (i) to a reference NOD2 encoding nucleotide sequence;

wherein the presence of a NOD2 encoding nucleotide sequence which does not comprise a loss-of-function mutation in the subject is indicative that the subject should be administered the NOD2 inhibitor to improve their response to anti-cancer therapy.

Anti-Cancer Therapy

As used herein, the term “cancer” refers to a disease characterized by the rapid and uncontrolled growth of aberrant cells. Cancer cells can spread locally or through the bloodstream and lymphatic system to other parts of the body. Examples of various cancers include, but are not limited to, thoracic cancer, including non-small cell lung cancer and small cell lung cancer, thymoma, thymic carcinoma, thyroid cancer and mesothelioma; head and neck cancer including of the oropharynx, nasopharynx and hypopharynx; melanoma including cutaneous and uveal; skin cancer including basal cell carcinoma, merkel cell carcinoma and squamous cell carcinoma; neurological cancer including glioma, astrocytoma, oligodendroglioma and rare brain tumours; germ cell cancers of any primary site; sarcoma including all sub-types of soft tissue and bone; hepatobiliary cancer including liver, cholangiocarcinoma and gall bladder cancer; upper gastrointestinal cancers including oesophageal, gastric, pancreas and small bowel; lower gastrointestinal cancers including colon, rectal and anal; breast Cancer; CNS cancer; gynaecological cancer including ovarian, primary peritoneal, endometrial and vulval; genitourinary cancer including testicular, penile, prostate, bladder and kidney; neuroendocrine and adrenal cancers including carcinoid; cancer of unknown primary; lymphoma including Hodgkin and non-Hodgkin lymphomas, T-cell and B-cell lymphomas of all sub-types; leukaemia including lymphoid and myeloid leukaemia of all sub-types and plasma cell neoplasms including multiple myeloma. In one example, the cancer is lung cancer. In one example, the cancer is melanoma. In one example, the cancer is colon cancer.

Further exemplary cancers are described herein. The term “cancer” includes all types of cancerous growths or oncogenic processes, metastatic tissues or malignantly transformed cells, tissues, or organs, irrespective of histopathologic type or stage of invasiveness. The terms “tumour” and “cancer” are used interchangeably herein. Both terms encompass solid and liquid, e.g., diffuse or circulating, tumours. As used herein, the term “cancer” or “tumour” includes premalignant, as well as malignant cancers and tumours.

As used herein, the term “anti-cancer therapy” refers to any treatment to stop, inhibit or prevent the progression of cancer. “Anti-cancer therapy” may also refer to treatments which may shrink or reduce the cancer. Examples of anti-cancer therapy include, but are not limited to, radiotherapy, immunotherapy, chemotherapy, targeted therapy, hormone therapy, precision medicine and stem cell transplant. Exemplary anti-cancer therapies include chemotherapy, immunotherapy, radiotherapy, or any combination thereof. In one example, the anti-cancer therapy is chemotherapy. In one example, the anti-cancer therapy is immunotherapy. In one example, the anti-cancer therapy is radiotherapy.

In one embodiment, the methods described herein further comprise treating a subject determined as being likely to be responsive to anti-cancer therapy based on their NOD2 status, wherein treatment comprises administering to the subject an anti-cancer therapy.

Examples of suitable anti-cancer therapy include, but are not limited to, administration of one or more anti-cancer agents selected from the group consisting of an immune checkpoint inhibitor, a targeted antibody immunotherapy, a CAR-T cell therapy, an oncolytic virus, a cytostatic drug and combinations thereof. The immune checkpoint inhibitor may be a PD-1/PD-L1 targeting agent or a CTLA-4 targeting agent. In one example, the method comprises administering to the subject a PD-1/PD-L1 targeting agent selected from the group consisting of pembrolizumab, atezolizumab, avelumab, durvalumab and nivolumab. In one example, the method comprises administering to the subject a CTLA-4 targeting agent selected from the group consisting of ipilimumab and tremelimumab.

Alternatively, or in addition, anti-cancer therapy may include administration of one or more anti-cancer agents selected from the group consisting of Yervoy (ipilimumab, BMS); Keytruda (pembrolizumab, Merck); Opdivo (nivolumab, BMS); MEDI4736 (AZ/MedImmune); MPDL3280A (Roche/Genentech); Tremelimumab (AZ/MedImmune); CT-011 (pidilizumab, CureTech); BMS-986015 (lirilumab, BMS); MEDI0680 (AZ/MedImmune); MSB-0010718C (Merck); PF-05082566 (Pfizer); MEDI6469 (AZ/MedImmune); BMS-986016 (BMS); BMS-663513 (urelumab, BMS); IMP321 (Prima Biomed); LAG525 (Novartis); ARGX-110 (arGEN-X); PF-05082466 (Pfizer); CDX-1127 (varlilumab; CellDex Therapeutics); TRX-518 (GITR Inc.); MK-4166 (Merck); JTX-2011 (Jounce Therapeutics); ARGX-115 (arGEN-X); NLG-9189 (indoximod, NewLink Genetics); INCB024360 (Incyte); IPH2201 (Innate Immotherapeutics/AZ); NLG-919 (NewLink Genetics); anti-VISTA (JnJ); Epacadostat (INCB24360, Incyte); F001287 (Flexus/BMS); CP 870893 (University of Pennsylvania); MGA271 (Macrogenix); Emactuzumab (Roche/Genentech); Galunisertib (Eli Lilly); Ulocuplumab (BMS); BKT140/BL8040 (Biokine Therapeutics); Bavituximab (Peregrine Pharmaceuticals); CC 90002 (Celgene); 852A (Pfizer); VTX-2337 (VentiRx Pharmaceuticals); IMO-2055 (Hybridon, Idera Pharmaceuticals); LY2157299 (Eli Lilly); EW-7197 (Ewha Women's University, Korea); Vemurafenib (Plexxikon); Dabrafenib (Genentech/GSK); BMS-777607 (BMS); BLZ945 (Memorial Sloan-Kettering Cancer Centre); Unituxin (dinutuximab, United Therapeutics Corporation); Blincyto (blinatumomab, Amgen); Cyramza (ramucirumab, Eli Lilly); Gazyva (obinutuzumab, Roche/Biogen); Kadcyla (ado-trastuzumab emtansine, Roche/Genentech); Perjeta (pertuzumab, Roche/Genentech); Adcetris (brentuximab vedotin, Takeda/Millennium); Arzerra (ofatumumab, GSK); Vectibix (panitumumab, Amgen); Avastin (bevacizumab, Roche/Genentech); Erbitux (cetuximab, BMS/Merck); Bexxar (tositumomab-I131, GSK); Zevalin (ibritumomab tiuxetan, Biogen); Campath (alemtuzumab, Bayer); Mylotarg (gemtuzumab ozogamicin, Pfizer); Herceptin (trastuzumab, Roche/Genentech); Rituxan (rituximab, Genentech/Biogen); volociximab (Abbvie); Enavatuzumab (Abbvie); ABT-414 (Abbvie); Elotuzumab (Abbvie/BMS); ALX-0141 (Ablynx); Ozaralizumab (Ablynx); Actimab-C (Actinium); Actimab-P (Actinium); Milatuzumab-dox (Actinium); Emab-SN-38 (Actinium); Naptumonmab estafenatox (Active Biotech); AFM13 (Affimed); AFM11 (Affimed); AGS-16C3F (Agensys); AGS-16M8F (Agensys); AGS-22ME (Agensys); AGS-15ME (Agensys); GS-67E (Agensys); ALXN6000 (samalizumab, Alexion); ALT-836 (Altor Bioscience); ALT-801 (Altor Bioscience); ALT-803 (Altor Bioscience); AMG780 (Amgen); AMG 228 (Amgen); AMG820 (Amgen); AMG172 (Amgen); AMG595 (Amgen); AMG110 (Amgen); AMG232 (adecatumumab, Amgen); AMG211 (Amgen/MedImmune); BAY20-10112 (Amgen/Bayer); Rilotumumab (Amgen); Denosumab (Amgen); AMP-514 (Amgen); MEDI575 (AZ/MedImmune); MEDI3617 (AZ/MedImmune); MEDI6383 (AZ/MedImmune); MEDI551 (AZ/MedImmune); Moxetumomab pasudotox (AZ/MedImmune); MEDI565 (AZ/MedImmune); MEDI0639 (AZ/MedImmune); MEDI0680 (AZ/MedImmune); MEDI562 (AZ/MedImmune); AV-380 (AVEO); AV203 (AVEO); AV299 (AVEO); BAY79-4620 (Bayer); Anetumab ravtansine (Bayer); vantictumab (Bayer); BAY94-9343 (Bayer); Sibrotuzumab (Boehringer Ingleheim); BI-836845 (Boehringer Ingleheim); B-701 (BioClin); BIIB015 (Biogen); Obinutuzumab (Biogen/Genentech); BI-505 (Bioinvent); BI-1206 (Bioinvent); TB-403 (Bioinvent); BT-062 (Biotest) BIL-010t (Biosceptre); MDX-1203 (BMS); MDX-1204 (BMS); Necitumumab (BMS); CAN-4 (Cantargia AB); CDX-011 (Celldex); CDX1401 (Celldex); CDX301 (Celldex); U3-1565 (Daiichi Sankyo); patritumab (Daiichi Sankyo); tigatuzumab (Daiichi Sankyo); nimotuzumab (Daiichi Sankyo); DS-8895 (Daiichi Sankyo); DS-8873 (Daiichi Sankyo); DS-5573 (Daiichi Sankyo); MORab-004 (Eisai); MORab-009 (Eisai); MORab-003 (Eisai); MORab-066 (Eisai); LY3012207 (Eli Lilly); LY2875358 (Eli Lilly); LY2812176 (Eli Lilly); LY3012217 (Eli Lilly); LY2495655 (Eli Lilly); LY3012212 (Eli Lilly); LY3012211 (Eli Lilly); LY3009806 (Eli Lilly); cixutumumab (Eli Lilly); Flanvotumab (Eli Lilly); IMC-TR1 (Eli Lilly); Ramucirumab (Eli Lilly); Tabalumab (Eli Lilly); Zanolimumab (Emergent Biosolution); FG-3019 (FibroGen); FPA008 (Five Prime Therapeutics); FP-1039 (Five Prime Therapeutics); FPA144 (Five Prime Therapeutics); catumaxomab (Fresenius Biotech); IMAB362 (Ganymed); IMAB027 (Ganymed); HuMax-CD74 (Genmab); HuMax-TFADC (Genmab); GS-5745 (Gilead); GS-6624 (Gilead); OMP-21M18 (demcizumab, GSK); mapatumumab (GSK); IMGN289 (ImmunoGen); IMGN901 (ImmunoGen); IMGN853 (ImmunoGen); IMGN529 (ImmunoGen); IMMU-130 (Immunomedics); milatuzumab-dox (Immunomedics); IMMU-115 (Immunomedics); IMMU-132 (Immunomedics); IMMU-106 (Immunomedics); IMMU-102 (Immunomedics); Epratuzumab (Immunomedics); Clivatuzumab (Immunomedics); IPH41 (Innate Immunotherapeutics); Daratumumab (Janssen/Genmab); CNTO-95 (Intetumumab, Janssen); CNTO-328 (siltuximab, Janssen); KB004 (KaloBios); mogamulizumab (Kyowa Hakko Kirrin); KW-2871 (ecromeximab, Life Science); Sonepcizumab (Lpath); Margetuximab (Macrogenics); Enoblituzumab (Macrogenics); MGD006 (Macrogenics); MGF007 (Macrogenics); MK-0646 (dalotuzumab, Merck); MK-3475 (Merck); Sym004 (Symphogen/Merck Serono); DI17E6 (Merck Serono); MOR208 (Morphosys); MOR202 (Morphosys); Xmab5574 (Morphosys); BPC-1C (ensituximab, Precision Biologics); TAS266 (Novartis); LFA102 (Novartis); BHQ880 (Novartis/Morphosys); QGE031 (Novartis); HCD122 (lucatumumab, Novartis); LJM716 (Novartis); AT355 (Novartis); OMP-21M18 (Demcizumab, OncoMed); OMP52M51 (Oncomed/GSK); OMP-59R5 (Oncomed/GSK); vantictumab (Oncomed/Bayer); CMC-544 (inotuzumab ozogamicin, Pfizer); PF-03446962 (Pfizer); PF-04856884 (Pfizer); PSMA-ADC (Progenics); REGN1400 (Regeneron); REGN910 (nesvacumab, Regeneron/Sanofi); REGN421 (enoticumab, Regeneron/Sanofi); RG7221, RG7356, RG7155, RG7444, RG7116, RG7458, RG7598, RG7599, RG7600, RG7636, RG7450, RG7593, RG7596, DCDS3410A, RG7414 (parsatuzumab), RG7160 (imgatuzumab), RG7159 (obintuzumab), RG7686, RG3638 (onartuzumab), RG7597 (Roche/Genentech); SAR307746 (Sanofi); SAR566658 (Sanofi); SAR650984 (Sanofi); SAR153192 (Sanofi); SAR3419 (Sanofi); SAR256212 (Sanofi), SGN-LIVIA (lintuzumab, Seattle Genetics); SGN-CD33A (Seattle Genetics); SGN-75 (vorsetuzumab mafodotin, Seattle Genetics); SGN-19A (Seattle Genetics) SGN-CD70A (Seattle Genetics); SEA-CD40 (Seattle Genetics); ibritumomab tiuxetan (Spectrum); MLN0264 (Takeda); ganitumab (Takeda/Amgen); CEP-37250 (Teva); TB-403 (Thrombogenic); VB4-845 (Viventia); Xmab2512 (Xencor); Xmab5574 (Xencor); nimotuzumab (YM Biosciences); Carlumab (Janssen); NY-ESO TCR (Adaptimmune); MAGE-A-10 TCR (Adaptimmune); CTL019 (Novartis); JCAR015 (Juno Therapeutics); KTE-C19 CAR (Kite Pharma); UCART19 (Cellectis); BPX-401 (Bellicum Pharmaceuticals); BPX-601 (Bellicum Pharmaceuticals); ATTCK20 (Unum Therapeutics); CAR-NKG2D (Celyad); Onyx-015 (Onyx Pharmaceuticals); H101 (Shanghai Sunwaybio); DNX-2401 (DNAtrix); VCN-01 (VCN Biosciences); Colo-Adl (PsiOxus Therapeutics); ProstAtak (Advantagene); Oncos-102 (Oncos Therapeutics); CG0070 (Cold Genesys); Pexa-vac (JX-594, Jennerex Biotherapeutics); GL-ONC1 (Genelux); T-VEC (Amgen); G207 (Medigene); HF10 (Takara Bio); SEPREHVIR (HSV1716, Virttu Biologics); OrienX010 (OrienGene Biotechnology); Reolysin (Oncolytics Biotech); SVV-001 (Neotropix); Cacatak (CVA21, Viralytics); Alimta (Eli Lilly), cisplatin, oxaliplatin, irinotecan, folinic acid, methotrexate, cyclophosphamide, 5-fluorouracil, Zykadia (Novartis), Tafinlar (GSK), Xalkori (Pfizer), Iressa (AZ), Gilotrif (Boehringer Ingelheim), Tarceva (Astellas Pharma), Halaven (Eisai Pharma), Veliparib (Abbvie), AZD9291 (AZ), Alectinib (Chugai), LDK378 (Novartis), Genetespib (Synta Pharma), Tergenpumatucel-L (NewLink Genetics), GV1001 (Kael-GemVax), Tivantinib (ArQule); Cytoxan (BMS); Oncovin (Eli Lilly); Adriamycin (Pfizer); Gemzar (Eli Lilly); Xeloda (Roche); Ixempra (BMS); Abraxane (Celgene); Trelstar (Debiopharm); Taxotere (Sanofi); Nexavar (Bayer); IMMU-132 (Immunomedics); E7449 (Eisai); Thermodox (Celsion); Cometriq (Exellxis); Lonsurf (Taiho Pharmaceuticals); Camptosar (Pfizer); UFT (Taiho Pharmaceuticals); Tecentriq (atezolizumab, Roche); Bavencio (avelumab, Merck KGaA/Pfizer/Eli Lilly and Company), IMFINZI (durvalumab, Medimmune/AstraZeneca) and TS-1 (Taiho Pharmaceuticals). In one example, the anti-cancer agent is pembrolizumab. In one example, the anti-cancer agent is nivolumab. In one example, the anti-cancer agent is atezolizumab. In one example, the anti-cancer agent is avelumab. In one example, the anti-cancer agent is durvalumab. In one example, the anti-cancer agent is avelumab. In one example, the anti-cancer agent is ipilimumab. In one example, the anti-cancer agent is tremelimumab.

Inhibiting Nod2 Function

The inventors have shown for the first time that that the presence of a loss-of-function mutation in a subject's NOD2 encoding nucleotide sequence or a mutation which inhibits NOD2 signalling through other genes involved in the NOD2 signalling pathway can improve the subject's response to anti-cancer therapy relative to if the subject had a reference NOD2 encoding nucleotide sequence. Exemplary loss-of function NOD2 variants are set forth in Tables 1, 2, 3, 4 and 5 and in SEQ ID Nos: 3-7.

Surprisingly, the inventors have also shown for the first time that inhibiting NOD2 function in a subject (e.g., a subject with normal NOD2 function) can improve that subject's response to anti-cancer therapy.

As used herein, the term “inhibit” shall be taken to mean hinder, reduce, restrain or prevent NOD2 function in a subject relative to a subject to whom the inhibitor of NOD2 has not been administered. Methods of measuring NOD2 function are known in the art. For example, NOD2 function may be measured through assays measuring its activity and/or expression. Any of the methods disclosed herein may be used to measure NOD2 function.

The inhibition of NOD2 function may be partial loss-of function or complete loss-of-function. For example, NOD2 function may be reduced at least by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% relative to the function of NOD2 in a subject to whom the inhibitor of NOD2 has not been administered.

Accordingly, in some examples, the methods described herein may further comprise administering a NOD2 inhibitor to a subject determined as being likely to respond poorly to anti-cancer therapy based on their NOD2 status.

It will be understood by a person skilled in the art that NOD2 function may be inhibited directly or by inhibiting upstream or downstream effectors of NOD2. Accordingly, the NOD2 inhibitor may be a direct inhibitor of NOD2 function or an indirect inhibitor of NOD2 function. The inhibitor may bind to NOD2 to inhibit its function by changing its conformation or by affecting its active site. Alternatively, the inhibitor of NOD2 function may inhibit a binding partner of NOD2 and thereby affect NOD2 function. In another example, the inhibitor of NOD2 may inhibit NOD2 signalling.

In one example, inhibiting NOD2 function may comprise genetic inhibition of NOD2. Examples of suitable genetic inhibitors include, but are not limited to, agents that affect (e.g., upregulate or downregulate) the expression of NOD2 or its binding partners. Thus, the NOD2 inhibitor may be a nucleic acid inhibitor.

Methods of designing suitable genetic inhibitors are known in the art. Suitable examples of genetic inhibitors include, but are not limited to, DNA (gDNA, cDNA), RNA (sense RNAs, antisense RNAs, mRNAs, tRNAs, rRNAs, small interfering RNAs (siRNAs), short hairpin RNAs (ShRNAs), micro RNAs (miRNAs), small nucleolar RNAs (SnoRNAs), small nuclear RNAs (snRNAs), ribozymes, aptamers, DNAzymes, antisense oliogonucleotides, vectors, plasmids, other ribonuclease-type complexes, and mixtures thereof. The gene sequences of NOD2 and its binding partners are publicly available and can be used to design suitable genetic inhibitors by methods known in the art. A reference nucleotide sequence of NOD2 is provided in SEQ ID NO: 1.

In one example, the genetic inhibitor is an RNAi agent. RNAi may be useful for specifically inhibiting the production of a particular protein. Without wishing to be bound by theory, this technology relies on the presence of dsRNA molecules that contain a sequence that is essentially identical to the mRNA of the gene of interest or part thereof, in this case an mRNA encoding NOD2. Conveniently, the dsRNA can be produced from a single promoter in a recombinant vector host cell, where the sense and anti-sense sequences are flanked by an unrelated sequence which enables the sense and anti-sense sequences to hybridize to form the dsRNA molecule with the unrelated sequence forming a loop structure, such as a short hairpin RNA (shRNA). The design and production of suitable dsRNA molecules for the present disclosure is well within the capacity of a person skilled in the art, particularly considering WO99/32619, WO99/53050, WO99/49029, and WO01/34815. Such dsRNA molecules for RNAi include, but are not limited to short hairpin RNA (shRNA) and bi-functional shRNA.

The nucleic acid inhibitor may be a small interfering RNA (“siRNA”) molecule. siRNA molecules comprise a nucleotide sequence that is identical to about 19-21 contiguous nucleotides of the target mRNA. For example, the siRNA sequence may commence with the dinucleotide AA, may comprise a GC-content of about 30-70% (for example, 30-60%, such as 40-60% for example about 45%-55%), and/or may not have a high percentage identity (for example, may have less than 70%, 80%, 90%, 95%, 98%, 99%, or 100%) to any nucleotide sequence other than the target in the genome of the mammal in which it is to be introduced, for example as determined by standard BLAST search.

The nucleic acid inhibitor may be an antisense nucleic acid. As used herein, the term “antisense nucleic acid” shall be taken to mean a DNA or RNA or derivative thereof (e.g., LNA or PNA), or combination thereof that is complementary to at least a portion of a specific mRNA molecule encoding a polypeptide as described herein in any example of the disclosure and capable of interfering with a post-transcriptional event such as mRNA translation. The use of antisense methods is known in the art (see for example, Hartmann and Endres (editors), Manual of Antisense Methodology, Kluwer (1999)).

Thus, an antisense nucleic acid of the disclosure will hybridize to a target nucleic acid under physiological conditions. Antisense nucleic acids include sequences that correspond to structural genes or coding regions or to sequences that effect control over gene expression or splicing. For example, the antisense nucleic acid may correspond to the targeted coding region of a nucleic acid encoding NOD2, or the 5′-untranslated region (UTR) or the 3′-UTR or a combination of these. It may be complementary in part to intron sequences, which may be spliced out during or after transcription, for example only to exon sequences of the target gene. The length of the antisense sequence should preferably be at least 19 contiguous nucleotides, for example, at least 50 nucleotides, such as at least 100, 200, 500 or 1000 nucleotides of a nucleic acid encoding NOD2. The full-length sequence complementary to the entire gene transcript may be used. The degree of identity of the antisense sequence to the targeted transcript should be at least 90%, for example, 95-100%.

The genetic inhibitor may be a nucleic acid aptamer (which may also be referred to as an adaptable oligomer). Aptamers are single stranded oligonucleotides or oligonucleotide analogs that are capable of forming a secondary and/or tertiary structure that provides the ability to bind to a particular target molecule, such as a protein or a small molecule, e.g., NOD2. Thus, aptamers are often considered to be the oligonucleotide analogy to antibodies. In general, aptamers comprise about 15 to about 100 nucleotides, such as about 15 to about 40 nucleotides, for example about 20 to about 40 nucleotides.

Oligonucleotides of a length that falls within these ranges can be prepared by conventional techniques.

An aptamer can be isolated from or identified from a library of aptamers. An aptamer library can be produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., binds specifically to NOD2) is selected. An aptamer with increased activity can be selected, for example, using SELEX (Sytematic Evolution of Ligands by EXponential enrichment). Suitable methods for producing and/or screening an aptamer library are described, for example, in Elloington and Szostak, Nature 346:818-22, 1990; U.S. Pat. No. 5,270,163; and/or U.S. Pat. No. 5,475,096.

In one particular example, the NOD2 inhibitor is an inhibitor of the NOD2 protein. Suitable NOD2 inhibitors are known in the art. In addition, suitable NOD2 inhibitors may be identified using known screen methods. For example, those methods may comprise contacting NOD2 with a test agent and determining the effect of the test agent on the level of expression and/or activity of NOD2. The function of NOD2 may be assessed using methods that include the determination of cytokine activity by in vitro stimulation using muramyl dipeptide (MDP) (Hsu et al., 2008).

The NOD2 inhibitor may be a small molecule inhibitor of NOD2. Examples of suitable inhibitors of NOD2 protein include, but are not limited to, a small molecule inhibitors of NOD2 (Rickard D J et al PLoS One. 2013 Aug. 1; 8(8):e69619 PMID 23936340) such as GSK717 (N-(2-(1-(2-(2,3-Dihydro-1H-inden-5-ylamino)-2-oxoethyl)-1H-benzo[d]imidazol-2-yl)ethyl)-N-methylbenzamide). Thus, the methods disclosed herein can be performed using the NOD2 inhibitor GSK717 (N-(2-(1-(2-(2,3-Dihydro-1H-inden-5-ylamino)-2-oxoethyl)-1H-benzo[d]imidazol-2-yl)ethyl)-N-methylbenzamide).

Inhibiting NOD2 signalling may also be achieved through small molecules which inhibit NOD2 interaction partners. For example, NOD2 inhibition may be achieved through inhibition of dual-specificity kinase, RIPK2 (RIP2). Knockout mice lacking RIPK2 are healthy and have a selective loss of response to the NOD2 ligand, MDP, and to the ligand for the related bacterial peptidoglycan receptor, NOD1, but normal innate and adaptive immune responses otherwise (Park et al 2007 J Immunol. 178:2380-6 PMID 17277144; Hall et al 2008 Eur J Immunol. 2008 January; 38:64-72 PMID 18085666; Fairhead 2008 Am J Transplant 8:1143-50 PMID 18522545).

Examples of suitable RIPK2 inhibitors include, but are not limited to, gefitinib; small molecule inhibitors of p38a (SB203580) and Src kinases (PP2) (Windheim M et al 2007 Biochem Jnl 404:179-190); OD36 (Calbiochem); WEHI-345 (N-(2-(4-amino-3-(p-tolyl)-1H-pyrazolo[3,4-d]pyrimidin-1-yl)-2 methylpropyl)isonicotinamide) (Nachbur Nat Commun. 2015 Mar. 17; 6:6442. PMID 25778803); erlotinib; Type II small molecule inhibitors of protein kinases, such as ponatinib, regorafenib and sorafenib (Canning P Chem Biol. 2015 Sep. 17; 22(9):1174-84 PMID 26320862).

The protein kinase LRRK2 has also been shown to be important for RIPK2 phosphorylation and activation in response to MDP and NOD2 (Yan & Ziu 2017 Protein Cell 8:55-66 PMID 27830463). Hence, it will be appreciated by those skilled in the art that LRKK2 is another target for inhibiting NOD2 signalling. Examples of suitable LRKK2 inhibitors include, but are not limited to, CZC 25146 (CAS no. 1191911-26-8), CZC 54252 hydrochloride (CAS no. 1784253-05-9), GNE-9605 (CAS no. 1536200-31-3), GNE-0877 (CAS no. 1374828-69-9), GNE-7915 (CAS no. 1351761-44-8), GSK2578215A (CAS no. 1285515-21-0), JH-II-127 (CAS no. 1700693-08-8), LRRK2-IN-1 (CAS no. 1234480-84-2), MLi-2 (CAS no. 1627091-47-7), PF06447475 (CAS no. 1527473-33-1) and URMC-099 (CAS no. 1229582-33-5).

Inhibition of NOD2 may also be performed at the level of ubiquitin ligases required for NOD2 signalling. In one embodiment, the ubiquitin ligase is selected from the group consisting of: XIAP, cIAP2, cIAP1 and Pellino 3. Human loss of function mutations in the ubiquitin ligase XIAP (BIRC4) cause colitis and X-linked lymphoproliferative syndrome 2 (Nielsen & Lacasse Genet Med. 2017 February; 19(2):133-143 PMID 27416006). Such mutations may interfere with NOD2 signalling including missense mutations in the second BIR domain that inhibit XIAP binding to RIPK2. The inhibitory effect of these on NOD2 signalling can be mimicked with SMAC-mimetic compounds that interfere with XIAP and cIAP1 and 2 (Damgaard EMBO Mol Med. 2013 August; 5(8):1278-95 PMID 23818254; Stafford C A Cell Rep. 2018 Feb. 6; 22(6):1496-1508. PMID 29425505) such as XB2d89 (Goncharov T et al Mol Cell. 2018 Feb. 15; 69(4):551-565 PMID 29452636).

Knockout mice lacking either of the K63 ubiquitin ligases, cIAP2 (BIRC3) or cIAP1 (BIRC2), are selectively unable to respond to NOD2 or NOD1 ligands, because these serve to activate RIPK2 (Bertrand et al 2009 Immunity 30(6):789-801 PMID 19464198). Subsequent studies from other groups have found less inhibition of NOD2 signalling by genetic inhibition of cIAP1 or cIAP2, which appear to function further downstream in the NOD2 signalling pathway (Stafford C A Cell Rep. 2018 Feb. 6; 22(6):1496-1508. PMID 29425505). Furthermore, loss of the ubiquitin ligase Pellino3 has also been shown to interfere with NOD2 signalling (Yang et al 2013 Nature Immunol 14(9):927-36 PMID 23892723). Hence, a skilled addressee would appreciate that inhibitors of cIAP1, cIAP2 and Pellino 3 may also be used to inhibit NOD2.

Accordingly, the inhibitor of NOD2 activity or function may be a NOD2 inhibitor, an RIPK2 inhibitor, an LRRK2 inhibitor, an inhibitor of a ubiquitin ligase required for NOD2 signalling activity, or any combination thereof. In one example, the inhibitor is an RIPK2 inhibitor. In one example, the inhibitor is an LRRK2 inhibitor. In one example, the inhibitor inhibits a ubiquitin ligase required for NOD2 signalling activity. In one example, the ubiquitin ligase is XIAP. In one example, the ubiquitin ligase is cIAP1. In another example, the ubiquitin ligase is cIAP2. In another example, the ubiquitin ligase in Pellino 3.

### EXAMPLES

### Example 1. Inhibition of NOD2 Promotes Immune Response Against Melanoma

A patient with a rare exceptional response to metastatic melanoma was investigated. At the time of radiation treatment, the patient had a dismal prognosis. One of the many metastatic lesions, which was in her brain, was treated with stereotactic radiation as a palliative procedure. Over the next six weeks all sites of melanoma metastases, including liver, skin and nodes, regressed and disappeared. The patient continues to remain free of melanoma now 20 years later.

This “abscopal response” to localised cancer therapy is exceedingly rare and occurs less than 1 in 400 in patients with metastatic melanoma that has already formed tumours in multiple sites in the body (Bramhall et al Eur J Surg Oncol. 2014 40(1):34-41. PMID 24139999). Uniquely, the patient's abscopal cancer response to radiotherapy was accompanied by development of an inflammatory disease of her gut, Crohn's disease. The concurrent development of intestinal inflammation is reminiscent of one of the main side effects of the immune checkpoint inhibitor, ipilimumab (anti-CTLA4), when it is given to melanoma patients to release a figurative brake on the immune response to the cancer cells.

Whole genome sequence analysis of the patient's germline DNA was performed. It was identified that she had inherited a loss-of-function mutation in one copy of her NOD2 encoding nucleotide sequence (alternative gene name is CARD15), p.Leu1007ProfsTer2 (rs2066847). The L1007fs mutation is carried by 2.6% of people worldwide based on publicly available data in the Exome Aggregation Consortium (ExAC) browser (http://exac.broadinstitute.org/variant/16-50763778-G-GC). Carrying one copy of this mutation increases the risk of Crohn's disease by a factor of four (M. Economou et al 2004 Am. J. Gastroenterol., 99, 2393-2404 PMID 15571588).

The genome sequence also revealed the patient inherited a second mutation on the other copy of NOD2/CARD15, p.Thr189Met (rs61755182), present in 0.51% of people in ExAC. This variant has been shown to significantly reduce NFKB signalling following MDP stimulation (R. Parkhouse, T. Monie, 2015, Front Immunol, 6: 521 PMID: 26500656).

The percentage of people expected to have both mutations is 0.51%×2.6%=0.013% of people (approximately 1 in 10,000).

The rarity of the patient's NOD2 genotype, the strong causal connection between genetic inhibition of NOD2 and Crohn's disease, and the development of Crohn's disease accompanying the immune eradication of her melanoma metastases, provides strong in hominum evidence that inhibition of NOD2 promotes a body-wide immune response against melanoma when coupled with local irradiation of one tumour.

### Example 2. Inhibition of NOD2 Promotes Immune Response Against Lung Cancer (I)

Patient Recruitment

A clinical research protocol was established and approved by the Sydney Local Health District Human Research Ethics Committee—Concord Repatriation General Hospital. Participating hospital Research Governance Offices undertook independent site-specific assessments of the protocol. The protocol was approved at five major hospitals across Sydney-Westmead, Blacktown, Concord, St Vincent's Sydney and The Chris O'Brien Lifehouse.

Patients with non-small cell lung cancer receiving single agent treatment with anti-PD-1 or PD-L1 were identified through direct physician referral, or analysis of clinic notes, pharmacy records and day therapy bookings.

Patients were consented in accordance with the International Conference on Harmonisation Good Clinical Practice guidelines. A single blood draw of 2×10 mL EDTA tubes was taken at the time of consent.

Medical records including clinical notes, radiology reports and images, histology and pathology results were accessed for collection of data points for consented patients.

Genome sequencing Patient genomes were sequenced on the Illumina HiSeq X platform using DNA isolated from whole blood.

Raw reads were aligned to the hs37d5 reference using BWA-MEM v0.7.10-r789, and sorted and duplicate-marked with Novosort v1.03.01. Local indel realignment and base quality score recalibration was performed using GATK v3.3-0-g37228af. gVCFs were generated with GATK HaplotypeCaller, and variants recalibrated using GATK Variant Quality Score Recalibrator (VQSR). Finally, VCF files were annotated with Variant Effect Predictor (VEP) v76 using the LoFTEE and dbNSFP plugins.

Variants were packaged into databases using GEMINI (v0.18.3) and imported into the variant filtration and annotation platform Seave. Variants were filtered on expected inheritance pattern, depth ≥10, QUAL ≥200, ≤1% in each of ExAC, 1000 Genomes and ESV, CADD ≥10 and medium or high impact (based on Ensembl classification categorisation). Resulting candidate variants were further prioritised using the PolyPhen, PROVEAN and SIFT in silico prediction scores and known association with disease as identified by ClinVar, OMIM and Orphanet. All reported variants were manually validated using IGV to verify their authenticity.

Sequence Analysis

The burden of rare and common variants in immune tolerance genes was analysed and compared to the Medical Genome Reference Bank (MGRB, comprising WGS of 1144 well-elderly individuals) and the Exome Aggregation Consortium (ExAC).

Comparisons were made with Fisher Exact test. Genetic risk scores for auto-immune conditions were calculated for these cohorts, MGRB and NSCLC patients included in The Cancer Genome Atlas (TCGA-LUAD). Scores were calculated using curated risk alleles and odds-ratio weightings derived from the NHGRI-EBI GWAS catalogue.

Results

Number of Exceptional Responders Identified

Exceptional responders were defined by two criteria:
1) Complete or partial response of more than 12 months OR stable disease of more than 24 months (per RECIST criteria)
AND
2) Concurrent immune-related adverse event of any grade.

From a treatment pool of over 400 patients, twenty cases were identified with an exceptional response to anti-PD1 or anti-PD-L1 agent.

Burden of Rare and Common Variants

The autoimmune toxicity of exceptional responders matched the frequency expected from trial data—i.e. the most common toxicities were thyroiditis and rash. Three of twenty patients had colitis as their toxicity.

Genetic inhibitors of NOD2 are frequent in lung cancer exceptional responders. The genome sequence analysis revealed that six of the twenty exceptional responders had mutations that inhibit NOD2, with one exceptional responder carrying two damaging alleles (allele count 7 of 40).

Lung cancer patients LH2 and TKCC6 had the same L1007fs mutation (rs2066847) found in the melanoma patient from Example 1 and in 2.6% of people in ExAC and 3.3% of well elderly people analysed by whole genome sequencing in the medical genome reference bank (MGRB). This mutation is known to abolish NOD2 function and delocalize it from cell membranes (Bonen D K 2003 Gastroenterology 124:140 PMID 12512038; Inohara 2003 J Biol Chem 278:5509 PMID 12514169; Barnich N 2005 J Cell Biol 170:21 PMID 15998797; Lecine P 2007 J Biol Chem 282:15197 PMID 17355968). Carrying one copy of this mutation increases the risk of Crohn's disease by a factor of four (M. Economou et al 2004 Am. J. Gastroenterol., 99, 2393-2404 PMID 15571588). Neither patient carrying the frameshift mutation experienced colitis as a toxicity of treatment. Patient LH2 experienced autoimmune haemolytic anaemia with cold agglutinins, pneumonitis and cutaneous lupus, and TKCC6 experienced hypothyroidism. The characteristics of the patients are shown in Table 1.

TABLE 1
Patient characteristics

| | | Best | PFS | |
|---|---|---|---|---|
| Patient | Genotype | Response | (months) | irAE |
| LH2 | L1007fs/wt | PR | 30 | AIHA/cut. |
| | | | | Lupus/cold |
| | | | | agglutinins/ |
| | | | | pneumonitis |
| LH7 | G908R/wt | CR | 43 | Rash |
| LH17 | R702W/wt | PR | 20 | Hypothyroid |
| WMH1 | R702W/wt | PR | 53 | Rash |
| CRGH4 | G908R/R702W | PR | 23 | Colitis |
| TKCC6 | L1007fs/wt | CR | 17 | Hypothyroid |

Lung cancer patients WMH1, LH17 and CRGH4 had the NOD2 p.Arg702Trp (R702W; rs2066844; SEQ ID NO: 3) mutation, which is present in 4.5% of people in ExAC and 9.2% of people in MGRB. The mutation inhibits NOD2 signalling (Bonen D K 2003 Gastroenterology 124:140 PMID 12512038; Inohara 2003 J Biol Chem 278:5509 PMID 12514169). Carrying one copy of the mutation increases susceptibility to Crohn's disease 2.2 fold (M. Economou et al 2004 Am. J. Gastroenterol., 99, 2393-2404 PMID 15571588). Patient CRGH4 carried this mutation in compound heterozygous form with Gly908Arg, and experienced colitis as toxicity (Table 1) WMH1 and LH17 experienced rash and hypothyroidism respectively (Table 1).

Lung cancer patients LH7 and CRGH4 had NOD2 p.Gly908Arg (G908R, applies to ENST00000300589; rs2066845; SEQ ID NO: 7), which is present in 1.8% of people in ExAC and 2.4% of people in MGRB (Table 2). G908R is also known to diminish NOD2 signalling (Bonen D K 2003 Gastroenterology 124:140 PMID 12512038; Inohara 2003 J Biol Chem 278:5509 PMID 12514169). Carrying one copy of the mutation increases susceptibility to Crohn's disease three-fold (M. Economou et al 2004 Am. J. Gastroenterol., 99, 2393-2404 PMID 15571588). Patient LH7 experienced rash as toxicity. Patient CRGH4, as mentioned above, was compound heterozygous for G908R and R702W and experienced colitis as toxicity. The percentage of people expected to carry both R702W and G908R mutations is 4.5%×1.8%=0.081% (approximately 1 in 1200).

TABLE 2
Expected versus observed allelic frequency
(AF) of NOD2-inhibiting variants

| | Expected AF | | | |
|---|---|---|---|---|
| | Allele count/number | Observed AF | | |
| Mutation | (AF %) | N/40 (%) | | p-value |
| G908R | 1204/121412 (0.99) | 2/40 | (5%) | 0.06423 |
| R702W | 2704/118890 (2.27) | 3/40 | (7.5%) | 0.07044 |
| L1007fs | 1584/121312 (1.31) | 2/40 | (5%) | 0.1022 |
| Total | 4.57% | 7/40 | (17.5%) | 0.004259 |

Using the ExAC frequencies is more accurate because they survey a larger number of individuals. Based on ExAC frequencies, the expected frequency of carrying one or other of the three NOD2 inhibiting mutations is 0.026+0.045+0.018=0.089 (8.9%). The inventors' finding that 30% of exceptional responders with NOD2 directly inhibited by uncommon mutations is significantly greater than expected.

Genetic Inhibitors of NOD2 Signalling Co-Factors in Lung Cancer Exceptional Responders

In addition to NOD2 inhibition by uncommon mutations in the NOD2 encoding nucleotide sequence itself, the exceptional responders cohort was enriched for common DNA variants affecting genes for other essential proteins for NOD2 signalling. 170 common DNA variants not involving the NOD2 encoding nucleotide sequence have previously been associated with increased susceptibility to Crohn's disease in two large genome wide association studies (GWAS).

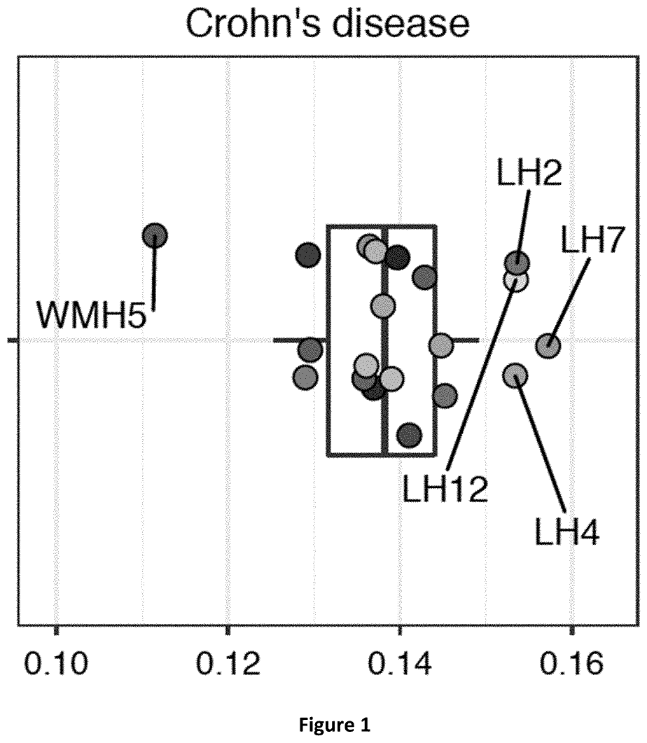

The burden of these common susceptibility DNA variants in each of the exceptional responders (coloured circles) and in each of 1144 well elderly people in the MGRB (Box plot, percentile markings 10, 25, mean, 75 and 90) was calculated. FIG. 1 shows that 20% of exceptional responders lie above the 90th centile for burden of these common Crohn's susceptibility variants compared to healthy controls in MGRB.

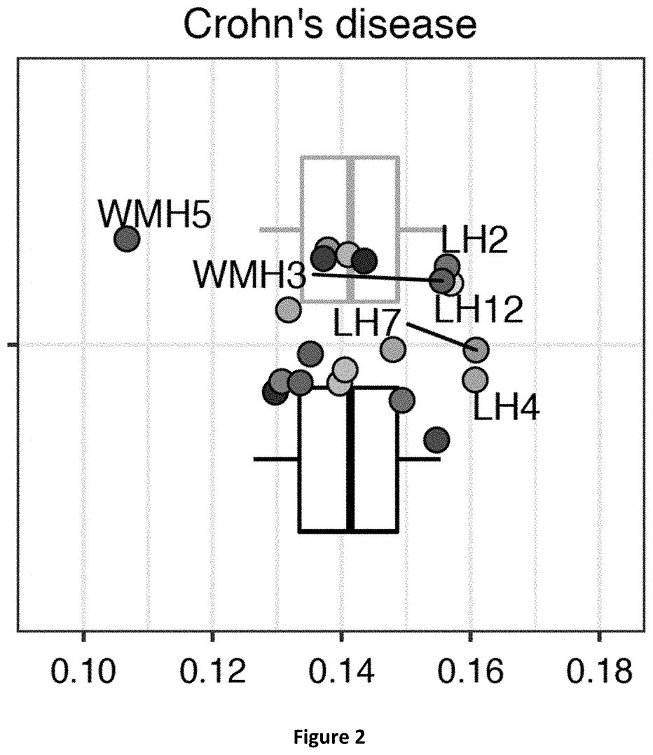

The same analysis was applied to lung adenocarcinoma patients included in The Cancer Genome Atlas (TCGA-LUAD). The TCGA-LUAD patients had similar centile placements as the MGRB patients, suggesting enrichment for NOD-2-associated variants is not a risk factor for developing lung cancer (FIG. 2).

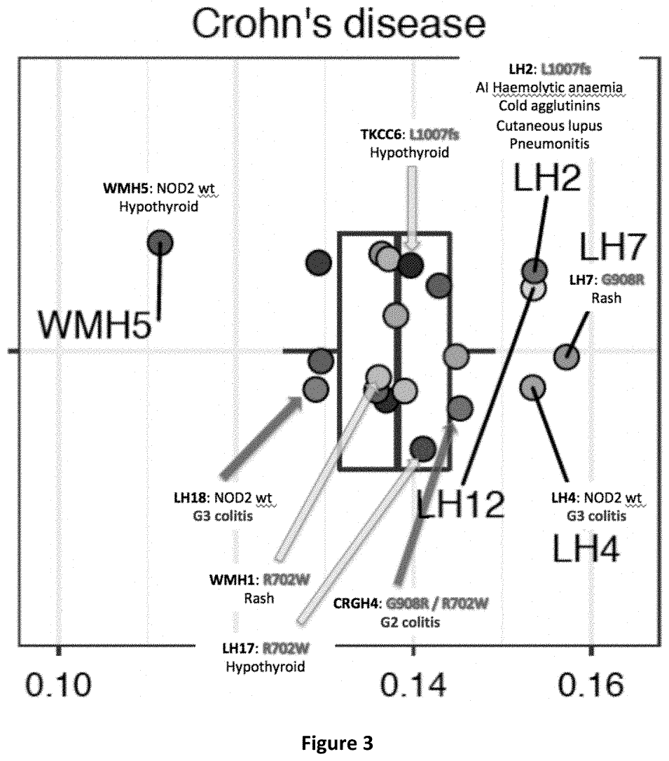

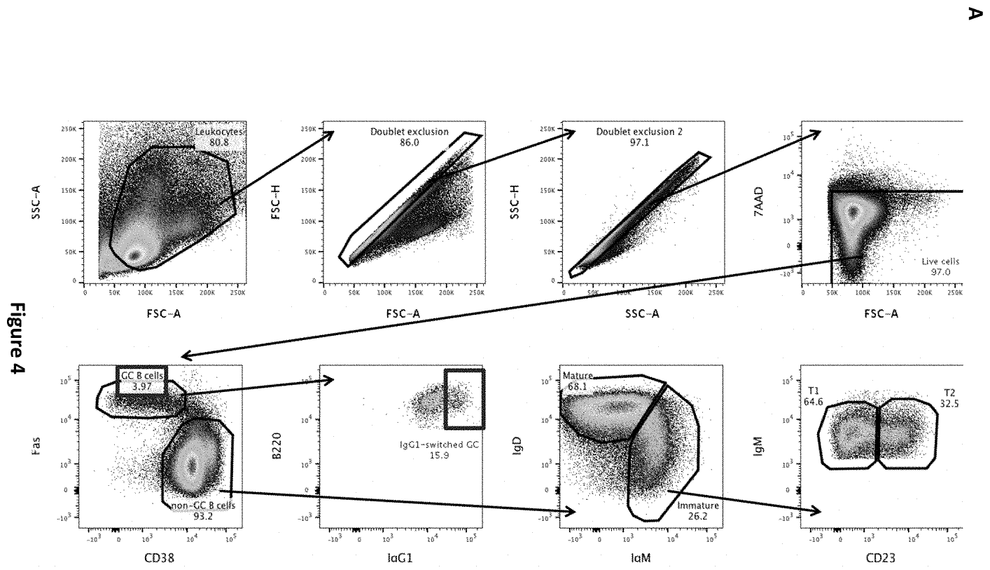

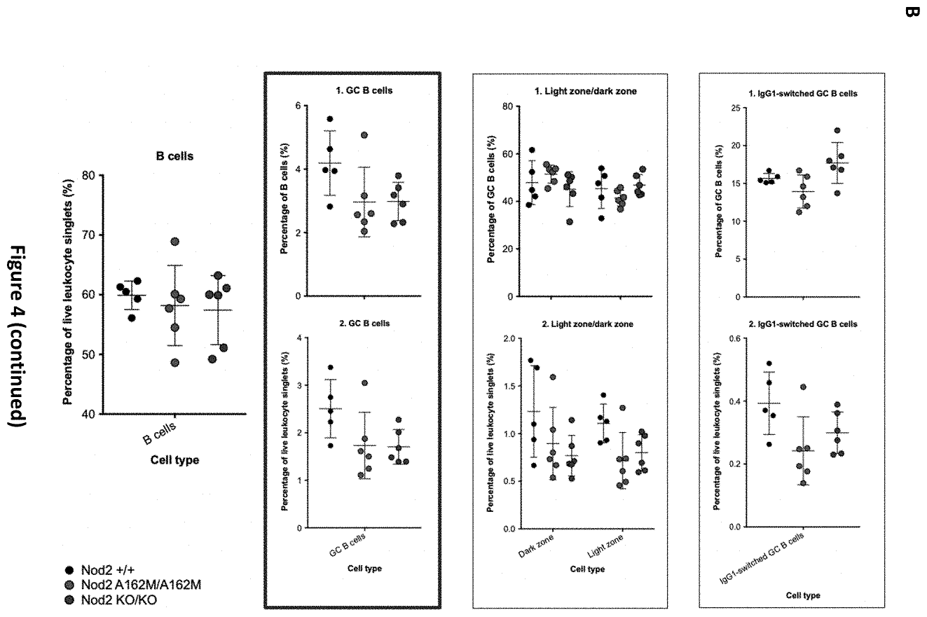

FIG. 3 shows that the three NOD2-inhibiting variants are included in risk scores, and are found in patients across the spectrum (above and below mean risk). NOD2 variation correlated with colitis in only one case (CRGH4, compound heterozygous).

The inventors then analysed which DNA variants contributed to this higher burden, by scoring each DNA variant (single nucleotide polymorphism, SNP) for its mean burden in the exceptional responders and in MGRB, and ranking them according to the difference in these mean burdens (Table 3). Of the top 15 variants, eight affected either NOD2 or its functional partners, LRRK2 and IRGM, which encode for proteins that interact with NOD2 in subcellular complexes and promote NOD2 signalling (Chauhan S et al 2015 Mol Cell 58:507 PMID 25891078; Zhang Q et al 2015 Nature Immunol 16:918 PMID 26237551; Yan & Liu 2017 Protein Cell 8:55-66 PMID 27830463; Wang H et al 2017 J Immunol 198(9):3729-3736 PMID 28330897).

TABLE 3
Genetic associations of top 15 SNPs contributing to Crohn's Disease genetic risk score

| | | | | Score | | |
|---|---|---|---|---|---|---|
| | | Score | Score | (TCGA- | Contribution | |
| SNP rsID | effectAllele | (responders) | (MGRB) | LUAD) | to risk score | Proximal genes (from UCSC) |
| rs6556412 | A | 0.087924609 | 0 | 0 | 0.087924609 | LOC285626 intron, IL12B 30 kb 5′ of start |
| rs11175593* | T | 0.086356483 | 0.011700398 | 0.020129716 | 0.074656086 | LRRK2 18 kb 5′ of start, MUC19 further away |
| rs7714584* | G | 0.125924296 | 0.051734632 | 0 | 0.074189664 | ZNF300 4 kb 3′ to stop, IRGM 45 kb away |
| rs2066847* | GC | 0.102011006 | 0.033884775 | 0 | 0.068126231 | NOD2 fs mutation |
| rs11747270* | G | 0.114071577 | 0.046865071 | 0.075781817 | 0.067206506 | ZNF300 4 kb 3′ to stop, IRGM 30 kb away |
| rs11564258* | A | 0.083082767 | 0.018882447 | 0.02582215 | 0.06420032 | MUC19 intron, LRRK2 ends 25 kb 5′ |
| rs1000113* | T | 0.129534725 | 0.066428064 | 0.100648582 | 0.063106661 | IRGM 12 kb 5′ of start |
| rs780093 | T | 0.167714331 | 0.108608712 | 0 | 0.059105619 | GCKR intron |
| | | | | | | IL18RAP intron, IL18R1 ends 40 kb away |
| rs2058660 | G | 0.13046498 | 0.08195877 | 0 | 0.048506211 | IL12RL2, IL18R1, IL1RL1, IL18RAP |
| rs7517847 | T | 0.419767832 | 0.372213714 | 0 | 0.047554118 | IL23R intron |
| rs12422544* | C | 0.0562706 | 0.009837517 | 0 | 0.046433083 | Intergenic. LRRK2 90 kb 3′ of stop |
| rs1260326 | T | 0.13838242 | 0.092234786 | 0 | 0.046147634 | Missense variant within GCKR exon |
| rs3764147 | G | 0.11718528 | 0.071557944 | 0.080777066 | 0.045627335 | LACC1 exon |
| rs224090 | T | 0.141858997 | 0.099089366 | 0.112128429 | 0.042769631 | ADO 20 kb 5′ of start, EGFR 30 kb 5′ of start |
| rs11741861* | G | 0.085375731 | 0.043533662 | 0.054396348 | 0.041842069 | Intronic variant ZNF300, IRGM 50 kb away |

*NOD2 functional partners

### Example 3. Inhibition of NOD2 Promotes Immune Response Against Lung Cancer (II)

A further analysis of the progression-free survival of the patients in Example 2 was performed.

From a treatment pool of over 600 patients, forty cases were identified with an exceptionally good response to anti-PD1 or anti-PD-L1 and auto-immune toxicity. Ten cases were identified with best response of progressive disease (non-responders).

Burden of Rare and Common Variants

The autoimmune toxicity of exceptional responders matched the frequency expected from trial data—i.e. the most common toxicities were thyroiditis and rash. Seven of forty patients had colitis as part of their toxicity.

Genetic inhibitors of NOD2 are frequent in lung cancer exceptional responders. The genome sequence analysis revealed that nine of the forty exceptional responders had mutations that inhibit NOD2, with one exceptional responder carrying two damaging alleles (allele count 10 of 80). There were no mutations that inhibit NOD2 within the non-responders (allele count 0 of 20). The allele count was 10/2288 (0.44%) for rs61755182 and 38/2288 (1.7%) for rs2066847. There were no subjects that carried both variant alleles, demonstrating that the alleles are not carried in linkage disequilibrium.

Lung cancer patients LH2 and TKCC6 had the same L1007fs mutation (rs2066847) found in the melanoma patient from Example 1 and in 2.6% of people in ExAC and 3.3% of well elderly people analysed by whole genome sequencing in the medical genome reference bank (MGRB). This mutation is known to abolish NOD2 function and delocalize it from cell membranes (Bonen D K 2003 Gastroenterology 124:140 PMID 12512038; Inohara 2003 J Biol Chem 278:5509 PMID 12514169; Barnich N 2005 J Cell Biol 170:21 PMID 15998797; Lecine P 2007 J Biol Chem 282:15197 PMID 17355968). Carrying one copy of this mutation increases the risk of Crohn's disease by a factor of four (M. Economou et al 2004 Am. J. Gastroenterol., 99, 2393-2404 PMID 15571588). Neither patient carrying the frameshift mutation experienced colitis as a toxicity of treatment. Patient LH2 experienced autoimmune haemolytic anaemia with cold agglutinins, pneumonitis and cutaneous lupus, and TKCC6 experienced hypothyroidism and colitis. The characteristics of the patients are shown in Table 4.

TABLE 4
Patient characteristics

| | | Best | PFS | |
|---|---|---|---|---|
| Patient | Genotype | Response | (months) | irAE |
| LH2 | L1007fs/wt | PR | 48 | AIHA/cut. |
| | | | | Lupus/cold |
| | | | | agglutinins/ |
| | | | | pneumonitis |
| LH7 | G908R/wt | CR | 59 | Rash |
| LH17 | R702W/wt | PR | 34 | Hypothyroid |
| WMH1 | R702W/wt | PR | 68 | Rash |
| CRGH4 | G908R/R702W | PR | 44 | Colitis |
| TKCC6 | L1007fs/wt | CR | 17 | Hypothyroid |
| WMH13 | R702W/wt | PR | 38 | Rash/ |
| | | | | hypothyroid |
| LH5 | L248R/wt | SD | 38 | Psoriasis/ |
| | | | | transaminitis/ |
| | | | | pancreatitis |
| BMDH36 | G908R/wt | PR | 18 | Transaminitis/ |
| | | | | rash/hypothyroid |

Lung cancer patients WMH1, LH17, CRGH4 and WMH13 had the NOD224T p.Arg702Trp (R702W; rs2066844) mutation, which is present in 4.5% of people in ExAC and 9.2% of people in MGRB. The mutation inhibits NOD2 signalling (Bonen D K 2003 Gastroenterology 124:140 PMID 12512038; Inohara 2003 J Biol Chem 278:5509 PMID 12514169). Carrying one copy of the mutation increases susceptibility to Crohn's disease 2.2 fold (M. Economou et al 2004 Am. J. Gastroenterol., 99, 2393-2404 PMID 15571588). Patient CRGH4 carried this mutation in compound heterozygous form with Gly908Arg, and experienced colitis as an early toxicity, with later development of inflammatory arthritis (grade 3 bilateral sacroiliitis on imaging), rash and worsening of pre-existing hypothyroidism (Table 4). WMH1 and LH17 experienced rash and hypothyroidism respectively, WMH13 experienced both rash and hypothyroidism (Table 4).

Lung cancer patients LH7, CRGH4 and BMDH36 had NOD2 p.Gly908Arg (G908R, applies to ENST00000300589; rs2066845), which is present in 1.8% of people in ExAC and 2.4% of people in MGRB (Table 2). G908R is also known to diminish NOD2 signalling (Bonen D K 2003 Gastroenterology 124:140 PMID 12512038; Inohara 2003 J Biol Chem 278:5509 PMID 12514169). Carrying one copy of the mutation increases susceptibility to Crohn's disease three-fold (M. Economou et al 2004 Am. J. Gastroenterol., 99, 2393-2404 PMID 15571588). Patient LH7 experienced rash as toxicity. Patient CRGH4, as mentioned above, was compound heterozygous for G908R and R702W and experienced multiple toxicities. The percentage of people expected to carry both R702W and G908R mutations is 4.5%×1.8%=0.081% (approximately 1 in 1200).

Lung cancer patient LH5 had NOD2 p.Leu248Arg (L248R, applies to ENST00000300589; rs104895423), which is present in 0.05% of people in ExAC and 0.04% of people in MGRB (Table 2). L248R shows near complete reduction of NFKB signalling following MDP-stimulation, similar in effect to L1007fs (R. Parkhouse, T. Monie, 2015, Front Immunol, 6: 521 PMID: 26500656). Patient LH5 experienced profound palmar psoriasis, transaminitis and pancreatitis as toxicity.

TABLE 5
Expected versus observed allelic frequency (AF) of NOD2-inhibiting variants in
Responders (n = 40, alleles = 80) and non-responders (n = 10, alleles = 20)

| | | Observed | Observed AF | Observed AF |
|---|---|---|---|---|
| Functional Variant | Expected AF | allele | Responders | Non responders |
| Bold = LoF | (ExAC) | count | (alleles = 80) | (alleles = 20) |
| R38M | 6.02E−05 | | | 0 |
| A105T | 1.28E−04 | | | 0 |
| D113N | 2.48E−05 | | | 0 |
| R138Q | 8.86E−05 | | | 0 |
| V162I | 1.03E−05 | | | 0 |
| T189M | 2.30E−03 | | | 0 |
| L248R | 5.20E−04 | 1 | 1.25E−02 | 0 |
| W355. | 3.98E−06 | | | 0 |
| D357A | 2.39E−04 | | | 0 |
| I363F | 4.77E−05 | | | 0 |
| D379A Control | 3.98E−06 | | | 0 |
| P463A | 0 | | | 0 |
| L550V | 0 | | | 0 |
| R702W | 2.59E−02 | 4 | 5.00E−02 | 0 |
| P727C | 0 | | | 0 |
| A755V | 2.52E−03 | | | 0 |
| E778K | 2.34E−04 | | | 0 |
| R790W | 8.18E−05 | | | 0 |
| N825K | 3.98E−06 | | | 0 |
| A849W | 7.97E−05 | | | 0 |
| W907R | 3.98E−06 | | | 0 |
| G908R | 1.09E−02 | 3 | 3.75E−02 | 0 |
| fs1007 | 1.52E−02 | 2 | 2.50E−02 | 0 |
| L1007P | 0 | | | 0 |
| R1019. | 2.12E−05 | | | 0 |
| Total | 5.84E−02 | 10 | 1.25E−01 | 0 |
| p-value | 0.012 | | | |
| (expected vs observed) | | | | |

Using the ExAC frequencies is more accurate because they survey a larger number of individuals. Based on ExAC frequencies, the expected frequency of carrying one or other of the described NOD2 inhibiting mutations is the sum of the individual frequencies listed in Table 5; 0.058 (5.8%). The inventors' finding that 12.5% of exceptional responders alleles carried a NOD2-inhibiting mutation is significantly greater than expected.

Genetic Inhibitors of NOD2 Signalling Co-Factors in Lung Cancer Exceptional Responders

In addition to NOD2 inhibition by uncommon mutations in the NOD2 encoding nucleotide sequence itself, the exceptional responders cohort was enriched for common DNA variants that inhibit genes essential for NOD2 signalling. The non-responder cohort, albeit small numbers, was enriched for a gain-of-function variant within an essential NOD2 signalling partner (hyper-activating).

The burden of these common susceptibility DNA variants in each of the exceptional responders (coloured circles) and in each of 1300 well elderly people in the MGRB (Box plot, percentile markings 10, 25, mean, 75 and 90) was calculated. FIG. 1 shows that 20% of exceptional responders lie above the 90th centile for burden of these common Crohn's susceptibility variants compared to healthy controls in MGRB.

The same analysis was applied to lung adenocarcinoma patients included in The Cancer Genome Atlas (TCGA-LUAD). The TCGA-LUAD patients had similar centile placements as the MGRB patients, suggesting enrichment for NOD-2-associated variants is not a risk factor for developing lung cancer (FIG. 2).

FIG. 3 shows that the three NOD2-inhibiting variants are included in risk scores, and are found in patients across the spectrum (above and below mean risk). NOD2 variation correlated with colitis in only one case (CRGH4, compound heterozygous).

Collectively, this evidence corresponds to a small clinical trial of highly specific NOD2 inhibition in conjunction with PD-1 blockade, showing that combining the two increases the immune response against the lung cancer to either shrink or prevent it growing.

### Example 4. Murine Model of NOD2 Inhibition

To test the physiological function of the loss-of-function frameshift mutation (L1007fs; rs2066847) and the rare functional variant found in the patient with melanoma, T179M (murine equivalent; A162M), C57B/L6, mice at the Mouse Engineering at Garvan/ABR (MEGA) facility were genetically modified using Clustered Regularly Interspaced Short Palindromic Repeats (CRISPR)/Cas9 technology.

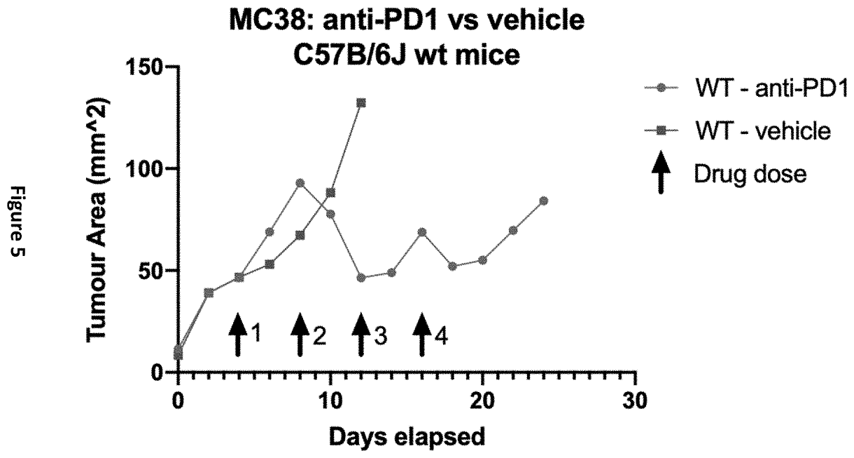

Baseline characterisation of homozygous frameshift and homozygous A162M mice compared with wild-type animals showed intact immune compartments, including similar distribution of leukocytes in bone marrow and spleen. This was retained in an aged cohort, and day 7 following sheep red blood cell immunisation, with a subtle but consistent reduction in germinal centre (GC) B cell response to stimulation (FIG. 4a b).

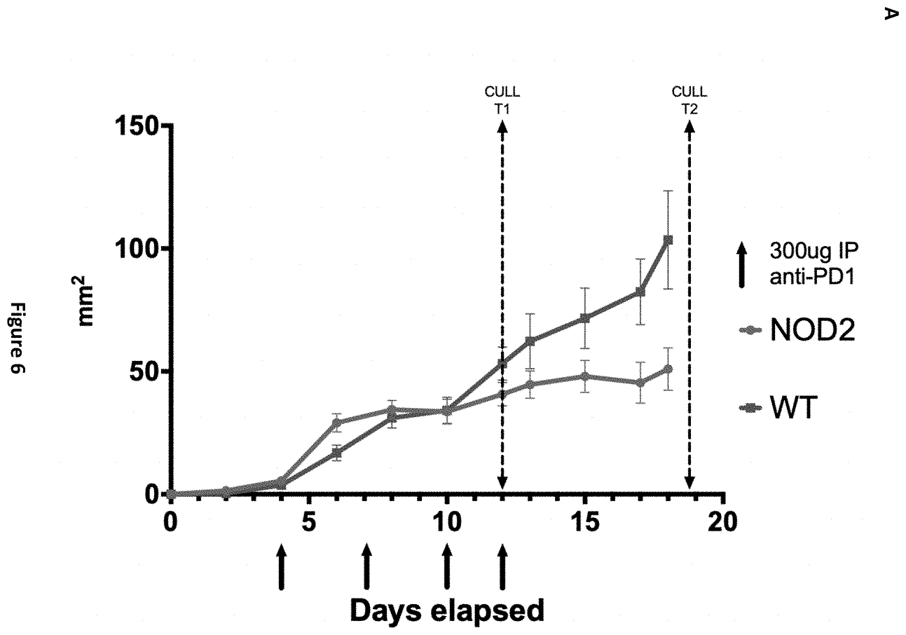

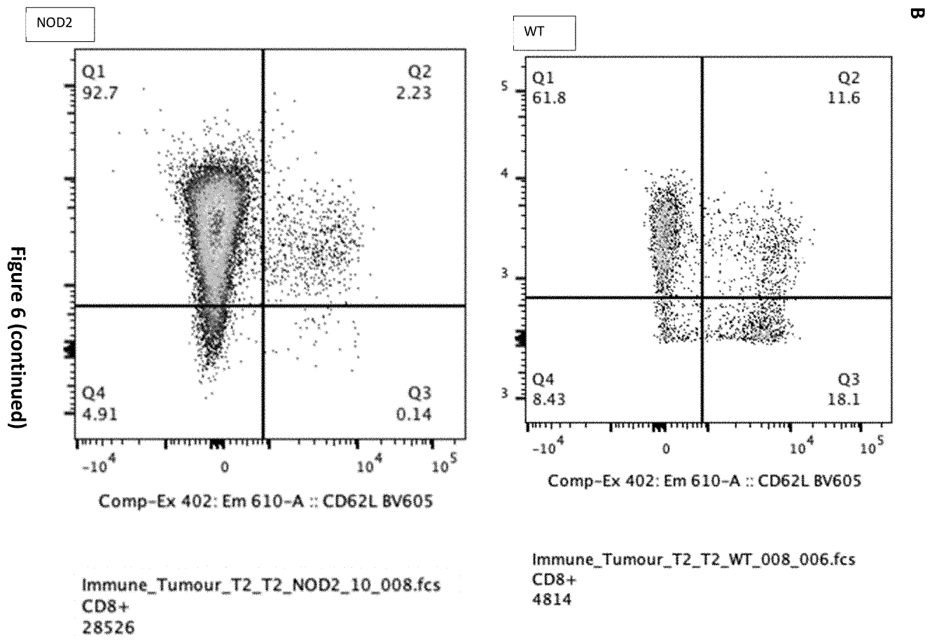

An immunogenic colon cancer cell line, MC38, was selected to investigate effect of NOD2 mutation on tumour growth and treatment with anti-PD1. Wt mice were inoculated with 1×10⁶ MC38 cells in 100 μL PBS in the sub-cutaneous flank, then treated with 300 μg in 200 μL PBS of either anti-PD1 (BioXCell InVivo anti-mouse PD-1) or isotype control (BioXCell InVivo MAb rat IgG2a) intra-peritoneally (IP) to establish baseline responsiveness to checkpoint inhibition. A stabilisation of tumour growth was observed in mice treated with active drug compared with vehicle (FIG. 5).

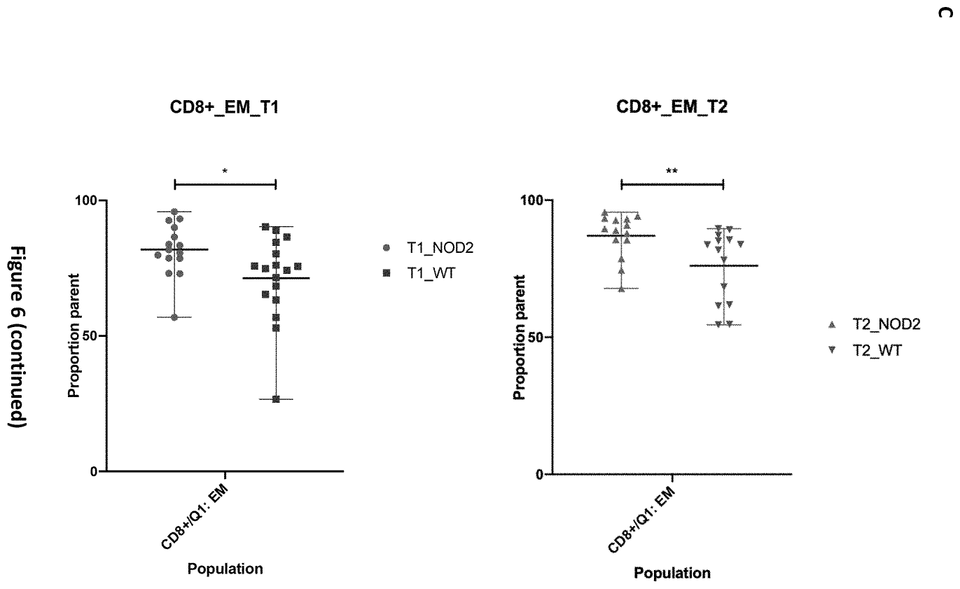

The sub-cutaneous flank of age-matched homozygous NOD2 frameshift mice and wt mice (n=12 per group, repeated in 4 experiments) were inoculated with 1×10⁶ MC38 cells in 100 μL PBS. Once tumours were palpable in >50% of mice (˜day 5), they were treated with 300 μg anti-PD1 in 200 μL PBS IP for four doses, given every three days. Tumours were measured every other day (perpendicular diameters, mm²). The mice were culled after two doses (T1) or four doses (T2). The tumour and spleen were analysed using flow cytometry (FIG. 6a). Tumour growth replicated previous experiments in that, in untreated animals, MC38 grew more rapidly in NOD2 mutant animals, however had greater depth and durability of response following treatment with anti-PD1 compared with wild-type animals (FIG. 6a).

Analysis of immune infiltrate into tumour showed enrichment for CD8 effector cells as a proportion of total CD8⁺ cells, within NOD2 mutant animals compared with wild-type (FIG. 6b, c). Effector CD8⁺ T cells have cytotoxic capacity and are essential for effective anti-tumour immunity (Farhood et al, 2019, J Cell Physiol, 234(6):8509-8521, PMID: 30520029). The CD8⁺ effector subset is thought to be the major type of immune cell augmented by treatment with checkpoint inhibitors including anti-PD1 (Topalian et al, 2016, Nat Rev Cancer; 16(5):275-87. PMID27079802).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All documents referred to herein are incorporated by reference in their entirety.

1. A method of selecting a subject for anti-cancer therapy, the method comprising:
(i) determining the sequence of a NOD2 encoding nucleotide sequence in the subject;
(ii) comparing the nucleotide sequence determined at (i) to a reference NOD2 encoding nucleotide sequence;
wherein the presence of a loss-of function mutation in the sequence determined at (i) relative to the reference NOD2 encoding nucleotide sequence indicates that the subject is likely to be responsive to anti-cancer therapy.

2. A method of predicting the response of a subject to anti-cancer therapy, the method comprising:
(i) determining the sequence of a NOD2 encoding nucleotide sequence in the subject;
(ii) comparing the nucleotide sequence nucleotide sequence determined at (i) to a reference NOD2 encoding nucleotide sequence;
wherein the presence of a loss-of-function mutation in the sequence determined at (i) relative to the reference NOD2 encoding nucleotide sequence indicates that the subject's response to anti-cancer therapy is likely to be improved relative to if the subject had the reference NOD2 encoding nucleotide sequence.

3. A method of stratifying a subject according to their likely response to anti-cancer therapy, the method comprising:
(i) determining the sequence of a NOD2 encoding nucleotide sequence in the subject; and
(ii) comparing the nucleotide sequence determined at (i) to a reference NOD2 encoding nucleotide sequence; and
(iii) stratifying the subject according to their predicted response to anti-cancer therapy, wherein the presence of a loss-of-function mutation in the sequence determined at (i) relative to the reference NOD2 encoding nucleotide sequence indicates that the subject's response to anti-cancer therapy is likely to be improved relative to if the subject had the reference NOD2 encoding nucleotide sequence, and wherein the absence of a loss-of-function mutation in the sequence determined at (i) relative to the reference NOD2 encoding nucleotide sequence indicates that the subject's response to anti-cancer therapy is likely to be the substantially the same as if the subject had the reference NOD2 encoding nucleotide sequence or poorer than if the subject had a loss-of-function mutation in their NOD2 encoding nucleotide sequence.

4. The method according to any one of claims 1-3, wherein the anti-cancer therapy is chemotherapy, radiotherapy, immunotherapy, or any combination thereof.

5. The method according to claim 4, wherein the immunotherapy comprises administering a checkpoint inhibitor selected from the group consisting of PD-1/PD-L1 targeting agents and CTLA-4 targeting agents.

6. The method according to claim 5, wherein the PD-1/PD-L1 targeting agent is selected from the group consisting of pembrolizumab, atezolizumab, avelumab, durvalumab and nivolumab, and the CTLA-4 targeting agent is selected from the group consisting of ipilimumab and tremelimumab.

7. The method according to any one of claims 1-6, wherein the subject is suffering from a cancer selected from the group consisting of: thoracic cancer, head and neck cancer, melanoma, skin cancer, neurological cancer, germ cell cancer, sarcoma, hepatobiliary cancer, upper gastrointestinal cancer, lower gastrointestinal cancer, breast Cancer, CNS cancer, gynaecological cancer, genitourinary cancer; neuroendocrine and adrenal cancers, cancer of unknown primary, lymphoma, leukaemia, colon cancer and plasma cell neoplasms.

8. The method according to claim 7, wherein the cancer is lung cancer, colon cancer or melanoma.

9. The method according to any one of claims 1-8, wherein the reference NOD2 encoding nucleotide sequence is:
(i) a wildtype NOD2 encoding nucleotide sequence;
(ii) a consensus sequence constructed from a population of individuals which do not possess loss-of-function mutations in their NOD2 gene;
(iii) a NOD2 encoding nucleotide sequence set forth in SEQ ID NO: 1.

10. The method according to any one of claims 1-9, wherein the method further comprises preparing a reference NOD2 encoding nucleotide sequence from a population of individuals.

11. The method according to any one of claims 1-10, wherein the subject is receiving, has been prescribed, and/or has received anti-cancer therapy.

12. A method of selecting a subject for anti-cancer therapy, the method comprising:
(i) determining the protein sequence and/or activity of NOD2 in the subject;
(ii) comparing the protein sequence and/or activity of NOD2 determined at (i) to a reference NOD2 protein sequence and/or activity;
wherein the presence of a loss-of function mutation in the subject's NOD2 protein sequence and/or reduced NOD2 protein activity indicates that the subject is likely to be responsive to anti-cancer therapy.

13. A method of selecting a subject for anti-cancer therapy, the method comprising:
(i) determining the level of expression and/or activity of NOD2 in the subject;
(ii) comparing the level of expression and/or activity of NOD2 determined at (i) to a reference level of expression and/or activity for NOD2;
wherein a reduced level of expression and/or activity of NOD2 determined at (i) relative to the reference level of NOD2 expression and/or activity indicates that the subject is likely to be responsive to anti-cancer therapy.

14. The method according to any one of claims 1-13, further comprising treating a subject determined as being likely to be responsive to anti-cancer therapy, wherein treating the subject comprises administering the anti-cancer therapy to the subject.

15. The method according to any one of claims 1-13, further comprising administering a NOD2 inhibitor to a subject determined as being less likely to be responsive to anti-cancer therapy based on their NOD2 status.

16. A method of treating cancer, comprising inhibiting NOD2 function in a subject in need thereof, wherein the subject is receiving, has been prescribed or has received anti-cancer therapy.

17. The method according to claim 16, wherein inhibiting NOD2 comprises administering a NOD2 inhibitor, an RIPK2 inhibitor, an LRRK2 inhibitor, inhibiting a ubiquitin ligase required for NOD2 signalling activity, or any combination thereof.

18. The method according to claim 17, wherein the ubiquitin ligase is selected from the group consisting of: XIAP, cIAP2, cIAP1 and Pellino 3.

19. The method according to any one of claims 16-18, wherein inhibiting NOD2 function comprises genetic inhibition of NOD2.

20. The method according to any one of claims 16-19, wherein the anti-cancer therapy is chemotherapy, immunotherapy, radiotherapy, or any combination thereof.

21. The method according to claim 20, wherein the immunotherapy comprises administering a checkpoint inhibitor selected from the group consisting of PD-1/PD-L1 targeting agents and CTLA-4 targeting agents.

22. The method according to claim 21, wherein the PD-1/PD-L1 targeting agent is selected from the group consisting of pembrolizumab, atezolizumab, avelumab, durvalumab and nivolumab, and the CTLA-4 targeting agent is selected from the group consisting of ipilimumab and tremelimumab.

23. The method according to any one of claims 16-22, wherein the cancer is a cancer selected from the group consisting of: thoracic cancer, head and neck cancer, melanoma, skin cancer, neurological cancer, germ cell cancer, sarcoma, hepatobiliary cancer, upper gastrointestinal cancer, lower gastrointestinal cancer, breast Cancer, CNS cancer, gynaecological cancer, genitourinary cancer; neuroendocrine and adrenal cancers, cancer of unknown primary, lymphoma, leukaemia, colon cancer and plasma cell neoplasms.

24. The method according to claim 23, wherein the cancer is lung cancer, colon cancer or melanoma.

25. Use of an inhibitor of NOD2 function in the manufacture of a medicament for treating cancer in a subject, wherein the subject is receiving, has been prescribed and/or has received anti-cancer therapy.

26. The use according to claim 25, wherein the NOD2 inhibitor is a NOD2 inhibitor, an RIPK2 inhibitor, an LRRK2 inhibitor, an inhibitor of a ubiquitin ligase required for NOD2 signalling activity, or any combination thereof.

27. The use according to claim 26, wherein the ubiquitin ligase is selected from the group consisting of: XIAP, cIAP2, cIAP1 and Pellino 3.

28. The use according to any one of claims 25-27, wherein the NOD2 inhibitor is a genetic inhibitor of NOD2.

29. The use according to any one of claims 25-28, wherein the anti-cancer therapy is chemotherapy immunotherapy, radiotherapy, or any combination thereof.

30. The use according to claim 29, wherein the immunotherapy comprises administering a checkpoint inhibitor selected from the group consisting of PD-1/PD-L1 targeting agents and CTLA-4 targeting agents.

31. The use according to claim 30, wherein the PD-1/PD-L1 targeting agent is selected from the group consisting of pembrolizumab, atezolizumab, avelumab, durvalumab and nivolumab, and the CTLA-4 targeting agent is selected from the group consisting of ipilimumab and tremelimumab.

32. The use according to any one of claims 25-31, wherein the cancer is a cancer selected from the group consisting of: thoracic cancer, head and neck cancer, melanoma, skin cancer, neurological cancer, germ cell cancer, sarcoma, hepatobiliary cancer, upper gastrointestinal cancer, lower gastrointestinal cancer, breast Cancer, CNS cancer, gynaecological cancer, genitourinary cancer; neuroendocrine and adrenal cancers, cancer of unknown primary, lymphoma, leukaemia and plasma cell neoplasms.

33. The use according to claim 32, wherein the cancer is lung cancer or melanoma.

34. An inhibitor of NOD2 function, for use in treating cancer in a subject, wherein the subject is receiving, has been prescribed or has received anti-cancer therapy.

35. A kit for predicting a subject's likely response to anticancer therapy and/or for selecting a subject who is suitable for anticancer therapy based on NOD2 status, said kit comprising:
(i) one or more reagents configured to determine the sequence of a NOD2 encoding nucleotide sequence in a subject; and/or
(ii) one or more reagents configured to detect the presence or absence of a loss-of-function NOD2 protein variant; and/or
(iii) one or more reagents configured to determine a level of expression and/or activity of NOD2 in a subject.

36. An antibody which binds to a NOD2 loss-of-function protein variant.
